(19) United States
(12) Patent Application Publication
(10) Pub. No.: US 2016/0051806 A1
(43) Pub. Date: Feb. 25, 2016

(54) Ductus side-entry jackets and prosthetic disorder response systems

(71) Applicant: David S. Goldsmith, Fort Bragg, NC (US)

(72) Inventor: David S. Goldsmith, Fort Bragg, NC (US)

(21) Appl. No.: 14/121,365

(22) Filed: Aug. 25, 2014

(51) Int. Cl. A61M 37/00; A61N 1/00; A61F 2/82; A61N 2/00

(52) U.S. Cl. CPC A61M 37/00; A61N 2/004; A61N 1/00; A61F 2/82

(57) ABSTRACT

Provided are means for the direct and continuous connection of a catheter to the lumen of any tubular anatomical structure, or ductus, without medically significant leakage. A port implanted at the body surface with piping to a periductal collar allows drug or radionuclide delivery that bypasses the upstream lumen. The port allows injection, infusion, or attachment of an automatic ambulatory pump. A superparamagnetic nanoparticle carrier-bound drug, for example, can be introduced into the lumen to pass downstream until the carrier particles, with or without the drug still bound, are drawn into the lumen wall by a magnetized jacket surrounding the ductus. Such constitutes a method of drug targeting whereby a segment of a vessel or the territory supplied by a branch of that segment can be circumscribed for exposure to the drug. A jacket with side-entry connector positioned in surrounding relation to a lesion requiring treatment can itself be magnetized.

### CROSS REFERENCE TO RELATED APPLICATION

This nonprovisional application follows and claims the benefit of Provisional Patent Application 61/959,560, filed on 27 Aug. 2013 under 35 U.S.C. 119(e). Ductus side-entry connection jackets as described herein can be used independently, in coordination, or unitized, with periductally positioned magnetic collars, as described in copending continuation-in-part application Ser. No. 13/694,835, now US 20140163664, entitled Integrated System for the Infixion and Retrieval of Implants with or without Drug Targeting, filed on 9 Jan. 2013.

### FEDERALLY SPONSORED RESEARCH OR DEVELOPMENT

None.

### PARTIES TO A JOINT RESEARCH AGREEMENT

None.

### SEQUENCE LISTING

Not applicable.

### SUMMARY TABLE OF CONTENTS

### 1. Background of the Invention [0006]

### 2. Summary of the Invention [0541]

### 3. Objects of the Invention [0543]

### 4. Description of the Drawings [0558]

### 5. Description of the Preferred Embodiments of the Invention [0597]

### 1. BACKGROUND OF THE INVENTION

A ductus side-entry jacket allows a secure junction to be established between a catheter and a bodily conduit, or ductus (pronounced “ductoos” when plural) over an indeterminate period suitable for the treatment of chronic disease. The jacket does this by closing off any path that a leak might take before the wall of the ductus, whether vascular, is breached upon the introduction of an opening (ostium, aperture, fenestration) in its side, thus allowing the creation of a continuous passageway between a catheteric and the native lumen. In simplest form as junction-type side-entry jackets, these can be used to establish fluid conducting junctions between a catheter or tissue engineered ductus with a native ductus as a safe, secure, and more versatile alternative to long term indwelling catheters such as a Hickman catheter. However, unlike an indwelling catheter, the junction, indeed multiple such junctions, will hold without risk of disconnection or injury to the patient, who is able to move freely.

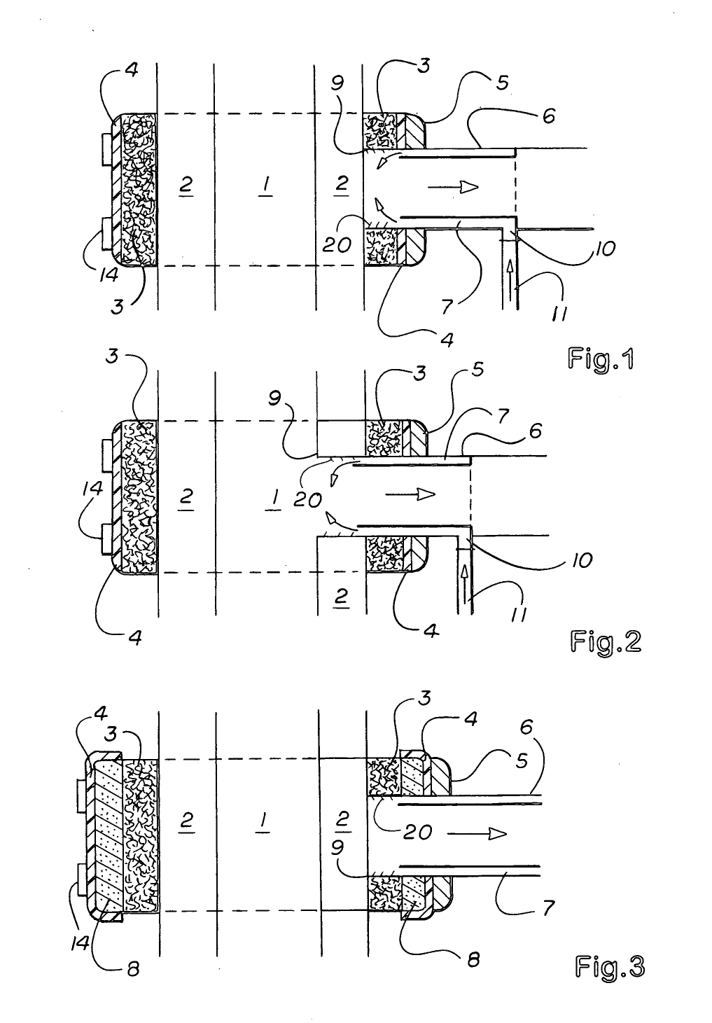

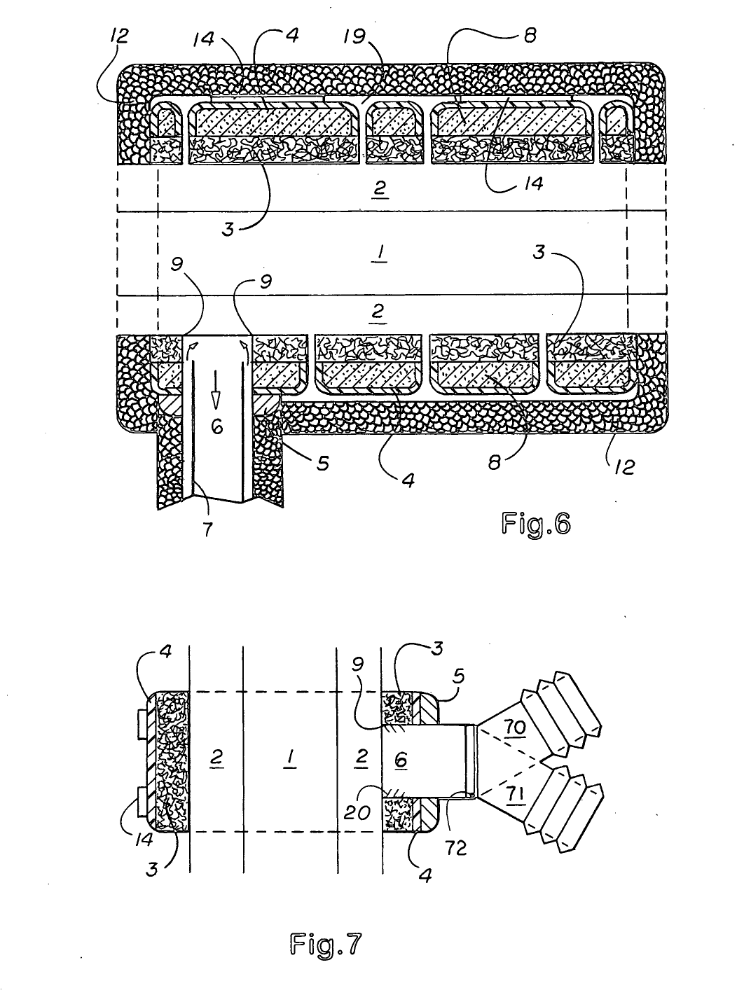

A simple junction ductus side-entry jacket of this kind, or simple junction jacket, as shown in FIG. 2 is connected to an automated drug delivering and extractate collecting power, control, and pump body-pack, or simply pump-pack, with a microcontroller that receives inputs from sensor implants and is programmed to function continuously under predictive control. Such a jacket or array thereof can serve as drug delivery points for the release of drugs and/or the extraction of a diagnostic test sample of lumen contents. The addition of a magnetized layer as shown in FIGS. 5 and 6 allows a drug bound to a magnetically susceptible carrier particle, magnetized fullerene, microsphere, or nanoparticle to be drawn against and into the wall surrounding the ductus, and when bound to a substance having a natural affinity for the analyte or cell so that the susceptible particle is bound to a particular type analyte or cell in this indirect way, a powerful magnet can be used to extract the analyte or cell targeted.

Where an indwelling catheter limits freedom of movement, disallows participation in activities that could result in dislodgement and injury, and is therefore unsuitable for the practical implementation of automatic ambulatory prosthetic disorder response systems, a ductus side-entry jacket is intended to support such function. With secure junctions to native ductus, such an ambulatory system can be used to target medication to specific segments or levels of any type bodily ductus or the territories or organs those supply. Moreover, the bidirectional capability and volumetric flow rate of junctions created with a side-entry connection jacket is able to exceed that of an indwelling catheter, expanding the potential applications for such a junction. Ductus side-entry jackets can be made to fit around any type of ductus and are lined with a foam which highly compliant and variable in thickness, allows ductus of any kind, regardless of intrinisic motility peristaltic or pulsatile, to be jacketed.

Jackets applied to ductus belonging to the same or different organ systems deliver drugs under the control of a centralized drug delivery pack suspended from a pants belt, for example. For this reason, the ductus, tissues, and organs treated may belong to different bodily systems, allowing the concurrent and coordinated automatically administered ambulatory treatment under centralized control of different syndromes and comorbidities. To treat syndromes and comorbid conditions that affect different organ systems, a pump and jacket set might include jackets sized to fit different vessels and a segment along the digestive tract, for example. Adaptive drug release responsive to sensor inputs to a microcontroller in the pack seeks to emulate endogenous adaptive function as exhibited in the release of vasodilators by the endothelial linings of the blood vessels, for example.

As will be addressed, more advanced applications of the invention include ambulatory magnetic apheresis and shunts with direct drug delivery. A ductus side-entry jacket can be used to minimize if not completely avoid direct connection between a catheter and a native ductus which induces an adverse tissue reaction and to avoid direct anastomoses along the digestive tract, where a segment harvested from the same individual is inserted as a graft elsewhere along the tract only to become infected and rejected if not leak, either resulting in failure or a need for drugs that cause complications. This is the usual result when a section of the small intestine is used as a graft following replacement of a resected or traumatically destroyed esophagus, for example.

Apheresis by the means to be described can deliver a ferrofluid containing drug carrying superparamagnetic iron oxide nanoparticles, or SPIONS, to target more than one analyte and/or cell type at a time. Such implanted means are not intended for use in voluntary apheresis that seeks to harvest healthy cells, but rather exclusively for the extraction of diseased cells or other analytes from patients who are severely sick. Severe an intractable hypercholesterolemia such as familial can be ameliorated if not alleviated by ambulatory plasmapheresis that removes low and very low density lipoprotein refractory to conventional medication through the gradual automatic continuous infusion and extraction of magnetized C60 (fullerenes, buckminsterfullerenes) and/or other superparamagnetic iron oxide nanoparticles carrying ligands for these targets.

Because this can continue around the clock, the patient may be spared the need for a portocaval shunt or partial ileal bypass, both of which can result in adverse sequelae, if not a liver transplant (see, for example, The Merck Manual, 18th Edition, page 1305), which necessitates the administration of immunocompromsing drugs for life with the patient placed at risk for infection. Therapeutic plasmapheresis and hemapheresis subsume numerous cellular and autoimmune protein targets for extraction—essentially all those responsible for autoimmune disease. The extraction ferrofluid is pumped from its respective reservoir, infused through a simple junction jacket placed about the blood supply to the target organ or gland.

Unlike conventional plasmapheresis, replacement plasma is not needed, because only the target analyte is extracted, the rest of the plasma continuing through the circulation. An infusate if needed would be pumped from another reservoir through another such jacket. Potentially, any analyte which can be directly or indirectly bound to magnetically susceptible nanoparticles through an intermediary or mutually conjugative or agglutinative substance with a natural affinity for an innate target cell or molecule that causes disease can be used to scavenge the affinate and extracted from the passing blood by extraction-electromagnets with the strength to attract the particles.

With an autoimmune condition that attacks a particular organ or gland, such as type 1 diabetes, hemolytic anemia, myasthenia gravis, or Hashimoto thyroiditis, for example—dozens of serious disorders fall under this category—extraction jackets placed at the blood supply or in an array over the organ or gland removes the mutein or anomalous hemoglobin which is replaced with hemoglobin containing the normal form of the analyte through a simple junction jacket placed upstream. The brief interval and limited distance over which these scavenger particles are allowed to circulate substantially reduces if not eliminates any toxic consequences that would otherwise ensue were these allowed to remain in the bloodstream. This factor fundamentally liberates the formulation of these particles. The corticosteroids, autoimmune medication, and anti-inflammatory drugs conventionally prescribed all pose significant problems.

Equally significant is the fact that when tightly targeted, the drug can be administered in a concentration far higher than would be allowed to circulate. The concentrated delivery of a statin to an atheroma to take advantage of its pleiotropic or nonliver mediated effects is just one example. To extract an anomalous hemoglobin responsible for autoimmunity selective of a certain organ, gland, or type tissue, the intermediary conjugate or natural affinate for delivering the susceptible nanoparticle to the target consists of a molecular substituent sufficient to effect bonding or an amount of the native target insufficient to neutralize the selectivity of the anomalous hemoglobin. Where the endogenous substance cannot be used, the fact that the process is transient as to elude toxicity makes it possible to use any exogenous or synthetic substance that mutually bonds to the carrier and target analyte despite toxicological convention.

Provided this can be extended to include immunoglobulin antibodies in plasma, automatic ambulatory intracorporeal plasmapheresis, whereby blood is not removed and then returned or replaced but continues to flow through the bloodstream during extraction without leaving the body is possible. Cytapheresis that delivers the cells to a reservoir allows the cells to be harvested and examined. Essentially, the means to be described allow the automatic ambulatory infusion of any therapeutic substance and/or the extraction of any substance that can be bound to a magnetically susceptible carrier particle, such conjugation mediated by a substance having a natural affinity for the target analyte. Ideally, such an intermediate can be incorporated into the polymer, such as dextran or polyethylene glycol, coating the nanoparticles (see, for example, Wahajuddin and Arora, S. 2012. “Superparamagnetic Iron Oxide Nanoparticles: Magnetic Nanoplatforms as Drug-carriers,” International Journal of Nanomedicine 7:3445-3471).

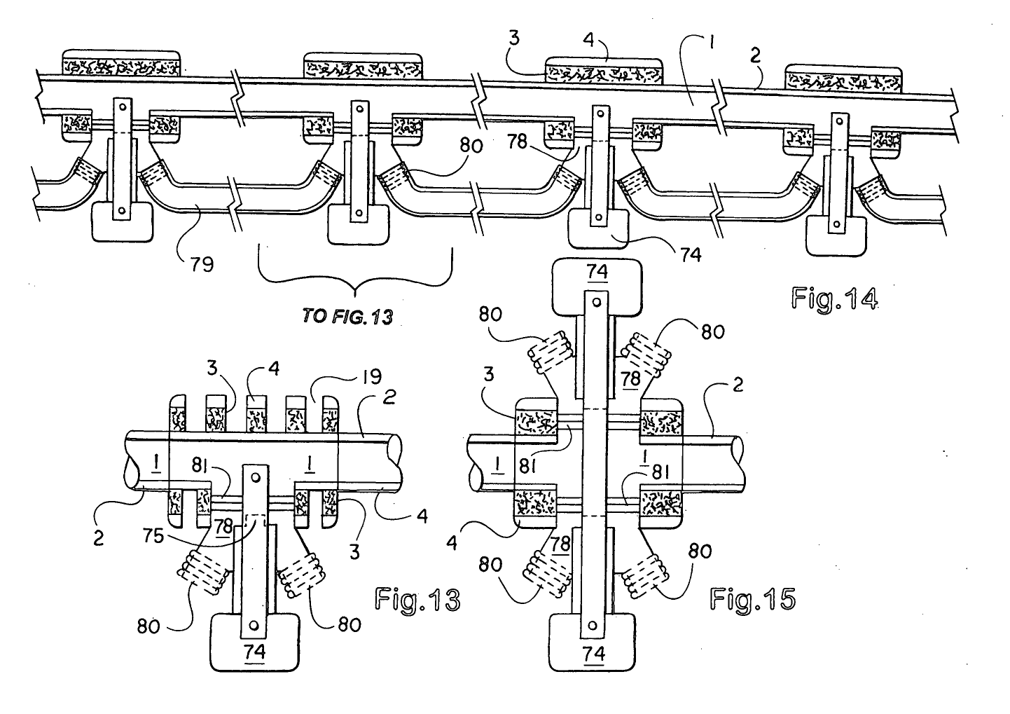

A simple junction type side-entry jacket allows the targeted delivery of a drug or other therapeutic substance as necessary to any shunt, bypass, or graft of immunosuppressive, immunomodulatory, anti-inflammatory, antispasmodic, anticoagulant, or antiseptic drugs, for example. If the drug is to drawn into the lumen wall, then the jacket or another one or more downstream are radially magnetized, removing these from the simple junction category. Remedial drug delivery is initiated by sensor implants used to supply feedback to the microcontroller in the extracorporeal (nonimplanted, external) pump-pack. When not fully taken up within the segment to be treated and the residue would best or must be removed, then a downstream jacket is used to release a reversal or neutralizing agent. If none exists, then the residual drug is itself targeted for extraction by magnetically susceptible drug-carrier particle scavenging, specialized extraction jackets shown in FIGS. 13 thru 15 and described under Description of the Preferred Embodiments.

Substantial restriction of the drug, especially those immunosuppressive, antibiotic, or steroidal, even in a higher concentration than would be allowed to circulate, to the target segment or anastomosis, materially reduces if not eliminates the drug from the systemic circulation, and in so doing, reduces if not eliminates adverse side effects, drug-food, and drug-drug interactions. In some patients, the sequelae of systemic administration may disallow the placement of the graft, posing grave consequences. A simple junction jacket such as shown in FIG. 2 placed just upstream to the anastomosis joining a transplant organ or gland, for example, can be used to target drugs and withdraw diagnostic test samples from the transplant.

If takeup within the transplant is not spontaneous, then clasp-magnets are attached about the outer surface of the transplant. When only one such magnet is needed, a permanent clasp-magnet is used. If comorbid disease recommends the magnetically targeted takeup of drugs at multiple sites or systemically so that using a permanent clasp-magnet would interfere with passage of drugs intended for other sites past it, a clasp-electromagnet is used. The differential energization of these by time offset makes possible any number of mutually noninterfering or nonconflicting takeup sites for particle bound drugs. A graft sustained by means of immunosuppressive and other drugs delivered through the systemic circulation must have its dose limited to avoid systemic consequences; however, systemic consequences such as impaired immunity often appear despite having limited the dose.

The means described herein allow only the transplant to be medicated, with the dose optimized for it and not conditioned based upon the context. If necessary, a second jacket is used to deliver a reversal agent or magnetically extract any objectionable residue. Any of the different drug delivery and/or analyte extraction jackets to be described can be placed under the unified control of the master microcontroller in the pump-pack. This allows the coordinated treatment of related or unrelated disease processes affecting different organs, glands, or organ systems by means of jackets the same or different in type, size, and volumetric flow rate of delivery, for example. While most applications for such jackets are simple and direct, the modular configuration of pump-pair and jacket set pump-pack plug-ins allows the positioning of sensors and jackets to deal with multiple conditions under coordinated control.

Where disease sequelary to that current is anticipated, the additional prepositioning of sensors and jackets eliminates the need for another invasive procedure. As a matter of terminology, impasse-jackets, permanent or direct current electromagnetic, are intended to draw a magnetically susceptible particle-bound drug or other therapeutic substance up against the internal surface of the wall surrounding the lumen, penetration into the wall contingent upon the strength of the magnetic field. Permanent magnet impasse-jackets which incorporate an extraction grating allow an extracorporeal dc electromagnet to draw the extractate out of the ductus but generally lack sufficient strength to act as extraction jackets without such assistance.

Where these can extract the analyte, such jackets may be referred to as extraction-jackets. Extraction jackets proper are electromagnetic, and since the field strength can be varied from zero to a maximum, these can also be used as impasse-jackets. Various combinations of side-entry line and dc electromagnet-incorporating jackets are addressed below. Most involve the detection of a condition or status programmed to automatically initiate the targeted delivery of one or more drugs and/or other therapeutic substances, while others necessitate the continuous extraction of an endogenous or exogenous (iatrogenic) chemical or cellular analyte from the bloodstream, for example. The disease treated may include related comorbidities, such as sequelae or complications which can be anticipated, the polynesic expressions of a single disease, or unrelated intercurrent conditions or superventions.

The treatment of dysmotility in the digestive tract, for example, can be coordinated with concurrent treatment of associated symptoms in ancillary digestive organs, such as the pancreas and gall bladder. The system is ambulatory and functions around the clock without control by the patient, who may be sound asleep. The period over which the apparatus can continue to deliver platelets and erythrocytes concurrent with leukapheresis by means of magnetic separation while a leukemic patient able to do so is free to move, for example, depends upon the rate volumes of extraction and delivery. These determine the volumes of infusates that will require safe and portable reservoir storage for replenishment, as well as the volumes of any extractates accumulated by system flushing. This burden is reduced through the use of the same reservoir or reservoirs to accept all of the extractates as the infusate is depleted whether or not allowing the extractates to mix.

In an emergency not programmed for response distant from the clinic, a preplaced pump-pack can transmit the emergency signal to be activated by remote control. The jackets to be described can be placed in encircling relation about ductus along the digestive and/or urogenital tracts, the vascular tree, and/or the airway, to form a continuous passageway through the lumen of a synthetic line and into the native lumen without significant leakage or trauma and with no portion of the junction endoluminal, or projecting into the lumen. This capability has implications for the treatment of disease on a continuous, automatic, sustained, and when necessary, immediately adaptive basis. This because the body consists of tissue pipelines and the tissues these supply. Except for absorption through the skin and oral mucosa, all intake into the body is through ductus.

Any tissue can be accessed through ductus; when a side-entry jacket can be placed at a level that substantially excludes other tissue, the tissue that will be supplied is effectively isolated for targeted delivery of medication. Moreover, because the wall surrounding ductus support many biochemical interactions and discharge sensory feedback signals that modulate the control of numerous functions, the ability to circumscribe only a certain segment along a ductus for the delivery of drugs can have significant physiological implications, the more so when that segment is diseased. In addition to communication affected by the autonomic nervous system, the luminal wall can release signaling proteins, such as chemokines and interleukins, and the luminal contents can include enzymes, hormones, cells containing cytokine signaling proteins, and so on, so that remote tissues are affected as well. As a result, there is no disease in which bodily conduits are uninvolved.

No bodily conduit is analogous to inert plumbing; all are integrated into a hierarchy of negative feedback loops from the individual cells to the brain to actively and appropriately interact with the constitution, pressure, and velocity of passing contents. Every bodily conduit communicates directly or indirectly with all the tissues in the body—not just by transmitting luminal contents, but by signalling local function to higher control centers. In endothelial function, for example, the linings of blood and lymphatic vessels actively secrete vasodilators such as relaxing factor, nitric oxide, bradykinin, potassium ions, and adenosine and vasopressors or vasoconstrictors such as endothelins, epinephrine, norepinephrine, dopamine, thromboxane, and insulin, all tied into coordinated feedback loops, which continuously adjust the degree of contraction, hence, the systemic blood pressure.

The atrial walls, aortic, and carotid sinus bodies (glomus caroticum, carotid glomus) contain chemoreceptors that detect blood gas and acidity levels, which transmitted to the medulla, signal the autonomic nervous system to adjust the respiratory and heart rates and the stroke volume. Similarly positioned baroreceptors, or pressoreceptors, detect the blood pressure, likewise transmitted to the brainstem, which regulates subsidiary feedback control loops. Placed along an artery, the level at which a simple junction jacket such as that shown in FIG. 2 and described below is positioned sets the supply territory or region. Advancing the jacket along the artery toward its end supply excludes more proximal branches to neighboring tissue, closing in upon and so narrowing the target zone or supply territory. By the same token, retreating along the artery admits side branches to neighboring tissue, thus expanding the zone.

The junction bidirectional, antegrade delivery into the native lumen, whether vascular, digestive, urinogenital, respiratory, for example, is usually of a drug, whereas retrograde delivery from the lumen is usually of a diagnostic test sample. Accordingly, automated ambulatory systems of pumps able to individually deliver any of a number of different drugs to jackets placed at different levels along a single ductus, different ductus belonging to the same bodily system, or ductus belonging to different bodily systems according to a programmed schedule and mediated by sensor implants have the potential to treat morbidities and comorbidities in a discretionary manner whereby each drug is delivered to the target tissue in a time coordinated sequence. Such treatment has the potential to outstrip any therapy dependent upon the systemic, hence, necessarily indiscriminate, administration of drugs.

Susceptible to primary disease, and supplying and draining every part of the body, the treatment of bodily conduits has application to any localized condition. Drug delivery through a side-entry jacket allows the upstream ductus and tissue it supplies to be avoided. When more effective, the drug can be increased in concentration for the target tissue while substantially reduced in dose compared to the systemic dose that would be needed to achieve the same dose at the target. Whether through the use of a reversal agent or an extraction-jacket, as will be described, if necessary any residue of the drug can be truncated from further circulation at a segment cutoff level. When a bodily conduit or ductus (singular) is itself diseased, effective and efficient treatment requires that medication be actively drawn into, not merely pass by it through the lumen with little uptake.

For disease within the wall of the ductus itself, the junction is extended to incorporate a magnetic collar of which the field strength is incrementally increased in the antegrade direction to achieve a more uniform penetration. Mechanically and magnetically based, the drug targeting spoken of here averts the contingency of discovering a substance that depends upon intrinsic properties and affinities for targeting therapy at the gross anatomical level. A drug must, for example, inhibit a destructive enzyme produced as the result of a genetic defect, such as the tyrosine hydroxylase inhibitor imatinib mesylate (STI-571; Novartis Gleevec®) to selectively target cancer cells. Or it must take advantage of an inherent affinity of an organ or gland for a substance, such as the thyroid gland for iodine. Here instead, the drug is contained while conducted to the treatment site, where it is forcibly drawn into the surrounding tissue, regardless of its inherent proclivities.

Allowing the medication to pass lesions within the wall surrounding the lumen, Or ductus-intramural lesions, without takeup wastes medication that if targeted would have contributed to an effective dose, exposes healthy tissue downstream to the wasted dose, and results in complications. Moreover, increasing the dose to achieve better absorption only increases the waste and the risk. Drug targeting substantially limits exposure to the drug to the tissue intended, isolating the drug from other tissue targeted elsewhere in the body by the same control system. This makes it possible to target a transplant organ without exposing the entire body to immunosuppressive or immunomodulatory medication, and can significantly reduce if not eliminate the damage to the immune system done by chemotherapy and radiation, for example.

The value of drug targeting with respect to the administration of immunosuppressive drugs, nonsteroidal anti-inflammatory drugs such as aspirin which used to treat arthritis, for example, often produce gastritis and ulcers, statins that induce myositis in susceptible patients, steroids which can produce moon facies and induce diabetes, for example, and the avoidance of adverse side effects, drug-drug and drug-food interactions across the entire array of pharmaceuticals, all of which carry such complications, is significant in eliminating such adverse sequelae (see, for example, Polyak, B. and Friedman, G. 2009. “Magnetic Targeting for Site-specific Drug Delivery: Applications and Clinical Potential,” Expert Opinion on Drug Delivery 6(1):53-70).

Currently, magnet implants are limited to permanent magnets used to secure dental and maxillofacial prostheses and cochlear implants, and implanted rings used to ligate and atrophy tissue by compression ischemia. Other applications of magnetism require the use of an extracorporeal electromagnet to direct the magnetic field toward the treatment site, which limits such use to the clinic. The importance of drug targeting with respect to preventing rejection in transplantation, for example, will be addressed. Drug targeting can also be of value in averting side effects in drug tolerance and intolerance. Jacket placement assumes that the medication will be required on a long term basis, would best not be taken orally, by injection, or injection that must be frequent as would promote patient noncompliance, and that accessibility to the site in order to implant the jacket and a port at the body surface to be described will not result in trauma more than negligible and transient.

When the dosage regimen frequent, and/or multiple drugs are needed making self administration problematic, drug delivery is not dependent upon patient compliance but rather automatic as programmed, through a direct catheteric pipeline to the jacket or through plural lines respective of plural jackets from a port implanted at the body surface. At the same time, the port is available to administer another drug in the clinic from a syringe, for example. In order to realize the benefits of drug targeting, it is essential to possess means for establishing secure connections to ductus. The long term indwelling of a catheter, needle, endoluminal implant, or prosthesis in a vessel often leads to adverse complications.

Subclavian, femoral, and internal jugular lines, and even peripherally inserted central catheters or PICCs, for example, are susceptible to infection, occlusion, breakage, and leaks (see, for example, Jumani, K., Advani, S., Reich, N. G., Gosey, L., and Milstone, A. M. 2013. “Risk Factors for Peripherally Inserted Central Venous Catheter Complications in Children,” JAMA Pediatrics 167(5):429-435; Barrier, A., Williams, D. J., Connelly, M., and Creech, C. B. 2012. “Frequency of Peripherally Inserted Central Catheter Complications in Children,” Pediatric Infectious Disease Journal 31(5):519-521; Shen, G., Gao, Y., Wang, Y., Mao, B., and Wang, X. 2009. “Survey of the Long-term Use of Peripherally Inserted Central Venous Catheters in Children with Cancer: Experience in a Developing Country,” Journal of Pediatric Hematology and Oncology 31(7):489-492).

Due to the risk of injury, air embolism, or the formation of a hematoma, maintaining multiple such diagnostic sampling and/or drug delivery points in different veins with indwelling catheters is not feasible, certainly not in an ambulatory patient, much less in one who is very young or very old. Moreover, even though direct access to the blood supply to an affected organ or region would afford considerable advantages both diagnostically and therapeutically, this cannot be done with respect to small much less major arteries, wherein the blood pressure is greater. However, the ability to form several secure junctions with arteries, even large ones, opens the way for targeting medication to, taking draws from, and inserting a diagnostic probe into the blood supply of the organs or tissues these supply.

While most applications of ductus side-entry connection jackets are simple and direct, a secure means for forming a junction with a ductus allows the application of a body area network with wireless transmission or telemetry (see, for example, RamRakhyani, A. K. and Lazzi, G. 2014. “Interference-free Wireless Power Transfer System for Biomedical Implants Using Multi-coil Approach,” Electronics Letters 50(12) 853-855; Yazicioglu, R. F., Torfs, T., Penders, J., Romero, I., Kim, H., and 4 others 2009. “Ultra-low-power Wearable Biopotential Sensor Nodes,” Conference Proceedings, IEEE Engineering in Medicine and Biology Society 2009:3205-3208; Yoo, H. J., Cho, N., and Yoo, J. 2009. “Low Energy Wearable Body-sensor-Network,” Conference Proceedings, IEEE Engineering in Medicine and Biology Society 2009:3209-3212; Young, D. J. 2009. “Wireless Powering and Data Telemetry for Biomedical Implants,” Conference Proceedings, IEEE Engineering in Medicine and Biology Society 2009:3221-3224; Panescu, D. 2008. “Wireless Communication Systems for Implantable Medical Devices,” IEEE Engineering in Medicine and Biology Magazine 27(2):96-101; further references provided below) to afford immediate diagnosis and targeted drug delivery at multiple locations in hard real time under automatic control.

By a prosthetic disorder response system is meant an apparatus that uses the feedback from one or a combination of chemical, thermal, electrical, and mechanical implanted physiological diagnostic sensors (microsensors; detectors), for example, to trigger adaptive drug dose computation, metering, and delivery through system conduits and unique junctions to ductus in response to a control program that is a prescription. The drugs are supplied from an extracorporeally worn pack and dispensed through catheteric lines connected directly to the lumina of the target ductus. Since the process of placing the junctions—the side-entry jackets—calls for a mainline and a supporting or subsidiary sideline, the sideline is left in place to serve as a second lumen, eliminating the need for a mainline with double lumen, for example. With a portable (wearable, ambulatory) prosthetic disorder response system, the clinician specifies the target ductus, the drugs to be delivered to each, the dose regimen, and any additional factors pertinent thereto.

A pharmacist programmer enters this into a program whereby each drug is provided in response to the conditions sensed. To deliver drugs automatically and adjust the dosing, the prescription, or adaptive drug delivery program, responds to diagnostic sensor feedback under the control of a medically adapted hierarchical (nodal, nested-levels) ‘intelligent’ hard real-time ‘pathfinding’ control system. Depending upon the intricacy and frequency of differential control required of either pump in the pump and jacket set, each node controls either one of the pumps or the modular plug-in pump-pair as a subsystem in the pump-pack. To the extent practical, where comorbid conditions must be treated, each such component disease is assigned to a respective node and modular plug-in pump-pair and jacket set, and the master microcontroller programmed to coordinate the delivery of drugs among the nodes.

The addition of a module is coordinated with the module or modules already inserted; however, because when targeted to specific tissue, most if not all drugs are kept separate, the regimen overall as administered by the master controller over the nodes usually needs not effect significant adjustments among these to accommodate the addition or removal of a pump-pack. Such a prescription program can be executed by a multicore microcontroller of which each core or cog is programmed as a time division multiplexed node in the control hierarchy. Where magnetically susceptible carriers with or without a carried extractate will be so small in volume as not to require removal, high energy product permanent magnets, ordinarily made of neodymium iron boron, are preferred.

Where this debris or detritus will be slight, a permanent magnet jacket that detains the debris has a side grating that allows the debris to be extracted with the aid of a powerful extracorporeal electromagnet is used. Generally the debris if at all toxic will be equally so in the tissue surrounding the ductus; however, when extracted, it can be dispersed so as to reduce the immediate burden or concentration to a tolerable level. If the extractate debris is more toxic or radioactive, then electromagnetic extraction-jackets such as shown in FIGS. 13 thru 15 and described below remove the extractate entirely from the body. The consecutive jackets along the ductus in FIG. 15, ordinarily a vessel, are connected by a flush-line from a supply to a separate waste reservoir in the pump-pack, washing the pole of each electromagnet where the debris accumulates along the way.

Electromagnets consume much battery power, adding weight to apparatus intended to be as unobtrusive and function as inconspicuously as possible, automatically, leaving the patient free to move about with few as possible interruptions to insert a fully charged battery or to be tethered by a power cord for recharging. However, suitably configured, sized, and supported magnets materially reduce the potential for annoyance. Its attractive force provided by an electromagnet rather than a permanent magnet, an impasse or extraction jacket with or without ductus side-entry line offers additional capabilities over jackets using permanent magnets.

Controllable from zero to the maximum field strength, electromagnets allow separate jackets or segmentally defined sectors along a multiple magnet jacket to be turned on or off or varied in field strength in synchrony with the inception of drug delivery by the pump of a magnetically susceptible carrier particle-bound drug or other therapeutic substance. The jackets addressed may be electromagnetic impasse-jackets with or without side-entry connector used to detain the carrier-bound drug alongside the wall surrounding the ductus, or with greater field strength, draw the drug into the wall, or extraction-jackets used to remove the carrier-bound drug out of the lumen where it is flushed out of the body. While various combined function or hybrid jackets are mentioned herein, clean separation and distinction among parts and functions, whereby each is clearly assigned to a specific modular subsystem for a component of the disease overall, is always to be preferred as minimizing the opportunities for human error.

The drug-carrier to drug bond can be broken upon delivery of a bond-breaking substance, whereupon the carrier alone is drawn and the drug freed to continue through the circulation. One pump in the pump-pair may be assigned to provide the drug and the other the reversal agent, for example. Having been infused through a simple junction jacket upsteam, for example, and substantially restricted from access to tissue of the ductus and tissue supplied by its branches outside the target segment, the diseased segment can be treated with drugs in combinations with component concentrations suitable for the diseased tissue alone.

Such differential treatment along a ductus is not to be construed as absolute: many conditions are systemic with only the most vulnerable and severely affected segments developing frank lesions. If left unenergized, an electromagnetic embodiment of the side-entry jacket shown in FIG. 5, for example, can still function as a simple junction jacket of the kind shown in FIG. 2, because unlike the jacket shown in FIG. 5, the electromagnetic impasse-jacket will allow a magnetic carrier-bound drug sent from the pump to pass without detaining it. Any jacket which is piped, that is, includes a side-entry connector, can function as a simple junction jacket to pass magnetically nonsusceptible fluids in either direction.

Electromagnetic impasse-jackets with or without side-entry or piping can thus differentially draw particular superparamagnetic iron oxide nanoparticles, or SPIONS, from the bloodstream, for example, according to which jackets are energized in coordination with the initiation of delivery of the drug at the pump. Electromagnetic impasse-jackets therefore open the way for the development of ferrofluids containing superparamagnetic magnetite or maghemite iron oxide drug-carrier nanoparticle-bound drugs (references cited below), to take advantage of a capability to differentially distribute the drugs to only certain of the jackets along one and the same artery, for example. Nonmagnetized drugs included in the ferrofluid freely pass magnetized jackets.

Siderophilous, or having an inherent affinity for the iron in erythrocytes or red blood cells, in polycythemia (erythrocytosis, polyemia, polyhemia), to include polycythemia vera, the superparamagnetic magnetite or maghemite predisposes the nanoparticles to bond with heme containing cells directly, rendering the erythrocytes susceptible to extraction erythrocytapheresis by means of magnetic separation. To extract cells not directly bound thus using one or more extraction-jackets to be described requires the bonding to the carrier of an intervening or interloper substance to which the cell does have a natural affinity. When introduced into the bloodstream in vivo, the target cells attach the natural affinate and therewith, the drug-carrier, and having been made magnetically susceptible, can be extracted. Automatic ambulatory apheresis using extraction-jackets such as shown in FIG. 15 can be applied to several different type target cells.

The ability to energize and continuously adjust the field strength of the electromagnet in extraction jackets allows SPION drug-carriers that if not extracted would induce toxicological or adverse consequences to be eliminated before toxic sequelae can take hold (see, for example, Wahajuddin and Arora, S. 2012. “Superparamagnetic Iron Oxide Nanoparticles: Magnetic Nanoplatforms as Drug-carriers,” International Journal of Nanomedicine 7:3445-3471; Hong, S. C., Lee, J. H., Lee, J., Kim, H. Y., Park, J. Y., Cho, J., Lee, J., and Han, D. W. 2011 “Subtle Cytotoxicity and Genotoxicity Differences in Superparamagnetic Iron Oxide Nanoparticles Coated with Various Functional Groups,” International Journal of Nanomedicine 6:3219-3231; Naqvi, S., Samim, M., Abdin, M., Ahmed, F. J., Maitra, A., Prashant, C., and Dinda, A. K. 2010. “Concentration-dependent Toxicity of Iron Oxide Nanoparticles Mediated by Increased Oxidative Stress,” International Journal of Nanomedicine 5:983-989).

The myeloproliferative disorders include polycythemia vera, chronic myeloid or myelocytic leukemia, and chronic idiopathic myelofibrosis (Merck, Op cit. Section 11, Chapter 141). Depending upon the initial current and duration or number of energizations, a pulse or pulses of reversed current can be applied at the end each energization or applied at intervals. Degaussing is incorporated into the control program to proceed automatically. If energized at the same time or a moment after the pump, the jacket acts as an impasse-jacket with adjustable field strength to control penetration of the magnetically susceptible carrier particle-bound drug or other therapeutic substance into the lumen wall.

Automatic ambulatory magnetic extraction cytapheresis such as thrombocytapheresis (thrombapheresis, plateletpheresis), leukapheresis, and plasmapheretic processes which seeks to bind an analyte other than a type cell to a biological affinate bonded to a magnetically susceptible nanoparticle allows the nanoparticles to remain in the bloodstream over too small an interval and leave little if any potentially toxic residue. Because of this transience, analyte extraction by such means should not pose problematic toxicity as attributed to iron oxide-based nanoparticles when used as presumed in literature cited herein to any significant degree.

Here the ferrofluid is infused upsteam through a simple junction jacket with a series of extraction jackets such as shown in FIG. 14 along the inferior vena cava, for example, to remove the iron oxide-based nanoparticles before these can produce a toxic effect. The nanoparticles are coated with dextran or polyethylene glycol to bind the drug to be carried, and if not incorporated with the initial coating, thereafter recoated with the biologically affinative substance for the binding in turn of the bound pair with the target cells or analyte, the identity of the affinative substance or substances depending upon that of the target cell or cells. Examples of inherent or natural affinities include, for example, the thyroid gland for iodine, the myocardium for digoxin, and adipose, or fat, tissue for benzodiazepines.

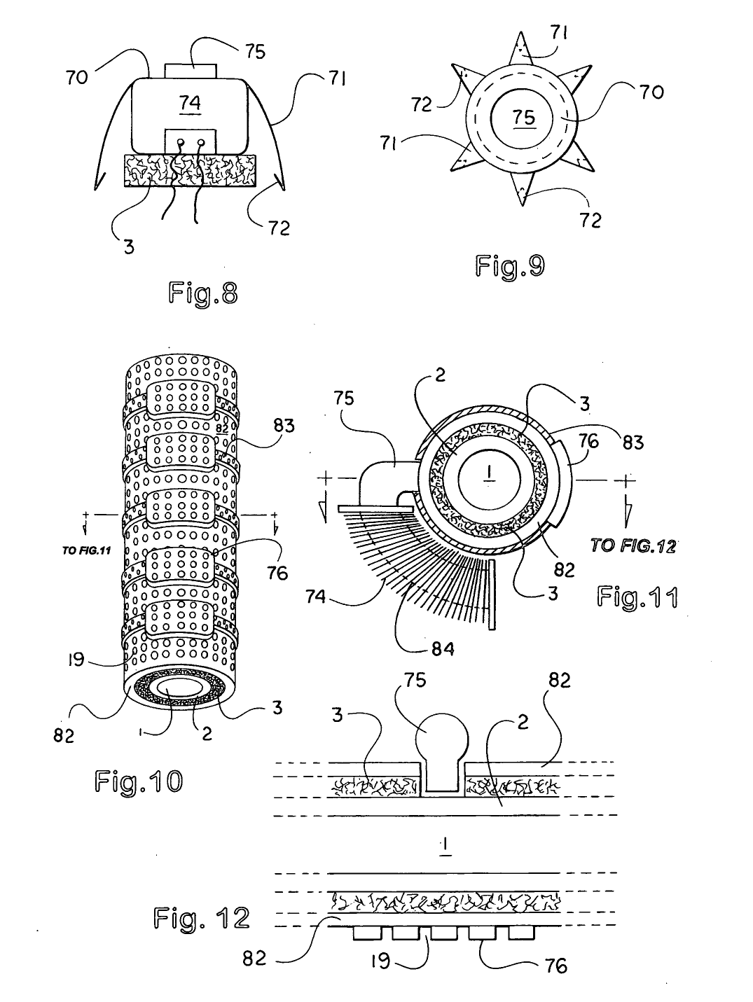

Similarly, an extraction jacket, hybrid extraction jacket, or chain thereof, all described below under Description of the Preferred Embodiments of the Invention, can extract not just bound blood cells passing through the ductus, but any bound analyte, whether previously drawn into the ductus wall and allowed to remain before a toxic effect ensues or can take hold. Moreover, a hybrid clasp extraction-magnet with trap and flush-line or chain thereof can accomplish the same for tissue within the parenchyma of an organ, whether the spleen, a lymph node, or kidney, for example, or an accessible gland, such as the thyroid, thymus, and adrenals. When the capsule of the organ is tough as to necessitate an overly large and heavy electromagnet, the substrate is prepared by stripping away this barrier. To provide an electromagnetic clasp extraction-magnet, an electromagnetic clasp magnet without flush line such as shown in FIG. 8 is provided with a debris collecting trap and flush-line such as shown in FIGS. 13 thru 15.

A single flush-line can course from the flushing fluid supply or source reservoir in the pump-pack, through each jacket and/or clasp extraction electromagnet placed at different sites along different system ductus, and return to the same waste flushing fluid catch or reservoir in the pump-pack. When, however, the contents of the waste reservoir are to be preserved for analysis, the implant or implants distinguished thus are flushed beginning with a single supply reservoir but separate flush-lines and catch or waste reservoirs. The availability of iron oxide particle steering and targeting implants which can be used to control the timing of bound particle infiltration into target tissue and the extraction of any residue before it poses a risk toxicity means that the simple fact of potential toxicity in the abstract may be little more than an indiscriminate generalization with little if any practical significance as would legitimately serve to discount such means.

Magnetic drug targeting is not an isolated chemical issue but must complement the hardware that will actually be needed to realize its application in optimized form. Foremost in this development is the formulation of magnetically susceptible nanoparticles to selectively bond to cells and bloodborne molecules to be extracted by means of magnetic separation. Iron oxide particles which are not formulated in response to the capabilities of practical steering and targeting implants will likely have limited application. Efficacy will eventually come down to an inherent reciprocal or dialectical relationship between the pharmacokinetics of the particle-bound drug as formulated and the function of the implants such that formulation would best have contemplated the use of the practical hardware available from the outset, the formulation for plasmapheretic extraction of anomalous antibody-agglutinating or conjugating nanoparticles mentioned above an example.

For this reason, the sooner the desiderata and preferable characteristics of practical implant hardware are clarified, the sooner will the pharmacology be able to follow in the optimal direction for the use of ferrofluid infusant particles to be used with that hardware. Ideally, the practical hardware and the chemistry are developed conjointly, each carrier-bound drug devised to complement the timing characteristics of its delivery as mediated by the hardware. For example, awareness of the ability to eliminate a residue before it can exert a toxic effect imparts significant latitude to the formulation of iron oxide drug carrier particles; the realization that toxicity beyond a certain interval need not be a deterrent is likely to remove a key chemical limitation.

Another consideration is that where the iron oxide would be taken up together with the drug it carries, the bond between the two if not broken spontaneously should be broken by a suitable solvent. As to clinical pharmacokinetics, or the action of the carrier-bound drug in the specific patient with one or more specific lesions, once the jacket implants have been placed, an initial test using the contrast dyed drug can be used to reveal the individual response. Because anatomy dictates the dimensions and disease dictates the positioning of the implants, it is the formulation of the carrier-bound drugs that has the wider latitude and will more often have to adapt for use with the practical steering means available.

For this reason, while the relationship between hardware and drugs is reciprocal, more often the drugs will have to be formulated to complement the hardware rather than the reverse. Electromagnetic extraction jackets, shown in FIGS. 13 thru 15, wherein the jackets are placed in series or a chain with a flush-line connecting the consecutive debris collecting traps can be used to remove leukocytes or an overabundance of blood cells, for example. Such a condition may be the result of a myeloproliferative disorder that would otherwise cause sludging of the blood and interfere with the production in the bone marrow of normal blood cells.

Where the volume of leukocytes is high but the patient is still able to function, an automatic ambulatory assist should allow a reduction in the frequency of visits to the clinic for centrifugation apheresis. In a leukemia, the malignant transformation of progenitor blood cells leads to anemia, thrombocytopenia, granulocytopenia, infiltration of tissue with diseased cells, and therewith, enlargement of the liver, lymph nodes, meninges, and other organs (see, for example, The Merck Manual, 18th Edition, page 1105). The flush-line is fed from a supply reservoir and pump in the pump-pack which is activated at the interval programmed to deliver the treated flushing fluid, wash water, or a hydrogel and carries off the cellular or other debris to a dump reservoir also in the pump-pack.

Leukapheresis is but one example of therapeutic cytapheresis which can be relegated to an automatic ambulatory system. Sickled erythrocytes and those affected by malaria, for example, can be extracted through noncentrifugal sedimentation erythrocytapheresis and replaced by normal cells (see, for example, The Merck Manual, 18th Edition, page 1143) infused through a simple junction jacket positioned upsteam. Other type blood cells such as platelets or plasma can be extracted and/or infused in the same way. In an ambulatory system with leukocyte-detecting sensors under automatic control, the object is to liberate an ambulatory patient from frequent visits if not confinement to the clinic.

In treating polycythemia vera, an upstream simple junction jacket can be used to deliver myelosuppressive drug therapy (see, for example, The Merck Manual, 18th Edition, pages 1104-1105) concurrent with extraction. The apparatus is not intended for an atypical temporary overabundance of type blood cells as might arise in a transient reduction in relative plasma volume (see, for example, Spivak, J. L 2005. “Polycythemia and Other Myeloproliferative Diseases,” in Harrison's Principles of Internal Medicine, New York, N.Y.: McGraw-Hill, pages 626-631) or in an infective state, or for essential thrombocytosis or thrombocythemia, for which cytaphersis by means of centrifugation has proven ineffective (ibid., page 631) but rather for chronic myeloproliferative disorders not controlled by an occasional, but not a frequent, phlebotomy and aspirin, for example, for patients with both myeloproliferative, sickle cell, and cardiovascular disease where phlebotomy is contraindicated, and for patients otherwise unable to undergo or not satisfactorily responsive to conventional treatment.

Remanence, or residual magnetization that would interfere with the special value in the use of electromagnets, in that these can be deenergized, effectively eliminating them from the carrier-bound drug steering path, is cleared (degaussed, depermed) by periodically transmitting a pulse or pulses of current through the electromagnet coils with revered polarity, that is, in the reverse or nonfunctional direction. As previously indicated, in an automatic ambulatory system, the magnetically susceptible carrier particle-bound drug is infused through a simple junction jacket such as that shown in FIG. 2 placed upsteam, while the flush-line is led from a clean water, flush solution or flush hydrogel supply reservoir in the pump-pack to a waste water reservoir in the pump-pack.

The number of extraction-magnets with trap and flush-line as shown in FIG. 14 required, and type, as to whether these are of the double magnet type shown in FIG. 15, depends upon the rate of residue accumulation. When larger volume extraction is required, as in a leukemia, polycythemia vera, or essential thrombocythemia, two-sided or double magnet extraction jackets such as shown in FIG. 15 are used. The magnets along either side are supplied from the same reservoir but use separate flush-lines to scrub and evacuate the successive poles and traps. Higher volume extraction generally requires supply and waste reservoirs in a pack separate from the pump-pack for less inconvenient replenishment of the flushing fluid and disposal of the effluent

Whereas a permanent magnet extraction jacket incorporates a grating so that an external electromagnet can be used to draw out accumulated debris to a safe location, an electromagnetic extraction jacket uses the inmate dc electromagnet to draw the extractate into a flush line. A permanent magnet extraction jacket may or may not be provided with a side-entry line, that is, piped or unpiped. Since electromagnets require a power source, and for ambulatory use this means heavy batteries, the use of these is limited to intermittent applications. Permanent magnets are good for use in impasse-jackets to serve as traps for preventing ferrous debris from further passage.

These must remain energized constantly without consuming power While closely related, unpiped impasse-jackets using electromagnets and peristalsis prosthesis jackets are not ductus side-entry jackets; however, electromagnetic extraction jackets, which include a side-entry are. Jacket types not covered herein are described in copending applications. Permanent magnets also afford an advantage in that these can be magnetized in any dimension, facilitating conformation to accommodate many anatomical situations. Such function is valuable where stenting miniballs were dislodged in an auto collision, for example, and the downstream prepositioning of two or more impasse-jackets eliminated the potential for embolization. However, for applications requiring variability in field strength from virtually zero to the maximum, this constancy, powerless operation notwithstanding, is a detraction that stands in opposition to the controllability of electromagnets.

For applications whereby a microcontroller operates a pump to deliver different magnetically susceptible particle bound drugs intermittently, the ability to energize specific electromagnetic impasse and extraction jackets by location in a time coordinated or sequential way affords a versatility that uncontrollable magnets cannot duplicate. Since any but simple medical conditions can take advantage of this control, the object of adapting electromagnets to such use pertains to the distinct majority of applications. Both types of magnets can be incorporated into a prosthetic disorder response system. In the treatment of comorbidities, for example, the use of permanent and electromagnetic jackets may be separated by bodily system or regionally.

It is also possible to combine permanent and electromagnets in the same system by limiting the magnetic susceptibility of the drug-carrier particles so that these pass the permanent magnets but not the stronger field strength presented by the electromagnets. Rather than requiring a high permeability core with a pole that is limited in area, the permanent magnet consists of material that is intrinsically magnetic, allowing presentation of a continuous attractive surface, which can, moreover, be graduated in magnetic field strength. With either type of magnet, differential delivery to each jacket can be made dependent upon the relative magnetic susceptibility of the drug-carrier intended for each jacket, this attribute distinguishing fractions in the ferrofluid.

Where the field strength of permanent magnets is invariable, requiring varying the susceptibility of the carrier to effect a distribution that is largely statistical, that of electromagnets allows continuous variability in the strength of each magnet for any carrier without varying the carrier susceptibility. Because the permanent magnet is graduated in strength along its length, it satisfies most needs. However, the adjustability in field strength afforded by electromagnets affords greater versatility. The ability of electromagnets to adjust for susceptibility over a large range makes possible the differential distribution of the same or a different susceptible carrier particle-bound drug or other therapeutic substance.

For implantation within the body, a literal application of electromagnets to ductus-encircling jackets has the detractions that the batteries, coils, and cores pose sizes and masses that interfere with the object of incorporation into an ambulatory system. Where constancy is unnecessary and the need for attractive force intermittent as concomitant with collateral action, such as pump delivery of a drug in synchrony with the pulse, electromagnets offer controllability where permanent magnets do not. The energized or on-times of the dc electromagnets used for the various applications delineated herein are brief, so that heat or power consumption inconsistent with the object of embodiment in an ambulatory system are not problematic.

If necessary, the extractant is withdrawn from such an impasse-jacket through an extraction grating by a powerful external (extracorporeal) electromagnet. Low toxicity permitting, extraction is normally into adjacent tissue. However, since permanent magnets continue to hold the extractate, and higher volume extraction or apheresis generates debris that must be periodically purged, that is, flushed out through an expulsion line, jackets used thus incorporating an electromagnet. Encapsulation of the potentially carcinogenic exposed pole of an electromagnet in an implant is essential. The electromagnet is rigidly connected to the jacket shell with its proximal pole nuzzled in the fork of the double arm type side-entry jacket with extraction trap flap valve to be described used.

The microcontroller deenergizes the electromagnet a moment before it actuates the pump to flush through the extraction line. Due to the Bernoulli principle, a segmentally distributed electromagnetic impasse-jacket such as that shown in FIGS. 10 thru 12, with magnetically susceptible plates opposite each magnet pole and a compressible substrate in place of the hard shell shown in FIGS. 1 thru 5, hybridized with an electromagnetic extraction jacket as shown in FIG. 13, used to constrict a large artery would reduce the magnetic gap from the pole to the magnetically susceptible drug-carrier particles to be extracted, only to cause the particles to pass at a higher velocity.

For this reason, merely applying a draw-plate and compressible substrate tube in lieu of a hard shell to an electromagnetic impasse-jacket to make it a hybrid impasse and contraction jacket without at the same time introducing means for reducing the velocity, pressure, and volumetric flow rate at which the luminal contents pass the jacket or jackets is unlikely to improve the carrier particle-bound drug extraction capability. If embedded within a tacky bolus with an opiate administered to slow peristalsis, however, constricting the ductus, here, the gut, to reduce the magnetic gap at the same time that the speed of passing is reduced will increase the extraction rate.

When the drug-carrier-bound substance is a radionuclide, extraction must be thorough. Along the vascular tree (see, for example, Cherry, E. M., Maxim, P. G., and Eaton, J. K. 2010. “Particle Size, Magnetic Field, and Blood Velocity Effects on Particle Retention in Magnetic Drug Targeting,” Medical Physics 37(1):175-182; Anor, T., Grinberg, L., Baek, H., Madsen, J. R., Jayaraman, M. V., and Karniadakis, G. E. 2010. “Modeling of Blood Flow in Arterial Trees,” Wiley Interdisciplinary Reviews. Systems Biology and Medicine 2(5):612-623; Haverkort, J. W., Kenjere\check{s}, S., and Kleijn, C. R. 2009. “Computational Simulations of Magnetic Particle Capture in Arterial Flows,” Annals of Biomedical Engineering 37(12):2436-4248), the choice of a counter-inotropic drug to reduce blood velocity and pressure over an interval no longer than necessary to facilitate extraction with the aid of an impasse, contraction, or extraction jacket is contingent upon the baseline condition of the heart and the presence of collateral disease.

The controller can be programmed to wait over the interval the drug requires to take effect before undertaking any further action. For uncomplicated, that is, normotensive and normorhythmic pulsation, some reduction in the heartrate can usually be obtained with adendsine, digoxin, and slow kinetic sodium channel blockers, such as the Vaughan Williams Class 1c drugs flecainide and propafenone (Merck Manual of Diagnosis and Therapy, 18th Edition 2006, Section 7, Chapter 75, “Arrythmias and Conduction Disorders,” pages 677-680). When a vena cava, for example, is already jacketed much as is the ascending aorta shown in FIG. 21, the control program delivers the drug or drugs through that jacket as a simple junction in advance by the interval before the drug or drugs will take effect before the jacket magnet is energized, whereupon the jacket is used as an electromagnetic impasse-jacket. A sequential arrangement of extraction jackets with flush line for higher volume extraction is depicted in FIG. 14.

Where sludging in the circulation, looms, as occurs with certain leukemias and other hematologic disorders, for example, sequential extraction jackets with the flush line coursing through each jacket in sequence, or ‘daisy-chained,’ the outlet of that upstream connected to the inlet of that downstream are used. Such a train of jackets may be conceived of as a unit, as can the peristaltic jacket shown in FIG. 10. Multiple or compound electromagnet-jackets such as the peristaltic jacket shown in FIG. 10 and the extraction jacket chain shown in FIG. 14 can be centrally controlled as a unit or incorporate a local subcontrol module analogous to a local ganglion that regulates spinal or digestive function in a subsidiary or hierarchically subordinate relation to higher centers in the brain.

The jacket shown in FIGS. 11 and 12 is an electromagnetic impasse-jacket that with a draw-plate on the opposite side of the ductus and a compressible backing instead of a hard shell such as shown in FIGS. 1 thru 5, has been converted into a contraction-jacket, configured to interact with the contents of the lumen by acting on the wall surrounding the lumen rather than attracting magnetically susceptible carriers passing through the lumen. When fluid lines as necessitate a pump-pack are not required, which is true of a sphincteric and/or peristaltic jacket, the independent module is fully implanted. While in use independently, the local control module, itself a microcontroller, and associated components nevertheless represent a single node of the overall prosthetic disorder response system governed by a central microcontroller.

The later addition of another system module, such as requires a pump-pack and fluid lines to deliver synthetic mucus and digestive enzymes, then requires the activation of another node. If the implanted or local control module is so capable, it continues to support the digestive module previously implanted, and has the new node added. When the digestive function need not be coordinated with the added function, it is most expeditious to allow the existing implant to continue to function independently. Otherwise, it is equally expeditious to place the previously implanted local control module as a node under the control of an added microcontroller in the pump-pack, or if the local controller is so capable, assign to it overall control. Later access governs the positioning of components.

### Types of Ductus Side-Entry Jackets

The table below summarizes the types of jackets with direct line feed (piping, side-entry) as addressed herein and those without side entry covered in copending application US 20140163664. Vascular and other ductus side-entry connection jackets conform to any of five basic configurations with overall dimensions and part sizes proportional to the anatomical structure to be treated.

| | | Magnetized | |
|---|---|---|---|
| | Unmagnetized | PERMANENT MAGNET | ELECTROMAGNETIC |
| Unpiped | CLASP-JACKETS | IMPASSE-JACKETS | IMPASSE-JACKETS |
| | | EXTRACTION JACKETS | CONTRACTION-JACKETS |
| | | STENT-JACKETS | |
| | | CLASP-MAGNETS | |
| Piped | SIMPLE JUNCTION JACKETS | IMPASSE-JACKETS | IMPASSE-JACKETS |
| | | PIPED STENT-JACKETS | EXTRACTION-JACKETS |
| | | | CONTRACTION-JACKETS |

These are 1. Simple junction jackets, unmagnetized and without a radiation shield, that is, a basic ductus side-entry joining connector or junction with one or more radially and/or longitudinally separated side-entry connectors; 2. With nonelongated or local nongradient magnet to treat a fully spanned lesion encircled by the jacket; 3. With elongated or longitudinally extended gradient-magnetized magnet; 4. With elongated or longitudinally extended gradient-magnetized magnet and overlying tungsten heavy alloy radiation shield; 5. With radiation shield but without magnet; and 6. Provided with a shield that disintegrates spontaneously or in response to positive action such as the application of heat, a solvent, or both.

While the jackets accord with the caliber of the ductus to be encircled, most other components are kept as standardized as possible. The diameter or caliber of a jacket is dictated by the ductus it is to encircle. Combination (hybrid, special-purpose) jackets with a fluid side-entry line added to an electromagnetic extraction, contraction, or multiple electromagnet contraction or peristaltic jacket, for example, are reserved for sites where the anatomy does not afford sufficient space to place more than a single jacket without encroaching upon neighboring tissue. To place small jackets requires magnification, and those very small a microsurgical stereoscopic or binocular microscope.

A jacket that combines the features of a pliant contraction jacket with an opposing magnetically susceptible draw-plate, such as shown in FIGS. 11 and 12, those of the extraction jacket with collection chamber or trap, as shown in FIG. 13, and a side-connector, as shown in FIGS. 1 and 2, represents a triple combination jacket for which, due to their small size, potential applications would likely be limited to neonates and small pets. Magnetized debris adherent to the pole, the trap is mostly for any nonmagnetic debris that enters with the magnetic debris. Since the extractate is drawn to the pole, the trap of an extraction-electromagnet serves primarily to collect fluid drawn out with the extractate, which is then flushed away to a waste reservoir in the pump-pack, as will be addressed.

Combination type jackets may also be justified when drug delivery and another jacket function would best be delivered at the same level or when to place numerous separate, functionally distinct jackets in a frail patient with multiple comorbidities could dangerously extend the intraoperative time under general anesthesia, for example. Radiation shielded but unmagnetized side-entry connection jackets can be used to pass through but are incapable of participating in the takeup of magnetically nonsusceptible therapeutic substances whether radioactive or not. Other than to provide a shielded junction for the radionuclide to pass through, a jacket with a shield but not a magnet is not functional as not serving to draw a radionuclide bonded to a magnetically susceptible carrier into the luminal wall.

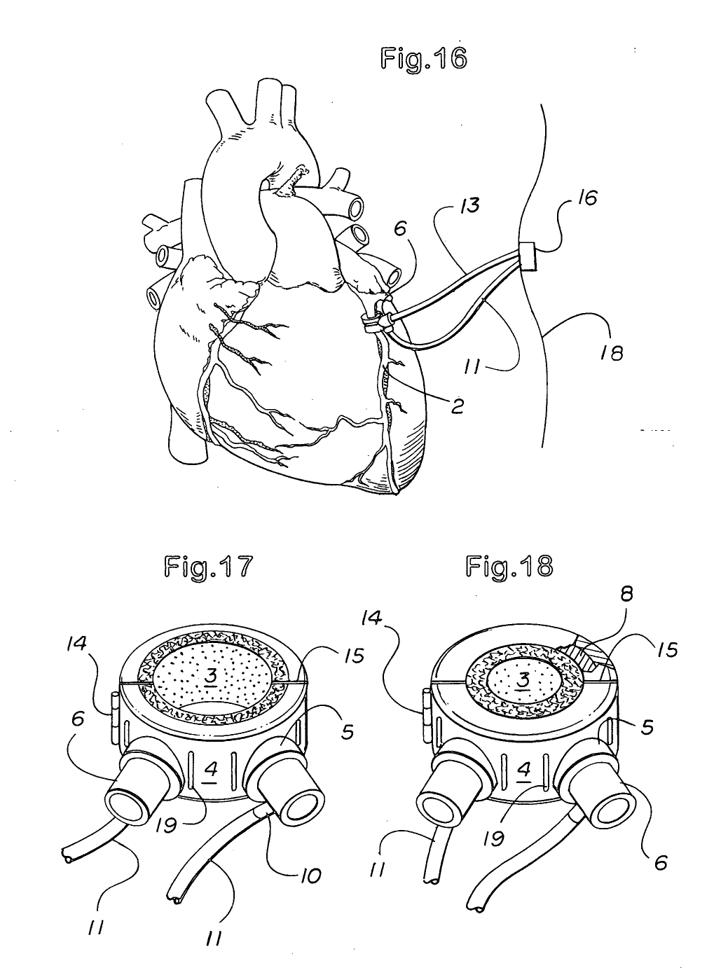

A simple junction jacket with radiation shielding but not magnetization assumes that the jacketed conduit will be shielded to a takeup terminus. For example, in FIG. 21, to treat a rare primary unmetastasized tumor within the cardium, lines 11, the jacket, side-connectors 6, and lines 13, here prosthetic right and left coronary end-arteries (but the same would apply in the application shown in FIG. 16 with native vessels), would all require shielding. For uniform takeup of the susceptible particles along the length of the jacket, the magnet is usually increased in magnetic field strength in the antegrade or downstream direction. As an optional alternative, the particulate can be prepared in fractions of varying magnetic susceptibility.

Any of these 4 basic types can be provided with more than a single side-entry connector at any radius about the circumference or separation along the longitudinal axis, and each side-entry connector can have one or more fluid conduction or water-jacket inlets for further use as service channels. In general jackets to be positioned along the vascular tree or other relatively thin-walled ductus, such as the ureters, are placed using only the cutting force of a vacuum, without circle-cutting action, with a medicated tacky hydrogel used to quench bleeding, whereas thick-walled ductus usually require rotation of the side-connector as well, with water or a medicated tacky hydrogel used.

Subsidiary sidelines or conduits, shown as part number 11 in the drawing figures, are not only useful during placement to deliver the fluid used to irrigate the insertion but remain usable thereafter for the delivery of drugs or other therapeutic substances or sensor leads to the synthetic or tissue-engineered conduit generated from autologous cells retained within the side-connector, part number 6 in the drawings. Fluid conduction or water-jacket inlet lines are normally smaller in diameter than the side-entry connector these support. To prevent the entry of fluids passed through the side-entry connector or one of its inlets into the other inlets, the inlets are kept filled with gel until used. Magnetized side-entry connection jackets will pass through without taking up magnetically nonsusceptible therapeutic substances but if any of these are radioactive, the jacket must also incorporate a radiation shield.

To avoid a need to reenter, the selection of a side-entry connection jacket therefore takes into account the prospective scope of type therapeutic substances and the rate of delivery of each that might reasonably become necessary during treatment, and a jacket with the necessary elements to cover the various contingencies, to include the number of water-jacket inlet lines or service channels is placed. The inlets are piped to a port implanted at the body surface that incorporates the number and gauge of the sockets required. Unless a comprehensively configured jacket would encroach on neighboring tissue, overspecification involves only added expense. Distinctions as to shield disintegration that is spontaneous, thermal, chemical, electrical, or combinations thereof are not considered of such import as to define additional jacket types. The incorporation of a permanent magnet and/or radiation shield is consistent with a need for uniform takeup over the length of the jacket of the magnetically susceptible particle bound drug or radionuclide.

A more uniform takeup with electromagnets necessitates the use of more than one. When the magnetism need not be completely stopped, concentrated takeup at the electromagnet pole can be ameliorated by placing the pole through an opening through an otherwise ordinary permanent magnet layer. The outer surface of all jacket types is made as rounded, smooth, and unobtrusive for neighboring tissue as possible. To lend support when the patient is recumbent, jackets that pose a weight problem may require the construction of a horizontally disposed tissue sling or harness. To include extension for prevention, shielded and unshielded magnetized jackets alike are made no longer than is necessary to provide a secure and leak free synthetic-to-native or native-to-synthetic conduit junction.

Ductus-encircling jackets with a radiation shield and with or without a subjacent magnet can also be used to shield a segment of the encircled ductus from radiation directed at neighboring tissue. Shielding is limited by the mass of shielding material which can be incorporated without encroaching upon neighboring tissue or causing the patient discomfort absent means of suspension. The jackets shown in FIGS. 13 thru 15 represent one of four types of electromagnetic ductus jackets, piped and unpiped impasse-jackets, piped extraction jackets, and unpiped contraction-jackets, specifically, the type configured to interact with the contents rather than with the wall surrounding the lumen.

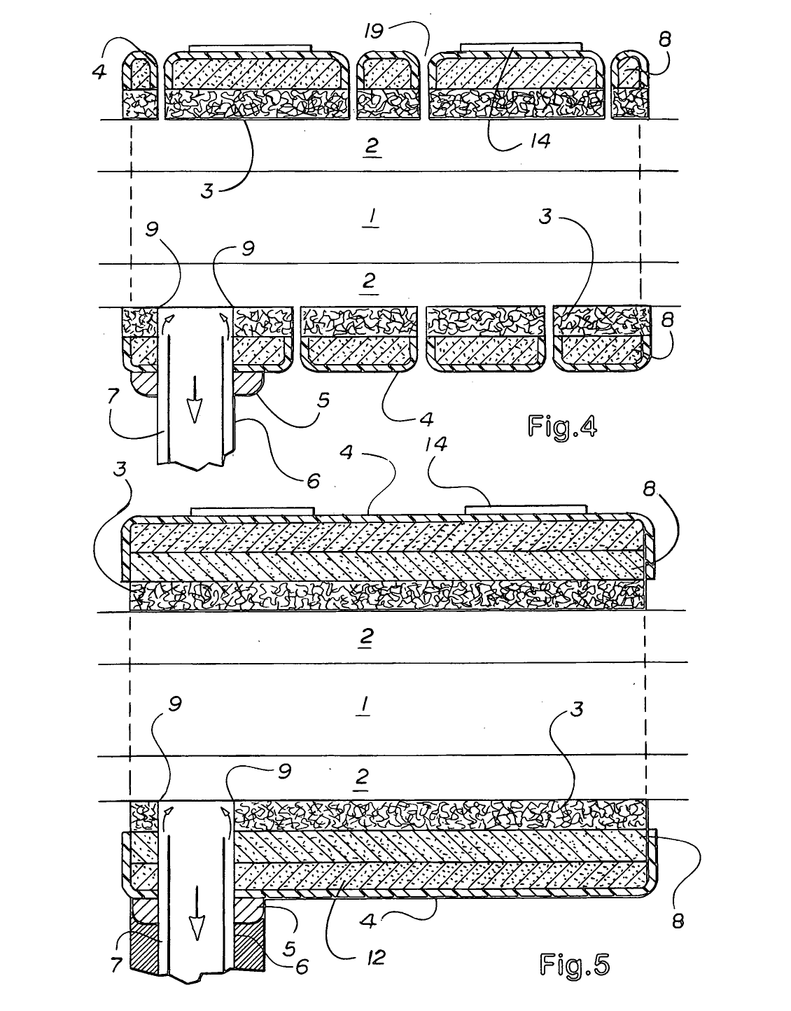

Piped contraction jackets as depicted in FIGS. 11 and 12 and piped extraction jackets as depicted in FIGS. 13 and 14 that deliver susceptible particle bound drugs or other therapeutic substances and constrict the lumen to reduce the gap in the magnetic circuit are not considered to have potential applications as would prompt defining these as separate types of jackets rather than modifications of those so defined. Automatic parenteral administration is usually through an upstream simple junction type jacket as shown in FIGS. 1 and 2 or through the oral route. Another basic type, the electromagnetic impasse-jacket without a side-entry line, and therefore, not a side-entry or piped jacket, is configured to draw a ductus contents-borne magnetically susceptible particle bound therapeutic substance into the wall surrounding the lumen. A third type, analogous to the jackets shown with a permanent magnetic layer in FIGS. 3 and 4, adds a side-entry line to an electromagnetic impasse-jacket.

A combination type jacket not mentioned above combines the configuration shown in FIGS. 13 and 15 with an electromagnetic impasse-jacket, allowing take-up into the wall of the magnetically susceptible particle bound therapeutic substance before the more powerful extraction electromagnet removes any residue. In such a jacket, the less powerful impasse section (portion, segment) precedes the extraction magnet. Another type of electromagnetic ductus jacket, used with radiation shielding to expose the lining of the lumen to a radionuclide, for example, combines a side-entry line with an extraction magnet, yet another with proximal side-entry line, and distal extraction magnet interposed by an impasse segment. As to further permutations, permanent and electrical magnets are not used in the same jacket, and the impasse section or segment of a jacket with or without a side-entry line precedes the extraction magnet.

In the train of extraction jackets shown in FIG. 14, only the flush line is connected in series; to allow the jackets to operate sequentially, the electrical connection of the controller to each jacket must be separate and direct. For most purposes, sequential operation of the jackets is consecutive in line. Otherwise, each can be operated in any sequence vis a vis the others. The sequence in which the magnets are energized can respond to sensor feedback, as will be addressed. Unlike the permanent clasp-magnets addressed in copending application Ser. No. 13/694,835 which draw drug-carrier bound particles continuously, a clasp-electromagnet as shown in FIGS. 8 and 9, fastened to the outer capsule or fibrosal tunic of a target bodily organ, allows the field strength to be turned on, off, or adjusted by the system master microcontroller.

FIGS. 8 and 9 show the clasp-magnet, permanent or electromagnetic, with the pole facing the prongs to engage tissue in facing relation (face-mounted; forward-mounted); however, clasp-magnets, permanent or electromagnetic, can be mounted to face sideways, that is, side-mounted; or backwards to face away from the tissue to which the magnet is engaged (rear-mounted). A compound clasp-magnet mounts multiple magnets on a common mounting. This is done with permanent magnets to adjust the orientation of each magnet by bending the mounting as necessary and with electromagnets for the same reason or to allow a linear array to be energized in a particular sequence. When the clasp-magnet, permanent or electromagnetic, individual or compound, must be mounted at an angle or the individual magnets angled in relation to one another, the mounting plate with integral prongs is metal to allow bending as necessary; otherwise, it is made of polyether ether ketone (PEEK) or a similar tissue compatible polymer.

Electromagnetic clasp-magnets can be used, for example, to detain passage of a superparamagnetic particle carrier-bound drug at a certain level along the ductus thereby increasing takeup in the wall surrounding the lumen, to boost the attractive force of an impasse-jacket that cannot be fit into the space available, or to boost and so bias the attractive force in a certain direction to treat an eccentric lesion. Provided an eccentric lesion is near-sided, an electromagnetic clasp-magnet can eliminate the need to encircle a tunneling coronary artery or other ductus which is resistant to dissection and difficult to encircle with an impasse-jacket.

For epicardial application, for example, where the beating heart would cause a proud-standing implant fastened to the visceral pericardium to abrade against the parietal pericardium, the electromagnetic clasp-magnet must be made least obtrusive with a squat core and profile, large number of coil or winding turns, and a smooth and rounded cover or cap. At a bifurcation, such as the division of the common into the external and internal carotids or the thoracic aorta into the common iliac arteries, the carriers can be diverted to pass along the one route rather than the other. Such magnets, separate from but under joint coordinated control with drug delivery through the jacket or jackets, allows accelerated penetration into or through the parenchyma or the cessation of attraction as necessary.

With both clasp-magnets and electromagnetic impasse-jackets, the ability to surge the attractive force allows forcible penetration of the carried drug or other affinate into the wall surrounding the lumen on a sudden pulsed or extended basis. The coil (solenoid, winding) can be configured to accommodate different anatomical spaces. The mounting for an electromagnetic clasp-magnet, or patch-magnet is the same as for the permanent magnet shown in FIGS. 25 and 26 of copending application Ser. No. 13/694,835. Referring now to FIGS. 13 and 14, because the electromagnet-incorporating jackets can be sequentially energized, to include consecutively along the ductus, the same train of jackets can be used for magnetically susceptible carrier bound drug delivery at an inception level along the ductus respective of any in the set without intrinsic nonaffinitive or magnetically coerced takeup until the first energized jacket is approached.

Crohns skip lesions can be treated by simultaneously or sequentially energizing the jackets to take up a carrier particle-bound steroid, for example, and the jacket encircling each lesion can be energized to take up the drug or drugs in proportion to the severity of the respective lesion. Adjustment of the field strength allows the drug to be drawn up against the lumen wall, drawn radially outward through the wall to the depth required, or completely extracted. With sequential energization, the same arrangement allows high leukocyte count blood to be remediated by allowing the extractate to be apportioned among the extraction jackets, thus accomplishing ambulatory leukaperesis through magnetic separation while avoiding clogging 2.

Because such applications are intended to be automatic and ambulatory, upstream infusion of the ferrofluid is through a simple junction side-entry jacket of the kind shown in FIG. Closely related to magnetized ductus side-entry or piped impasse-jackets are nonpiped electromagnetic impasse-jackets and compound or multielectromagnet peristalsis jackets. Positioned opposite magnetically susceptible plates as shown in FIGS. 10 and 11, an appropriately positioned set of clasp-electromagnets as shown in FIGS. 8 and 9 can be used to reinstate impaired peristalsis in the capsule of an organ, primarily the kidney. The plates are usually die cut from a thin ferromagnetic stainless steel sheet stock, deburred, and if necessary, edged with a polymeric border to prevent incisions into the substrate ductus, whether native with weakened intrinsic motile function or prosthetic.

FIG. 10 shows an multielectromagnet-jacket mounting magnets without integrated extraction trap and flush line in an independent jacket as are those depicted in FIGS. 13 thru 15. Spacing and consecutively energizing clasp-electromagnets such as shown in FIGS. 8 and 9 with opposing plates at points along the surface of an organ, or encircling a ductus with a multielectromagnet-jacket such as shown in FIGS. 10 and 11 allows assisting impaired peristalsis or the imparting of peristalsis to a prosthetic or graft ductus. Because both prostheses made of artificial materials and transplanted segments, even autologous, tend to fail at the anastomoses or can be sustained only with immunosuppressant drugs that place the patient at life-long risk for other disease, such prostheses and grafts are better joined to native tissue by means of simple junction jackets of the kind shown in FIGS. 2, 21, and 22.

These not only avoid direct contact between native and prosthetic or graft ductus but allow the direct delivery of medication to the junctions, and if the graft consists of native or engineered tissue, the graft segment. Contraction-jackets are intended for imparting simulated or intrinsic-equivalent normotensive motility to tissue-engineered graft segments of digestive, urinary, and reproductive tract prostheses and native segments that due to disease or innate deficiency, require peristaltic or sphintreric function or function reinforcement respectively. When generated from autologous cells, normally peristaltic structures elude rejection but fail to develop normal motile function.

While one report is encouraging (Sjöqvist, S., Jungebluth, P., Lim, M. L., Haag, J. C., Gustafsson, Y., Lemon, G., Baiguera, S., and 16 others 2014. “Experimental Orthotopic Transplantation of a Tissue-engineered Oesophagus in Rats,” Nature Communications 5:3562), tissue engineered esophagi by and large have tended to stenose, and exhibit other deficits (see, for example, Luc, G., Durand, M., Collet, D., Guillemot, F., and Bordenave, L. 2014. “Esophageal Tissue Engineering,” Expert Review of Medical Devices 11(2):225-241; Maghsoudlou, P., Eaton, S., and De Coppi, P. 2014. “Tissue Engineering of the Esophagus,” Seminars in Pediatric Surgery 23(3):127-134. Totonelli, G., Maghsoudlou, P., Fishman, J. M., Orlando, G., Ansari, T., Sibbons, P., Birchall, M. A., Pierro, A., Eaton, S., and De Coppi, P. 2012. “Esophageal Tissue Engineering: A New Approach for Esophageal Replacement,” World Journal of Gastroenterology 18(47):6900-6907; Saxena, A. K. 2014. “Esophagus Tissue Engineering: Designing and Crafting the Components for the “Hybrid Construct” Approach,” European Journal of Pediatric Surgery 24(3):246-262).

Engineered vascular tissue does, however, exhibit the capacity to regenerate when injured and grow (see, for example, Cho, S. W., Kim, I. K., Kang, J. M., Song, K. W., Kim, H. S., Park, C. H., Yoo, K. J., and Kim, B. S. 2009. “Evidence for In Vivo Growth Potential and Vascular Remodeling of Tissue-engineered Artery,” Tissue Engineering. Part A 15(4):901-912; Boerckela, J. D., Uhriga, B. A., Willetta, N.J., Huebschb, N., and Guldberga, R. E. 2011. “Mechanical Regulation of Vascular Growth and Tissue Regeneration In Vivo” Proceedings of the National Academy of Sciences of the United States 108(37): E674-E680; Cummings, I., George, S., Kelm, J., Schmidt, D., Emmert, M. Y., Weber, B., Zünd, G., and Hoerstrup, S. P. 2012. “Tissue-engineered Vascular Graft Remodeling in a Growing Lamb Model: Expression of Matrix Metalloproteinases,” European Journal of Cardiothoracic Surgery 2012 41(1):167-172; Kelm, J. M., Emmert, M. Y, Zürcher, A., Schmidt, D., Begus Nahrmann, Y., Rudolph, K. L., Weber, B., and 8 others 2012. “Functionality, Growth and Accelerated Aging of Tissue Engineered Living Autologous Vascular Grafts,” Biomaterials 33(33):8277-8285; Hoerstrup, S. P., Cummings, I., Lachat, M., Schoen, F. J., Jenni, R., Leschka, S., Neuenschwander, S., and 6 others 2006. “Functional Growth in Tissue-engineered Living, Vascular Grafts: Follow-up at 100 Weeks in a Large Animal Model,” Circulation 114(1 Supplement):I159-I166). Until a tissue engineered esophagus is developed, one solution is to engineer esophageal tissue as flat stock or sheeting which can be rolled into a tube and bonded at the free side edges to coalesce by healing, creating a living ductus of the correct strength and pliancy and apply the assist device described herein. Essentially, if the tube is little more than viable, it can be used. For now, such represents a tissue engineered esophagus as the term is used herein.

Motile dysfunction is likewise central to numerous pathological conditions of the native digestive tract whereby the segment has satisfactory structure but motile and probably secretory deficits, so that the lack of a cure and the nonfeasability of a fully functional graft preclude resection and replacement. However, deformity that would require a graft in any event, and weakness or dysfunction that would impart motile and secretory function through reinforcement of the native conduit without the need for resection and grafting plainly justify prostheses which impart such function. Whether in a tissue-engineered or a malfunctioning native gut or segment thereof, if the myenteric or Auerbach's plexus, which controls peristalsis, fails to develop normally, then it is probable that neural development is impaired generally, with the submucosal or Meissner's plexus, which controls secretion, and sensation generally, likewise impaired.

While a lack of sensation may be advantageous as reducing or eliminating annoyance during operation, side-entry jackets and suitable timing controls make possible both prosthetic secretory and contractile function, different means therefor addressed below. In prosthetic vessels, prosthetic endothelial function is achieved by delivery of replacement substances through a simple junction jacket placed upstream. Where the segment involved would additionally benefit from a drug or drugs, and the target area for the drug need not be tightly circumscribed, a simple junction jacket such as shown in FIG. 2 placed upstream will serve. If the graft or native segment is small, the target area for the drug is best restricted to a certain segment whether that equates to the segment to be encircled, or the segment is to be encircled over the least length possible, or the surrounding anatomy allows little clearance, a combination or special-purpose contraction or peristaltic jacket with fluid side-entry, and if appropriate, electrical line or lines added is used.

The need for a sphincteric assist device alone in a neonate infrequent, whether applied to a tissue engineered graft or native dysfunctional tract as a jacket, the device will often incorporate both esophageal and sphincteric components with implanted control module as a unit. For a neonate, the graft is generated from autologous stem or progenitor cells harvested prenatally from fetal sources such as umbilical cord blood, amniotic fluid,—and chorionic villi as are cardiovascular tissue engineered graft cells (see, for example, Weber, B., Zeisberger, S. M., and Hoerstrup, S. P. 2011. “Prenatally Harvested Cells for Cardiovascular Tissue Engineering: Fabrication of Autologous Implants Prior to Birth,” Placenta 32 Supplement 4:S316-S319). Based upon tissue engineered vascular tissue, a perfected tissue engineered esophagus would probably grow in proportion to the rest of the body (Cho) and heal if injured (Boerckel).

In an adult, irremediable damage necessitating a functional prosthesis incorporating both components may result from ischemia (see, for example, De Praetere, H., Lerut, P., Johan, M., Daenens, K., Houthoofd, S., Fourneau, I., Maleux, G., Lerut, T., and Nevelsteen, A. 2010. “Esophageal Necrosis after Endoprosthesis for Ruptured Thoracoabdominal Aneurysm Type I: Can Long-segment Stent Grafting of the Thoracoabdominal Aorta Induce Transmural Necrosis?,” Annals of Vascular Surgery 24(8):1137.e7-e12), compression (see, for example, Ruzmetov, M., Vijay, P., Rodefeld, M. D., Turrentine, M. W., and Brown, J. W. 2009. “Follow-up of Surgical Correction of Aortic Arch Anomalies Causing Tracheoesophageal Compression: A 38-year Single Institution Experience,” Journal of Pediatric Surgery 44(7):1328-1332; Bonnard, A., Auber, F., Fourcade, L., Marchac, V., Emond, S., and Révillon, Y. 2003. “Vascular Ring Abnormalities: A Retrospective Study of 62 Cases,” Journal of Pediatric Surgery 38(4):539-543), surgical resection (see, for example, Lerut, T. 2001. “Indications and Outcome of Esophageal Resection,” in Tilanus, H. W. and Attwood, S. E. A., Barrett's Esophagus, New York, N.Y.: Springer, pages 317-324), or accidental trauma.

A sphincter is normally closed with its smooth muscle lax and opened when the muscle is energized. When the sphincter is too loose, luminal contents reflux, and when stenotic if not atresic, fails to open when necessary, obstructing the gastric outlet, for example. Because the sphincteric jacket keeps the lumen fully closed until its electromagnet is energized, and fully open when the magnet is energized, conditions both of stenosis and patency are alleviated by the same jacket. Such a jacket may be thought of as an adaptation of a break contact relay, where the living tissue of the ductus is interposed between the contacts. The sphincteric jacket to be described keeps the sphincter closed and opens it only when its respective node as a module or subsystem in the multicore microcontroller is signaled by sensors implanted along the digestive tract that a bolus has entered.

Effective treatments having long existed, the application of individual, or sphincteric contraction-jackets to counteract a sphincter that fails to open the passageway through a hypertrophic or stenotic sphincter as an expansion, dilation, or distension jacket is not ordinarily required. Such a condition as congenital achalasia or hypertrophy of the lower esophageal sphincter is ordinarily amenable to remedial treatment by pneumatic balloon dilation, botulinum toxin injection, or more durably by surgical correction with a Heller myotomy or a variant thereof, and hypertrophic pyloric stenosis (pyloristenosis, pylorostenosis), conventionally resolved by an endoscopic longitudinal pyloromyotomy (Fredet-Ramstedt operation, Ramstedt-Fredet operation, Ramstedt's operation, Ramstedt pyloromyotomy) or a pyloroplasty.

Neither is the placement of a sphincteric jacket proposed where reflux is the result of an hiatal hernia which can be simply and safely repaired. The placement of a functional prosthesis may prove preferable to an open surgical transduodenal sphincteroplasty necessitated by scarring due to repeated endoscopic treatment and dysfunction as the result of a gastric bypass operation or the resection of an ulcer or tumor, for example. The graft is first anastomosed in position, or the dysfunctional native sphincter sphincteroplastied to allow placement of the jacket endoscopically, the balance of the procedure performed through the same access portal or opening. The final decision to debulk, incise, or only place the sphincteric jacket assist device to an hypertrophied sphincter is reserved should be made only once the sphincter has been exposed. The jacket is fastened about the sphincter with hook and loop bands wetted on both internal and external surfaces with substances that will forestall if not eliminate an adverse tissue reaction, to include phosphorylcholine, and/or dexamethasone, or curcumin.

These should at least make exposure to the polymeric materials gradual, allowing for adaptation. When the end outer diameter of an hypertrophied sphincter cannot be predetermined or the diameter is irregular or tapered, a sphincteric jacket with magnet pole outside the adventitia as shown in FIG. 12 or an extraction jacket with flap-valve flush thereto such as shown in FIG. 13 is selected for the largest prospective internal diameter is angled to a point about the circumference of the ductus where the diameter is greatest and additional strips of viscoelastic polyurethane foam lining supplied with the jacket used to build up the thickness of the lining of a jacket where the internal surface of the foam falls short of the adventitia. Adhesion of the added layer or layers of foam is by wetting the interface between layers with cyanoacrylate cement. The placement of a sphincteric jacket to alleviate dysfunction as stenosis or patency is reserved for patients not considered good candidates for such a procedure.

Patients considered poor risks for the surgery and those who have not benefited from a previous surgical procedure are well served by a fully implanted assist device that functions automatically to meter stomach contents at the entry and/or outlet, and requires a relatively minor laparoscopic procedure under local anesthesia to be placed. Ordinarily, conditions involving inadequacy or the lack of contractive force, such as in Hirschspring's disease, where surgical intervention often leads to adverse sequelae, are addressed. An independently used sphincteric or peristaltic assist device is a module that can be incorporated in a more comprehensive ambulatory prosthetic disorder response system assigned one of the nodes in the microcontroller. When used alone, the device is fully implanted, with only a connector at the body surface to recharge the battery.

The laparoscopic procedure is relatively simple, less susceptible to complications, and with a strain gauge sensor designed to be slid into the outer tunic of the esophagus, for example, less demanding of dissection and surgical expertise than the Ramstedt operation. Sensors suitable for signaling intrinsic motility include piezoresistors, nanoparticle based resistive strain gauges, long life mercury in rubber strain gauges, and fiber optic strain gauges. Intraoperative time is less, the procedure usually performed under local anesthesia even on a neonate, with less dissection, and the elimination of an accidental duodenotomy or gastrotomy as a distinct risk in a tiny patient (see, for example, Oldham, K. T., Coraqn, Q. G., and Wesley, J. R. 1997. “Pediaatric Abdomen,” Chapter 103 in Greenfield, L. J., Mulholland, M. W., Oldham, K. T., Zelenock, G. B., and Lillemoe, K. D. (eds.), Surgery: Scientific Principles and Practice, page 2067) than even the usually dependable Ramstedt operation. Broadly, such means are intended to apply where disease or malformity which has eluded medical treatment or a previous attempt at surgical correction would otherwise necessitate resection and/or replacement of the native structure.

Contraction and peristaltic jackets and controls are best devised as integral components of a more inclusive disorder response system which collaterally treats unrelated or related coexisting disease, but can be separated as a distinct module within the more comprehensive disorder response system as singular for a patient without collateral disease. When applied by itself, a pump-pack is dispensed with, the controller and battery then also implanted. This is not the case, however, with a patient, primarily one elderly, who is likely to develop other disease as will then require a pump-pack. Even when a proximal or upstream simple junction jacket is needed to deliver medication or synthetic mucus, for example, or the jacket is to include a ductus side-connector for delivery of a therapeutic substance or substances through a commercially available port or the port described below at the body surface, necessitating the addition of a pump-pack, whenever possible, the procedure is performed endoscopically or laparoscopically under local anesthesia, as is any other procedure contemplated herein.

To avoid an unpleasant esthetic result, pump-pack fluid and electrical lines are generally not tunneled subcutaneously. When fluid support is needed an a separate jacket upsteam is not wanted, sphincteric and peristaltic jackets include a side-entry connector with mainline and sideline. Such fluids typically include synthetic mucus, any deficient digestion hormones, such as cholecystokinin and secretin, and enzymes, such as amylase or a protease. An excessively constrictive or stenotic lower esophageal sphincter such as one hypertrophic is ordinarily correctable through a fundoplicaton or Nissen fundoplicaton. However, a weakened one, where the smooth muscle is impaired or a hiatal hernia reduces the available contractive force, leaves the inlet to the stomach partially open. This is a common cause of backwashing or gastroesophageal reflux disease, which can lead to Barrett's esophagus, metaplasia and esophageal cancer.

The means described herein are intended for cases that do not respond well to medical intervention, such as in patients who do not tolerate proton pump inhibitors and histamine blockers well. Other sphincters that cut off rather than properly modulate flow are correctable through relatively safe and simple surgical procedures, such as a pyloromyotomy. However, a weakened pylorus allows duodenogastric reflux and prechymal gastric contents to pass, often leading to ulceration and early dumping syndrome. A dysfunctional ileocecal valve (ileal valve, Tulp's valve) promotes ileocecal incompetence with the return entry (reflux, regurgitation) of colonic contents into the ileum. When standard of care methods are contraindicated or prove inadequate, a sphincteric or peristaltic contraction-jacket, to include implantable sensors and when drug delivery is necessary, a wearable power pack and control module as described below in the section entitled Description of the Preferred Embodiments of the Invention, may provide a remedy.

When used alone and without fluid delivering capability, impaired or graft sphincter or peristaltic assist devices are packaged separately and fully implanted. When the system must serve collateral disease so that additional modules addressing other dysfunction are required, a pump-pack is used. Whether a separate module or several are required, each module is preferably controlled within a standardized control regime. It is assigned a respective node and treated and programmed as a semi-independent subsidiary module of a more inclusive prosthetic disorder response system. Supportable with prosthetic distension (expansion, dilation, dilatation) or contractile (constrictive) function no less than tissue-engineered segments are native conduits and segments deficient in contractile function, but which would best be left intact were means available to reinforce or provide the missing contractive force.

Jackets applied thus are actuated on the basis of sensors such as strain gauge-based which follow and signal passage of the bolus to the respective control node. Such deficits, those which result from congenital deformaties such as congenital esophageal atresia, congenital intestinal atresia (Christmas tree, maypole, or apple tree deformity), duodenal, jejunoileal, ileal, and colon atresia, defects in neuromuscular development, remedial drug-habituation, as well as the desirability for a functional graft following resection dictated by disease or injury, as when following the removal of a long segment from the gut, responsive to refractory Crohn's disease or ileocolitis, for example. When discovered in utero, the foam lining the tissue-engineered prosthesis with peristalsis multiple electromagnet peristalsis jacket is made as thick as possible to allow for growth. Following resection for familial adenomatous polyposis, for example, as much of the colon down to the perineum as possible is replaced.

With respect to grafts tissue engineered with autologus cells, because the deficits of motor function obtained are due to myenteric plexus and/or associated neural maldevelopment, such grafts are also likely to be associated with deficits in sensory function. Provided neighboring tissue is not encroached upon, irritation if any from the operation of an implanted sphincteric or peristaltic device should not prove prohibitive. Since a tissue engineered or harvested autologous graft is defective in sensory function, the weight of the magnet will not be sensed. Nevertheless, when considered heavy, the magnet should be suspended with suture or an improvised sling harness. Referring now to FIG. 10, a lower esophageal or a pyloric sphincter that proves untreatable or untreatable without heavy medication can be resected and replaced with a tissue-engineered replacement. Sphincteric motility is imparted by a single contraction-electromagnet jacket such as shown in FIGS. 11 and 12, briefly described below and more fully described in the section entitled Sphincteric Jackets under Description of the Preferred Embodiments of the Invention.

FIG. 11 shows an isolated peristaltic electromagnetic contraction-jacket. A sphinteric jacket differs from such a peristaltic contraction-jacket magnet, in that the engineered tissue graft ductus is encircled within a hard outer shell having a strong permanent disk magnet mounted to, inlaid or inset into, or embedded within its outer surface. The magnet is magnetized in its long axis or parallel to length, and if not embedded, must be encapsulated within a polymeric outer layer, such as polytetrafluoroethylene, that will not abrade neighboring tissue and is not susceptible to hydrolytic or enzymatic degradation. This permanent magnet is positioned in diametrical opposition to the pole of the electromagnet, and a magnetic stainless steel or iron plate encapsulated with a polymer impervious to hydrolytic or enzymatic dissolution is sutured to the magnet pole side of the graft. The peristaltic jacket also differs in requiring internal sequential timing control. With both, however, strain gauge sensor implants proximal to the sphincter signal passage of a bolus to the control node.

When a prosthetic esophagus includes the lower esophageal sphincter, control of peristalsis and dilatation of the sphincter are controlled as a unit These sensors can be placed in the pharynx and along the esophagus in sensor-jackets, which must meet the desiderata for a compliant lining and fenestrations, for example. The propulsive action of peristalsis over a distance requires the action of multiple contraction-electromagnets coordinated to step along leap-frog fashion to simulate an advancing wave of constriction or extrusion. FIG. 10 shows a peristalsis assist device jacket with five contraction-electromagnets to replace a relatively short segment along the digestive, urinary, or reproductive tract, the dimensions of the jacket, its number and spacing apart of magnets dictated by the application. To the extent possible, timing control over magnet energization simulates that normal, the more longitudinal peristalsis of a ureter, for example, different in character than that along the digestive tract.

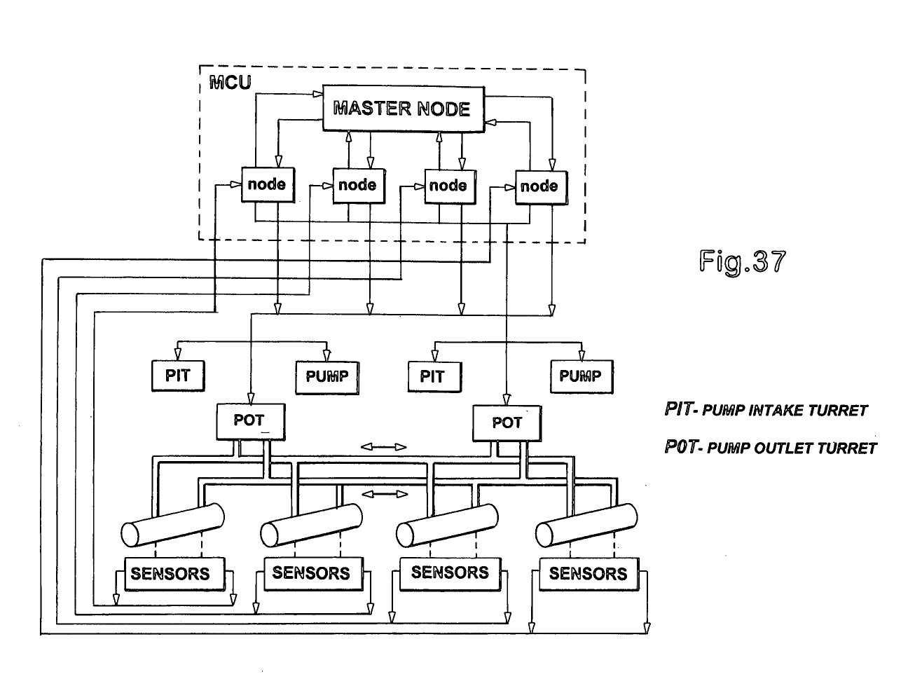

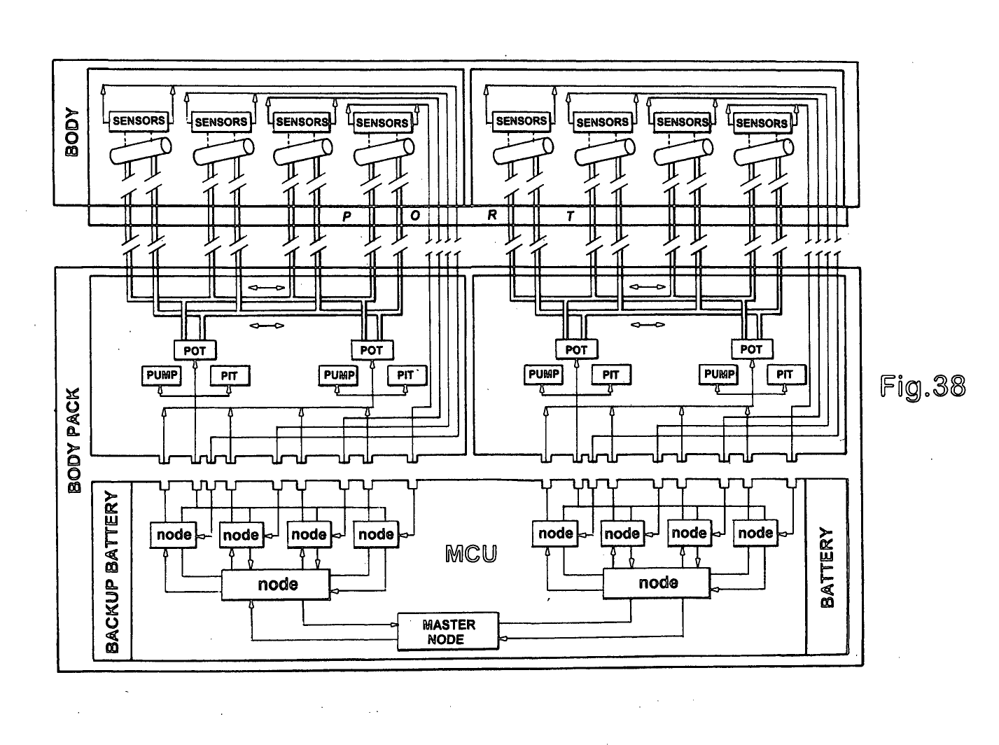

The esophagus, for example, is replaceable or supportable along its entire length distal to the upper esophageal sphincter. When used to bolster intrinsic function, the jacket is placed about the native ductus; when a graft is required, the jacket is placed about the graft, one tissue-engineered using autogenous or autologous cells preferred as least likely to be rejected. Replacement of the esophagus and pylorus, for example, combines a multiple contraction or peristaltic jacket for the esophagus and an individual peristaltic jacket for the pylorus and places these under the control of a higher control node for jacket to jacket synchronization. When no other microcontroller modules are needed to treat collateral disease in the same patient, control of either or coordinated control of both the peristaltic and sphincteric jackets is relegated to local control, the microcontroller then implanted with only the one node used as a local control module. Otherwise, the multicore microcontroller is housed within the pump-pack, subsidiary or subordinate control nodes of the microcontroller shown in FIGS. 37 and 38.

Absence in peristaltic or sphintrerate function results from congenital deformities (see, for example, Pansky, Ben 1982. Review of Medical Embryology, Macmillan USA), usually the result of prenatal ischemia, and impairments from nervous and/or muscular deficits due to disease congenital or acquired, or injury, surgical or accidental see, for example, Smout, A. and Fox, M. 2012. “Weak and Absent Peristalsis,” Neurogastroenterology and Motility 24 Supplement 1:40-47). Provided safe and dependable prostheses are available, irremediably nonfunctional segments can be reinforced, or if unavoidable for collateral reasons, resected and replaced, and congenitally missing segments provided. Whether the native ductus is encircled for reinforcement or a tissue-engineered graft is used, myenteric plexus inadequacy can be compensated for through the delivery of drugs, enzymes and/or synthetic mucus, contraction-synchronized if necessary.

With shorter segments, these can be delivered through an upstream simple junction type side-entry jacket. For short segments, synthetic muciferous, and natural or synthetic enzymatic, and hormonal secreta can be delivered as necessary through a simple junction jacket placed proximally to the affected segment or by adding a side-entry connector to the contraction jacket, which represents a kind of hybrid or combination jacket. When this is inadequate with a peristalsis jacket such as shown in FIG. 10, simple junction jackets can be positioned perpendicularly to the contraction jackets. Exact synchronization rather than simple simultaneity by the control system of the secretory with the contractile function is unnecessary. Whereas such a condition as comorbid or associated with other symptoms can be treated together with the other symptoms by the control system delineated herein, isolated and uncomplicated contractile dysfunction can be treated with a contraction jacket or shorter peristaltic contraction-jacket with dedicated microcontroller and battery pack.

The control program can be adapted to compensate for segmental dysfunction such as to tighten the lower esophageal sphincter. Whether the dysfunction is continuous or intermittent, the introduction of a food bolus, or in a secretory ductus, the buildup of pressure due to accumulation of secretion, causes the strain gauge or equivalent sensors implanted along the ductus to input the activating signal. Ductus with peristalsis include the ureters, glandular ducts, and gamete transmitting conduits, as well as the gastrointestinal tract, and the renal pelvic wall (Pruitt, M. E., Knepper, M. A., Graves, B., Schmidt-Nielsen, B. 2006. “Effect of Peristaltic Contractions of the Renal Pelvic Wall on Solute Concentrations of the Renal Inner Medulla in the Hamster,” American Journal of Physiology. Renal Physiology 290(4):F892-F896) and uterus (Kunz, G., Beil, D., Huppert, P., and Leyendecker, G. 2006. “Control and Function of Uterine Peristalsis During the Human Luteal Phase,” Reproductive Biomedicine Online 13(4):528-540; Kunz, G. and Leyendecker, G. 2002. “Uterine Peristaltic Activity During the Menstrual cycle: Characterization; Regulation, Function and Dysfunction,” Reproductive Biomedicine Online 4 Suppl 3:5-9).

Applied thus, two or three larger clasp-electromagnets such as shown in FIGS. 8 and 9 can be used to treat gastroparesis, for example. Applied distad along the gastrointestinal tract, a multielectromagnet-jacket as shown in FIG. 10 allows assisting intrinsic function and averting paralytic ileus as the result of a chronically slow wave rate refractory to correction, whether due to ischemia, neuroendocrine, neuroelectrical, enteric neuromuscular, psychosomatic, genetic, environmental, immunological, or iatrogenically induced dysfunction caused by opioids or sedatives, for example. When the intrinsic rate is slow, the consecutive magnets are energized to least interfere with residual intrinsic function by using implanted strain gauge bolus sensors, a timing circuit, and prior art pacing electronics, for example, to interpose an additional contraction midway between those intrinsic.

Intrinsic contractions can be reinforced in transit at the magnet sites. When modified as indicated above, the separate type electromagnet-jackets shown in FIGS. 13 thru 16 consolidate the abilities to assist peristalsis mechanically and deliver drugs such as cerulein and neostigmine, established in vitro to be prokinetic (Fruhwald, S., Herk, E., Hammer, H. F., Holzer, P., and Metzler, H. 2004. “Differential Reversal of Drug-induced Small Bowel Paralysis by Cerulein and Neostigmine,” Intensive Care Medicine 30(7):1414-1420), where not to directly target these would prove disruptive to other function. Drug delivery through the flush-out line along the train shown in FIG. 14 can be accomplished by pulsing the pump to force open the flap-valves, or if a ferrofluid, then with the aid of a diametrically positioned electromagnet. Discretionary delivery to each side-connector requires that each be provided with a side-connector sideline accessory or service-channel such as appears as part numbers 10 and 11 in FIGS. 1 thru 3 and 16 thru 22.

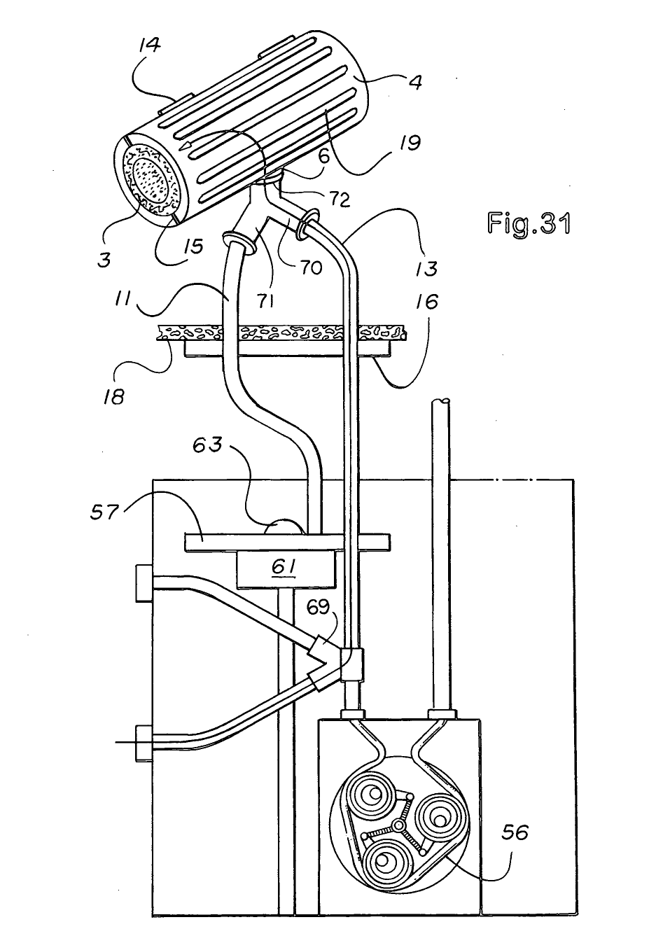

Use to directly assist a weakened pulse by placement of a hybrid jacket along the aorta in the position where a different type of jacket is shown in FIG. 16 and about the pulmonary trunk, will often have been surpassed by implantable ventricular assist devices already in use. This approach can, however, be used to boost the pulse peripherally. Even though the flush line pump and jacket to jacket connections in series, as shown in FIG. 14, can be made strong enough, a drug transmitted through the flush line is limited to sequential delivery, and to avoid each successive opening being passed as posing greater resistance to inflow, would require costly supplementation. Instead of nondiscretionary and substantially simultaneous delivery, which offers no advantage, delivery from turrets as shown in FIGS. 31, 32, and 36 is made critically more versatile in timing and dose by providing the pump or pumps with an outlet turret that allows switching among different lines to each jacket or side-connector sideline.

To date, tissue-engineered esophagi and intestine lack normal neural plexus development and therefore peristaltic function (Totonelli, G., Maghsoudlou, P., Fishman, J. M., Orlando, G., Ansari, T., and 5 others 2012. “Esophageal Tissue-engineered ing: A New Approach for Esophageal Replacement,” World Journal of Gastroenterology 18(47):6900-6907; Saxena, A. K., Baumgart, H., Komann, C., Ainoedhofer, H., Soltysiak, P., Kofler, K., and Höllwarth, M. E. 2010. “Esophagus Tissue-engineered ing: In Situ Generation of Rudimentary Tubular Vascularized Esophageal Conduit Using the Ovine Model,” Journal of Pediatric Surgery 45(5):859-864), and autologous grafts harvested from the distal gastrointestinal tract have proven too susceptible to “leakage, infection and stenosis at the implanted site, which leads to severe morbidity and mortality (Kuppan, P., Sethuraman, S., and Krishnan, U. M. 2012. “Tissue-engineered ing Interventions for Esophageal Disorders—Promises and Challenges,” Biotechnology Advances 30(6):1481-1492; Shen, Q., Shi, P., Gao, M., Yu, X., Liu, Y., Luo, L., and Zhu, Y. 2013. “Progress on Materials and Scaffold Fabrications Applied to Esophageal Tissue-engineered ing,” Materials Science and Engineering. C, Materials for Biological Application 33(4):1860-1866; Nakase, Y., Nakamura, T., Kin, S., Nakashima, S., Yoshikawa, T., Kuriu, Y., Sakakura, C. and 5 others 2008. “Intrathoracic Esophageal Replacement by in Situ Tissue-engineered Esophagus,” Journal of Thoracic and Cardiovascular Surgery 136(4):850-859; Longmire, W. P. and Ravitch, M. M. 1946. “A New Method for Constructing an Artificial Esophagus,” Annals of Surgery 123(5):819-834).

Rejection the central problem with grafts and end to end anastomoses along the digestive tract, the placement of a simple junction type side-entry jacket as shown positioned for fixation in place in FIG. 1 and fixed in position in FIG. 2 proximally or upstream to the graft can be used to deliver a magnetically susceptible drug-carrier particle bound immunosuppressant and\or adverse tissue response drug or drugs or any combination thereof. Targeted so that uninvolved tissue is not exposed, these drugs can be delivered in higher concentration than might be circulated. Steroids, for example, are used to reduce inflammation, but carry risks such as the inducement of Addison's disease and moon facies. The proximal magnet-plate pairs deenergized once a bolus passes, a bolus to follow enters the lumen without the need for the prosthetic ductus to incorporate resilient means as would make it elastic so that it self-dilates.

Similarly, statins in higher concentration can induce myopathy, and immunosuppressive drugs leave the patient vulnerable to infection. If necessary, a second jacket to release a reversal agent likewise best targeted, or an extraction jacket such as shown in FIG. 13, positioned distally or downstream to the graft can be used to remove any residue of the bound drug and particulate not taken up within the target segment. This can be applied to the reevaluation of transplants and prosthetic approaches whether tissue-engineered or made of synthetics. Restricting delivery of the drug or drugs to the graft critically reduces if not eliminates side effects, drug food, and drug drug interactions, which can be especially problematic in comorbid conditions. The control system can regulate the delivery of different drugs through jackets placed at different locations in the body, averting interactions.

Hence, an arrangement such as that shown in FIG. 10, where a peristalsis dysfunctional (see, for example, Martinucci, I., de Bortoli, N., Giacchino, M., Bodini, G., and 4 others 2014. “Esophageal Motility Abnormalities in Gastroesophageal Reflux Disease,” World Journal of Gastrointestinal Pharmacololgy and Therapeutics 5(2):86-96; Smout, A. and Fox, M. 2012. “Weak and Absent Peristalsis,” Op cit.; Bredenoord, A. J., Fox, M., Kahrilas, P. J., Pandolfino, J. E., Schwizer, W., Smout, A. J; and 17 collaborators 2012. “Chicago Classification Criteria of Esophageal Motility disorders Defined in High Resolution Esophageal Pressure Topography,” Neurogastroenterology and Motility 24 Supplement 1:57-65; Roman S. and Kahrilas, P. J. 2011. “Challenges in the Swallowing Mechanism: Nonobstructive Dysphagia in the Era of High-resolution Manometry and Impedance,” Gastroenterology Clinics of North America 40(4):823-835) or tissue-engineered esophagus not yet capable of intrinsic motility but not rejected as are autologous grafts or remediated as indicated above, can function independently of intrinsic motility if any without the need to be synchronized with residual peristaltic function.

Provided esophageal cancer is detected before metastasis, an autologous engineered replacement with motile function that was not rejected would represent a cure. The magnets are situated at intervals along the prosthesis and act upon magnetically susceptible bands on the opposite outside surface. The action while not sensed normally will slightly move adjacent tissue, which should eventually supplant the intrinsic sensation of swallowing, much as the wearer adapts to the quality of sound through a cochlear implant. Despite decades of experimentation, prosthetic esophagi made of metals and plastics have failed over time, dehiscing at the anastomoses, leaking, and becoming infected.

An intestinal prosthesis imposes the additional requirement of appropriate absorption and passage of nutrients through the mesentery or directly into the portal vein (Sugano, K., Nabuchi, Y., Machida, M., and Aso, Y. 2003. “Prediction of Human Intestinal Permeability Using Artificial Membrane Permeability,” International Journal of Pharmaceutics 257(1-2):245-251). A successful intestinal prosthesis would be tissue-engineered ed, and would probably require assisted motility until perfected. A prosthetic ureter has been stated to require (Graw, M. and Bahl, H. U. 1986. “An Active Artificial Ureter with Autonomous Energy Supply,” Urologia Internationalis 41(1):9-15)) and not to require (Desgrandchamps, F. and Griffith, D. P. 2000. “The Prosthetic Ureter,” Journal of Endourology 14(1):63-77) peristaltic function. In vitro fertilization eclipses the need for an artificial fallopian tube.

Since they place their fingers and everything else in their mouths, infection is inevitable in young patients born with a defect replaced with a prosthesis made of alloplastic (nonbiological) materials. Pending the ability to produce autologous tissue-engineered esophagi and intestines with peristalsis, these tissue compatibility sequelae can be averted, while the motive means described herein is used. Prostheses for insertion along the digestive tract made of alloplastc (nonbiological, artificial) materials may be well engineered as stand-alone items; but in end-to end anastomosis with alloplastic, or artificial, materials, the bacteria-laden lumen predisposes to adverse tissue responses that exceed those seen when the lumen is uninvolved, intrinsic defenses as in the bloodstream are numerous, and at sites where remedial substances are easily applied.

This results in rejection (see, for example, Taira, Y., Kamiya, K., Shiraishi, Y., Miura, H., Shiga, T., Hashem, M. O., Yamada, A., Tsuboko, Y., Ito, T., Sano, K. 2014. “Achievement of Peristaltic Design in the Artificial Esophagus Based on Esophageal Characteristic Analysis of Goats' Specimen,” 15th International Conference on Biomedical Engineering IFMBE Proceedings 43:372-374, New York, N.Y.: Springer; Liang, J. H., Cai, P., Luo, Z. R., Liang, X. L., and Zhou, X. 2012. “Effect of Feeding Regulation Measures for Establishing Esophageal Channel Function in Neoesophagus Created with a Nitinol Artificial Esophagus,” International Journal of Artificial Organs 35(9):671-678; Liang, J. H., Zhou, X., Zheng, Z. B., and Liang, X. L. 2010. “Long-term Form and Function of Neoesophagus after Experimental Replacement of Thoracic Esophagus with Nitinol Composite Artificial Esophagus,” American Society for Artificial Internal Organ Journal 56(3):232-234; Mild, H., Okuyama, T., Kodaira, S., Luo, Y., Takagi, T., Yambe, T., and Sato, Y. 2010. “Artificial-esophagus with Peristaltic Motion Using Shape Memory Alloy,” International Journal of Applied Electromagnetics and Mechanics 33(1-2):705-711; Watanabe, M., Sekine, K., Hori, Y., Shiraishi, Y., Maeda, T., Honma, D., Miyata, G., Saijo, Y., and Yambe, T. 2005. “Artificial Esophagus with Peristaltic Movement,” American Society for Artificial Internal Organ Journal 51(2):158-161).

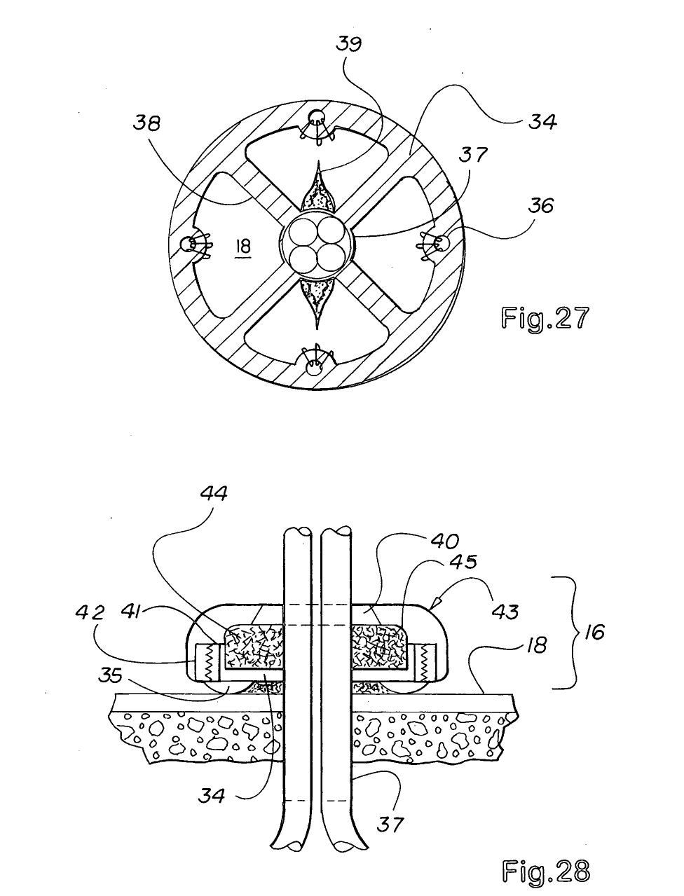

Bare electrodes uninvolved, intracorporeal wiring to coordinate the action of the pump or pumps in the pump-pack, connect implanted sensors to the microcontroller, and so on is similar to that used for other electrical implants, the wires passed through a port implanted at the body surface shown in FIGS. 27 and 28, each biocompatibly insulated and through a common outer conduit, once intracorporeal, to be separated as dictated by the anatomy. Variable control over the field strength of a magnet or magnets integral to a side-entry jacket or jackets if often beneficial. Examples include the ability to adjust field strength to achieve the temporary detention or forcibly drawing of a magnetically susceptible particle bound drug through tissue. With contrast and imaging equipment, the depth of penetration of the carrier bound drug into the tissue can be controlled.

In addition to the advantages of variable control, electromagnets allow the coordinated release of an accumulated extractate so that it can be flushed through a flushout or purge line without the need for high pump pressure that would increase the rate of battery drainage, necessitating heavier batteries in an ambultary apparatus. FIGS. 13 thru 15 show side-entry jackets with one or more integral electromagnets that will allow to pass downstream, detain, draw radially outward through the wall surrounding the lumen, or extract through the wall or the opening made in the side of the ductus a magnetically susceptible carrier particle-bound drug or other therapeutic substance. Ambulatory leukaperesis is but one such application, magnetic separation apheresis, for example, applicable to any analyte that can be bound to a magnetically susceptible drug-carrier.

Preferably, this process is undergone under the control of implanted sensors and the microcontroller in vivo with the patient oblivious to the process, infusion of the fluid containing the binding particles upsteam through a simple ductus junction jacket of the kind shown in FIG. 2. Such jackets can also be used to remove an analyte before reaching downstream or territory tissue or an organ. Unlike the common shaft or trunk of a double arm side-connector without an electromagnet as shown in FIG. 7, which round in cross-section, can include a rotary joint, the rotational angle of the double arm side-connector or connectors and the flap valve toward its adductal terminus to the long axis of the ductus is not round and not rotated. Instead it is made at different fixed angles and cross-sections appropriate for the site.

A separate clasp-electromagnet as shown in FIGS. 8 and 9 for fastening to the surface or outer capsule of an organ, gland, or lymph node, for example, allows an analyte delivered through an upstream side-entry jacket to be detained or drawn into the parenchyma over a controlled timetable. Provided the organ does not have an outer capsule that is very hard, jackets with integrated electromagnet such as shown in FIGS. 13 thru 15 can, over time, extract the susceptible particles with or without the drug still bound, through the fibrosa, where an accumulation thereof can be flushed away. Alternatively, plural small double arm side-connectors with electromagnets can be longitudinally arranged in coaxial relation to the long acid of the ductus.

Alternatively, separate microcontrollers are assigned to each node in the control tree. The object is to optimize drug delivery while least interfering with freedom of movement. The sensors, the positioning of these, and the control desiderata among the nodes signaling a pump-pair depend upon the disease under treatment and vary widely. Except during placement of the implanted elements and drug replenishment or reloading, the patient remains ambulatory. Such an automated drug delivery system can function as a prosthetic disorder response system to compensate for defects in intrinsic adaptive responses, and where an intrinsic response does not exist, is inadequate, or does not squarely target the etiology, as a bionic disorder response system.

In a tertiary medical center with the patient stationary, this scheme can be expanded so that diagnostic sensor feedback initiates and regulates not only ongoing dosing from among clinician prescribed drugs loaded, but can select as well as deliver drugs from among an unlimited number of drug supply reservoirs. While the drugs delivered must be compatible, which is readily accomplished when delivery is targeted, such a system seeks to detect and return analytes such as the level of metabolites, antibodies, antigens, and organic or inorganic substances, to homeostatic balance without necessarily ascribing combinations of imbalances to a particular syndrome. Acting directly upon the basis of sensor inputs, such a system is inherently theranostic, or individualized, and is not susceptible to erroneous presuppositions or errors in treatment to which erroneous presuppositions often lead.

A portable system is loaded with a limited set of specific drugs to treat a diagnosed or predictable condition. By contrast, a stationary system need not be limited thus and does not require a preestablished diagnosis, so that correction expeditious, the risk of misdiagnosis is less. The direct delivery of drugs without relationship to a specific diagnosis allows immediate response to reasonably predictable intercurrent disease, especially valuable when comorbities are likely. For example, with no change in behavior, metabolic syndrome, or the combination of abdominal obesity, hypertriglyceridemia, lowered high density lipoprotein serum level, elevated plasma fasting glucose and low density lipoprotein levels, and hypertension, progression to diabetes and cardiovascular disease is predictable, but not as to time of onset.

Such represents the internalization and rendering immediate of point of care detection (see, for example, Chikkaveeraiah, B. V., Bhirde, A. A., Morgan, N.Y., Eden, H. S., and Chen, X. 2012. “Electrochemical Immunosensors for Detection of Cancer Protein Biomarkers,” ACS [American Chemical Society] Nano 6(8):6546-6561; Rusling, J. F. 2012. “Nanomaterials-based Electrochemical Immunosensors for Proteins,” The Chemical Record 12(1):164-176; Rusling, J. F., Kumar, C. V., Gutkind, J. S., and Patel V. 2010. “Measurement of Biomarker Proteins for Point-of-care Early Detection and Monitoring of Cancer,” The Analyst 135(10):2496-2511; Choi, Y. E., Kwak, J. W., and Park, J. W. 2010. “Nanotechnology for Early Cancer Detection,” Sensors (Basel) 10(1):428-455. Liu, G. and Lin, Y. 2007. “Nanomaterial Labels in Electrochemical Immunosensors and Immunoassays,” Talanta 74(3):308-317).

For such patients with both portable and stationary systems, prepositioning sensor implants to detect and loading the stationary dispensing system with drugs to treat the additional symptoms associated with congestive heart failure, for example, allows the system to respond to these additional symptoms upon onset. Large in number, with additional drugs appearing often, the complement of drugs dispensed by such a stationary system is reduced to those for each purpose which clinical trials have shown to be safe and effective. The automatic drug selection and delivery control program or prescription data switches the drug reservoir catheters connected to each target ductus from among an unlimited number of drug supply reservoirs. In this, a body area network under ‘intelligent’ complex or hierarchical adaptive control can also be made to transmit data through a wireless network.

Much as a vaccine confers artificially acquired immunity, such a system effectively serves as an adjunct or nonintrinsic suppressive or negative feedback response loop for adapting to an anomalous condition. By comparison, automatic ambulatory insulin pumps deliver insulin subcutaneously, hence, systemically following a time delay, without targeting ability, and intravenous drug delivery is unsuited to an active life. Implant cardioverter defibrillators deliver electrical current, not fluid drugs, and ventricular assist devices provide mechanical action. Many genetic defects result in a failure to produce an essential enzyme or protein, or to produce the substance in the normal form and/or amount. A disorder response system that supplements or substitutes for a defective intrinsic response constitutes a physiological prosthesis, whereas a system placed to compensate for a genetic defect that evokes no innate adaptive mechanism is bionic.

A metabolic defect such as failure to produce an enzyme in the proper form often results in a failure for a substance to be assimilated so that an innate adaptive mechanism is obstructed, and in some instances, the defective substance produces a chemical imbalance of insufficiency or of excess that accumulates in tissues or the blood, amyloidosis, the tendinous xanthomas of familial hypercholesterolemia, the eyelid and other xanthomas of hypertriglycerinemia (hyperchylomicronemia), and hemochromatosis examples. In some cases, a defect of metabolism can initiate a cascade or chain reaction of chemical failures that if not truncated result in death. In others, such as Tay-Sachs disease, death is more direct and quicker. Prosthetic and bionic disorder response systems with or without diagnostic sensors and programming are intended to reinstate homeostatic balance.

The development of such prosthetic and bionic systems to supplement or replace innate compensatory adaptive feedback loops has been obstructed by the lack of suitable means for securely joining synthetic fluid lines to anatomical ductus. Provided suitable drugs are available, secure and if necessary, targeted delivery can be established. Here means are described for allowing the direct connection and entry into any native lumen through a securely mounted periductal jacket configured to avoid placement of a foreign object in the lumen and having features to avert intra and postoperative complications, to include bleeding and the leaking of luminal contents that if septic, could result in life-threatening infection. Anatomical clearance allowing, a ductus side-entry connection jacket, or simply side-entry jacket, as described herein, can be connected to a ductus at right angles to form a T-joint normal or perpendicular to the ductus.

Where the anatomy affords no clearance and in blood vessels where hemodynamic and coagulative factors must be accommodated, the jacket is attached to the ductus at an angle. A ductus side-entry connection jacket can be extended or elongated in the antegrade direction to present a magnetized gradient and so form a piped impasse-jacket, whereby the inlet catheter or pipe allows delivery of a fluid drug through a port implanted at the body surface to a periductally mounted jacket. At a minimum, an impasse-jacket is intended to stop magnetically susceptible particles bound to the drug or other therapeutic substance until an external electromagnet can be used to extract these. Among other factors, notably, the alteration in penetrability caused by the lesion itself, the extent of penetration achievable without the aid of an external magnet depends upon the chemistry of the drug or other therapeutic substance carrier-bound particles, as well as the energy product of the magnet material and its dimensions.

Along the vascular tree, penetration at cell depth level to initially treat the intima or where only shallow penetration is wanted can be enhanced through endocytotic methods, such as the binding of lipids or a response-inducing but innocuous virus drawn into endothelial caveolar lipid rafts (see, for example, Kirkham, M. and Parton, R. G. 2005. “Clathrin-independent Endocytosis: New Insights into Caveolae and Non-caveolar Lipid Raft Carriers,” Biochimica et Biophysica Acta 1746(3):349-363; Parton, R. G. and Richards, A. A. 2003. “Lipid Rafts and Caveolae as Portals for Endocytosis: New Insights and Common Mechanisms,” Traffic (Copenhagen) 4(11):724-738). If necessary, a powerful external (extracorporeal) electromagnet is later used to draw the carrier particles radially outward through the lumen wall and/or resituate or extract any residue by force.

Along the vascular tree, primary tumors of the lumen wall are infrequent. However, with susceptible carrier binding and an additional boost through the application of magnetic force, the leaky immature vasculature of tumors along the gastrointestinal tract, for example, allows these to be thoroughly penetrated (see, for example, Holgado, M. A., Martin-Banderas, L., Alvarez-Fuentes, J., Fernandez-Arevalo, M., and Arias, J. L. 2012. “Drug Targeting to Cancer by Nanoparticles Surface Functionalized with Special Biomolecules,” Current Medicinal Chemistry 19(19):3188-3195; Bertrand, N and Leroux, J. C. 2012. “The Journey of a Drug-carrier in the Body: An Anatomo-Physiological Perspective,” Journal of Controlled Release 161(2):152-163; Arias, J. L., Clares, B., Morales, M. E., Gallardo, V., and Ruiz, M. A. 2011. “Lipid-based Drug Delivery Systems for Cancer Treatment,” Current Drug Targets 12(8):1151-1165; Taratula, O., Garbuzenko, O., Savla, R., Wang, Y. A., He, H., and Minko, T. 2011. “Multifunctional Nanomedicine Platform for Cancer Specific Delivery of siRNA by Superparamagnetic Iron Oxide Nanoparticles-Dendrimer Complexes,” Current Drug Delivery 8(1):59-69; Chen, B., Wu, W., and Wang, X. 2011. “Magnetic Iron Oxide Nanoparticles for Tumor-targeted Therapy,” Current Cancer Drug Targets 11(2):184-189; Tseng, Y. C. and Huang, L. 2009. “Self-assembled Lipid Nanomedicines for siRNA Tumor Targeting,” Journal of Biomedical Nanotechnology 5(4):351-363; Koning, G. A. and Krijger, G. C. 2007. “Targeted Multifunctional Lipid-based Nanocarriers for Image-guided Drug Delivery,” Anticancer Agents in Medicinal Chemistry 7(4):425-440; Muller, R. and Keck, C. 2004. “Challenges and Solutions for the Delivery of Biotech Drugs—A Review of Drug Nanocrystal Technology and Lipid Nanoparticles,” Journal of Biotechnology 113 (1-3): 151-170). When an orally administered drug is to be drawn radially outward and into the wall surrounding the lumen without takeup or the need to avoid the lumen upstream, an unpiped magnetized or impasse-jacket is placed to encircle the target segment.

To treat the esophagus, the ferrofluid is administered in food; to treat the airway and lungs, administration is in the form of an aerosol where the circumtracheal or circumbronchial jacket diverts the magnetically susceptible nanoparticles from the aerosol against and into the lumen wall (see, for example, Tewes, F., Ehrhardt, C., and Healy, A. M. 2014. “Superparamagnetic Iron Oxide Nanoparticles (SPIONs)-loaded Trojan Microparticles for Targeted Aerosol Delivery to the Lung,” European Journal of Pharmaceutics and Biopharmaceutics 86(1):98-104; Hasenpusch, G., Geiger, J., Wagner, K., Mykhaylyk, O., Wiekhorst, F., and 6 others 2012. “Magnetized Aerosols Comprising Superparamagnetic Iron Oxide Nanoparticles Improve Targeted Drug and Gene Delivery to the Lung,” Pharmaceutical Research 29(5):1308-1318; Rudolph, C., Gleich, B., and Flemmer, A. W. 2010. “Magnetic Aerosol Targeting of Nanoparticles to Cancer: Nanomagnetosols,” Methods in Molecular Biology 624:267-280; Dames, P., Gleich, B., Flemmer, A., Hajek, K., Seidl, N., Wiekhorst, F., Eberbeck, D., and 6 others 2007. Targeted Delivery of Magnetic Aerosol Droplets to the Lung,” Nature Nanotechnology 2(8):495-499).

If takeup is to be along the esophagus, the usually superparamagnetic particle bound drug (see, for example, Barrefelt, Å., Saghafian, M., Kuiper, R., Ye, F., Egri, G., and 6 others 2013. “Biodistribution, Kinetics, and Biological Fate of SPION Microbubbles in the Rat,” International Journal of Nanomedicine 8:3241-3254. Ling, D. and Hyeon, T. 2013. “Chemical Design of Biocompatible Iron Oxide Nanoparticles for Medical Applications,” Small 9(9-10):1450-1466; Gu, L., Fang, R. H., Sailor, M. J., and Park, J. H. 2012. “In Vivo Clearance and Toxicity of Monodisperse Iron Oxide Nanocrystals,” ACS [American Chemical Society] Nano 6(6):4947-4954; Wahajuddin and Arora, S. 2012. “Superparamagnetic Iron Oxide Nanoparticles: Magnetic Nanoplatforms as Drug-carriers,” International Journal of Nanomedicine 7:3445-3471; Xu, C., and Sun, S. 2013. “New Forms of Superparamagnetic Nanoparticles for Biomedical Applications,” Advanced Drug Delivery Reviews 65(5):732-743; Guo, L., Liu, G., Hong, R. Y., and Li, H. Z. 2010. “Preparation and Characterization of Chitosan Poly(acrylic Acid) Magnetic Microspheres,” Marine Drugs 8(7):2212-2222) is administered in a bolus of food formulated to avoid breakdown when masticated and mixed with the oral enzymes and bacteria. Any toxic residue incurred with state of the art iron oxide nanoparticles is addressed below in the sections entitled Hybrid Impasse and Extraction jackets and Extraction jackets under Description of the Preferred Embodiments of the Invention.

If takeup is to take place in the gut, then the bolus must be formulated to withstand breakdown when exposed to the gastric juice. When an orally administered drug is injected or infused for takeup into the wall of a particular segment of a vessel without takeup or the need to avoid the lumen upstream then an impasse-jacket is placed about the segment of the blood vessel to be treated. When, however, proximal portions of the ductus ought not to be exposed to the drug, such as when the drug is radioactive or would injure healthy tissue, piping the drug directly to the segment where it is to be taken up is accomplished by means of a side-entry jacket. If takeup within the segment is spontaneous or metabolic without the need for magnetic force, or the jacket is proximodistally positioned to target a particular supply territory, or the jacket is used to form a shunt or bypass, then the addition of a magnetic layer is unnecessary.

The jacket used then is of the kind shown in FIG. 2. When takeup is not spontaneous, a concentric magnetic layer is added to the jacket, creating a piped side-entry impasse-jacket as shown in FIGS. 3 thru 6, where FIG. 5 also includes a radiation shield, and FIG. 6 includes a radiation shield made of overlapping bits of tungsten each chemically isolated by encapsulation and bound together by an absorbable adhesive. The usually glycolic acid based adhesive is formulated to disintegrate over an interval past that requiring the shielding. Depending upon the site, this is accomplished through spontaneous or iatrogenic hydrolytic and or enzymatic degradation that disintegrates the shield once the radiation has abated. Disintegration of the temporary shield then exposes apertures in the jacket shell.

The use of shielding which disintegrates allows the drugs needed to avert the harmful effects of complete ductus enclosure which are targeted to the jacket to be terminated as soon as possible. In contrast to the shielding layer, the magnet layer includes the perforations or apertures. The toxic material of the magnet must, however, be encapsulated within a polymeric coat which lines the apertures. Regardless of the sequence of jacket assembly as to the inclusion of the apertures in the magnet as originally formed, once the jacket has been assembled, the apertures must be recoated to assure that the chemical isolation is complete. Drugs targeted to jackets are formulated to achieve the maximum safe concentration that optimizes efficacy, and thus allows the volume of the drugs to be minimized, extending the period for free movement pending the need for replenishment.

Should, however, a drug in the form of a ferrofluid be required in a larger volume, then provided the rate of accumulation allows it, the magnetized side-entry or piped impasse-jacket is provided with an extraction grating or grid, as delineated in copending continuation-in-part application Ser. No. 13/694,835, entitled Integrated System for the Infixion and Retrieval of Implants with or without Drug Targeting. At still larger volumes, continuous evacuation of an analyte, whether endogenous, introduced, chemical, or cellular, for example, is accomplished by means of a double arm type side-entry connection jacket such as that shown in FIG. 7 with longitudinally elongated side-connector and a bidirectional passive elastic flap type valve at the adductal or distal terminus, as will be described.

FIGS. 1-7 assume that a tacky hydrogel will be used to quench extravasation or exsanguination, so that valving is by introducing a separate valve. However, any of these jackets will often incorporate a flap-valve as integral with the trepan or die cutter leading edge of the side-connector used to excise a tissue plug from the side of the ductus. To minimize if not eliminate a thrombogenic space increasing the need for, an anticoagulant, the flap-valve is no more recessed abductally along the side-connector than is necessary to avoid interfering with the leading trepan or die cutter edge. When an integral flap valve and trepan are incorporated into the side-connector, recurved prongs shown as part number 20 used to prevent a separate valve-plug from migrating into the native lumen are omitted.

Where a double arm side-connector will be used to steer a guidewire or cabled device into the native lumen so that recurved prongs would interfere, but a valve at the opening in the side of the ductus is necessary or preferred, the double-arm side-connector incorporates a flap-valve integral with the trepan that serves as its surround. The flap valve acts as a barrier until the threshold force required to open it, which iatrogenic or responsive to the automatic function of the apparatus, exceeds the physiological or pathological forces present. The force or pressure required to pass through the valve may be directionally biased, so that along the venous tree, for example, extraction requires little more force than that of the blood pressure, whereas the pressure posed by flushing through the line will not be sufficient to drive the tacky hydrogel or flush water used into the ductus.

Obtaining directional bias usually involves little more than scoring the flaps horizontally toward the edge bonded to the die cutter surrounding frame. Ductus side-entry jackets are placed individually, regardless of interconnections established among these once placed. Jackets of the kind shown in FIG. 7 with two or more double arm side-connectors about the circumference incorporate in each such side-connector a die-cutting or trepan leading edge as the surrounding frame of the flap-valve. When connected in series, jackets with a circumferential or unitized trepan and flap-valve that extended entirely about the circumference could be connected to the aspiration pump to cut a segment out of the lumen wall. However, even with cutting edges running parallel to the longitudinal axis of the native lumen at intervals about the circumference, the added complexity of extracting the circumferentially divided ring of tissue, and more particularly, the likelihood of eccentricities in hardness as of calcification in different arcs argues for separate placement of each jacket.

During placement along the vascular tree, the flap-valve prevents excessive exsanguination despite the use of an anticoagulant to prevent clotting. Both to accommodate the core of the electromagnet and reduce the pressure head-on force of flushing, the angle of the double arm connector with flap-valve situated toward the imaginary or virtual apex, shown in FIG. 13 thru 15 as eliminated to create a collection chamber or trap for accumulated debris extracted, is made as obtuse as practicable. Double arm side-connector jackets as shown in FIGS. 13 thru 15 facilitate 1. The extraction of magnetic particle-bound analytes or cells, 2. The passing of a guidewire or cabled device into the lumen in either direction, and 3. Expedite jacket to jacket connection in series, or chaining, whereby the outlet arm of the jacket just upstream is connected to the inlet arm of the jacket downstream in a continuous train or ‘daisy chain.’ This line can be used to flush through the line or deliver medication to each jacket in sequence.

Improbably, if numerous enough to eventually induce iron overload, the soft iron cores of the electromagnets are potentially carcinogenic, necessitating permanent encapsulation to isolate the iron from surrounding tissue (see, for example, Steegmann-Olmedillas, J. L. 2011. “The Role of Iron in Tumour Cell Proliferation,” Clinical and Translational Oncology 13(2):71-76; Toyokuni, S. 2009. “Role of Iron in Carcinogenesis: Cancer as a Ferrotoxic Disease,” Cancer Science 100(1):9-16; Galaris, D. and Pantopoulos, K. 2008. “Oxidative Stress and Iron Homeostasis: Mechanistic and Health Aspects,” Critical Reviews in Clinical Laboratory Sciences 45(1):1-23; Papanikolaou, G. and Pantopoulos, K. 2005. “Iron Metabolism and Toxicity,” Toxicology and Applied Pharmacology 202(2):199-211; Emerit, J., Beaumont, C., and Trivin, F. 2001. “Iron Metabolism, Free Radicals, and Oxidative Injury,” Biomedicine and Pharmacotherapy 55(6):333-339). Noble metal plating susceptible to pitting over time, plating must be supplemented with sputtering or vapor deposition of the metal used, or an encapsulating layer of a chemically stable polymer applied.

Double arm jackets used for low volume analyte extraction on an occasional basis can include a permanent magnet layer to detain the bound analyte, an external (extracorporeal) electromagnet used for extraction. However, for larger volume analyte extraction, the jackets incorporate a small electromagnet that faces a flap-valve to be described and shown in FIGS. 13 thru 15. A double arm extraction electromagnet-jacket side-connector primarily intended to facilitate steering in either direction as shown in FIGS. 13-15 usually has an opening and flap valve leading into the lumen of the ductus that is round, with the inner sides of the arms continued adductally to meet at an apex. When intended primarily for higher volume magnetic apheresis such a leukapheresis, the double arm connector, as shown in FIGS. 13-15, will usually truncate the inner sides of the arms to create a collection chamber or trap along the flush line that affords more space for the accumulation of debris as well as a landing for the small electromagnet, which is deenergized when the flush pump is actuated.

In distributed jacket extraction, the use of an outer body vest or harness to position a magnet in facing relation to each jacket, thereby drawing higher volume extractates such as leukocytes in leukaperesis into the wash out (flush out, flush through, extraction, purge) line to the reservoir should be considered only when internal placement poses too high a risk. The internal faces of the flap valve and that of the collection chamber are given extremely smooth surfaces, the adluminal faces of the flaps cambered, rounded, or bifold angles so as not to trap extractate particles, and made to open only when forced to do so by the magnet. Generally, this is accomplished by the force of the extracted particles against the luminal face of the flaps, although some slight magnetically susceptible material can be incorporated into the flaps.

Along the vascular tree, flap opening must be no more than necessary to pull out the extractate or blood will leak into the line necessitating more frequent flushing and reducing battery life. Flushing with a hydrogel containing heparin, for example ameliorates and complication due to an accumulation of thrombus. Radially asymmetrical, electromagnets must be stabilized in position and add weight, and if, the additional weight of the battery or batteries required. The apparatus preferrably functions automatically and constantly, during sleep and while bathing, the need for extraction or flushing limited to the clinic not consistent with this purpose. Division and spacing apart or distributing the jacket and magnets distributes the weight of the magnets and improves the degree of analyte removal.

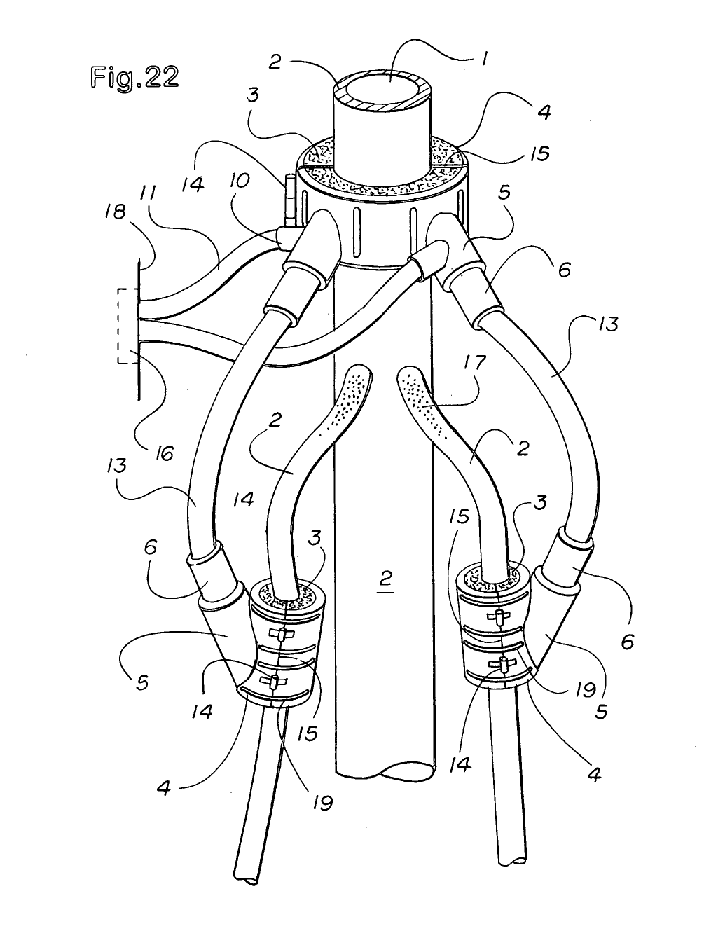

Whether electromagnets are used or used in some positions depends upon the relative weight as well as power consumption. A single arm inline port or clean-out local to the entry incision as shown in FIG. 22 with the arm directed adductally can serve as a piggyback style inline port to allow clean-out or the passage of a guidewire or cabled device during placement. Once the access incision is closed, access is through the pump-pack as shown in FIG. 31. Along an artery, the pulse may make it necessary to use two or more impasse-jackets at intervals. To allow paths for an external electromagnet to extract a carrier-bound radionuclide once depleted to a level safe for the adjacent tissue, a jacket with such a grid requires a radiation shield that disintegrates when the level of radiation decreases to the safe level. Shielded jackets necessitate the use of shielded lines and other elements of containment such as vials.

In some situations, the positioning within the lumen wall of a magnetically susceptible residue may be the primary object, the application of a radiofrequency alternated magnetic field used to heat the encircled segment, killing the cells of a tumor within the surrounding lumen wall, for example, by thermoplasty. In others, the impasse-jacket can be used to align the particulate to an invasive lesion such as a tumor from outside the ductus, while in others still, the opportunity for thermoplasty may arise incidentally, or as a collateral treatment modality. Extravascular patch or clasp-magnets can be placed to increase the field strength or to steer the susceptible particles. The need for continued delivery of a radioactive substance requires radiation shielding of jacket, fluid lines, and the other parts exposed to the radiation, and demands adjuvant treatment to avert the adverse sequelae of completely enclosing the ductus.

Such shielding if permanent induces deterioration of the substrate ductus, necessitating counteracting medication which can itself cause complications. The shielding is ordinarily made to disintegrate following depletion of a low dose rate radionuclide, for example, to a safe level. When the radiation is sent to a side-entry jacket with magnet layer for takeup local to the point of delivery, the supply vial or reservoir, jacket, and piping leading to the jacket must be enclosed within radiation shielding. When delivery is to an upstream level for takeup in the segment of the ductus lying between the entry level and the level where the drug-carrier is extracted, the intervening or treated segment must also be shielded.

Whereas a permanent shield normally consists of a solid layer of tungsten just inside the outer protective shell, which precludes the jacket from including openings, fenestra or apertures, a disintegrating shield consists of overlapping or imbricated particles of tungsten, each encapsulated within a nonbiodegradable polymeric shell applied outside a shell with openings so that complete enclosure-offsetting adjuvant medication need be given only over the period before the shield disintegrates. In the drawing figures, the need for each layer in a given jacket varies independently only to the extent that a simple junction type jacket such as that shown in FIG. 2 does not require a magnet layer, and unless the magnet layer is to attract a radioactive bound drug, neither is a radiation shielding layer.

A radiation shield layer must, however, by accompanied by a magnet layer, since the radioactive substance must not flow through the protected segment within the jacket to irradiate the tissue downstream. A radiation shield is required whenever a radioactive substance is to be delivered, regardless whether the substance is delivered in a ferrofluid that necessitates the use of a magnet layer, for example. In a disintegrating radiation shield, the particles are bonded together with an adhesive blend of polymers of the kind used to make absorbable suture according to the rate of depletion or half-life of the radionuclide. Once a safe level of depletion has elapsed, the particles, outside the outer shell, are free to drop away. To allow the jacket to open freely, the portion of the shield over the hinges is not bonded, allowing it to bulge out as the jacket is opened.

An unmagnetized ductus side-entry jacket can deliver a magnetic carrier bound drug for distribution to a number of separate impasse-jackets downstream where each successive jacket presents a somewhat greater field strength. The applicability to the skip lesions of ileocolitis, where steroids, for example, can do havoc, is obvious. Low dose rate radionuclides must be delivered through separate jackets each with magnetized extension and shielded from source vial or reservoir to the point of release. Such a jacket can be used to attract magnetically susceptible carrier particle-bound drugs radially outward, or centrifugally, from the lumen into and through the lesioned wall.

Lesions within the wall are therefore exposed to the drug or drugs. Many conditions require only the systemic, usually subcutaneous, delivery of drugs as typified by insulin pumps long on the market. In more complex situations, where the body affords no intrinsic adaptive response to compensate for a chemical imbalance, for example, several such jackets, each targeting a segment of a ductus, organ, or the region supplied by each segment, can be integrated into a hierarchically hard real time controlled prosthetic compensatory system, predictive or anticipatory as needed, to supplement and parallel that intrinsic, or physiological.

In a body area network consistent with free movement (see, for example, Darwish, A, and Hassanien, A. E. 2011. “Wearable and Implantable Wireless Sensor Network Solutions for Healthcare Monitoring,” Sensors (Basel) 11(6):5561-5595; Konstantas, D. 2007. “An Overview of Wearable and Implantable Medical Sensors,” Yearbook of Med Informatics 2007:66-69), diagnostic sensors provide feedback data used to automatically adjust the dosing and interval for the delivery of each drug. The sensors can be implanted ductus intramurally; inside the jackets; to line the jacket with a matrix gradient; straddle thereby to measure transmissivity through the ductus; attached at the body surface; or situated at remote locations. Situated without immediate incisive insertion as when forcing an electrode or probe into tissue, the formation of cicatricial (scar) tissue about the sensor causing its gradual desensitization is eliminated (see, for example, Karp, F. B., Bernotski, N. A., Valdes, T. I., Bohringer, K. F., and Ratner, B. D. 2008. “Foreign Body Response Investigated with an Implanted Biosensor by in Situ Electrical Impedance Spectroscopy,” IEEE Sensors Journal 8(1): 104-112). However, sensors can be implanted anywhere pertinent symptoms can be detected.

This may or may not be within or adjacent to tissue the ductus supplies, adjacent or encircling another type ductus that might exhibit symptoms, or considerably up or downstream from the side-entry jacket supported (see, for example, Vasylieva, N. and Marinesco, S. 2013. “Enzyme Immobilization on Microelectrode Biosensors,” in Marinesco, S. and Dale N. (eds.), Microelectrode Biosensors. Neuromethods, Volume 80, pages 95-114, New York, N.Y.: Humana Press; Palaniswamy, C., Mishkin, A., Aronow, W. S., Kalra, A., and Frishman, W. H. 2013. “Remote Patient Monitoring in Chronic Heart Failure,” Cardiology in Review 21(3):141-150; Dey, R. S., Bera, R. K., and Raj, C. R. 2013. “Nanomaterial-based Functional Scaffolds for Amperometric Sensing of Bioanalytes. Analytical and Bioanalytical Chemistry 405(11):3431-3448; Kotanen, C. N., Moussy, F. G., Carrara, S., and Guiseppi-Elie, A. 2012. “Implantable Enzyme Amperometric Biosensors,” Biosensors and Bioelectronics 35(1):14-26; Iost, R. M., da Silva, W. C., Madurro, J. M., Madurro, A. G., Ferreira, L. F., and Crespilho, F. N. 2011. “Recent Advances in Nano-based Electrochemical Biosensors: Application in Diagnosis and Monitoring of Diseases,” Frontiers in Bioscience (Elite Edition) 3:663-689; Tallaj, J. A., Singla, I., and Bourge, R. C. 2011. “Implantable Hemodynamic Monitors,” Cardiology Clinics 29(2):289-299; Merchant, F. M., Dec, G. W., and Singh, J. P. 2010. “Implantable Sensors for Heart Failure,” Circulation, Arrhythmia, and Electrophysiology 3(6):657-667)

This not only places the sensor closer to the tissue eventually affected but alleviates the requirement for extreme miniaturization, crowding, and the risk of clogging (see, for example, Goetzinger, D. J. and Najafi, N. 2010. Delivery System, Method, and Anchor for Medical Implant Placement, U.S. Pat. No. 7,860,579). Moreover, in conformation, dimensions, and principle of operation, situation of sensors apart from the jacket or jackets supported allows sensor implant design freedom and surface treatment to minimize if not avert adverse tissue responses or foreign body reactions (see, for example, Vaddiraju, S., Tomazos, I., Burgess, D. J., Jain, F. C., and Papadimitrakopoulos, F. 2010. “Emerging Synergy between Nanotechnology and Implantable Biosensors: A Review,” Biosensors and Bioelectronics 25(7):1553-1565). Generally, only the sensing elements of a sensor are implanted, any associated electronics relegated to the pump-pack. Any therapeutic or diagnostic substance in fluid form, to include nanomedical and superparamagnetic, can be targeted to any level of any type ductus which can be jacketed with sensors local or remote.

Implantable sensors and microsensors, under study for decades, are suitable or embody principles of operation suitable for adaptation to allow placement at a distance from the side-entry jacket or jackets whose effect the sensor is used to measure (see, for example, Abraham, W. T. 2013. “Disease Management: Remote Monitoring in Heart Failure Patients with Implantable Defibrillators, Resynchronization Devices, and Haemodynamic Monitors,” Europace 15 Supplement 1:i40-i46; Lee, S. H., Sung, J. H., and Park, T. H. 2012. “Nanomaterial-based Biosensor as an Emerging Tool for Biomedical Applications,” Annals of Biomedical Engineering 40(6):1384-1397; Abraham, W. T., Adamson, P. B., Bourge, R. C., Aaron, M. F., Costanzo, M. R., Stevenson, L. W., Strickland, W., and 7 others 2011. “Wireless Pulmonary Artery Haemodynamic Monitoring in Chronic Heart Failure: A Randomised Controlled Trial,” Lancet 377(9766):658-666, erratum 2012 379(9814):412; Adamson, P. B., Abraham, W. T., Aaron, M., Aranda, J. M. Jr., Bourge, R. C., and 4 others 2011. “CHAMPION Trial Rationale and Design: The Long-term Safety and Clinical Efficacy of a Wireless Pulmonary Artery Pressure Monitoring System,” Journal of Cardiac Failure 17(1):3-10; Iost, R. M., da Silva, W. C., Madurro, J. M., Madurro, A. G., Ferreira, L. F., and Crespilho, F. N. 2011. “Recent Advances in Nano-based Electrochemical Biosensors: Application in Diagnosis and Monitoring of Diseases,” Frontiers in Bioscience (Elite Edition) 3:663-689; Song, H. S. and Park, T. H. 2011. “Integration of Biomolecules and Nanomaterials: Towards Highly Selective and Sensitive Biosensors,” Biotechnology Journal 6(11):1310-1316; Fritz, B., Cysyk, J., Newswanger, R., Weiss, W., and Rosenberg, G. 2010. “Development of an Inlet Pressure Sensor for Control in a Left Ventricular Assist Device,” American Society for Artificial Internal Organs Journal 56(3):180-185; Carlson, R. E., Weller Roska. R. L., and Brose, S. A. 2009. Sensors Employing Combinatorial Artificial Receptors, WO 2009073625 A1/US 2009/0203980 A1; Verdejo, H. E., Castro, P. F., Concepción, R., Ferrada, M. A., Alfaro, M. A., Alcaíno, M. E., Deck, C. C., and Bourge, R. C. 2007. “Comparison of a Radiofrequency-based Wireless Pressure Sensor to Swan-Ganz Catheter and Echocardiography for Ambulatory Assessment of Pulmonary Artery Pressure in Heart Failure,” Journal of the American College of Cardiology 50(25):2375-2382; Bullister, E., Reich, S., D'Entremont, P., Silverman, N., and Sluetz, J. 2001. “A Blood Pressure Sensor for Long-term Implantation,” Artificial Organs 25(5):376-379; Nitta, S., Katahira, Y., Yambe, T., Sonobe, T., Hayashi, H., and 5 others 1990. “Micro-pressure Sensor for Continuous Monitoring of a Ventricular Assist Device,” International Journal of Artificial Organs 13(12):823-829).

A body area network is the optimal intracorporeal complement to a wireless body area network. Feedback sensors can be diagnostic, reporting the level of the analyte or metabolite before and after drug delivery, with or without accompanying sensors to indicate the drug level. Where a sensor or sensors immediately associated with the jacket cannot accommodate the analytical technique, the apparatus is capable of drawing and delivering a sample of the lumen contents to an analytical device contained within the pump-pack or to a technician when needed (see, for example, Marko-Varga, G. A., Nilsson, J., and Laurell, T. 2004. “New Directions of Miniaturization within the Biomarker Research Area,” Electrophoresis 25(21-22):3479-3491).

The cost in a lack of suitable means for introducing therapeutic substances directly into ductus is apparent from the limitation in the rapid advancement in physiological sensors and biotelemetry, or sensor implants, to diagnostic monitoring and a conspicuous absence of parallel and immediate therapeutic ability through the use of effector or motor implants (see, for example, Meng, X., Browne, K. D., Huang, S.-M., Mietus, C., Cullen, D. K., Tofighi, M.-R. and Rosen, A. 2013. “Dynamic Evaluation of a Digital Wireless Intracranial Pressure Sensor for the Assessment of Traumatic Brain Injury in a Swine Model”, IEEE Transactions on Microwave Theory and Techniques 61(1)316-325; Cao, H., Landge, V., Tata, U., Seo, Y.-S., Rao, S., Tang, S.-J., Tibbals, H. F., Spechler, S., and Chiao, J.-C. 2012. “An Implantable, Batteryless and Wireless Capsule with Integrated Impedance and pH Sensors for Gastroesophageal Reflux Monitoring,” IEEE Transactions on Biomedical Engineering 59(11):3131-3139; Mahfouz, M., To, G., and Kuhn, M. 2011. “No Strings Attached,” IEEE Microwave Magazine 12(7): S34-S48; Zhang, F., Hackwoth, S. A., Liu, X., Li, C., and Sun, M. 2010. “Wireless Power Delivery for Wearable Sensors and Implants in Body Sensor Networks,” Conference Proceedings IEEE Engineering in Medicine and Biology Society 692-695; Poon, A. S. 2009. “Miniaturization of Implantable Wireless Power Receiver,” Conference Proceedings IEEE Engineering in Medicine and Biology Society: 3217-3220; Young, D. J. 2009 “Wireless Powering and Data Telemetry for Biomedical Implants,” Conference Proceedings IEEE Engineering in Medicine and Biology Society 3221-3224; Valdastri, P., Rossi, S., Menciassi, A., Lionetti, V., Bernini, F., Recchia, F. A., and Dario, P. 2008. “An Implantable ZigBee Ready Telemetric Platform for in Vivo Monitoring of Physiological Parameters,” Science Direct. Sensors and Actuators A Physical 142(1):369-378).

Instead of ambulatory means for continuously, automatically, immediately and autonomously responding to the condition sensed, readings are relayed to a specialist for review and the writing of a prescription. The delay in this process is a conspicuous deficiency. Usually, the overall sequence in which the drugs are delivered to each side-entry jacket is maintained whether the jackets are placed along a single ductus, or where interrelated and interdependent organ systems are affected, along ductus belonging to different organ systems. In more complex situations, nested levels of program control, or nodes, each supporting a jacket incorporating symptom and remedial substance delivery and level measuring sensors, are used.

The nodes usually consist of time division multiplexed cores of a multicore microcontroller (see, for example, Schoeberl, M., Brandner, F., Sparse), J., and Kasapaki, E. 2012. “A Statically Scheduled Time-Division-Multiplexed Network-on-Chip for Real-Time Systems,” pages 152-160, Networks on Chip (NoCS), 2012 Sixth IEEE/ACM International Symposium on, Lyngby, Denmark, available at http://www.jopdesign.com/doc/s4noc.pdf; Sparso, J. 2012. “Design of Networks-on-Chip for Real-Time Multi-processor Systems-on-Chip,” in 12th International Conference on Application of Concurrency to System Design, Hamburg, Germany pages 1-5; Paukovits, C. and Kopetz, H. 2008. “Concepts of Switching in the Time-triggered Network-on-chip,” in Proceedings of the 14th IEEE International Conference on Embedded and Real-Time Computing Systems and Applications (RTCSA '08), Kaohsiung City, Taiwan, Republic of China, pages 120-129; Schoeberl, M. 2007. “A Time-triggered Network-on-chip,” in International Conference on Field-Programmable Logic and its Applications (FPL 2007), pages 377-382; Kopetz, H. and Bauer, G. 2003. “The Time-triggered Architecture,” Proceedings of the IEEE, 91(1):112-126; Wiklund, D. and Liu, D. 2003. “SoCBUS: Switched Network on Chip for Hard Real Time Embedded Systems,” in Proceedings of the 17th International Symposium on Parallel and Distributed Processing (IPDPS '03), Los Alamitos, Calif., IEEE Computer Society, page 78a), which communicate with the higher node or core programmed to function as master or ‘supreme’ node or controller, and if pertinent, directly with one another.

The distribution of control between the brain and subordinate circuits and ganglions a salient feature of the nervous system, such a hierarchical scheme may be seen as analogous to the relation between the motor cortex and subsidiary or more localized control circuits in the spinal cord, for example. Here such a control tree receives feedback from the sensors associated with each jacket to continuously adjust and coordinate the dosing of the drug delivery program in detail, and overall. Subordinate or ‘intimal’ nodes can consist of cores dedicated to different drugs, different jackets, and/or jacket sets in different locations. This surveillance and therapy continues around the clock, independently of the mental competence or state of wakefulness of the patient and without the need for a professional attendant.

A magnetized collar encircling a ductus, or impasse-jacket, will draw any magnetically susceptible particle-bound drug outward and through the surrounding lumen wall. The position of the side-entry jacket thus targets the level along the ductus, and the magnetic force targets the intramural lesion in that segment. A lesion such as an atheroma is therefore ‘washed over’ and penetrated by the drug, which can be released continuously or at intervals throughout the day. That nonemergency and emergency treatment continues around the clock regardless of the location or mental state of the patient conscious or unconscious, presents benefits deriving from immediacy of symptom detection and initiation of drug delivery. When the number of drugs to be loaded into the pump-pack based upon the preliminary general diagnosis can be accommodated, the need for a specific diagnosis as to the condition provoking the immediate crisis is eliminated.

Vasospasm of an epicardial coronary artery, the left anterior descending shown in FIG. 16, for example, can be interdicted coincidentally with detection of the initial vasotonic mechanical, electrical, and/or chemical indicators, just as, if not before, overt pain is experienced, nitrates, for example, having already been targeted at the site of spasm when sublingual absorption would just have been initiated. The object is to avert spasm before it is experienced as pain, much less can result in ischemic injury. This immediacy applied to the placement of a side-entry jacket at or near to the source of the disease process can similarly interdict symptoms before or critically before these can take effect. A prosthetic disorder response system best mimics or parallels that innate, and a bionic system best simulates an innate system.

Depending upon its mechanism and consistent with minimizing trauma, any drug to treat a more serious chronic condition that would be more efficacious were it delivered more closely to an innate response mechanism is a candidate for delivery thus. For example, insulin is delivered as near to the normal source as possible, before processing in the liver. Because it best parallels the natural process, a side-entry jacket applied to the hepatic portal vein to deliver an insulin will best simulate normal function in providing first-pass insulin to the liver. In this, the delivery of the insulin as close to the normal origin as practicable should most quickly avert the inducement of the symptoms associated with an abnormal level of blood glucose. Glucose sensors implanted in various locations signal an abnormal elevation, whereupon the system initiates insulin delivery. Chronic forms of hepatitis for which effective drugs are available are likewise introduced through the portal vein, thereby reducing side effects and interactions.

In this way, the abnormal condition whether due to insulin resistance, nonabsorption, or any other reason for gauging low is detected and responded to instantly, not after a delay to test the blood and disperse from the subcutaneous injection site. Existing pumps deliver insulin subcutaneously, delaying dispersal throughout the systemic circulation. Since with a prosthetic response system such as that to be described, insulin levels can be monitored continuously, a failure of takeup for any reason, to include lipdystrophy, can be negotiated by programming to effect delivery through an alternative port, which can be placed in anticipation of such an eventuality need should it appear. Using the approach described herein, elevated glucose is detected and responded to as close to the natural location and in the same if not less amount of time as occurs with normal function.

Direct venous infusion also avoids such delay as well as the adverse consequences of injection that is intramuscular rather than subcutaneous (see, for example, Henriksen, J. E., Djurhuus, M. S., Vaag, A., Thye-Rønn, P., Knudsen, D., Hother-Nielsen, O., and Beck-Nielsen, H. 1993. “Impact of Injection Sites for Soluble Insulin on Glycaemic Control in Type 1 (Insulin-Dependent) Diabetic Patients Treated with a Multiple Insulin Injection Regimen,” Diabetologia 36(8):752-758; Vaag, A., Handberg, A., Lauritzen, M., Henriksen, J. E., Pedersen, K. D., and Beck-Nielsen, H. 1990. “Variation in Absorption of NPH [Neutral Protamine Hagedorn] Insulin Due tolntramuscular Injection,” Diabetes Care 13(1):74-76; Vaag, A., Pedersen, K. D., Lauritzen, M., Hildebrandt, P., and Beck-Nielsen, H. 1990. Intramuscular versus Subcutaneous Injection of Unmodified Insulin: Consequences for Blood Glucose Control in Patients with Type 1 Diabetes Mellitus,” Diabetic Medicine 7(4):335-342; Frid, A., Gunnarsson, R., Güntner, P., and Linde, B. 1988. “Effects of Accidental Intramuscular Injection on Insulin Absorption in IDDM [Insulin Dependent (Type 1) Diabetes Mellitus],” Diabetes Care 11(1):41-45) but is unsuited to free movement.

For the purpose of minimizing the risk of insulin lipodystrophy, not just a complication in itself but interfering with absorption of the drug, delivery can be rotated among ports of the type to be described where each port is implanted at a different location at the body surface. The reason is that even though using such a port, the insulin is never in contact with tissue, insulin lipodystrophy can present with continuous insulin infusion, indicating that this complication may not depend upon contact (see, for example, Mokta, J. K., Mokta, K. K., and Panda, P. 2013. “Insulin Lipodystrophy and Lipohypertrophy,” Indian Journal of Endocrinology and Metabolism 17(4):773-774; Ihlo, C. A., Lauritzen, T., Sturis, J., Skyggebjerg, O., Christiansen, J. S., and Laursen, T. 2011. “Pharmacokinetics and Pharmacodynamics of Different Modes of Insulin Pump Delivery. A Randomized, Controlled Study Comparing Subcutaneous and Intravenous Administration of Insulin Aspart,” Diabetic Medicine 28(2):230-236; Radermecker, R. P., Piérard, G. E., and Scheen, A. J. 2007. “Lipodystrophy Reactions to Insulin: Effects of Continuous Insulin Infusion and NewInsulin Analogs,” American Journal of Clinical Dermatology; 8(1):21-28; Johansson, U. B., Amsberg, S., Hannerz, L., Wredling, R., Adamson, U., Arnqvist, H. J., and Lins, P. E. 2005. Impaired Absorption of Insulin Aspart from Lipohypertrophic Injection Sites,” Diabetes Care 28(8):2025-2027; Raile, K., Noelle, V., Landgraf, R., and Schwarz, H. P. 2001. “Insulin Antibodies are Associated with Lipoatrophy but also with Lipohypertrophy in Children and Adolescents with Type 1 Diabetes,” Experimental and Clinical Endocrinology and Diabetes 109(8):393-396). Since the drug is piped through the body surface, a lipodystrophic response, although to be avoided as adverse in itself, cannot, however, interfere with delivery and takeup.

Continuously adaptive response with the capability for predictive or anticipatory control is best supported by sensors implanted in different sites and tissues. Numerous glucose sensor techniques are under development (see, for example, Scognamiglio, V. 2013. “Nanotechnology in Glucose Monitoring: Advances and Challenges in the Last 10 Years,” Biosensors and Bioelectronics 47:12-25. Balaconis, M. K. and Clark, H. A. 2013. “Gel Encapsulation of Glucose Nanosensors for Prolonged in Vivo Lifetime,” Journal of Diabetes Science and Technology 7(1):53-61; Heo, Y. J. and Takeuchi, S. 2013. “Towards Smart Tattoos: Implantable Biosensors for Continuous Glucose Monitoring; Advanced Healthcare Materials 2(1):43-56. Hu, R., Stevenson, A. C., and Lowe, C. R. 2012. “An Acoustic Glucose Sensor,” Biosensors and Bioelectronics 35(1):425-428; Billingsley, K., Balaconis, M. K., Dubach, J. M., Zhang, N., Lim, E., Francis, K. P., and Clark, H. A. 2010. “Fluorescent Nano-optodes for Glucose Detection,” Analytical Chemistry 82(9):3707-3713; Cash, K. J. and Clark, H. A. 2010. “Nanosensors and Nanomaterials for Monitoring Glucose in Diabetes,” Trends in Molecular Medicine 16(12):584-593; Domschke, A. M. 2010. “Continuous Non-invasive Ophthalmic Glucose Sensor for Diabetics,” Chimia (Aarau) 64(1-2):43-44).

The jacket to deliver the insulin is situated as close as practicable to the pancreas as the normal source of insulin. The hepatic portal vessels of the mesentery that normally transport nutrients other than amino acids and simple sugars absorbed directly through the gut therefore function normally, albeit without the normal level of insulin. However, the blood that flows through these vessels is substantially void of glucose, so that the fact that the insulin is delivered downstream through the jacket is without adverse effect; the insulin is not needed until the liver is reached. The relatively normal position for the release of insulin reduces fluctuations in blood glucose to within normal levels without interfering with first-pass processing of drugs.

This eliminates the need for the subcutaneous injection of an insulin, with a time lag that compared to central release, is considerable. No subcutaneous injection, oral antihyperglycemic drug, or inhaled formulation of insulin, metformin, or any other drug can approximate the ability of an instant response system with multisensor input under hierarchical control to modulate blood glucose to within the normal range. Insulin overdose or overproduction should it arise is remediable by releasing metalloprotease insulin-degrading enzyme (insulysin, insulinase) or glucose directly into the hepatic portal vein or glucose into the bloodstream.

The initiation of such treatment after diabetes mellitus has set in, strictly exemplary and cited for its prevalence, typifies treatment by such means of a disease cascade, here hyperglycemia that would have led to chronic vascular inflammation, nephropathy, retinopathy, medial calcification, and abnormal hematology, leading in turn to thrombosis and cerebral and myocardial infarction. Transient diabetes such as gestational where future pregnancies are not anticipated is not treated thus. If necessary, such treatment when desired due to more than one transient disease process can be implemented using components that are absorbed or disintegrate.

Chronic conditions involving aberrant insulin production, to include insulin rebound (posthypoglycemic hyperglycemia, chronic Somogyi rebound, Somogyi effect), idiopathic postprandial syndrome (reactive hypoglycemia, idiopathic hypoglycemia) are instantly and automatically regulated back to within normal limits, as are comparable transients in any comorbid disease also under treatment by the system. By eradicating the early symptoms of diabetes—glycosuria inducing polyuria, polydipsia, fatigue, blurred vision, hyperinsulinemia, and dyslipidemia; the diverse sequelae that ensue over time, such as peripheral numbness neuropathy, retinopathy, nephropathy, vascular disease (atherosclerosis, angina, cardiomyopathy), venous thrombosis, predisposition to infection, and so on, that often lead to death, are precluded from originating.

Since “An episode of hyperglycemia in a patient with diabetes can require hours or days of intensive therapy to return the blood glucose value to normal,” (Klonoff, D. C. 2007. “The Benefits of Implanted Glucose Sensors,” Journal of Diabetes Science and Technology 1(6):797-800), the ability to prevent the entry of excessive glucose into the systemic circulation under predictive or anticipatory control using sensor implant inputs from different locations and tissues rather than secondarily or reactively having to remedy the excess with medication is especially beneficial.

When the disease process or a sequel induced by it is not focal so that it can be targeted, such as atherosclerosis sequelary to diabetes, a background level of evolocumab, ezetimibe, a statin, and apixaban (see for example, Agnelli, G., Buller, H. R., Cohen, A., Curto, M., Gallus, A. S., Johnson, M., Porcari, A., Raskob, G. E., Weitz, J. I.; with 397 collaborators 2013. “Apixaban for Extended Treatment of Venous Thromboembolism,” New England Journal of Medicine 368(8):699-708;), for example, can be circulated, usually at a lower dose. Multicomponental disease or comorbidity, whether the components may be ascribed to a distinct causal chain, each is produced by etiologically distinct conditions, or some combination thereof are treated by placement of the jacket or magnetized jacket, with or without clasp-magnet support, at or as close to the origin of each disease component as the avoidance of trauma will allow.

Often, this will involve situating the jacket at the level along the supporting vessel or vessels to target the supply territory where the disease arises or exerts an effect. In this way, the components of comorbidities whether cooriginal and sequential or substantially unrelated can be treated. As compared to a systemic dose, targeted medication is small, even when more concentrated than might be allowed to circulate. Moreover, in situations where the condition is systemic with only salient lesions targeted, a background dose for circulation can be significantly reduced. The ability to reduce without eliminating a background systemic level of a steroid, for example, can significantly alleviate its adverse side effects.

Moreover, because such a system is able to treat multiple disease conditions related or unrelated, immediately and simultaneously, it is able to suppress not only diabetes and collateral disease for which diabetes is responsible, but to treat disease that induces or facilitates the development of hyperglycemia and diabetes, such as Alström syndrome, Werner's syndrome, acanthosis nigrans, pineal hyperplasia syndrome, ataxia telangiectasia, lipodystrophic disorders that induce insulin resistance, cystic fibrosis, pancreatitis, and hemochromatosis; or, endocrinopathies, such as Cushing syndrome, acromegaly, and pheochromocytoma (see, for example, Kishore, P. 2012. “Diabetes Mellitus (DM),” the Merck Manual online at http://www. Merckmanuals.com/professional/endocrine_and_metabolic_disorders/diabetes_mellitus_and_disorders_of_carbohydrate_metabolism/diabetes_mellitus_dm.htm).

Acting over time, the continuous attraction of a periductally, hence, immediately placed magnetized collar to act upon superparamagnetic drug-carrier nanoparticles or microparticles can replace the need for a powerful and heavier patch-magnet or electromagnet placed subcutaneously or held in position by an extracorporeal harness. Where the anatomy does not afford the clearance required to achieve the field force or pull strength necessary, separate intracorporeal patch-magnets, or neodymium magnets mounted on a tissue clasp or an electromagnet mounted at the body surface are used to support the magnetized jacket.

Ordinarily the magnetized collar or jacket is magnetized to present longitudinally and radially symmetrical gradient field strength along the longitudinal axis of the encircled ductus. Hypothetically, the attraction from entirely about the circumference therefore cancels out along the axis, although the asymmetries of real ductus negate this. Due to movement and the imperfect radial symmetry of the lumen, however, magnetic particles never remain centered thus long enough to escape being attracted to one side or the other at every level. However, a separate magnetized collar, or impasse-jacket, or one integrated into a ductus side-entry jacket, can be made to match or complement the eccentricity of the lesion.

For example, an arc can be unmagnetized or magnetized less strongly. This is usually accomplished by introducing an insert made of a nonmagnetic material such as a plastic into the arcuate gap or by magnetizing the arc separately from the rest. When takeup of a magnetically nonsusceptible or nonmagnetic drug is not complete and the residue is to be prevented from flowing past a certain level, provided a reversal agent or antagonist is available, a segment of the ductus can be targeted for treatment by delivering the reversal agent through a second simple junction type ductus side-entry jacket or releasing the agent from an impasse-jacket at the level downstream.

Such action with an impasse-jacket is best spontaneous due to the intrinsic chemical relationship of the reversal agent to the residue, so that it responds to the residue without the need for further action. When used for the direct targeted delivery of drugs, a primary object in the use of side-entry connection jackets is to align drug delivery with network feedback, if necessary, by means of a hierarchical control system. Thereby, automatic and immediate targeted point drug therapy is achieved—not just diagnostics at one or multiple sites in the body so that the clinician remains limited to drug delivery that is neither immediate nor targeted. If the patient is not to be bedridden or the condition is chronic, a number of needled catheters cannot be used. Ductus side-entry connection jackets afford secure connection to the ductus, and in so doing, enable not just single point direct-to-ductus drug delivery, but the implementation of such a prosthetic supplementary disease-process compensation system.

The ductus side-entry connection pump-pair and jacket sets to be described thus make possible the targeted delivery of drugs through automatic response that is immediate. Were the condition to exceed the range of adjustment for which the system had been set, the exigent readings can be transmitted to a clinician able to adjust the dosing by remote control. Hierarchical control has been available for decades; however, with no means for safely converging with ductus through a secure junction, the relation of hierarchical networked feedback to automatic drug delivery has remained elusive. Because the sensors are associated with collocated means for the targeting of drugs to the location respective of each, immediate and automatic remedial drug delivery, not just information as to the status of the patient, are obtained.

This immediacy of therapy and not just a providing of diagnostics can be a critical factor in the response to a medical emergency Such a system is able to continuously monitor the level of an analyte and initiate and/or adjust the delivery rate of a corrective drug passed through the jacket and into the ductus directly from an ambulatory pump. Many drugs are affected by interaction with constituents of food, other drugs, and circadian factors. The anticoagulant action of warfarin, for example, is reversed in proportion to the intake in food of vitamin K₁, necessitating periodic testing of clotting time (prothrombin ratio; international normalization ratio). Implantation allowing extreme miniaturization in sensors over external meters for home use currently available, prothrombin, vitamin K₁, and other diagnostic sensor probe readings will be relegated to apparatus small enough to implant rather than to manipulate.

Such a system is able to assess dose sufficiency, and warfarin taking effect after a significant interval, extrapolate from short term deviation to deliver the adjusted dose at or close to the optimal time. Circadian circumstances calling for adjustment in the dose are instantly responded to throughout the day. Such a system is able to ‘learn,’ making it possible for control over drug delivery to be anticipatory. An unmagnetized or simple side-entry connection jacket can be placed about a native or transplanted tubular anatomical structure (conduit, ductus) to allow a synthetic tube, or catheter, or an artificial or tissue-engineered vessel to open directly into, without entering, a native or graft lumen, thereby significantly reducing the risks of clotting, hemorrhage, leakage, or accidental injury associated with existing means. A ductus side-entry jacket seals off potential paths of extravasation or leakage before the ductus wall is breached by excising a plug of tissue from its wall.

A simple drug delivery and lumen takeout application is shown in FIG. 16, which depicts a ductus side-entry jacket used to deliver drugs to an end-arterial coronary vessel, where entry into the systemic circulation as might result in adverse side effects or a residue that ought not to be released to other tissue is bypassed. The line shown in FIGS. 16 and 22 to a port shown in FIGS. 27 and 28 at the body surface can be tunneled subcutaneously as are ports used with insulin pumps. Less simple applications involve shunting around or straddling a diseased segment, tumor, or other lesion along the ductus, or the tissue the ductus supplies for exposure or for isolation from a drug or drugs. As shown in FIGS. 17 thru 22, ductus side-entry connection jackets can also be used to join synthetic shunts to native ductus, as will be further addressed. Applied to a coronary artery of a patient lacking a suitable autologous graft, placement thus avoids the need for a direct anastomotic junction between alloplastic and native termini and allows the delivery of anticoagulants.

The pumps are never used to move blood, so that hemolysis and its further complications of organ system injury, anemia, hepatic insufficiency, coagulopathy, and platelet damage cannot arise from this source (see, for example, Sapirstein, J. S. and Pierce, W. S. 1997. “Mechanical Circulatory Support,” Chapter 66 in Greenfield, L. J., Mulholland, M. W., Oldham, K. T., Zelenock, G. B., and Lillemoe, K. D. (eds.), Surgery: Scientific Principles and Practice, page 1554). In an application such as that depicted in FIG. 13, where blood is pumped through synthetic coronary bypasses by the heart, lining the bypasses with a textured surface of sintered titanium microspheres or textured polyurethane, for example, fosters the formation of a physiologically active pseudointima (Sapirstein, J. S. and Pierce, W. S. 1997 Op cit. page 1556; Dasse, K. A., Chipman, S. D., Sherman, C. N., Levine, A. H., and Frazier, O. H. 1987. “Clinical Experience with Textured Blood Contacting Surfaces in Ventricular Assist Devices,” Transactions of the American Society for Artificial Internal Organs 33(3):418-425).

Autologous vessels suitable for transplant or a tissue-engineered conduit may be unavailable. If available, either may benefit from if not require the structural support and secure attachment afforded beyond that allowed through the use of suture alone. Unlike an anatomosis, such a jacket also allows an automatic pump to deliver medication directly to the junction at precise intervals around the clock. Magnetized jackets are positioned at the sites of lesions within the wall of ductus to draw magnetically susceptible carrier bound drugs radially outward into the wall and any intramural or transmural lesion. More complicated applications involve alternately targeting and withholding a drug or drugs from consecutive segments along a ductus, such as ‘skip’ lesions along the gut, for example. In the arterial tree, alternation thus is to selectively skip over the ostia leading to an organ or organs or supply territories to be excluded.

A drug to be restricted thus is delivered at the level desired, and if to be taken up within the surrounding lumen wall, drawn radially outward by a side-entry jacket having a magnet layer extension as shown in FIGS. 4 thru-6, where those in FIGS. 5 and 6 also include radiation shielding. In the vascular tree, extension is ordinarily in the antegrade direction, and for uniform takeup along the jacket and lesion it encircles, gradually increased in field strength. With permanent magnets, this is prepared by magnetizing separate half-cylinders at progressively higher intensities, then sectioning the half-cylinders into half-rings and bonding progressively stronger half-rings into the half cylinders that comprise the jacket magnet. With electromagnets, this is accomplished by positioning the magnets in a linear array wherein each is separately wired, allowing the current transmitted to each consecutive magnet to be adjusted as necessary.

Whereas takeup of a drug delivered through the jacket side-connector is antegrade, for takeup of a carrier bonded drug delivered upsteam, the magnet can be extended in either or both the retrograde and antegrade directions. Permanent magnet jackets cannot simply be reversed in direction, because the field strength gradient is then reversed as well. Electromagnetic jackets are not limited thus. When the interval separating the entry level from the level for coercive takeup is longer, consecutive magnetized jackets or impasse-jackets are placed downstream. If the residue is to be prevented from passing past a certain level, then a second simple junction type jacket as shown in FIG. 2 can be used to release a reversal agent, or antagonist, at the level for cutoff. The residue must be innocuous to intervening tissue or tissue to which a passed ostium or orifice leads, and where appropriate, extraction by means of an extracorporeal electromagnet if necessary is feasible.

If such an agent is unavailable, takeup within the jacketed segment not a factor, and the delivering jacket is unmagnetized, then the drug used is magnetically susceptible particle bound prior to administration and a magnetized jacket used to extract it, the extraction or downsteam jacket ordinarily an unpiped, or impasse-jacket, with extraction grid or grating. Since the local anatomy may not afford adequate clearance for a surrounding collar of the thickness necessary to provide the field strength necessary to draw and hold the susceptible particles against the forces imposed by passing contents, one or more patch or clasp-magnets with rounded edges and corners and situated at angles with minimal encroachment upon the surrounding tissue may be needed to attain the required field strength.

Still other applications call for the extraction of endogenous or intrinsic substances which may be dispersed throughout the bloodstream, for example, the object being to accomplish continuous small scale, low volume magnetic or immuno-magnetic separation, noncentrifugation, nonsedimentation, and nonfiltration ambulatory apheresis with the need for extraction or resituation with the aid of an external or extracorporeal electromagnet as infrequently as possible. Provided means for bonding the superparamagnetic nanoparticle or microparticle drug-carrier to the target analyte are available, magnetic separation, which comprehends chemical properties, exceeds forms of separation based upon size and mass. Continuous analyte delivery and extraction for ambulatory apheresis is accomplished by means of a special higher volume analyte extraction jacket and a powerful magnet held in position at the body surface by a harness, such a jacket and harness to be described.

A relatively static means for extracting the targeted substance or cells without filtration or centrifugation, for example, significantly increases the potential for miniaturization essential for embodiment in an ambulatory system. In some instances, the targeting agent can be synthetic (see, for example, Zhang, M., He, X., Chen, L., and Zhang, Y. 2011. “Preparation and Characterization of Iminodiacetic Acid-functionalized Magnetic Nanoparticles and its Selective Removal of Bovine Hemoglobin,” Nanotechnology 22(6):065705; Zhang, M., Cheng, D., He, X., Chen, L., and Zhang, Y. 2010. “Magnetic Silica-coated Sub-microspheres with Immobilized Metal Ions for the Selective Removal of Bovine Hemoglobin from Bovine Blood,” Chemistry Asian Journal 5(6):1332-1340; Thomas, L., Mansour, V., Jain, R., Kulcinsk, i D., Loefler, K., Carter, C., and Hardwick, A. 1995. “Use of the CS-3000 Plus to Prepare Apheresed Blood Cells for Immunomagnetic Positive Cell Selection,” Journal of Hematotherapy 4(4):315-321)

When the endogenous or intrinsic substance or analyte eschews direct binding to magnetically susceptible carrier particles, binding is to a substance having an inherent selective affinity for the target endogenous substance. When introduced or reintroduced into the body, the magnetically susceptible carrier particle bound substance with an inherent affinity for a particular biological target or intracorporeal analyte seeks out and latches onto the target, making possible its extraction with the aid of a magnet. Substances that can be extracted from the blood by this means include cell types, inorganic atoms, and organic molecules, to include enzymes, hormones, other proteins, nucleotides, peptides, polypeptides, and introduced contrast, stain, dye, any of which may occasionally be radioactive.

The extracorporeal or in vitro binding of endogenous or other biological substances to magnetically susceptible particles in order to extract these or to extract analytes to which these bind with the aid of an external magnet as in immunomagnetic separation has been in use for decades and undergone much progress, warranting ambulatory implementation (see, for example, Chen, P., Huang, Y. Y., Hoshino, K., and Zhang, X. 2014. “Multiscale Immunomagnetic Enrichment of Circulating Tumor Cells: From Tubes to Microchips,” Lab on a Chip 14(3):446-458; Magbanua, M. J., and Park, J. W. 2013. “Isolation of Circulating Tumor Cells by Immunomagnetic Enrichment and Fluorescence-activated Cell Sorting (IE/FACS) for Molecular Profiling,” Methods [San Diego, Calif.] 64(2):114-118; Herrmann, I. K., Schlegel, A., Graf, R., Schumacher, C. M., Senn, N., Hasler, M., Gschwind, S., and 5 others 2013. “Nanomagnet-based Removal of Lead and Digoxin from Living Rats,” Nanoscale 5(18):8718-8723; Hoeppener, A. E., Swennenhuis, J. F., and Terstappen, L. W. 2012. “Immunomagnetic Separation Technologies,” Recent Results in Cancer Research 195:43-58; Brimnes, M. K., Gang, A. O., Donia, M., Thor Straten, P., Svane, I. M., and Hadrup, S. R. 2012. “Generation of Autologous Tumor-specific T Cells for Adoptive Transfer Based on Vaccination, in Vitro Restimulation and CD3/CD28 Dynabead-induced T Cell Expansion,” Cancer Immunology Immunotherapy. 61(8):1221-1231; Herrmann, I. K., Bernabei, R. E., Umer, M., Grass, R. N., Beck-Schimmer, B., and Stark, W. J. 2011. “Device for Continuous Extracorporeal Blood Purification Using Target-specific Metal Nanomagnets,” Nephrology, Dialysis, Transplantation 26(9):2948-2954; Yoshino, T., Maeda, Y., and Matsunag, T. 2010. “Bioengineering of Bacterial Magnetic Particles and Their Applications in Biotechnology,” Recent Patents in Biotechnology 4(3):214-225; Takahashi, M., Akiyama, Y., Ikezumi, J., Nagata, T., Yoshino, T., Iizuka, A., Yamaguchi, K., and Matsunaga, T. 2009. “Magnetic Separation of Melanoma-specific Cytotoxic T Lymphocytes from a Vaccinated Melanoma Patient's Blood Using MHC/Peptide Complex-conjugated Bacterial Magnetic Particles,” Bioconjugate Chemistry 20(2):304-309; Takahashi, M., Yoshino, T., Takeyama, H., and Matsunaga, T. 2009. “Direct Magnetic Separation of Immune Cells from Whole Blood Using Bacterial Magnetic Particles Displaying Protein G,” Biotechnological Progress 25(1):219-226; Yoshino, T., Hirabe, H., Takahashi, M., Kuhara, M., Takeyama, H., and Matsunaga, T. 2008. “Magnetic Cell Separation Using Nano-sized Bacterial Magnetic Particles with Reconstructed Magnetosome Membrane,” Biotechnology and Bioengineering 101(3):470-477; Witzens-Harig, M., Heilmann, C., Hensel, M., Kornacker, M., Benner, A., Haas, R., Fruehauf, S., and Ho, A. D. 2007. “Long-term Follow-up of Patients with Non-Hodgkin Lymphoma Following Myeloablative Therapy and Autologous Transplantation of CD34+-selected Peripheral Blood Progenitor Cells,” Stem Cells [Dayton, Ohio] 25(1):228-235; Chen, H., Bockenfeld, D., Rempfer, D., Kaminski, M. D., and Rosengart, A. J. 2007. “Three-dimensional Modeling of a Portable Medical Device for Magnetic Separation of Particles from Biological Fluids,” Physics in Medicine and Biology 52(17):5205-5218; Chen, H., Ebner, A. D., Bockenfeld, D., Ritter, J. A., Kaminski, M. D., Liu, X., Rempfer, D., and Rosengart, A. J. 2007. A Comprehensive in Vitro Investigation of a Portable Magnetic Separator Device for Human Blood Detoxification,” Physics in Medicine and Biology 52(19):6053-6072; “Chen, H., Kaminski, M. D., Liu, X., Mertz, C. J., Xie, Y., Tomo, M. D., and Rosengart, A. J. 2007. “A Novel Human Detoxification System Based on Nanoscale Bioengineering and Magnetic Separation Techniques,” Medical Hypotheses 68(5):1071-1079; Gu, H., Xu, K., Xu, C., and Xu, B. 2006. “Biofunctional Magnetic Nanoparticles for Protein Separation and Pathogen Detection,” Chemical Communications [Cambridge, England] 9:941-949; Ito, A., Shinkai, M., Honda, H., and Kobayashi, T. 2005. “Medical Application of Functionalized Magnetic Nanoparticles,” Journal of Bioscience and Bioengineering 100(1):1-11; Kuhara, M., Takeyama, H., Tanaka, T., and Matsunaga, T. 2004. “Magnetic Cell Separation Using Antibody Binding with Protein A Expressed on Bacterial Magnetic Particles,” Analytical Chemistry 76(21):6207-6213; Hardwick, R. A., Kulcinski, D., Mansour, V., Ishizawa, L., Law, P., and Gee, A. P. 1992. “Design of Large-scale Separation Systems for Positive and Negative Immunomagnetic Selection of Cells Using Superparamagnetic Microspheres,” Journal of Hematotherapy 1(4):379-386).

The ability to form secure junctions with vessels and miniaturization make possible the incorporation into a wearable pump-pack of apheretic techniques that allow magnetically nonsusceptible target substances to be bound to susceptible carriers endogenously in response to implanted sensor feedback under predictive control without the need for periodic return to the clinic for an invasive procedure. If the biological substance is bound to superparamagnetic nanoparticle or microparticle carriers, then once connected to the affinate, the analyte will have been rendered susceptible to a magnetic field. This makes it susceptible to seizure and resituation within the body by an impasse-jacket and extraction from the body with the aid of a periductal electromagnet. A local extraction jacket incorporating an electromagnet can accomplish either or both actions.

Numerous substances have been prepared for such application (see, for example, Barbucci, R., Giani, G., Fedi, S., Bottari, S., and Casolaro, M. 2012. “Biohydrogels with Magnetic Nanoparticles as Crosslinker: Characteristics and Potential Use for Controlled Antitumor Drug-delivery,” Acta Biomaterialia 8(12):4244-4252; Paulino, A. T., Pereira, A. G., Fajardo, A. R., Erickson, K., Kipper, M. J., Muniz, E. C., Belfiore, L. A., and Tambourgi, E. B. 2012. “Natural Polymer-based Magnetic Hydrogels: Potential Vectors for Remote-controlled Drug Release,” Carbohydrate Polymers 90(3):1216-1225; Marszall, M. P. 2011. “Application of Magnetic Nanoparticles in Pharmaceutical Sciences,” Pharmaceutical Research 28(3):480-483; Marszall, M. P., Moaddel, R., Kole, S., Gandhari, M., Bernier, M., |and Wainer, I. W. 2008. “Ligand and Protein Fishing with Heat Shock Protein 90 Coated Magnetic Beads,” Analytical Chemistry 80(19):7571-7575; Ito, A., Shinkai, M., Honda, H., and Kobayashi, T. 2005. “Medical Application of Functionalized Magnetic Nanoparticles,” Journal of Bioscience and Bioengineering 100(1):1-11; Gupta, A. K. and Gupta, M. 2005. “Synthesis and Surface Engineering of Iron Oxide Nanoparticles for Biomedical Applications;” Biomaterials 26(18):3995-4021). Ultimately, drug-carrier particle binding, performed in vitro at the outset, will also be internalized with the use of extravascular jackets.

The magnet may be local in the form of an impasse-jacket encircling the ductus, or if the anatomical clearance does not allow sufficient field strength, then an impasse-jacket with support from patch-magnets, a powerful external tractive electromagnet, or these in combination. When the implanted jacket and any other magnets have sufficient field strength to hold the analyte, the completion of analyte resituation or extraction by an external neodymium iron boron permanent or an electromagnet can follow after an interval. The magnetically susceptible carrier varies in number according to the mass of the intracorporeal target, so that to target a certain type of blood cell so that it can be manipulated while intact on a one for one carrier to target basis requires a larger number per target than does the extraction of a nucleotide, for example.

A few examples of such counter-specific pairing, essentially pertinent to any situation wherein one substance effectively neutralizes or cancels out, or selectively fastens onto the receptors of another, to inactivate it are, the flow rate through the ductus and reaction time allowing, antigen-antibody, such as virus antibody, antibody-antigen, enzyme-substrate, substrate-enzyme, enzyme inhibitor-reversible enzyme inhibitor, hormone-receptor protein, and the relation mutuating between Type 1 and Type 2 cytokines. These same relations, along with reversal agents to neutralize drugs delivered through a jacket upstream, can be released from a downstream jacket to reverse the effect of endogenous substances or drugs.

In metastatic renal cancer, for example, the direct targeting of the primary tumor and segments along the great vessels allows any residual distributed disease to be treated with a reduction in the systemic dose and/or other chemotherapeutic means that remain essential to kill shed or daughter cells (see, for example, June, C. H. 2007. “Adoptive T Cell Therapy for Cancer in the Clinic,” Journal of Clinical Investigation 2007; 117(6):1466-1476; Boldt, D. H., Mills, B. J., Gemlo, B. T. Holden, H., Mier, J., Paietta, E. and 8 others 1988. “Laboratory Correlates of Adoptive Immunotherapy with Recombinant Interleukin-2 and Lymphokine-activated Killer Cells in Humans,” Cancer Research 48(15):4409-4416; Rosenberg, S. A., Lotze, M. T., Muul, L. M., Leitman, S. Chang, A. E., Ettinghausen, S. E, Matory, Y. L, Skibber, J. M., and 5 others 1985. “Observations on the Systemic Administration of Autologous Lymphokine-activated Killer Cells and Recombinant Interleukin-2 to Patients with Metastatic Cancer,” New England Journal of Medicine 313:(23)1485-1492). Derivative tumors are treated likewise.

In this context, the ability to straddle each kidney to deliver in vitro-incubated interleukin-2 (Prometheus Laboratories Proleukin®) lymphokine-activated T-cells, or T-lymphocyte killer cells, tumor-infiltrating lymphocytes, or cytotoxic T-lymphocytes (see, for example, Merck Manual of Diagnosis and Therapy, 18th Edition 2006, Section 11, Chapter 148, “Tumor Immunology,” Immunotherapy, page 1156), into the renal artery or arteries as inlets and extract these at the renal veins as outlets; with another delivery jacket placed about the ascending aorta as shown in FIG. 21 and extraction jacket such as that shown in FIG. 13 applied to the abdominal aorta, with others as necessary applied to the venae cavae, trunk, pulmonary arteries, or pulmonary veins averts side effects of intermittent large dose administration that can result in severe side effects and even death (see, for example, Merck Manual of Diagnosis and Therapy, 18th Edition 2006, section 148, “Tumor Immunology,” page 1154; Wang, D., Zhang, B., Gao, H., Ding, G., Wu, Q., Zhang, J., Liao, L., and Chen, H. 2014. “Clinical Research of Genetically Modified Dendritic Cells in Combination with Cytokine-induced Killer Cell Treatment in Advanced Renal Cancer,” BMC [BioMed Central] Cancer 14(1):251; Jiang, J., Wu, C., and Lu, B. 2013. “Cytokine-induced Killer Cells Promote Antitumor Immunity,” Journal of Translational Medicine: 11:83; Ostanin, A. A., Chernykh, H. R., Leplina, O Y., Shevela, E. Y., Niconov, S. D., and Kozlov, V. A. 1997. “IL-2-Activated Killer Cells and Native Cytokines in the Treatment of Patients with Advanced Cancer,” Russian Journal of Immunology 2(3-4):167-176). Moreover, continuous delivery produces better results than does administration by injection every eight hours (Thompson, J. A., Lee, D. J., Lindgren, C. G., Benz, L. A., Collins, C., Shuman, W. P., Levitt, D., and Fefer, A. 1989. “Influence of Schedule of Interleukin 2 Administration on Therapy with Interleukin 2 and Lymphokine activated Killer Cells,” Cancer Research 49(1):235-240).

The cytotoxic T-lymphocytes or T-lymphocyte killer cells are bound to magnetically susceptible superparamagnetic nanoparticles before loading in the drug vial or reservoir switching turret shown in FIGS. 31, 32, and 36 for infusion through the side-entry jacket or jackets and extraction by means of impasse-jackets. Switching among pump inputs is shown as accomplished by means of a turret for pictorial clarity, alternative means of switching, such as with a relay possible. In any condition whereby a stenosed segment of a major supply artery limits perfusion but allows placement of a synthetic bypass, such a jacket will provide access for the direct delivery of medication in support of the junction itself, to prevent coagulation in the bypass, and/or to treat the territory the bypass supplies, as well as support the junction structurally.

Stenosed vessels with origin at the aortic arch which cause neurologic deficits or paresthesias (see, for example, Messina, L. M. and Zelenock, G. B. 1997. “Cerebrovascular Occlusive Disease,” Chapter 80 in Greenfield, L. J., Mulholland, M. W., Oldham, K. T., Zelenock, G. B., and Lillemoe, K. D. (eds.), Surgery: Scientific Principles and Practice, page 1757) are no less treatable thus than are aortofemoral or ileofemoral bypasses, for example, as mentioned below. The jacket can provide a simple and secure junction for passing a cabled therapeutic or imaging device through it and into the native lumen. This allows introducing medication or retrieving diagnostic test samples, for example. The jacket as merely a junction can be extended to incorporate a surrounding magnet with a field intensity gradually increased in the antegrade direction to draw a magnetically susceptible carrier bound drug or other therapeutic substance radially outward through the lumen wall.

Cladding the jacket and lines leading to it with radiation shielding further allows the direct delivery through the lines to the jacket of less intensively superparamagnetic drug-carrier bound radionuclide, for example, which are then drawn radially outward through the lumen wall and into contact with any lesion there. Moreover, because the jacket is periductal with the radiation shield the outermost component, the radiation shield can be made of overlapping particles of tungsten, each encapsulated within a chemically isolating nonabsorbable polymeric casing, for example, and bound to its neighbors with a biodegradable bonding agent so that it disintegrates once the radiation is depleted.

Since layers of the jacket, within or subjacent to a radiation shield if used, can be perforated or fenestrated, the adverse effects of long term isolation from the internal mileau can be averted. That primary or intrinsic neoplasms and other intramural lesions treated with radiation seldom appear in blood vessels means that the need for radiation shielding is infrequent. Moreover, should such a lesion appear in a vessel, the shielded jacket allows the direct targeting of the affected segment with drugs such as a statin and steroids to suppress the rapid atherosclerotic degradation otherwise seen. In other type ductus, it is the relative half-life and time that the ductus may remain encircled that determines the allowable interval for use and radiation level of the radionuclide.

The jacket is used to establish catheteric, or artificial conduit to native end-to-side, rather than native end to native end anastomosis by sutured connection, distinguishing such a junction from most if not all prior art vascular connectors. The synthetic conduit can then be used to deliver therapeutic substances directly from a port implanted at the body surface to the native lumen or to divert contents from one native lumen to the same native conduit downstream as a bypass or to another ductus in the same or a different bodily system as a shunt. As used herein, a ‘port’ or ‘portal’ can be of the conventional subcutaneously implanted or ‘portacath’ type used with a central venous catheter, but will more often be of an ‘open’ type to be described.

The passageway leading from such an open-type port at the body surface through a connecting tube, usually a catheter, to the side-entry connection jacket used to form a junction with the native conduit or ductus is uniform in diameter throughout, is unobstructed or readily cleared, and can be angled. This makes it possible not only to move fluids in either direction, to deliver drugs and aspirate diagnostic test samples, but to pass cabled diagnostic and therapeutic instruments through the junction on a regular basis, and allow connection to apheresis—leukapheresis, plateletpheresis, potentially even plasmapheresis—apparatus, as well as allow the use of synthetic tubing to create internal shunts and bypasses. Such a port is no less capable of conventional applications, such as delivering radiopaque contrast agents, chemotherapeutic, antineoplastic, autoimmune suppressive, clotting factor, alpha 1-antitrypsin deficiency replacement, and antimicrobial drugs, and will support total parenteral nutrition as well as hemodialyisis and peritoneal dialysis.

Since once placed, routine entry into the lumen involves neither puncturing the skin nor entry into a body cavity, access with such means is effectively noninvasive. Connection directly to the vascular tree thus allows the use of intravascular ultrasound, for example, while connection along the gastrointestinal tract, for example, allows the withdrawal of solid biopsy test samples, for example. Periductal implants avoid the placement of a foreign object within the lumen, invariably associated with the risk of adverse consequences. Such implants can communicate with a port at the body surface, can be used to deliver any therapeutic substance prepared in a flowable form, and when magnetized, can draw magnetic drug-carrier bound drugs radially outward through the tunics to reach inflammation or lesions within the ductus wall.

Indissolubly bound to the carrier and drawn radially outward, the drug will reach the lesion regardless of the axifugal or abaxial depth or distance of the lesion from the lumen. Used to administer chemotherapy along the small intestine, for example, the drug would first reach a primary carcinoma of the mucus epithelium, then an adenoma in the submucosa, then a sarcoma in the outer muscle layers whether primary or incurred by metastatic invasion. No drug eluting stent absorbable or nonabsorbable can approach this depth of penetration. Moreover, takeup is accomplished without dependence upon the presence of receptors for the drug by the target as limits the choice of substance, radionuclide, or monoclonal antibody to one having an intrinsic affinity for the target tissue, such as that of iodine or iodine 131 for the thyroid gland.

Application to achieve the targeted treatment of a neoplasms with antimitotic and antiangiogenic drugs that are toxic to all tissue with or without heating, for example, is clear, as is the avoidance of side effects. All drugs can induce side effects, making the ability to limit delivery to only the tissue intended especially beneficial. For example, until a gene therapy technique proves successful, every drug given to treat inflammatory bowel disease, or regional enteritis (ileocolitis, Crohn's disease, ulcerative colitis), will continue to pose risks for inducing collateral pathology (see, for example, Young, V. B., Kormos, W. A., Chick, D. A., and Goroll, A. H. 2010. Blueprints: Medicine, Philadelphia, Pa.: Lippincott, Williams, and Wilkins, page 187).

The circulation of steroids to treat Cushing's disease, for example, often results in moon facies, and of antimitotic drugs to treat neoplasms, for example, often results in baldness, just to name two. An impasse-jacket can not only directly target a site of disease ordinarily accessible solely through systemic administration—and then weakly, with little of the dose delivered as intended—but the drugs released to or from it can be delivered at concentrations—and if radioactive, at dose rates—with a potency far higher than might be allowed to circulate. Little if any entering the circulation, the drug is concentrated where its effect is intended, exposure to other tissue and the adverse side effects this causes minimized if not eliminated.

With systemic disease that can induce lesions elsewhere, such as atherosclerosis or a vasculitis able to induce localized obstructions, this local focusing does nothing to interfere with administering a background dose of the same or another drug in the circulation. In the treatment of nonsystemic or localized disease, the inability to closely target drugs to sites within the body where the drugs are intended to take effect, avoiding the general circulation and metabolism by the liver, often disallows, entirely or in concentration, in all or in only certain patients, the use of drugs that would otherwise have potential value. Moreover, numerous drugs exert beneficial local effects when directly applied to the diseased or lesioned tissue.

Limited exposure of more severely atherosclerosed arteries or segments thereof to statins with or without adjuvants takes advantage of the pleiotropic effects of these drugs, which targeted, can be administered in higher concentration than the risk of myopathic or other side effects would allow (see, for example, West, A. M., Anderson, J. D., Meyer, C. H. Epstein, F. H., Wang, H., Hagspiel, K. D, Berr, S. S. and 7 others 2011. “The Effect of Ezetimibe on Peripheral Arterial Atherosclerosis Depends upon Statin Use at Baseline,” Atherosclerosis 218(1):156-162; Zhou, Q. and Liao, J. K. 2010. “Pleiotropic Effects of Statins.—Basic Research and Clinical Perspectives,” Circulation Journal 74(5):818-826; Sastry, P., and Kaski, J. C. 2010. “Atherosclerotic Plaque Regression—The Role of Statin, Therapy”. Drugs Today (Barcelona) 46(8), 601-608; Araujo, D. B., Bertolami, M. C., Ferreira, W. P., Abdalla, D. S., Faludi, A. A., Nakamura, Y., and Bricharello, L. P. 2010. “Pleiotropic Effects with Equivalent Low-density Lipoprotein Cholesterol Reduction: Comparative Study between Simvastatin and Simvastatin/Ezetimibe Coadministration,” Journal of Cardiovascular Pharmacology 55(1):1-5; Taylor, A. J., Villines, T. C., Stanek, E. J., Devine, P. J., Griffen, L., Miller, M., Weissman, N. J., and Turco, M. 2009. “Extended-release Niacin or Ezetimibe and Carotid Intima—Media Thickness,” New England Journal of Medicine 361(22):2113-2122; Zhou, Q. and Liao, J. K. 2009. “Statins and Cardiovascular Diseases: From Cholesterol Lowering to Pleiotropy,” Current Pharmaceutical Design 15(5):467-478; Wang, C. Y., Liu, P. Y., and Liao, J. K. 2008. “Pleiotropic Effects of Statin Therapy: Molecular Mechanisms and Clinical Results,” Trends in Molecular Medicine 14(1):37-44; Kastelein, J. J., Akdim, F., Stroes, E. S., Zwinkerman, A. H., Bots, M. L., and 23 others 2008. “Simvastatin With or Without Ezetimibe in Familial Hypercholesterolemia,” New England Journal of Medicine 358(14):1431-1443; Landmesser, U., Bahlmann, F., Mueller, M., Spiekermann, S., Kirchhoff, N., Schulz, S., Manes, C., and 6 others 2005. “Simvastatin Versus Ezetimibe: Pleiotropic and Lipid-lowering Effects on Endothelial Function in Humans,” Circulation 111(18):2356-2363; Liao, J. K. and Laufs, U. 2005. “Pleiotropic Effects of Statins,” Annual Review of Pharmacology and Toxicology 45:89-118; Liao, J. K. 2005. “Clinical Implications for Statin Pleiotropy,” Current Opinion in Lipidology 16(6):624-629; Wolfrum, S., Jensen, K. S., and Liao, J. K. 2003. “Endothelium-dependent Effects of Statins,” Arteriosclerosis, Thrombosis, and Vascular Biology 23(5):729-736; Takemoto, M and, Liao, J. K. 2001. “Pleiotropic Effects of 3-Hydroxy-3-Methylglutaryl Coenzyme A Reductase Inhibitors,” Arteriosclerosis, Thrombosis, and Vascular Biology 21(11):1712-1719; Laufs, U., La, F., Plutzky, J., and Liao, J. K. 1998. “Upregulation of Endothelial Nitric Oxide Synthase by HMG [Hydroxy Methylglutaryl] CoA [Coenzyme A] Reductase Inhibitors,” Circulation 97(12):1129-1135).

Similarly, the local application of a combination of drugs which includes mammalian target-of-rapamycin, or mTOR, inhibitors, such as sirolimus (rapamycin) and everolimus appears to reduce the vulnerability to rupture of plaque (see, for example, Martinet, W., De Loof, H., and De Meyer, G. R. 2014. “mTOR Inhibition: A Promising Strategy for Stabilization of Atherosclerotic Plaques,” Atherosclerosis 233(2):601-607; Martinet, W., De Meyer, I., Verheye, S., Schrijvers, D. M., Timmermans, J. P., and De Meyer, G. R. 2013. “Drug-induced Macrophage Autophagy in Atherosclerosis: For Better or Worse?,” Basic Research in Cardiology 108(1):321; Martinet, W., Verheye, S., De Meyer, I., Timmermans, J. P., Schrijvers, D. M., Van Brussel, I., Bult, H., and De Meyer, G. R. 2012. “Everolimus Triggers Cytokine Release by Macrophages: Rationale for Stents Eluting Everolimus and a Glucocorticoid,” Arteriosclerosis, Thrombosis, and Vascular Biology 32(5):1228-1235; Croons, V., Martinet, W., and De Meyer, G. R. 2010. “Selective Removal of Macrophages in Atherosclerotic Plaques as a Pharmacological Approach for Plaque Stabilization: Benefits versus Potential Complications,” Current Vascular Pharmacology 8(4):495-508; Martinet, W., Verheye, S., and De Meyer, G. R. 2007. “Everolimus-induced mTOR Inhibition Selectively Depletes Macrophages in Atherosclerotic Plaques by Autophagy,” Autophagy 3(3):241-244).

The gastrointestinal gastritis and ulcers that result from long term use of nonsteroidal anti-inflammatory drugs such as aspirin are avoided, as is the risk of urticaria or anaphylaxis responsive to certain drugs such as antibiotics, for example. (see, for example, Merck Manual, 18th edition, pages 974 and 1360). Reciprocally, drugs that must be limited in concentration for systemic dispersal because of the side effects and drug-drug interactions that loom when other tissue is exposed can be increased in concentration or potency to the optimal dose for direct delivery from the standpoints not only of efficacy but of portability and power conservation or battery life for use in an ambulatory system.

Without targeting, drugs or other remedial substances that might be used to treat symptoms must sometimes be discounted or limited in strength to avoid conflict with those used to treat the etiology, and those used to treat a disease in one part of the body may conflict with those used to treat another disease in another part of the body. Targeting also makes it possible to eliminate the need for secondary drugs that must be prescribed merely to counteract side effects produced by primary drugs. For example, tightly controlled delivery of drugs that act directly upon a lesion and do not require systemic dispersal to be processed by the liver, for example, minimizes the extent of contact with nontargeted tissue and therefore the opportunities for the drugs to bind covalently with or otherwise affect serum or tissue proteins, identified as a mechanism underlying the iatrogenic inducement of autoimmune disorders.

Where the condition treated is chronic or recurrent as to justify the placement of a direct access line, the use of a drug, existing or yet to be developed, to be preferred if it did not induce autoimmune disease when taken systemically, can be sustained (see, for example, Chang, C. and Gershwin, M. E. 2010. “Drugs and Autoimmunity—A Contemporary Review and Mechanistic Approach,” Journal of Autoimmunity 34(3):J266-J275; Brown, R. J., Rother, K. I., Artman, H., Mercurio, M. G., Wang, R., Looney, R. J., and Cowen, E. W. 2009. “Minocycline-induced Drug Hypersensitivity Syndrome Followed by Multiple Autoimmune Sequelae,” Archives of Dermatology/JAMA Dermatology 145(1):63-66; Merck Manual of Diagnosis and Therapy, 18th Edition 2006. “Autoimmune Disorders” and “Drug Hypersensitivity,” pages 1361-1363; Elkayam, O., Yaron, M., and Caspi, D. 1999. “Minocycline-induced Autoimmune Syndromes: An Overview,” Seminars in Arthritis and Rheumatism 28(6):392-397).

No endoluminal stent, absorbable or eluting, can restrict delivery so finely as to nearly if not completely eliminate the risk of such sequelae; indeed, the alternative is to administer drugs systemically where exposure to other tissues and organs must be indiscriminate. The use of extravascular implants enlists relatively minor invasive surgery in the service of medical management through the positioning of implants that allow drugs to be precisely targeted rather than administered in higher doses through the systemic circulation, exposing every tissue and organ. Indiscriminate delivery thus is the source of many adverse side effects and drug-drug interactions. Where the use of certain drugs at one anatomical site ordinarily disallows the use of other drugs at another, minimizing if not eliminating drug interaction fundamentally expedites the treatment of regionally concentrated if not distinct comorbidities.

More specifically, the availability of means for substantially isolating certain tissue for the delivery of certain drugs frees the clinician to select the best drugs for treating each of two or more concurrent but separate disease processes, or comorbidities, whether pathophysiologically related or unrelated without much concern for drug drug interactions. Since clinical trial protocols of new drugs apply criteria that omit comorbidities (Jones, R. 2010. “Chronic Disease and Comorbidity,” British Journal of General Practice 60(575):394, the ability to target drugs to a certain lesion preserves the validity of trial findings in eliminating or substantially eliminating the effect of collateral disease. The jackets may be of different sizes, different types, and applied to ductus belonging to different organ systems.

The principle or index diseases seen most often in comorbid conditions are cardiovascular or malignant (Gijsen R., Hoeymans, N., Schellevis, F. G. Ruwaard, D., Satariano, W. A., and van den Bos, G. A. M. 2001. “Causes and Consequences of Comorbidity: A Review,” Journal of Clinical Epidemiology 54(7):661-674), both having systemic implications and expression, but inducing localized lesions amenable to the targeted delivery of drugs. Studies of comorbidity tend to be relatively few (see, for example, Valderas, J M., Mercer, S. W., and Fortin, M. 2011. “Research on Patients with Multiple Health Conditions: Different Constructs, Different Views, One Voice,” Journal of Comorbidity 1:1-3; Diederichs, C., Berger, K., and Bartels, D. B. 2011. “The Measurement of Multiple Chronic Diseases—A Systematic Review on Existing Multimorbidity Indices,” The Journals of Gerontology. Series A, Biological Sciences and Medical Sciences 66(3):301-311; Fortin, M., Lapointe, L., Hudon, C., and Vanasse, A. 2005. “Multimorbidity is Common to Family Practice. Is it Commonly Researched?” Canadian Family Physician 51:244-245; Valderas, J. M., Glynn, L., Ferrer-Menuina, X., Johnson, R., and Salisbury, C. 2011. “Diseases that Come in Multiples: A Systematic Review of Multi-morbidity Profiles,” Family Medicine 43(Supplement 1); Mercer, S. W., Smith, S. M., Wyke, S., O'Dowd, T., and Watt, G. C. M. 2009. “Multimorbidity in Primary Care: Developing the Research Agenda,” Family Practice 26(2): 79-80).

Since a side-entry connection jacket can incorporate more than a single side-entry connector, a catheteric or synthetic drug feed fluid line, or simply line, from the surface can be connected to a side-entry connector used as an inlet into the ductus the jacket encircles, while another, ordinarily larger synthetic outlet catheter is connected to a second side-entry connector to serve as the outlet into a prosthetic bypass that reenters the native conduit distal to or beyond an obstruction. Alternatively, the drug delivery line can be connected by a separate jacket to the native conduit upstream to the bypass outlet jacket or downstream from the synthetic bypass outlet jacket. This versatility makes it possible to differently target segments of the native conduit upstream or downstream to the synthetic-native connection for the delivery of drugs.

For targeting a segment along a conduit, a drug is suitable in proportion to the promptness of its effect at the point or level of and following delivery. A single side-entry connection jacket can incorporate more than one side-connector, any or all of which can be of either type. Whether for diagnostic or therapeutic purposes, when the jacket is to serve primarily for the frequent passage into the native lumen of a cabled device or catheter, a side-entry jacket with a double arm side-connector as shown in FIG. 7 expedites steering in either direction to the native lumen, a double arm clean-out or inline port, as shown in FIGS. 30 and 31 affording extracorporeal entry through the pump-pack as shown in FIG. 31.

When the adductal terminus, or distal end, of the side-connector is substantially round, it can be placed using essentially the same procedure to be described for the side-connectors shown in FIGS. 1-6. That is, the side-connector can be rotated as a circle-cutter as well as the application of vacuum force. Such need is substantially limited to the thick walls of lumina along the digestive tract. More often, however, this type of side-connector, elongated along the axis of the ductus at the distal end, is used for the continuous or frequent infusion or extraction of analytes or cells. To deliver medication into the native lumen when the hole, that is, the ostium or stoma, excised from the side of the ductus is covered by a fluid resistor such as a flap-valve to be described, requires additional exertion by the pump.

When the action must be continuous or frequent as with ambulatory apheresis, this will reduce the battery life and meantime between servicing and therewith, the duration of the period during which the patient will be able to move freely. To forestall such interruptions while minimizing the weight of the apparatus, the patient is advised to have fully recharged batteries available. This embodiment has a permanent elastic slit membrane or depending upon the forces involved, extraction trap flap valve at its distal end. For extractive use, such as ambulatory apheresis of an analyte or cells, the jacket is used in conjunction with a tractive electromagnet held in the correct position with its weight supported at the body surface with the aid of a harness.

The superior conductivity of silver offset by its greater mass and expense, the coil of the electromagnet is wound with copper wire. Subcutaneous placement affords stability for which deeper or periductal placement will usually necessity stabilization with suture to a pad or patch stitched to neighboring tissue. This insertion may itself require to be stabilized with suture to neighboring tissue. Depending upon the clearance surrounding the substrate ductus, distributing and balancing the weight of a larger magnet is sometimes ameliorable through the use of an extraction jacket having more than one double arm side-connector, flap valve, and separate electromagnet at points about the circumference. Connecting the outlet arm of each jacket to the inlet arm of that adjacent with a single flush out line in series simplifies placement and extraction.

To prevent kinking, the sections of piping from jacket to jacket are made of thick-walled polytetrafluoroethylene (Teflon® E.I. Dupont de Nemours). Experience with ventricular assist devices and total artificial hearts indicates that the patient acclimates to the presence of a moderately obtrusive implant, probably with the aid of adaptive tissue development such as toughening or induration of tissue at the points of suspension, attachment, or abutment. Acceptance is promoted by minimizing size and weight and by maximizing stabilization. Subcutaneous placement is therefore expeditious but in increasing the magnetic gap, increases considerably the magnetic strength and power required and thus the weight of the magnet and the battery as power source.

A ductus side-entry jacket with this type of side-connector is placed with the pump generating a vacuum force that draws the tissue plug over the cutting die trepan edge surrounding the slit membrane or flap valve and pulls the excised plug through the membrane. The magnet remains energized during extraction and is deenergized at the same time that the pump to flush the tissue plug out the line is energized. To prevent the catching of particles on the internal surfaces of flap valves, these are similarly provided with a smooth surface, usually vapor deposited or laminated polytetrafluoroethylene, special materials such as these used where necessary. To minimize the accumulation of debris, the internal surface of a double arm side-connector, flap valve, and fluid line used for extraction on a sustained basis is made of or lined with a slippery substance such as polytetrafluoroethylene.

Especially tacky extracts may necessitate the use of lining surface materials even more slippery (see, for example, Peng, W., Guan, C., and Li, S. 2013. “Ultrasmooth Surface Polishing Based on the Hydrodynamic Effect,” Applied Optics 52(25):6411-6416; Wong, T.-S., Kang, S. H., Tang, S. K., Smythe, E. J., Hatton, B. D., Grinthal, A., and Aizenberg, J. 2011. “Bioinspired Self-repairing Slippery Surfaces with Pressure-stable Omniphobicity,” Nature 477(7365):443-447, comment Nosonovsky, M. 2011. “Materials Science: Slippery When Wetted,” Nature 477(7365):412-413; Barredo, D., Calleja, F., Nieto, P., Hinarejos, J. J., Laurent, G., Vázquez de Parga, A. L., Farías, D., and Miranda, R. 2008. “A Quantum-Stabilized Mirror for Atoms,” Advanced Materials, 20(18):3492-3497; Logeeswaran V. J., Chan, M.-L., Y. Bayam, Saif Islam, M., Horsley D. A., Li X., Wu, W., Wang, S. Y., and Williams, R. S. 2007. “Ultra-smooth Metal Surfaces Generated by Pressure-induced Surface Deformation of Thin Metal Films,” Applied Physics A. Materials Science and Processing 87(2):187-192).

To avoid the pulse and arterial blood pressure which interfere with the extraction of an analyte and increase the field force required, depleting the battery or batteries more quickly, a continuous analyte extraction and/or delivery jacket such as used for ambulatory apheresis is placed along a major vein (see, for example, Cherry, E. M., Maxim, P. G., and Eaton, J. K. 2010, Op cit.). For any analyte to be extracted before entering the liver, placement along the portal vein affords first-pass interdiction. To prevent injury to the outer tunic (tunica externa, adventitia, fibrosa), the adductal edges of the side ends of the outer shell of the jacket are rounded.

Nevertheless, so that the edges do not come into contact with the outer surface of the ductus, the viscoelastic polyurethane foam lining is made thick enough to accept the radially outward pulsatile or peristaltic excursion due to the intrinsic motility of the ductus. Additionally, so that cutting into the ductus wall to excise the plug of tissue affords sufficient foam around the trepan that the foam will accept the full thickness of the ductus wall as the sharp cutting edge enters, the foam must be no less thick than the thickness of the wall surrounding the ductus lumen. Placement along the pulmonary trunk, pulmonary arteries, and or the superior vena cava addresses the pulmonary circulation, and the inferior vena cava the somatic circulation. Further increasing the thickness of the foam lining allows for growth in a young patient, as does the compliance of the jacket spring hinges, keyed to the ductus excursive force.

Thickening the foam lining also allows conformity to a ductus that may be inconsistent or exhibit an irregularity or irregularities in its outer diameter or surface. Relief from the need for diametrical uniformity also allows some tissue surrounding the ductus, such as periadventitial fat, to be included in the jacket. The foam lining a ductus side-entry jacket is of a density as to noncompressingly invest the tiny nerves and vessels that support the ductus to be treated. The thickness of the foam contemplates changes in diameter along the ductus whether structural or due to intrinsic motility, pulsation, or protrusion of a tumorous lesion in addition to affording considerable latitude in the diameter of the conduit that might be treated with a side-entry connection jacket of given internal diameter.

The sustained permeation of the foam layer through the intermittent delivery of an adverse tissue reaction retardant such as phosphorylcholine through a line from a pump to the foam lining through the jacket shell and magnet and radiation shielding if present is facilitated through the use of an open cell foam. Gradations in foam density afford yet greater latitude in this regard. Small and singular areas on the outer surface of the side-entry connector and the inner surface of the locking collar or bushing in which the ductus side-entry connector is journaled are raised, roughened, and placed in complementary relation to mesh or interdigitate and thus lock the side-entry connector in position when intentionally lapped or interfaced.

Except for this small area, the side-entry connector slides and rotates freely. Most if not all procedures are performed under local anesthesia without the risks of general anesthesia. Whether before or after the jacket has been placed about the vessel as the operator finds more expeditious, an aspiration line or hose leading to a vacuum pump is attached to the side-entry connector. When the connector is continuous with the catheter leading to it, the catheter serves this purpose. The vacuum is used to hold the outer surface, or adventitia, fibrosa, or serosa, of the tubular structure against the razor sharp end of the side-entry connector despite intrinsic motility or pulsation, allowing the operator to rotate and gently advance the side-entry connector as a circle-cutter to incise a circular plug from the structure wall.

With adequate vacuum pressure, the razor sharp adductal edge of the side-entry connector will cut through a thick tissue wall without assistance from the operator; however, if the vacuum is adjusted too high or improperly synchronized to intrinsic movement in the ductus, the sides of the tissue plug may then veer more radially inward with increased depth of cut as the lumen is approached. This can result in the formation of a triangular gutter in the lumen wall surrounding the adductal edge of the connector. Such a gutter is best avoided in the gut, ureters, and other nonvascular ductus as a weak spot not only susceptible to the accumulation of detritus as a trap, but also to the leakage of septic debris. Except in larger muscular arteries, this should seldom occur, and if it does, any accumulation of thrombus in the gap is replaced by intimal tissue.

Nevertheless, if the operator suspects that the risk is present and could result in initiating turbulent, hence thrombogenic, flow, the same precaution should be employed. To prevent this complication, the sides of the side-entry connector are wetted by a swellant formulated to encourage and become supplanted by tissue. Continuity of the side-connector and the supply line tends to make initial placement, substitution, or eventual replacement of the line more awkward, but is not discounted. The connector to a synthetic line is not intended to be applied by the operator or a technician but supplied as a manufactured article. Use of a magnetized side entry connection jacket such as that shown in FIG. 3 to draw the ferrofluid infusate long axifugally outward against, into, and through the lumen wall eliminates the need to perforate the sides of the side-connector apposite the wall to allow direct perfusion into the lumen wall.

While reverse or hepatofugal flow may necessitate reversing the upstream position of the inlet jacket and the downstream position of the outlet or extraction jacket, the reduced force advantages in venous application are not significantly reduced when idiopathies, individual peculiarities, disease, or surgery induces pulsatile flow (see, for example, Demirtürk, O. S., Güvener, M., Co [symbol] kun, I., and Yildirim, S. V. 2013. “Results of Additional Pulsatile Pulmonary Blood Flow with Bidirectional Glenn Cavopulmonary Anastomosis: Positive Effect on Main Pulmonary Artery Growth and Less Need for Fontan Conversion,” Heart Surgery Forum 16(1):E30-E34; Machare-Delgado, E., Decaro, M., and Marik, P. E. 2011. “Inferior Vena Cava Variation Compared to Pulse Contour Analysis as Predictors of Fluid Responsiveness: A Prospective Cohort Study,” Journal of Intensive Care Medicine 26(2):116-124; Solhjoo, E., Mansour-Ghanaei, F. Moulaei-Langorudi, R., and Joukar, F. 2011. “Comparison of Portal Vein Doppler Indices and Hepatic Vein Doppler Waveform in Patients with Nonalcoholic Fatty Liver Disease with Healthy Control,” Hepatitis Monthly 11(9):740-744; Goncalvesova, E., Lesny, P., Luknar, M., Solik, P., and Varga, I. 2010. “Changes of Portal Flow in Heart Failure Patients with Liver Congestion,” [in English] Bratislayské Lekárske Listy [Bratislava Medical Journal] 111(12):635-639; Neema, P. K., Sethuraman, M., Krishnamanohar, S. R., and Rathod, R. C. 2009. “Superior Vena Cava Syndrome after Pulsatile Bidirectional Glenn Shunt Procedure: Perioperative Implications,” Annals of Cardiac Anaesthesia 12(1):53-56; Görg, . C, Riera-Knorrenschild, J., and Dietrich, J. 2002. “Pictorial Review: Colour Doppler Ultrasound Flow Patterns in the Portal Venous System,” British Journal of Roentgenology 75(899):919-929; Görg, C., Wollenberg, B., and Beyer, J. 2001. “Reversed Portal Vein Pulsatility on Doppler Ultrasound Secondary to an Iatrogenic Mediastinal Haematoma,” British Journal of Roentgenology 74(886):962-964; Rengo, C., Brevetti, G., Sorrentino, G., D'Amato, T., Imparato, M., Vitale, D. F., Acanfora, D., and Rengo, F. 1998. “Portal Vein Pulsatility Ratio Provides a Measure of Right Heart Function in Chronic Heart Failure,” Ultrasound in Medicine and Biology 24(3):327-332; Gallix, B. P., Taourel., P., Dauzat, M., Bruel, J. M., and Lafortune, M. 1997. “Flow Pulsatility in the Portal Venous System: A Study of Doppler Sonography in Healthy Adults,” American Journal of Roentgenology 169(1):141-144; Duerinckx, A. J., Grant, E. G., Perrella, R. R., Szeto, A., and Tessler, F. N. 1990. “The Pulsatile Portal Vein in Cases of Congestive Heart Failure: Correlation of Duplex Doppler Findings with Right Atrial Pressures,” Radiology 176(3):655-658; Hosoki, T., Arisawa, J., Marukawa, T., Tokunaga, K., Kuroda, C., Kozuka, T., and Nakano, S. 1990. “Portal Blood Flow in Congestive Heart Failure: Pulsed Duplex Sonographic Findings,” Radiology 174(3 Part 1):733-736; Applefeld, M. M. 1990. “The Jugular Venous Pressure and Pulse Contour,” Chapter 19 in Walker, H. K, Hall, W. D., and Hurst, J. W. (eds.) Clinical Methods: The History, Physical, and Laboratory Examinations, Bethesda, Md.: Butterworth Division, Reed Publishing).

Therapeutic substances and drugs that act quickly without the need to pass through the liver are piped directly to a side-entry connection jacket encircling the native conduit at the target level; otherwise, piping must be to a separate jacket positioned upstream. With sustained delivery so that difference in distance to each jacket is insignificant, like kind lesions on multiple ductus or at intervals along a single ductus can be encircled with separate side-entry connection jackets, each supplied by a branch from the same supply line rather than multiple ports. Rather than acting only after having been processed in the liver, the drugs for use thus are direct-acting; drugs that do are delivered in preprocessed form. A drug such as an anticoagulant can be delivered to, or at a distance upstream to, a synthetic bypass at the smallest dose necessary, dispersion in the circulating volume of blood diluting it as to be ineffectual. If necessary, a second jacket can be used to counteract or neutralize any unwanted residue.

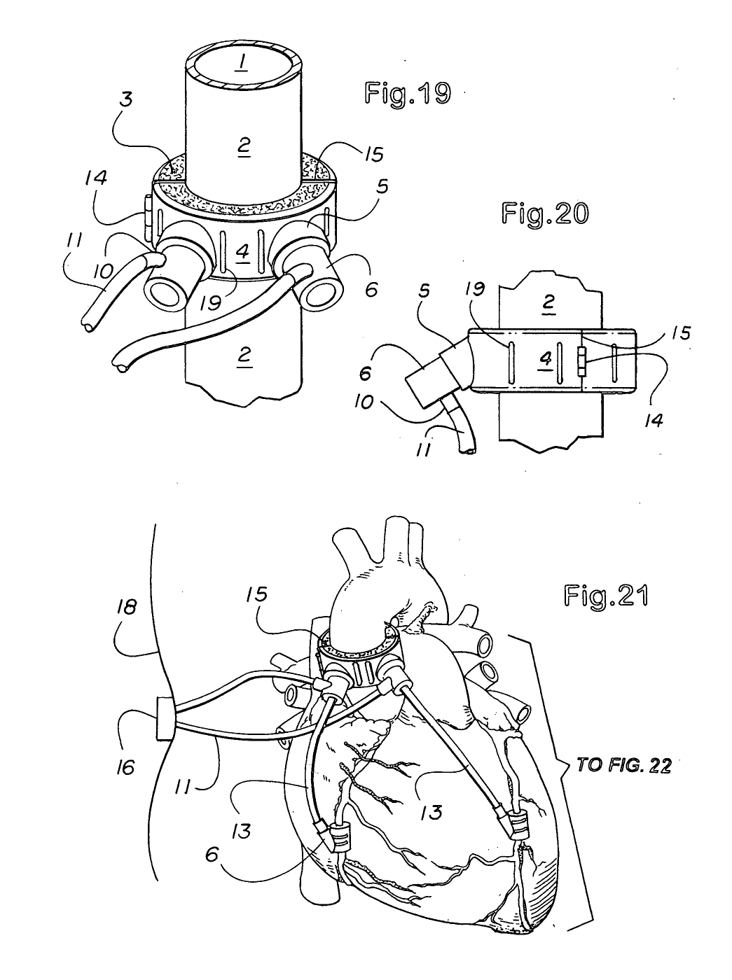

Incorporated into the pump-pair plug-in module pump-pack base or receiver, seen as part number 54 in FIG. 19, sensors used to adapt drug delivery through one or more pump-pair and jacket sets can belong to a wireless body area network with a single body central unit (see, for example, Khan, J. Y., Yuce, M. R., Bulger, G., and Harding, B. 2012. “Wireless Body Area Network (WBAN) Design Techniques and Performance Evaluation,” Journal of Medical Systems 36(3):1441-1457; Chen, M.; Gonzalez, S., Vasilakos, A., Cao, H., and Leung, V. 2010. “Body Area Networks: A Survey,” Mobile Networks and Applications (MONET) 16(2):1-23; Ullah, S., Shen, B., Islam, S. M., Khan, P., Saleem, S., and Kwak, K. S. 2010. “A Study of MAC [Medium Access Control] Protocols for WBANs [Wireless Body Area Networks],” Sensors (Switzerland); 10(1):128-145; Ullah, S, Higgins, H., Braem, B., Latre, B., Blondia, C., Moerman, I., Saleem, S., Rahman, Z., and Kwak, K. S 2010. “A Comprehensive Survey of Wireless Body Area Networks: On PHY [Physical], MAC [Medium Access Control], and Network Layers Solutions,” Journal of Medical Systems 36(3):1-30; Schmidt, R., Norgall, T., Morsdorf, J., Bernhard, J., and von der Grün, T. 2002. “Body Area Network BAN—A Key Infrastructure Element for Patient-centered Medical Applications,” Biomedical Technology 47(1):365-368).

Incorporation into the targeted drug delivery system to be described is intended to allow immediate adjustment in the delivery of medication and not just telemetric alerts for followup by remote clinicians. Individual and paired difference-measuring sensors can be positioned within the wall of the ductus, inside the jacket, such as within the inlets to and/or between the foam lining and outer layers of the jacket, or at the surface of the body, and the jackets can be placed along a single ductus, at separate locations along the circulatory system, or to span the inlet and outlet of an organ, for example. Ductus-intramural sensors individual or multiple can be of stay conformation. If necessary, non-stay type microsensors positioned in the jacket foam lining can project a microprobe into or entirely through the ductus wall to extend level and flush with the intima or mucosa.

Paired sensors can detect differences in tissue properties such as revealed by heat transmissibility or a small DC resting potential separating two points. Precautionary positioning of suitable jackets and a drug delivery system in a patient with a known predisposition for a condition distinguished by characteristic tissue degradation such as familial hypercholesterolemia, hypertriglyceridemia, Barrett's esophagus, ileocolitis, and numerous other disorders allows the use of definitive change in temperature, force, or pressure response, transmissivity, conductivity, or chemical indicia to signal the inception and degree of progression in abnormal or nonphysiological metaplastic transition. Once placed, the prosthetic drug response system can respond immediately and automatically by targeted delivery of appropriate drugs to the reporting site.

Any difference in a blood analyte, intrinsic or introduced, for which the sensor technology is available can be detected at separated points along a ductus, at distances about the circulatory system, or entering and departing an organ. The applicability to the development of suitable sensors of microcantilever immunoassay and microchip sensors, for example, is clear (see, for example, Mohammed, M. I. and Desmulliez, M. P. 2011. “Lab-on-a-chip Based Immunosensor Principles and Technologies for the Detection of Cardiac Biomarkers: A Review,” Lab on a Chip 11(4):569-595; Osiri, J. K. and Shadpour, H. 2010. “Toward Point-of-care Microchip Profiling of Proteins,” Bioanalysis 2(10):1745-1754; Tran, N. T., Ayed, I., Pallandre, A., and Taverna, M. 2010. “Recent Innovations in Protein Separation on Microchips by Electrophoretic Methods: An Update,” Electrophoresis 31(1):147-173). When the jacket cannot accommodate the sensor or sensors, a sample of the lumen contents can be delivered to the pump-pack for analysis therein or retrieval by a laboratory technician.

Metaplastic transition tends to be gradual rather than exigent; however, such a system can respond immediately and automatically to an emergency condition much as an implanted defibrillator can respond to an arrhythmia otherwise likely to result in a sudden arrest. For example, a jacket placed about an internal carotid artery can sense and signal the need for delivery of a drug to the brain as well as deliver the drug through the jacket into the artery, while an outlet jacket about a jugular vein can transmit the blood serum level of the drug in the effluent to indicate takeup in the brain as well as deliver a reversal agent if needed. If the drug would cause adverse side effects in patients generally or in this patient in particular, provided a reversal agent or counteractant is available, a downstream jacket can be used to discharge the reversal agent, substantially withholding entry of the drug into the general circulation.

This can make it possible for drugs not approved due to side effects to be used or used in certain patients. Changes in established conditions to be stressed; however, familial disorders with genomic confirmation may justify intervention prior to materialization. Where the implanted sensor is to be positioned concentrically rather than longitudinally into the ductus wall, and especially where numerous sensors or pickups are to be implanted, better concentricity is obtained through the use of a stay insertion tool, as described in copending application Ser. No. 13/694,835. A periductally mounted magnetized jacket, or impasse-jacket, also described therein, positioned downsteam prevents any accidental entry into the circulation of a sensor from resulting in an embolism. Such a jacket incorporates an outer extraction grid allowing extraction of the object to a location outside the lumen. Where the jacket is magnetized, the sensors must be capable of delivering input within the magnetic field.

Numerous implantable sensors usable thus have been developed or remain in development (see, for example, Russell, D. M., Garry, E. M., Taberner, A. J., Barrett, C. J., Paton, J. F., Budgett, D. M., and Malpas, S. C. 2012. “A Fully Implantable Telemetry System for the Chronic Monitoring of Brain Tissue Oxygen in Freely Moving Rats,” Journal of Neuroscience Methods 204(2):242-248; Sarkar, D. and Banerjee, K. 2012. “Proposal for Tunnel-Field-Effect-Transistor as Ultra-Sensitive and Label-Free s”, Applied Physics Letters 100(14):143108, Krishnamurthy, V., Monfared, S. M., and Cornell, B. 2010. “Ion Channel sensors—Part I: Construction, Operation, and Clinical Studies, IEEE Transactions on Nanotechnology 9(3):303-312; Part II: Dynamic Modeling, Analysis, and Statistical Signal Processing,” IEEE Transactions on Nanotechnology 9(3):313-321; Bazzu, G., Puggioni, G. G., Dedola, S., Calia, G., Rocchitta, G., and 5 others 2009. “Real-time Monitoring of Brain Tissue Oxygen Using a Miniaturized Biotelemetric Device Implanted in Freely Moving Rats,” Analytical Chemistry 81(6):2235-2241; Zhou, M., Liu, W., Wang, G., Sivaprakasam, M., Yuce, M. R., Weiland, J. D., and Humayun, M. S. 2006. “A Transcutaneous Data Telemetry System Tolerant to Power Telemetry Interference,” Conference Proceedings IEEE Engineering in Medicine and Biology Society 1:5884-5887; Güler, N. F. and Ubeyli, E. D. 2002. “Theory and Applications of Biotelemetry,” Journal of Medical Systems 26(2):159-178; Vo-Dinh, T. and Cullum, B. 2000. “Biosensors and Biochips: Advances in Biological and Medical Diagnostics,” Fresenius' Journal of Analytical Chemistry 366(6-7):540-551).

By comparison, ferrofluids taken by mouth for entry into the circulation will remain dependent upon patient compliance. The drug delivery means to be described make possible the targeting of different drugs available now and in the future to different organs, tissues, and vessels with strictly coordinated timing throughout the day, with no attendant present and regardless of patient wakefulness, attentiveness, or mental state, and in a way fundamentally distinct from and more capable than anything allowed by the prior art. Not only is coordinated drug administration under the control of a full time medical professional impracticable under any conditions, but manual control over drug administration employing the means to be described will quickly achieve a complexity to invite human error by trained personnel. The state of pharmacy in part dependent upon the capability to deliver drugs, the availability of such means makes possible advancements in pharmacy not previously possible, not seen before, and unpredictable.

For this reason, the fact that the technology of drug delivery rapidly outstrips what is considered medically practical for the time should not constrain the range of capability that the technology makes possible; rather, the technology should be developed, stimulating the state of pharmacy to advance to the level of sophistication that the new technology makes possible. Furthermore, the fact that the delivery of each drug is timed allows dose delivery to be sequenced, hence coordinated, among different bodily systems, organs, tissues, and vessels. Targeted drugs substantially eliminate adverse drug drug and drug food interactions as well as side effects. Another area the capability to be described will advance, seen in the INR [international normalization ratio] example, is that of implantable microsensors essential to monitor the level of disease indicia analytes for automated response by the prosthetic response system.

Implantation eliminates manual sampling and the need for apparatus of a size suitable for manipulation, allowing extreme reduction in both the size of the apparatus, and given the immediacy of response, in time to treatment. The fact that each drug is targeted and timed averts the adverse side effects that can result from the dispersal of much larger systemic doses, to include drug-drug interactions and exposure of unintended tissue. These complications often prevent or truncate the use of otherwise preferred drugs whether alone or in combination. Drug targeting seeks to substantially limit medication to the tissue that requires it. When successful, it allows the circumscribed and focused application of a drug to a diseased part or lesion at a concentration, hence potency, that if circulated would prove toxic, damaging to other tissue, or could result in adverse interactions with other drugs.

The ability to target tissue eliminates systemic dispersal as mandating increased dosing to achieve the dose needed at the site desired at the same time that exposure to other tissues by systemic dispersal limits the drug concentrations that may be used. The waste of greatly diluting costly drugs that might have been restricted to the target thus results in weakened efficacy and increases adverse drug-drug interactions and side effects. Where, as in the treatment of Cushing disease addressed below, limiting the concentration of a drug limits its use to no more than forestalling the need for an adrenectomy, the relatively minor endoscopic surgery needed to allow the dysfunctional tissue to be targeted and the circulation avoided can allow the side effects of the enzyme inhibitors used to be averted, hence, use of these drugs continued, the need for an adrenalectomy long deferred if not negated.

The direct delivery of drugs to limited segments along the middle jejunal or distal ileal segment of the small intestine allows the more proximal portions of the digestive tract to be bypassed. The implications of drug targeting for the practice of medicine are significant and pertain to every bodily system, organ, gland, and region. The body consists of tissue pipelines and the tissues these supply. Nowhere is the potential for drug targeting more relevant than as pertains to vessels and ducts. Every tissue in the body is either part of and therefore directly, or supplied by and therefore indirectly, accessible through vessels and/or ducts. There is no disease in which vascular and other supply and drainage lines are uninvolved and signal the local dysfunction to higher control centers.

Symptoms even appear in bodily systems that would seem qualitatively unlike and remote from that of origin. Regional enteritides can induce arthritis. Osteoporosis and Paget's disease of bone (osteitis deformans), for example, are disorders often secondary to endocrine disease that affect the skeleton. If arterial applications are stressed, it is because of the disproportional involvement of vessels in death from disease. No bodily conduit, to include the smallest, is analogous to inert plumbing; all are integrated into a hierarchy of negative feedback loops from the brain down to the individual cells to actively interact with the passing contents (see, for example, Jameson, J. L. 2005. “Principles of Endocrinology,” in Harrison's Principles of Internal Medicine, New York, N.Y.: McGraw-Hill, page 2072).

In endothelial function, for example, the linings of blood and lymphatic vessels actively secrete vasodilators such as relaxing factor, nitric oxide, bradykinin, potassium ions, and adenosine and vasopressors or vasoconstrictors such as endothelins, epinephrine, norepinephrine, dopamine, thromboxane, and insulin, all tied into coordinated feedback loops, which continuously adjust the degree of contraction, hence, the blood pressure. That vessel wall, segment, and organ drug targeting has not progressed beyond the drug eluting stent is due to an inadequacy of methods and means for limiting drug delivery to the site that requires treatment and would allow different drugs to be delivered in doses not limited by intolerances beyond the target area. Whether access through ‘keyhole’ incisions at the body surface is more invasive than transluminal access may not be true.

When a bodily conduit is itself diseased, effective and efficient treatment requires that medication be actively drawn into, not merely pass through the line. Allowing the medication to pass lesions wastes medication that if targeted would have contributed to an effective dose, exposes healthy tissue downstream to the wasted dose, results in complications, and increasing the dose to increase absorption only increases the waste and the risk. When the supply zone or territory or downsteam segment becomes diseased, the contents passing through the line should be adjusted or supplemented to promote healing. While therapeutic agents are often best restricted to frankly diseased tissue, the pathways in which the affected tissue participates, and therewith the far-reaching relations of that tissue to other tissue, means that the propagation of disease from that tissue to other tissue is not so restricted.

For example, the central negative feedback loops that govern angiotension flow along the hypothalamic-pituitary-adrenal axis. The central loops incorporate, integrate, and drive subsidiary loops more and more local in level down to the individual cells. Tied into the neuroendocrine and autonomic nervous systems, the hypothalamic-pituitary-adrenal axis responds to systemic blood volume by continuously regulating the blood serum levels of steroid hormones produced in the adrenal cortex, such as cortisol, and in the kidney, such as angiotensin II. Angiotensin II directly effects vasoconstriction and secondarily effects the release of aldosterone to regulate the balance between sodium and potassium in the blood, thus enlisting osmolar support to regulate water retention.

Collaterally, vasopressin, or antidiuretic hormone, produced in the hypothalamus and released by the pituitary gland in response to a decrease in blood volume exerts a pressor effect and acts as a diuretic by reducing the volume of urine, thereby conserving the volume of blood. That the caliber of blood vessels, for example, is adaptive locally as well as systemically demonstrates that control is effected by a hierarchy of control loops wherein those subordinate interact with those progressively more encompassing until the center just above the brainstem is reached. Central mechanisms initiate the release of circulating vasoconstrictors or vasodilators that cause the linings of blood vessels to contract or relax in response to the condition of the circulatory system, which includes cardiac output, partial pressures of oxygen and carbon dioxide, and the existing concentration of hormones and electrolytes in the blood.

Blood pressure as the product of cardiac output and peripheral resistance subsumes numerous interrelated contributory closed loop actions responsive to emotional state, level of exertion, temperature, metabolism as affected by ingesta, disease, medication, and gas exchange in the lungs effected by cellular level feedback between every cell and its immediate environment. Maintaining normal function in the walls of bodily conduits is thus central to and inseparable from maintaining normal function. Much the same hierarchical integration mutuates between the wider physiological context and any other bodily conduit, whether ureter, gamete transporting duct, the airway, choroid plexus and arachnoid villi, or lymphatic vessel.

Secreting enzymes and mucus, all segments along the digestive tract interact with and actively condition the passing contents—until disease interferes with this process. The linings of vessels, ducts, and other type bodily conduits are properly conceived of as distributed organs and glands that participate in regulatory feedback loops in their own right. A diseased condition of the wall surrounding the channel or the lumen is not just a matter of local structural degradation but signifies disruption in the many important chemical regulatory pathways or loops in which the cells within the wall participate. As a result, no disease in the wall of a bodily conduit is properly viewed as merely local; left untreated, what appears local disease will eventually initiate a cascade of dysfunction that advances to encompass more and more of the body, and as most convincingly exemplified by cancer, can result in death.

While the body is able to compensate for numerous forms of degradation, such as those associated with aging, a failure to produce an essential enzyme or to produce an essential protein as the result of a genetic defect or progressively emerging alteration, for example, is sufficiently anomalous that the body lacks sufficient responsive measures. Thus, up to the degree of deviation that can be accommodated, an atherosclerosed artery is continuously remodeled, preserving its luminal diameter, for example, but the inadequate synthesis of insulin, resulting in diabetes, or tyrosine, resulting in phenylketonuria, for example, demand human intervention.

Such anomalous defects, to which the body has limited if any adaptive or accommodative compensatory response, account for much of internal medical practice. Application to controlled steering of a prosthetic hand has been addressed (Light, C. M., Chappell, P. H., Hudgins, B., and Engelhart, K. 2002. “Intelligent Multifunction Myoelectric Control of Hand Prostheses,” Journal of Medical Engineering and Technology 26(4):139-146; Chappell, P. H. and Kyberd, P. J. 1991. “Prehensile Control of a Hand Prosthesis by a Microcontroller,” Journal of Biomedical Engineering 13(5):363-369), but nowhere does there appear the application of hierarchical control to continuous adjustment in the execution of a prescription or any end motion-unrelated medical use.

Where, due to adverse side effects and/or drug-drug interactions, conventional means for overcoming the intrinsic defect prove problematic, the solution will often lie in the targeted delivery of the drug or drugs to the site or sites of the origin and/or symptoms of the disease. A prosthetic adaptive or compensatory system to supplement that intrinsic requires secure means for connecting to ductus, and the ability to deliver drugs automatically according to a program that allows some latitude in the timing and dose of each based upon feedback. A review of copending application entitled Integrated System for the Infixion and Retrieval of Implants with or without Drug Targeting will make clear that the use of ductus side-entry connection jackets, whether simple junction or piped impasse jacket in type, can be coordinated with unpiped magnetic impasse jackets.

Side-entry connection jackets can be used to deliver a drug directly to a certain level along a conduit, and when the side-entry connection jacket incorporates a magnet, the drug can be drawn into the wall of the conduit where it is delivered, or a magnetic jacket downstream can take up the drug after it has passed over the conduit lining. The ability to deliver or release drugs and reversal agents at any level along a conduit means that any segment along the conduit can be targeted, sparing other tissue from exposure to a drug or radionuclide. Placed along an artery, the level of the jacket sets the supply territory or region treated, so that advancing the jacket along the arterial tree closes in on and narrows the distal target supply territory by excluding branches to neighboring tissue, whereas retreating includes other branches and thus expands the target zone.

To avoid encroaching on neighboring tissue, the side-entry jacket selected should be as narrow and short as possible, and positioned to avoid the aortic body or to encroach upon the pulmonary trunk, pulmonary arteries, or the superior vena cava, for example. When clinical judgment is to place greater importance upon avoiding the systemic distribution of the drug, service channel lines, or sidelines, seen as part number 11 in the accompanying drawing figures, accessed through the port or ports implanted at the body surface, are used to deliver heparin and/or other medication to suppress the formation of thrombus and shear stress-induced endothelial dysfunction and intimal hyperplasia.

In adrenocorticotropic hormone or ACTH-independent endogenous (nonpharmaceutical, noniatrogenic) Cushing syndrome, it is essential to selectively lower the production of serum cortisol and not the serum concentration of ACTH or other corticosteroids produced by the adrenal cortex in response to stimulation by ACTH. In this situation, ACTH input to the adrenal cortex itself should not be affected. Therefore, unmagnetized or simple junction piped jackets are placed about the most suitable of the 3 suprarenal artery inlets to the adrenal gland to release metyrapone, ketoconazole, and/or aminogluthimide in higher concentration and potency than might be prescribed for systemic use. The higher concentration of these enzyme inhibitors then more effectively blocks cortisol synthesis without exposure to unintended tissue.

The side effects resulting from this exposure having limited the opportunity for medical management to a brief interval preceding adrenalectomy (see, for example, Young, V. B., Kormos, W. A., Chick, D. A., and Goroll, A. H. 2010. Blueprints: Medicine, Philadelphia, Pa.: Lippincott, Williams, and Wilkins, pages 238-239), a need for the more radical and complication prone procedure should be averted. Restriction of ketoconazole for example, to the adrenal gland allows the overproduction of corticosteroid hormone to be suppressed without risk of hepatotoxicity (Merck Manual, 18th edition, page 1214). Were no blocking but a reversal agent available, the impasse-jacket would be placed at the outlet—proximate to the glands about the suprarenal veins to release a substance selectively neutralizing for the hypersecreted or overproduced cortisol.

In this situation where the Cushing's is pituitary-independent, any increase in ACTH secretion will increase 11-deoxycortisol, which is considerably less potent than cortisol. In systemic doses, metyrapone and ketoconazole can produce adverse side effects. Targeted however, the dose is too low to cause complications. The direct application of a statin to atheromas for the pleiotropic local and nonhepatic benefits and of glucocorticoids, immunosuppressants, and antibiotics to treat a regional enteritis are examples of substances that can be delivered in far higher concentration, hence potency, when directly targeted at lesions while kept from the rest of the body, the drug concentrations used thus toxic if circulated.

However, autoimmune disease genetic and lifelong in accordance with the general guidelines for the use of ductus side-entry jackets to treat long-term or chronic disease, when immunosuppressant drugs can be targeted at circumscribed tissue as by release directly into the ductus, usually an artery, supplying that tissue, immunosuppression that renders the patient susceptible to intercurrent disease is avoided. Since the substance used is drawn into and taken up within the lesion so that absolute amount of the dose in whole-body terms is minute, the need to pre-position a second jacket downstream to release a reversal agent should seldom arise. For intermittent or spaced apart lesions such as the ‘skip’ lesions in ileocolitis or plaques in atherosclerosis, each jacket is placed to encircle a lesioned segment, any propensity to migrate (move, displace, dislodge), additionally suppressed by connecting the jackets in a train.

Interruption or sectionalization thus achieves not only drug delivery optimization through discriminatory and focused targeting, but allows flexion, leaves intervening segments unenclosed and open to the chemical mileau, and allows reduction in the extent of dissection essential for access and placement. Avoiding bands of connective tissue, mesentery, and the uterine latus or broad ligament, for example, preserves not just mechanical means of support but nerves and vessels. Reducing trauma and disruption to function by avoiding the need to section supporting tissue advances the object of minimizing trauma central to the use of minimally invasive technique. Unshielded jackets about the gut, for example, can straddle small nerves and arteries to avoid Plaque vulnerable to rupture or calcified as would block penetration is treated at the margins.

The addition of a radiation shield to the jacket disallows perforations, cutouts, or division into segments shorter than the lesion for flexion, reduction in weight, and aeration. Were not the radiation shield used formulated to disintegrate, these factors would limit the use of a shielded jacket to conditions where conventional surgery would pose greater risk, such as resection likely to induce paralytic ileus. Jacketing either renal artery targets the kidney to that side; the portal vein, the liver; internal carotid artery, the supply territory on that side of the brain; the internal thoracic artery, the nipple-areola complex on that side; and a coronary artery, its supply territory within the myocardium. To treat pulmonary tuberculosis or asthma, jackets about the bronchi are used with a nebulizer or inhaler that releases susceptible drug-carrier particle-bound drugs to treat the air passages, while a jacket about the pulmonary trunk is used to treat the blood entering the lungs.

Accessing the bronchi by direct jacketing with or without thermoplasty and terminal bronchioles and alveoli thus and by supply artery, restricting the use of drugs used to treat asthma such as omalizumab to the site intended averts the dizziness, earache, arthralgia, and several other side effects remote from the treatment site associated with the drug. To treat a solitary pulmonary nodule or tumor in a lung, a jacket about the pulmonary artery on that side is used. Jackets can have an arc omitted or include cutouts to gain clearance. Provided a malignant tumor such as a basal cell carcinoma—which does not shed daughter cells spread by hematogenous, or lymphatic dispersal—can be directly targeted with highly toxic chemotherapeutic agents, the adverse side effects associated with conventional chemotherapy will be significantly reduced if not eliminated.

Tumors known to spread by direct extension are treated by corresponding extension of the jacket or jackets. However, metastatic—and micrometastatic disease where malignant cells may have been shed by the tumor that were too small to be detected—demands systemic treatment. Moreover, to destroy daughter cells shed by a primary tumor and any metastases generally requires systemic chemotherapy at the same concentration as is used to treat the primary tumor. In such a situation, targeting to the extent of infusion directly into an end organ, isolated limb perfusion, or external beam radiation likewise require backup systemic chemotherapy; however, as with alternative methods for targeting a lesion expressed by a systemic disease, the higher concentration directed at the primary tumor can reduce the time that the tumor is able to shed daughter cells, and therewith, the duration of treatment.

For disease that is systemic but nonmetastatic such as atherosclerosis or regional enteritis, a background systemic or digestive tract dose is essential but can be much reduced compared to that forced into the lesions by the jackets. Except with metastatic cancer, where treatment must be systemic and uniform, the same or other substances can be differentially delivered to impasse-jackets without interfering with the concurrent administration of a background systemic dose administered by infusion, injection, or ingestion. In fact, a fraction of the dose delivered thus can be the carrier bound drug.

That the targeted portion represents a concentrated dose does, however, allow the systemic dose to be less concentrated, averting numerous complications such as adverse drug-drug interactions and dose related side effects. With atherosclerosis or a vasculitis able to induce localized obstructions the qualification stated with respect to metastatic disease, that the systemic dose must be as concentrated as that intended for the primary tumor does not apply; the background dose can be much reduced, eliminating myopathy regardless which statin is used. Limitation to the jacket can substantially isolate a drug from adverse drug-drug interactions with another drug in the general circulation and supplement or reduce the level of any blood constituent that passes.

To minimize return flow and spillage when lines must be disconnected and/or entered to pass through a scope, for example, drug-containing and inert filler materials delivered through a sideline, that is, a side-entry line or a service channel seen in the drawing figures as part number 11, especially when more than one service channel connects to a single side-entry connector, are ordinarily prepared in the form of a viscid flowable substance. Such includes crushed tacky nonalcohol nonacetone hydrogels, or aquagels, sufficiently durable as not to liquify or transition to the sol state when driven or aspirated, jellies, syrups, cellulose gums, slurries, ice slurries, and pressure pumpable semisolids, such as petrolatum, all of which can be formulated to include medication.

Ordinarily, the side-entry connector, shown as 6 in the drawing figures, has two inlet lines, the primary of mainline of the side-connector itself, seen in the drawing figures as 13, and a subsidiary or accessory water-jacket and service channel sideline 11, connected to water-jacket 7. These lines are ensheathed within a common catheter that extends from the paired pumps, packaged as a unit, to the jacket, but can be cut away to part and separately route the lines within the body, that is, distal to the port implanted at the body surface. At a given time, each jacket mainline and sideline is connected to an independently controllable miniature bidirectional or reversible variable speed positive displacement pump. Fluid lines leading from the pump-pack to the port at the body surface are protected by ensheathment within flexible, such as ‘gooseneck’ conduit.

For lesser volume delivery in relatively simple applications such as the simple junction jacket depicted in FIG. 16, a commercially available syringe driver based infusion pump can be used; however, this assumes placement of the jacket using a separate pump in the clinic and that adjuvant delivery is through compounding the drug delivered through the single line. If a jacket positioned as that depicted in FIG. 16, for example, were to surround a segment of a ductus containing a lesion such as a plaque, then a magnetized jacket such as those shown in FIG. 4 would allow a magnetically susceptible particle bound statin, for example, to be targeted into the arterial wall over the jacketed segment for its anti-inflammatory and other pleiotropic properties.

If the drug or diagnostic agent to be targeted were radioactive, then radioactive shielding would be applied along the delivery path from drug reservoir or refill cartridge to the artery, the long term administration of a radionuclide by, means of a jacket of the kind shown in FIG. 5, and shorter term use with a jacket of the kind shown in FIG. 6, which fenestrated or perforated to allow the adventia to ‘breathe,’ can remain in place to deliver medication or take test samples as necessary. Similarly, for higher volume medicinal or nutrient delivery in a relatively simple and straightforward application, connection is by insertion of a hose or line from an external reservoir which is inserted into a socket of the turret supplying the rotary peristaltic or roller-head type pump, to be addressed in greater detail.

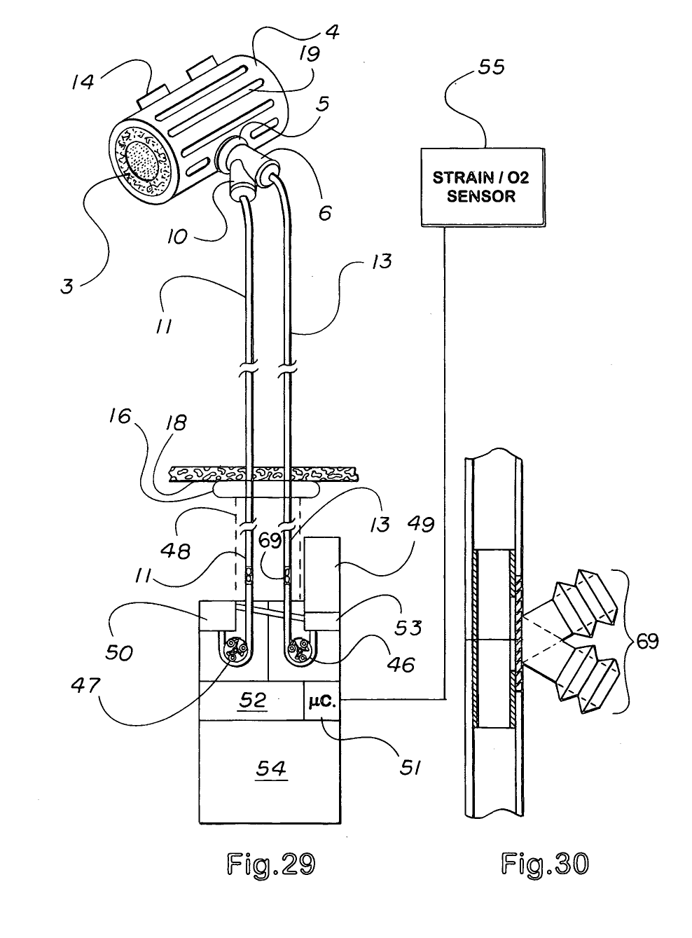

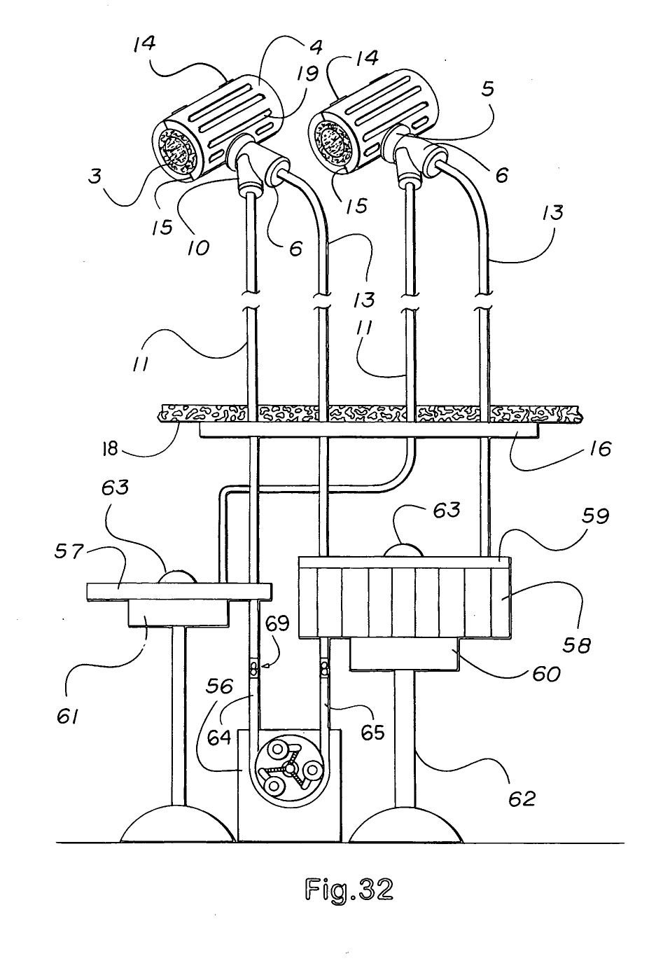

However, to treat multiple established comorbid and unanticipated intercurrent conditions served by multiple side-entry connection jackets positioned along the same or different type ductus, and to do so in a comprehensive and coordinated manner, the connections between inlet and outlet lines to each pump in each mainline and sideline paired pumps, or pump-pair, are made switchable in timed relation to each of the others. Switching the jacket destination of a given pump in a given pair obviates the relation of the pair to the mainline and sideline of a specific jacket as pertains to the relatively simple application depicted in FIGS. 31 and 32. Since ordinarily, the mainline, or side-entry connected line 13, and the sideline, or water-jacket inlet line 11, are used differently, the ductus side-entry connection jacket, as shown in FIG. 29, is supported by a pump-pair wherein one pump is usually assigned to the one line and the other pump to the other line.

While various relay or valve type switches can be used to switch different therapeutic substances from one jacket to another, for pictorial clarity, the figures depict line switching as effected by means of turrets. These lines are permanently connected to their respective jackets; however, each jacket is provided with as many side-connectors 6 and accessory or water-jacket inlets 10 as necessary. As may be discerned from FIG. 32, which shows line switching turrets at the intake and outlet lines of each pump, either or both inlets to each jacket can be switched to a different drug vial or reservoir. Depending upon the connections made between pumps and jackets, a pump or pump-pair can support one or more jackets, and more than one pump-pair can support a single jacket.

Simple mechanical methods for flushing fluid lines include passing a guidewire with round brush or swab through a clean-out type line port accessed through the pump-pack as shown in FIG. 31 or passing an aspiration catheter connected to a vacuum pump through the line. Provided to do so will not affect a drug in a line, the inmate pump can be used. For the patient not required to return to the clinic on a frequent basis, flushing is better automated. Using the arrangement shown in FIGS. 32 and 36 with successive drug source vials or reservoirs switched while the delivery lines are fixed to the inlets of either jacket, the dose intended for either jacket must take into account any residue left along the internal walls of the lines. Adhesion will vary according to the properties of the substance delivered. When the residue is significant and mixture with a succeeding drug is to be avoided, or when the line is to be left clean, the residue is flushed either forward into jacket inlet 6 or 10 to complete the intended dose or out of the line, usually to a reservoir.

The lines can also be switched to allow a hydrogel, solvent, or wash water, for example, to be recirculated through the closed circuit past the opening in the ductus and into a reservoir. Ordinarily, the relation of pumps to jackets is simple and direct on a one for one basis, but if necessary, a pump or pump-pair can be switched to different jackets, association thus typified in a pump-pair and jacket set wherein a pump-pair support four side-entry jackets. Using the arrangement shown in FIG. 29, to recirculate either pump outlet through a closed circuit past the opening in the ductus in either direction requires the coordinated control over both pumps, the flow passed up through either pump line and back through the other, with completion of the circuit through a reservoir inlet and outlet (not shown).

When the ductus infusion flow rate of two therapeutic substances in fluid form is to be equal or immediacy of tracking in either the antegrade or retrograde direction are paramount, a double arm or bidirectional side-entry connector similar to the clean-out or inline port shown in FIG. 30 and described below is used. Such a double side entry connector is placed the same way as those shown in the drawing figures but does not require a water-jacket, either of the arms available for this role during placement and for use as an accessory inlet following placement. A double armed side-entry connector differs from the same general configuration as used in a pump inline port or clean-out, or double arm bidirectional line lumen access fitting, in that the clean-out is placed inside the line lumen after the line has been die cut to accept it and is connected to a surround used to shield the pipe lumen from gouging in either direction, while the double arm side-entry jacket side-connector is a part of the jacket.

The jacket, however, because it surrounds a native ductus and an object of the invention is to eliminate the need for any foreign object to remain in the lumen, requires that the tip of a guidewire, cabled device, or catheter be blunt nosed and wetted with a specialty lubricant such as ACS Microslide®, Medtronic Enhance®, Bard Pro/Pel® or Hydro/Pel®, Cordis SLX®, or Rotaglide.® Either a single or double arm side-connector can be connected to a single pump to obtain closed circuit recirculation. To prevent leakage while allowing a cabled device, guidewire, or fluid drug under pressure to enter a native lumen in either direction without hesitation, a ductus double armed side-entry connector has a slit elastic membrane as a bidirectional check valve at physiological forces covering the opening into the jacket. Similarly, to allow entry of a guidewire into a pump line in either direction with minimal leakage, a clean-out, or double armed bidirectional lumen access inline port, as shown in FIG. 30 also uses such a membrane.

The double arm inline port or bidirectional pump line clean-out or shown in FIG. 30 also allows a fluid to be delivered into the pump line for delivery to the ductus side-entry jacket by an external pump. The external pump is plugged into the opening at the back of the pump-pack for insertion in the pumpward or ductusward direction as desired to deliver or aspirate fluid such as to deliver a drug not included in the turret or obtain a diagnostic test sample of the native lumen contents. With the pump lines leading from the pump-pack disconnected, the port fastened at the body surface with elastic slit membranes covering the loose end and port openings is available as a unidirectional clean-out type inline port distad, toward the jacket. When treatment is of a vessel, whether a tacky gel in the line that resists spilling out when the line is disconnected allows dispensing with slit membranes depends upon whether any gel spills out of the line risking gas embolism when the line is reattached.

Inserting a cabled device, guidewire, or external pump line through the opening at the port leading to side-connector 6 allows access into the ductus. However, the need to disconnect the lines plugged into the port is eliminated by placing double arm clean-out inline ports in the intake and outlet lines of each pump according to the probability of a tissue plug jam or the accumulation of debris. Since the clean-out type inline port is entered at the side rather than at the end of the line as is the body surface port, it allows the insertion of a cabled device such as a fine endoscope, aspiration catheter, or a special corkscrew-tipped guidewire to extract a tissue plug, for example, in either direction without the need to disconnect the line. Insertion can be in the intracorporeal direction toward the jacket or in the extracorporeal direction toward the pump. Drug concentration often a benefit of targeted delivery, substances to be moved through the lines should be adjusted in viscosity and susceptibility to avoid buildup or clogging.

Lines can be flushed through by recirculating wash water from one reservoir to another through a circuit closed by a line connecting the reservoirs as shown in FIG. 29, for example. This reduces if not eliminates the need to enter lines through a port, whether at the body surface or a clean-out type inline port, in order to flush these through. The need for servicing or maintenance is also reduced through the use of side-entry connection jackets having more than one side-entry connector and/or accessory inlet (service channel, water-jacket, or sideline), through the use of a double-arm type side-connector, or using a jacket with both a conventional side-connector as shown in FIGS. 1-6 and a double-arm type as shown in FIG. 7. Since the double arm or bidirectional clean-out type inline port shown in FIG. 30 allows insertion directly into the line lumen, there is neither a need nor an advantage in using a port for access to line lumina at intracorporeal levels.

Access through inline ports in the pump-pack to any level up to the native lumen eliminates the need for access to intracorporeal levels of the line that requires entry through the surface port after the lines from the pump-pack have been disconnected. Furthermore, because entry into a line lumen except through the body surface port with pump-lines disconnected must be through the side of the line, direct intracorporeal access would require an additional aperture in the body surface port leading into the space between the lines. And since the sheath enclosing the intracorporeal lines from the surface port can extend no more distally than the level at which the lines diverge, the jacket inlet line and when present, a port situated thus to allow switching between either of two input lines to the same jacket inlet are limited to this distance. By contrast, the clean-out type inline port allows entry into any line to any distance from an insertion point within the pump-pack.

When the application would benefit from frequent endoluminal examination by means of a fine fiberscope or intravascular ultrasound probe, for example, both the line entry port in the pump line within the pump-packet and the side-connector are of the double arm type. Entering the lower opening at the back of the pump-pack will lead the catheter or cabled device up the pump line. If the pump line is connected to the upper arm of the side-connector, the nose or probe is steered to reverse direction with little if any hesitation. Once the lines emerge from the sheath behind the surface port, tissue may backfill in and around the lines. A packaged single jacket pump-pair and jacket set includes lines permanently connected to either pump outlet and omits pump outlet turrets. A packaged pump-pair and jacket set usually contains a single standardized pump-pair, wherein each pump has intake and outlet turrets to allow switching any drug at the intake turret to any one jacket in the set at a time.

### Capability and Standardization of Control

For economy of manufacture, components of the pump-pair and jacket set other than the number of jackets connected at any one time up to the maximum are kept substantially standard, a microcontroller with the same maximum number of cores drawn from the same family and the programming or programming tree written using the same language. Provided the control system is adequate for the medical condition, the action required of the turrets and pumps reduces to so many rotational increments. For this purpose, open loop driven stepper motors, one each per turret and pump, are able to satisfy the most critical applications. Moreover, since the pumps and turrets are fully enclosed and protected from outside contact, the additional cost of closed loop control to allow spontaneous adaptation is not justified.

### Control System

Control therefore is preferably of sensor response adaptive closed loop control over the delivery of each drug in the turret, control of the pump and turret stepper motors under open loop controlled. While the same degree of complexity and expense is not warranted in less serious cases, in a patient with an unstable life-threatening condition, adaptive response justifies the implantation of sensors tied into closed loops in a wireless body area network with automatic adaptive response in the dosing of each drug by means of a hard real time adaptive hierarchical or nested complex control system.

Such a system is analogous to the kind used to control a remote vehicle, for example (see, for example, Findeisen, W. 1984. “The Essentials of Hierarchical Control,” in Thoft-Christensen, P. (ed.), System Modelling and Optimization. Lecture Notes in Control and Information Sciences 59:38-61; Findeisen, W.; Bailey, F. N., Brdys, M., Malinowski, K., Tatjewoki, P., and Wozniak, A. 1980. Control and Coordination in Hierarchical Systems, New York, N.Y.: John Wiley and Sons, Issue 9 of the International Series on Applied Systems Analysis, Wiley-Interscience; Meystel, A. M. and Albus, J. S. 2002. Intelligent Systems, New York, N.Y.: John Wiley and Sons; Albus, J. S. 1995. “RCS: A Reference Model Architecture for Intelligent Systems, Association for the Advancement of Artificial Intelligence Technical Report SS-95-02, available at http://aaaipress.org/Papers/Symposia/Spring/1995/SS-95-02/SS95-02-001.pdf; Albus, J. S. 1993. “A Reference Model Architecture for Intelligent Systems Design,” Chapter 2, pages 27-56 in Antsaklis, P. J. and Passino, K. M., eds., An Introduction to Intelligent and Autonomous Control, Baltimore, Md.: Wolters Kluwer Academic Publishers; Aguilar, J., Cerrada, M., Mousalli, G., Rivas, F., and Hidrobo, F. 2005. ““A Multiagent Model for Intelligent Distributed Control Systems,” 191-197; additional references provided below).

With the proper sensors, such a system can be programmed to assimilate or ‘learn’ events as these are experienced, such as the action of a drug at an interval other than expected (Albus, J., Bostelman, R., Hong, T., Chang, T., Shackleford, W., and Shneier, M. 2006. “The LAGR [Learning Applied to Ground Robots] Project: Integrating Learning into the 4D/RCS [4 Dimensional Remote Control System] Control Hierarchy,” International Conference in Control, Automation and Robotics—ICINCO 06, Setubal, Portugal, available at http://www.nist.gov/customcf/get_pdf.cfm?pub_id=822702). Unless interrupted by an adverse event, the drug regimen continues unaffected. In such a hierarchical control system, the processors of a monolithic integrated circuit or microchip multicore microcontroller are partitioned to support one control node each, that programmed to function at the highest level of control as the master node sent the inputs from and having a time horizon comprehensive of the subordinate nodes, of which each contributes inputs to the pumps and jackets of the set based upon the sensors that feed it.

More than a single subordinate control level exceptional, a pump-pair and jacket set that includes three jackets, for example, requires a microcontroller with at least four cores (referred to by Parallax, Inc., whose multicore microtroller chips have the individual cores arranged in a circle or ‘hub’ for access to shared memory, ‘cogs’). Magnetic gradient-incorporating side-entry jackets, or piped impasse-jackets, already capable of drawing superparamagnetic carrier bound drugs radially outward through a ductus wall, patch-magnets are placed not to encircle ductus, but attached to the outer capsule of an organ supplied by the ductus and subject to the disease process under treatment. In most instances, the sensors are packaged in the form of stays configured for concentric insertion into the wall of the ductus or parenchyma before the jacket or patch-magnet is applied, so that these sit beneath or within the jacket or patch-magnet.

Minute diagnostic sensor implants respective of each jacket, patch-magnet, or other type implant provide feedback to the lower level nodes respective of each jacket, patch-magnet, or other type implant feedback site, thereby adjusting the dose of the drug respective of each within the prescribed drug delivery context, or the prescription as maintained by the master node. Highly stable conditions may require no more than one sensor closed feedback loop if any. Significant cost reduction may be achieved by limiting control software and hardware to the nonadaptive where more complex control and artificial intelligence are unnecessary. Whether control is nonadaptive or complex, the drivers remain standardized interchangeable pump-pair and jacket set open loop-driven stepper motors. The program automatically and immediately adjusts the delivery of medication for the present condition.

To cover different ranges of disease severity, the multicore microcontroller stores more than one program or prescription. Upon receiving appropriate sensor feedback through one or more subordinate nodes, the master node automatically transfers the program for the out of range node and jacket or the entire set. Should the feedback signals reflect a condition outside the drug delivery response range of the apparatus, a wireless body area network transmits an alarm to the clinic by emergency band or a conventional communication means, such as a text message. Depending upon the urgency, the input can be applied to dispatch an ambulance, alert the patient to return to the clinic, or instruct the patient to connect the pump-pair intake turret lines to higher capacity tabletop drug reservoirs containing the same or different drugs and switch to a different prestored control program.

The set produced as a unit, a single jacket set omits pump-pair outlet turrets, while multi jacket sets with a reasonable limit of four jackets provide pump outlet turrets to allow switching the pump outlets to any one jacket at any one time. More elaborate line switching as would permit simultaneous outlet switching to more than a single jacket at a time is possible but elusive of practical medical purpose, needlessly complex and costly, and inviting human error. In any such set, the lines connecting the pump-pair to each jacket is permanently fastened to the main and sidelines of each jacket, pump outlet switching among jacket inlets in the set accomplished at the pump outlet turret where any line to any jacket in the set can be rotated into alignment with the pump outlet.

A pump or pump-pair and jacket set thus constitutes a unit apparatus, of which portions proximal to the port implanted at the body surface remain outside the body, or extracorporeal, with those distal to the port implanted, hence, intracorporeal. Since individual jackets in a given standardized pump-pair and jacket set can be different sizes, can be placed along different type ductus in different parts of the body, and the one pump-pair supporting the jacket set allows the delivery of any drug to any jacket in the set in any sequence at any time, to further admit the inter-switching of lines among different pump and jacket sets only causes confusion. Jackets belonging to different pump-pair and jacket sets can be interposed with drug delivery times controlled by the multicore microcontroller in the multipump-pair power and control housing, or base into which the pump-pair plug-in modules insert. However, the need for more than one such set should prove rare and limited to cases of severe multiorgan disease or extensive injury.

At the pump intakes, the individual drug vials or external reservoir inlet pipes insert into an exchangeable rotary magazine that fits onto and is engaged by a turret drive gear. During production and use, each vial and external reservoir inlet hose is identified with a barcode or magnetic stripe running down the side, preloaded cartridges barcoded and sealed. Further to prevent human error, pre-loaded rotary cartridges or sectional trays keyed to prevent incorrect insertion in the turret. While the jackets in a given jacket set can be different sizes and applied along different type ductus, to achieve standardization within the foreseeable scope of medical practicality, the pumps in any given pair are the same, the pumps in different pairs the same, and the drug vial, refill cartridge, or inlet pipe receiving turrets built in and the same. To avoid pump stalls, the spaces or lands separating adjacent vial wells is minimized.

The standardized pump-pair with jacket or jackets in a jacket set plugs into a power and control housing made to accept one or more standardized pump-pair and jacket sets, the power and control housing enclosing a power source and multicore microcontroller commensurate with the number of pump-pair jacket set units it can accept. As shown in FIG. 29, standard or packaged production unit pump-pair and jacket sets that include a single jacket to receive a single drug do not require pump outlet turrets, whereas those with more than one jacket must provide jacket inlet lines leading to each jacket from either pump outlet turret. The pump-pair or pairs and power and control housing are worn in a pump-pack suspended from a waistbelt. Only highly prevalent conditions warrant the production of self-contained standalone units wherein the jacket or jackets are unitized with the power and control housing in a packaged unit.

For the patient provided with more than one pump-pair and jacket set, standardization affords a measure of fail safety in redundancy, different pump characteristics not having to be accounted for as additional variables. In a drug intake turret, the intake of either pump remains stationary as the drug vial or remote drug reservoir inlet line rotates into pump inlet alignment. In a drug outlet turret, the outlet of either pump remains stationary as the jacket inlet lines rotate into pump outlet alignment. This redundancy allows the program to comprehend a larger number of automated responses to inputs, routine or emergency. Because the advantages gained in providing at least one side-entry connector and one accessory line are considerable, and because the flexibility imparted by line switching between the pumps allows noninvasive responses to exigencies, pumps are always paired.

Furthermore, the pumps in a given pump-pair are the same; for all practical purposes, each is assigned to support the same jacket or jackets positioned along the same or a like ductus or to ductus belonging to different bodily systems but having approximately the same diameter, such as one jacket on a renal artery and the other on a ureter. Only differences in the volume and rate of drug delivery to either of two jackets so pronounced that adjustment in concentration and/or the rate of drug delivery to either could not compensate for the different delivery rates would justify the use of a second pump-pair differing in capacity. Such an eventuality is not foreseen. With pump standardization, identification to the program of the relative position of each pump and pump-pair inheres in the electrical connection associated with its socket position, no further distinction required.

This much simplifies programming. When more than one pump-pair is placed under unified control so that the output line of any pump can be redirected to feed into mainline or sideline nominally assigned to another jacket and pump-pair, distinction as a pump-pair becomes one of joint housing, of structure, and often but not always, of function. Since the sideline will often be used to introduce adjuvant medication in relatively small doses and at lower rates than will be delivery through the mainline, and because no application of a side-entry connection jacket may reasonably be expected to eschew the need for this mode of drug delivery, and because the simplest or purest method for allowing different volumetric flow rates and directions is simply to provide two pumps, the pump-pair, rather than the individual pump, is preferred as the unit of pumping componentry manufacture.

As shown in FIG. 29, this allows one pump to be connected to the side-entry connector, or mainline, and the other pump to the water-jacket, or sideline. Provided each pump is provided with a turret, each can, however, be switched between lines to the same or other side-entry connection jacket, allowing, for example, recirculation around a closed circuit driven by a single pump, that on the left-hand side in FIG. 29. The order of increasing utility therefore begins with the pump-pair as effectively irreducible from a practical standpoint; next, optimizing the functionality of each pump in the pair by incorporating means to allow switching inputs from containers, hoses, or other repositories of different drugs or other therapeutic substances; next to incorporating means such as a turret for switching the output of each pump to different lines, therewith realizing the full scope of flexibility obtainable from a single plug-in module pump-pair.

In terms of existing drug regimens, a single pump-pair and jacket set, or jacket supported by a single plug-in module pump-pair, will support any less complex medical requirement. For this reason, an individual side-entry connection jacket and plug-in module pump-pair as applies to FIG. 16 is considered a standard basic pump-pair and jacket set unit configuration for production. When no more than one plug-in module pump-pair is needed, the power and control module receiver, or base, contains only one pump-pair socket and requires a battery and multicore microcontroller sufficient to control only the one pump-pair. Next in order of increasing utility, physiological sensors, or s, that generate closed loop feedback signals programmed to active adaptive control over drug delivery are incorporated.

Next in order, prompting future developments, comes furnishing more than a single plug-in module pump-pair, both with intake and outlet turrets, and a power and control module able to coordinate the pump and pump turret motions under synchronous control. Survival-dependent applications that require a single pump-pair are usually duplicated in a two pump-pair receiving base for redundancy. In this case, one of the pump-pairs is a standby that takes over when energized by an automatic transfer switch, and the power and control housing need not support more than one pump-pair at a time. Ordinarily, the battery and multicore microcontroller or mixed digital analogue signal field programmable gate array and microcontroller must be capable of powering and controlling the number of pump-pair plug-in modules inserted into it at any given time.

Most to benefit from automatic drug delivery are patients with multiple disorders who are incapable of or inattentive to prescription compliance. Adherence to a more complicated drug regimen, especially one tightly scheduled, when some drugs are to be swallowed, others absorbed through the oral mucosa, and others still injected, for example, can elude even a mentally unimpaired patient. Commercially available automatic infusion and insulin pumps are not designed to do this. Moreover, to wear separate pumps would impede freedom of movement, and the action of each pump would proceed independently of the others, denying the ability to deliver drugs to the different jackets in a strategically coordinated or neoadjuvant manner throughout the day. By contrast, here substance delivery is under the coordinated and synchronized control of an embedded multicore microcontroller which can be overridden, if necessary, remotely by the physician in response to a distress call.

Regardless of the number and type of components used, portable components are housed within a strong, compact, and light in weight wearable, or ambulatory, aluminum alloy or plastic enclosure and provided with connections for the use of stationary apparatus. Light weight pump-packs are suspended from a waist belt. To distribute the weight and dimensions, a combination of mainline and sideline pump-pairs too awkward or heavy to house together are divided among separate smaller pump-packs or unified and carried in a backpack or rucksack, coordination among the pump-pairs remaining under unified control by connection to the microcontroller. Either pump in a given mainline and sideline pump-pair can be a syringe driver or a rotary peristaltic pump, the latter more likely to be supported by automatic line switching.

While negligible advantage is to be gained from the reversibility and variability in speed of each pump in a pump-pair at the current state of pharmaceutical science, this can be expected to change: For example, an advantage in the ability to switch each pump in a pump-pair between mainline and sideline in the single jacket application shown in FIG. 16 would be exceptional, the added complexity of an automated crossover capability only adding to the cost. When only one side-entry connection jacket is required, the pump-pair is made in different sizes with inmate battery and controller as a standard unit. Such a unit, sold with lines of set length and port, diagrammatically represented in FIG. 29, for example, with four jackets provided, requires only that the final overall, or extracorporeal and intracorporeal, length of each line after placement be changed by manual entry of the new values into memory thus changing these values from the preinstallation values that had been based upon the total length of the lines when produced.

More intricate line switching, such as relatively quick changing from the line connections of the kind shown in FIG. 29, depicting instant connections during the recirculation of medicated crushed tacky gel or wash water during installation, to connections of the kind shown in FIG. 31 for the ongoing support of the application shown in FIG. 16, for example, are susceptible to human error and entrusted to the microcontroller. When more than one jacket is required, the number and capabilities of the pump-pairs depends upon whether the nature of the disease, its distribution in the body, or comorbidities require or recommend that each pump-pair be coordinated and synchronized with each of the others. Referring to FIGS. 32 and 36, for example, this synchronization includes the coordinated control over the rotatory indexing of the drug supplying turret that determines the drug to be delivered to the connected jacket and the pumping action of the pump.

If housing two or more pump-pairs in the same enclosure results in a pump-pack that is too heavy, then the pump-pairs are housed separately, or distributed, each with inmate power source; however, to coordinate the action among the pumps when necessary, the separate pump-packs are placed under the centralized and unified control of a microcontroller housed in one of the pump-packs. If each pump-pair in a distributed set can function autonomously, then each has a power and control housing with inmate microcontroller. For treating chronic but stable conditions, a need to adjust the control program might not arise over the lifespan of the patient or the apparatus. However, a change or changes in the medical condition can never be predicted with confidence.

Where the consequences of a change in the condition would be grave, the need for sensors to monitor the pertinent signs and a control program or subroutine and microcontroller able to readily adapt to the change is clear. The program must execute the pump and turret actions essential to carry out the prescription for the unique combination of drugs loaded in the refill cartridges, ampules, or vials at each pump intake turret, that is, coordinate the delivery times and rates of each, hence the speed and runtime of each pump in the combination. Since the possible combinations of drugs is prescribed from among the drugs available at the time for the one or more conditions diagnosed at the outset, the library of available programs is prepared in the order of prevalence and limited to a given disease or combination of diseases, uncommon and rare conditions necessitating adaptation, combination, or revision if not the preparation of a special program.

The length of the lines connecting each pump from each side-entry connection jacket is substantially constant and stored in memory. As shown on the right hand side of FIGS. 32 and 36, a pump that must deliver multiple drugs is provided with a revolver-cylinder configured turret pump intake mechanism, or loader, which arranges the socket for each drug vial or refill cartridge at intervals about a concentric to the plane of the turret such that paired rotary solenoids or a stepper motor under the control of a microcontroller or multicore microcontroller can index any vial into alignment at the pump intake. To eliminate human errors in administration, the prescription, or instant drug delivery protocol the program is to execute is triggered by the initial load, or combination of drug vials, refill cartridges, or lines led from a separate reservoir inserted in each pump feed mechanism and any changes made, requiring that each vial self-identify upon insertion into its respective socket in the turret.

A similar mechanism at the pump outlet allows the drug to be directed to any of a number of side-entry connectors or water-jacket intake lines, although to reduce the risk of errors, drugs are constrained to the jacket or jackets of the pump-pair and jacket set. For recirculating wash water through a closed circuit to a single jacket whereby the pump outlet is connected to the water-jacket inlet and the pump intake to the side-entry connector, one pump intake turret socket position provides a line permanently connected to the pump intake. Use of the other intake turret socket positions are for drug refills or where the volume is high, an inlet line from an attached reservoir. The dose adjusted for body weight and the specific condition by the control program, common syndromes that call for the same standard of care drug regimen allow packaging the drugs in a preloaded rotational cartridge or sectional tray for insertion in the drug pump intake turret.

Further to reduce the risk of errors during production, drugs and compounded drugs can be identified by an optional QR code reader. Where standardized cartridge vials are loaded into the pump intake turret, the code can be used to set the program automatically. When the patient must not be allowed to adjust the apparatus and the drugs require prompt change, the pump-pack in use is disconnected and an alternative pump-pack preloaded with the prescribed drugs is connected to the jacket lines by a trusted party. An optional alternative means allows for electronic confirmation that the pump intake turret is properly loaded, and if necessary, corrects the identity for the program. FIG. 36 includes a miniature identity code reader positioned alongside the pump intake drug turret. Such a built in identity code reader can identify a drug set prepackaged in a rotational cartridge or sectional tray.

The cartridge keyed to cause the code bearing vial to face the reader when inserted in the turret, the identity of the drug set and each drug therein can be signaled to the program. Such a built in reader is not a standard component of the pump-pair and jacket set, however, because the number of conditions that may be responded to with a standardized drug regimen is relatively few, and for an individual patient, the information can be no more than partial, specific doses and dose intervals not broadly generalizable. Implementation of the prescription by a local pharmacist preserves the flexibility of drug dispensing thus. The pharmacist loads the turret sectional trays, or rotational cartridges, with the prescribed drugs, enters the prescription into a routine or program generation terminal, and offloads the routine onto a durable recording medium, such as an optical disc. The program, read into the microcontroller through a conventional data port, controls the action sequence of the pumps and turrets.

Although one or more pump-pair and jacket set plug-in modules may be idle at any given time, the power and control module, or base, into which the pump-pair and jacket set plug-in module or modules insert contains a battery or power source and microcontroller capable of synchronously controlling the pumping and turret actions of the plug-in modules inserted at the time. Since jackets belonging to different sets can be interposed along a ductus, by coordinating these actions, the microcontroller can cause the same or a different drug to be delivered to adjacent segments simultaneously or sequentially, for example. Dispersed jackets allow drug delivery to different type ductus in different parts of the body in any sequence. Patch-magnets fastened onto the outer capsule of an organ allow magnetic carrier bound drugs released into the supply artery of that organ to be drawn radially outward through the parenchyma.

An optional barcode reader included in FIG. 36 confirms that the vial or hose with similar code along its side facing the reader is that specified in the prescription The barcode or stripes may additional encode and confirm the correct flow rate for the viscosity of the substance and the caliber of the line and are read at corresponding reading stripes about the internal surface of the socket, the information transmitted centrally to the microcontroller in the pump-pack to adjust the pump speed. Whereas in a simple embodiment the relation of the drug or gel, for example, to the line remains constant, line switching necessitates that the program, here substantially synonymous with the prescription, adjust control for changes in line length and diameter, along with the flow rate of the drug.

Since microcontroller and multicore microcontroller input pins are needed to set the program, additional pins to input collateral functions such as those from sensors placed to signal changes in medical conditions and outputs to execute the program, and a significant storage capacity needed to record potential changes, the microcontroller in any given pump-pair plug-in pump-pack or the equivalent in a distributed set of pump-packs under unified control must provide a number of pins and performance capacity consistent with industrial multicore microcontrollers. The PICoPLC program ladder logic editing, simulating, and compiling tool that can generate native code for 8-bit and 32-bit microcontrollers, such as the Parallax, Inc. Propeller and Microchip Technology PIC16 central processing units, from a ladder diagram, effectively gaining in a microcontroller a level of integrative capability associated with programmable logic controllers.

For these and other microcontrollers, further reduction in size and power consumption are afforded through discretization, whereby the continuous steam of data is converted into a sequence of data points with sufficient accuracy preserved for control purposes. Sensor inputs that justify proportional-integral-derivative closed loop feedback from implanted sensors may be discretized. Conversion of closed loop physiological or life-sign input data into a sequence of points then overcomes the need for an expensive and larger programmable logic controller able to perform the ongoing calculation essential to control the continuous process as such (see, for example, Uzunovic, T. and Turkovic, I. 2012. “Implementation of Microcontroller Based Fuzzy Controller,” 6th IEEE International Conference on Intelligent Systems, Sofia, Bulgaria, available at ieeexplore.ieee.org; Velagic, J., Kuric, M., Dragolj, E., Ajanovic, Z., and Osmic, N. 2012. “Microcontroller Based Fuzzy-PI Approach Employing Control Surface Discretization,” 20th Mediterranean Conference on Control and Automation, Barcelona, Spain, available at ieeexplore.ieee.org; Avery, S., Gracey, C., Graner, V., Hebel, M., Hintze, J., LaMothe, A., Lindsay, A., Martin, J., and Sander, H. 2010. Programming and Customizing the Multicore Propeller Microcontroller: The Official Guide, New York, N.Y.: McGraw-Hill; Nass, M. 2010. “Xilinx Puts ARM Core into its FPGAs,” Embedded, available at http://www.embedded.com/electronics-products/electronic-product-reviews/embedded-tools/4115523/Xilinx-puts-ARM-core-into-its-FPGAs; McConnel, T. 2010. “ESC—Xilinx Extensible Processing Platform Combines Best of Serial and Parallel Processing,” Electronic Engineering Times, available at http://www.eetimes.com/document.asp?doc_id=1313958; Cheung, K. 2010. “Xilinx Extensible Processing Platform for Embedded Systems,” available at http://fpgablog.com/posts/arm-cortex-mpcore/; Kanagaraj, N., Sivashanmugam, P., and Paramasivam, S. 2009. “A Fuzzy Logic based Supervisory Hierarchical Control Scheme for Real time Pressure Control,” International Journal of Automation and Computing 6(1):88-96; Keckler, S. W., Olukotun, K., and Hofstee, H. P. 2009. Multicore Processors and Systems, New York, N.Y.: Springer; Scanlan, D. A. and Hebel, M. A. 2007. “Programming the Eight-core Propeller Chip,” Journal of Computing Sciences in Colleges 23(1):162-168). Linear stage motors usually steppers, other type motors are not to be excluded.

The ‘inertia’ and delay in affecting some physiological parameters considerably greater than it is for others, depending upon the application, no individual or composite form of control, to include model predictive, fuzzy, and proportional-integral-derivative can be ruled out. In general, using different controllers in each type pump-pack is more costly than is the use of a standard microcontroller and development environment; nevertheless, provided simple applications and embodiments prevail for a given type pump-pack, the smaller cost of a simple or hobby grade controller is preferable.

However, less feedback adaptive response capability, the requirement placed on the microcontroller as a motion controller consists only of synchronizing the rotatory motion of the pump and pump turret motors. In less critical applications, both the pump and pump turret movers can be open loop controlled direct current stepper motors. The degree of refinement as to the type control but not the motors used rises as the criticality of dose precision and timing (see, for example, Johansson, A. and Stigborg, M. 2013. “Analogue versus Digital Solution for Motor Control,” Thesis, Jönköpings Tekniska Högskola [Jönköping School of Engineering, Jönköping, Sweden] [available at http://www.diva-portal.org/smash/get/diva2:632203/FULL TEXT01.pdf).

Unless the increased cost for a high capability controller to be embedded in pump-packs used to treat well-defined and predictable conditions is not warranted, a universally applied controller able to support a complex pumping and fluid line switching protocol is used in all pump-packs produced at a time. More specifically, a pump-pack that accommodates a two pump-pairs is used when only a single pump-pair is required, with the hard and software already in place should a second pump-pair be required. Where intercurrent disease or comorbidity necessitate a third pump-pair, this is accomplished by using a second hard and software standardized pump-pack. Preferably, a standardized microcontroller, pump-pairs, and programming method is used across all embodiments from the simplest to the most complex.

This not only affords the greatest flexibility for adapting the program to changing conditions without the need to replace the apparatus, but has the advantage of eliminating the relatively greater cost of nonstandardization in the use of diverse apparatus and control programming, the increased cost for a more versatile microcontroller readily overcoming the added cost per unit and the greater susceptibility to human error. Distributed pump-packs change only in terms of spatial separation or division; each coordinated with the others by joint connection to a central microcontroller housed in one of the pump-packs. The combination of jacket pump-pair plug-in module magnetic barcode or stripe patterns inserted in the power and control pack or packs if distributed, at any given moment sets the microcontroller for coordinated control over the delivery of the combination.

The pump rate of delivery varies with the application, and the duration of battery power varies with the volume. If the dosing does not permit all medication to be delivered from a wearable device, then connection is on an as needed basis to a home tabletop or clinical pump or other apparatus. Additional sidelines connected to the water-jacket allow different adjuvant drugs, for example, to be delivered through the water-jacket to enter into the mainline, seen as part number 6 in the drawing figures. Where complete segregation among the drugs is medically insignificant so that these can be delivered in direct sequence through the same line, the drugs can be supplied from vials mounted about a turret, the pump-pack-embedded microcontroller used to control the rotation of the turrets and pumps.

Depending upon the criticality of the application, bidirectional rotation of the turrets can be accomplished two rotary solenoids positioned to turn the turret in opposite directions, a stepper motor, or a sensorless dc motor, the need for tight feedback control applied to the driving paths as opposed to any physiological parametric paths not warranted. While the sequential targeting of different drugs to a certain organ, lesion, or organ system is commonly practiced (see, for example, Calvo, E., Ravaud, A., and Bellmunt, J. 2013. “What is the Optimal Therapy for Patients with Metastatic Renal Cell Carcinoma Who Progress on an Initial VEGFr-TKI [Vascular Endothelial Growth Factor-Tyrosine-Kinase Inhibitor]?,” Cancer Treatment Reviews 39(4):366-374), as in neoadjuvant therapy, the targeting of organs or tissues belonging to the same or different systems in different locations, such as to stimulate or depress certain pathways in an organ in one part of the body in preparation for the targeted delivery of a drug to an organ or tissue in another part of the body, has been broached barely if at all.

The placement of side-entry connection jackets at different locations in the body makes possible the development of therapy that uses simultaneous or timed sequential targeted delivery of the same or different drugs to the same or different side-entry connection jackets to treat the same or different diseases. Where entry of the drugs into the systemic circulation is at most inconsequential, administration thus can be applied no less to the administration of drugs previously specified as not for simultaneous use. Using the means described herein, the simultaneously or sequentially coordinated delivery of drugs, to include those specified as not for use in the same patient, can proceed frequently in different organs and tissues throughout the day under automatic control.

While the line from a given pump might be divided to supply the mainlines or sidelines of different jackets for treatment of the same or a different condition, or the output of two pumps might be converged into the same line, for example, because errors in the administration of drugs are common under any circumstances, such merging and crossover is discouraged. Numerous hypothetical connections between pumps, lines, ports, and jackets possible, reducing these to the medically pertinent and least costly includes packaging jacket mainline and sideline pump-pairs with lines, port, and jacket as either independent for use of a single jacket or as modules that plug into a receiver pack with shared battery and controller. Within the constraints imposed by miniaturization, dosing, and patient comfort, the pump-pack includes a jacket pump-pair plug-in module for each jacket.

Lines that call for the delivery of medication in small doses continuously or at frequent intervals are relegated to a wearable pump-pack, while lines that call for the delivery of medication in large doses at infrequent intervals may be relegated to a stationary apparatus at home or in the clinic. Ideally, each plug-in module allows not only drug delivery after jacket placement but is used to place the jacket. To assure proper dosing, time coordinated control of the mainline and sideline pumps within each plug-in module inserted in the pump-pack for delivery to one of the differently located side-entry connection jackets is placed under the unified control of a microcontroller embedded within the pump-pack.

Medical pumps in general can deliver medication with precise dosing frequently or continuously even when the dose size is very small, administration thus by a professional in constant attendance impracticable and subject to human error. When, exceptionally, different type pumps are to be inserted into the same pump-pack, human error is avoided through a self-identifying keyed insertion base pin pattern. When plugged into the socket in the pump-pack, the pin pattern of the pump-pair plug-in modules set the microcontroller for coordinated drug delivery from each plug-in module in the combination. With the tissue plug excised, the pump pressure through both the mainline and sideline or sidelines antegrade, and nothing to block the way through it, adjuvant medication converges with that in the mainline to pass through the opening into the ductus.

With antegrade pumping stopped and passage through the opening impeded if not prevented by the blood pressure or luminal contents and a thick and sticky substance to block the opening, the substance takes the course of least resistance by flowing into the side-connector. In excision of the tissue plug from the side of the ductus, the water-jacket is ordinarily started first, and water or a crushed tacky hydrogel, medicinal or medically neutral but usually containing an antimicrobial and anti-inflammatory, is passed through the water-jacket, seen as 7 in the drawing figures, to flush over or irrigate the ductus wall at the prospective opening (ostium, fenestra), to return through the mainline. Only one sideline pump is needed to drive water or a crushed tacky hydrogel through the water-jacket. Irrigation continued, a pulsed vacuum is applied to the side-entry connector along the mainline 13.

A pulsed vacuum is generated by running the pump in reverse or abductally (abcorporeally) and replacing the pump vial or refill cartridge turret cylinder with another having openings spaced about the circumference at intervals to generate the pulses at the frequency desired for the turret rotation speed written into the microcontroller program. The vacuum draws the ductus wall outward, compressing a layer of viscoelastic polyurethane foam lining the jacket, allowing the cutting edge of the side-entry connector to be driven through the lumen wall, excising a plug of tissue. Within the limit set by its thickness, the foam not only allows the cutting edge to be driven through the ductus wall to excise the tissue plug but allows compliance in the internal diameter of the jacket with intrinsic movement in the ductus whether peristaltic or pulsatile.

Where the use of closed cell foam results in a buildup of heat and irritation, or interferes with permeating the lining with a liquid medication, an open cell, hence, porous, viscoelastic polyurethane foam is used. Critically, the lining avoids compression of the fine vessels and nerves about the adventitia or fibrosa that in an artery, for example, would induce atherosclerosis, as addressed below. The lining also compensates for anatomical, inflammatory, or lesion caused deviations in ductus caliber and prevents contact of any part of the jacket, such as a, from coming into with the adventitia or fibrosa. Yet another advantage in the lining is that in a small child, it extends the usable life of the jacket by permitting an increase in ductus diameter due to growth. Should the radially outward excursion of the lumen wall exceed that allowed by the lining, the spring hinges used to fasten the two half cylinders of the jacket will extend the range for expandability.

In a tight location such as that depicted in FIGS. 16 and 21, the combined expandability afforded by the lining and the spring hinges makes it possible to situate the jacket where the normal growth would otherwise interrupt the indwelling time and treatment much sooner (see, for example, Voges, I., Jerosch-Herold, M., Hedderich, J., Pardun, E., Hart, C., and 5 others 2012. “Normal Values of Aortic Dimensions, Distensibility, and Pulse Wave Velocity in Children and Young Adults: A Cross-Sectional Study,” Journal of Cardiovascular Magnetic Resonance 14:77; Kaiser, T., Kellenberger, C. J., Albisetti, M., Bergstrasser, E., and Valsangiacomo Buechel, E. R. 2008. “Normal Values for Aortic Diameters in Children and Adolescents—Assessment in Vivo by Contrast-enhanced CMR-Angiography,” Journal of Cardiovascular Magnetic Resonance 10:56; Machii, M. and Becker, A. E. 1997. “Morphologic Features of the Normal Aortic Arch in Neonates, Infants, and Children Pertinent to Growth,” Annals of Thoracic Surgery 64(2):511-515); however, numerous diseases and genetic defects can markedly distort the developing aorta.

The ability to target drugs to specific lesions and thus avoid the circulation and exposure to untargeted tissue is often the more importance in the treatment of small children with radioactive substances, for example. Radiation shielded side-entry connection jackets allow superparamagnetic drug-carried radionuclide nanoparticles for example, to be targeted directly to lesions. To prevent the tissue plug from remaining partially attached or ‘hanging up’ at the prospective site of the opening, the foam lining must be greater in thickness than the ductus. For this reason, the site should have been well imaged and studied, especially since jacket dimensions are to be kept to a minimum when neighboring tissue would be encroached upon. Placement must control leakage through the opening created even when the tissue plug hangs at the opening.

In general, leakage or extravasation is stopped by denying space outside the opening, by filling the space outside the opening, preferably, with a tacky crushed hydrogel or by forcible restraint using pressure washing or flushing through the water-jacket. The installation process proceeds automatically under a routine stored in the microcontroller read only memory, which outputs prompts for manual intervention if necessary to a separate clinic display. Referring now to FIG. 29, the jacket having been positioned about the ductus before the plug of tissue is removed, the intake of pump 46 if not open is switched from gel drug or wash water reservoir 49 to open, and that of pump 47 from the turret to fill-gel reservoir 54. As will be described, since the output of any pump can be switched to the same or any other pump mainline or sideline, wash water or any therapeutic substance can be recirculated through a closed circuit.

Starting pump 46 in reverse, or counterclockwise, evacuates line 13, drawing the ductus wall over the sharp forward edge or trepan of side-entry connector 6, incising a circular opening. Thicker and harder ductus may require loosening side-entry connector 6 for use as a manual circle-cutter. Even with calcified plaque present, when forced against the razor sharp front edge of the side-entry connector, the plug should fracture and come away clean. If a thicker plaque does not fracture cleanly and avulses or pulls some tissue about the opening into the side-entry connector, then the foam lining and optionally, the addition of an anti clotting agent to the fill gel in reservoir 54 should still allow a functional junction. A transient or sudden reduction in resistance to the vacuum causes the installation program stored in the controller to start pump 47, driving crushed tacky medicinal or medically inert hydrogel from reservoir 54 through line 11 and the water-jacket.

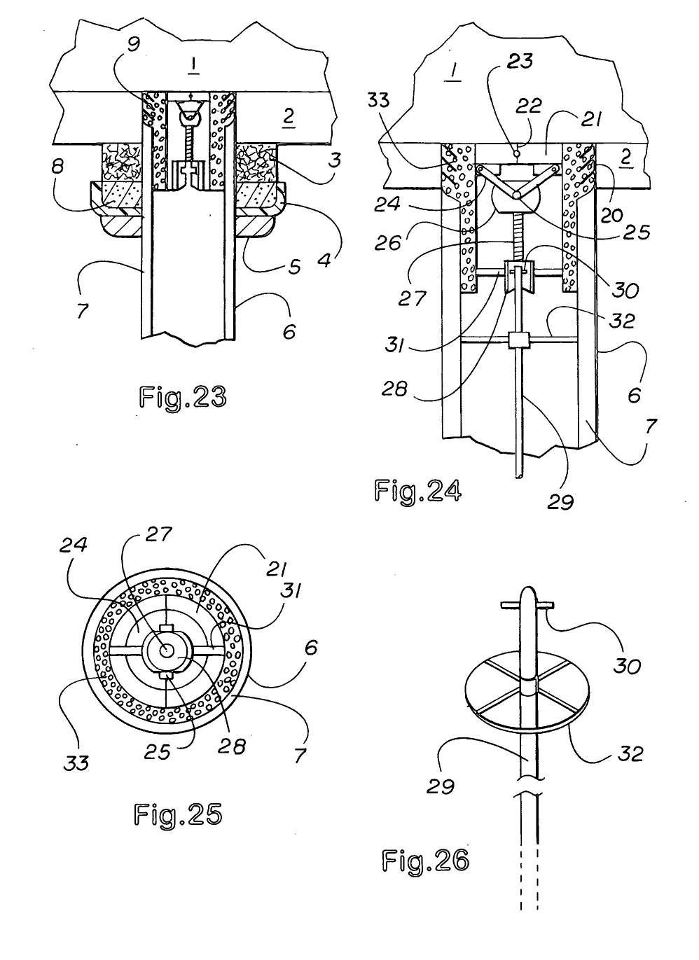

Two types of valves are suitable for use in lines 13 and 11, each having certain advantages. One type is a polymeric bidirectional elastic slit type, which depending upon the force of the pressure head can range in elasticity and thickness from a membrane to a set of apposite or overlapping flaps having an elasticity consistent with the pressures and fluids used, the other a double winged mechanical throttle and shutoff valve-plug such as that shown in FIGS. 23 thru 25. In most instances, a side-entry connector will have a permanently bonded elastic slit membrane at the adductal or distal terminus. Not properly a valve (which connotes adjustability), such a bidirectional passive and ordinarily nonadjustable terminal fluid resistor simplifies placement or installation by allowing the application of greater vacuum force. Flap valves used in aperetic applications in particular must have flaps with adluminal surfaces of a very smooth material, usually polytetrafluoroethylene.

### Bidirectional Slit and Flap Valves

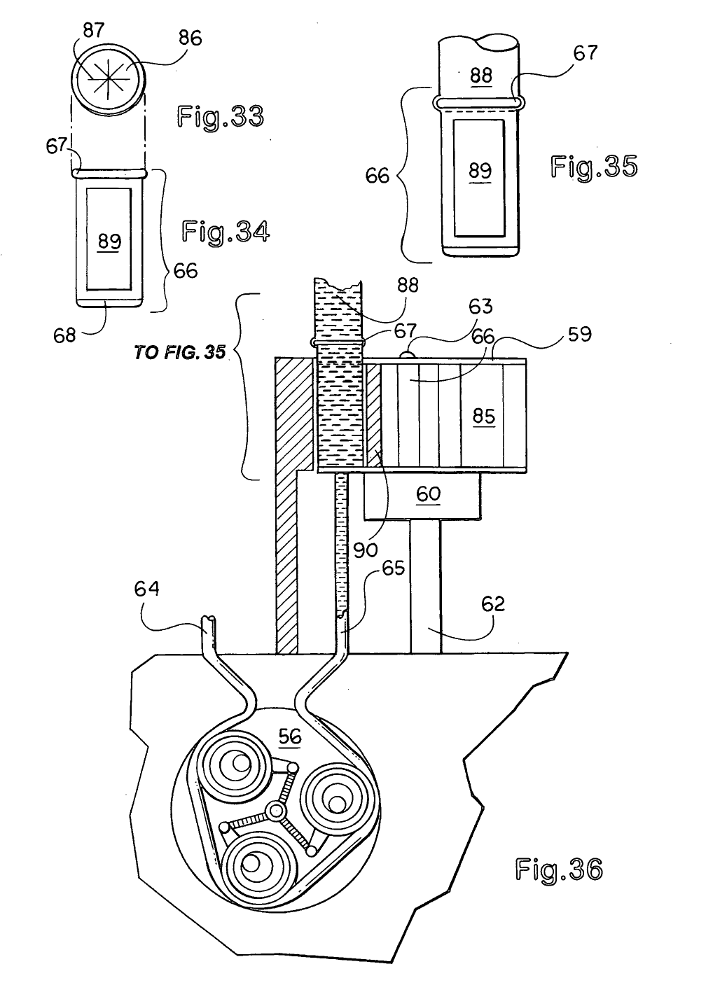

A slit type membrane valve is shown in FIG. 33. This valve has more central pie-cut sectors separated by slits, leaving a common frame bonded to the surround. Membrane valves are used to prevent flow or leakage at drug vial, reservoir, and hose connections. By contrast, a flap valve is placed at the opening made into the lumen of the substrate ductus. The flap type valve to abut upon the adventitia or fibrosa, special measures are taken to prevent harm. The leading (adductal, forward) edge of the flap type valve surround is a cutting die used to excise a plug of tissue from the side of the ductus. This leaves the plug distal to, that is, to the far side, of the valve. The plug is therefore extracted with the same vacuum force used to excise it.

To expedite extraction through the valve, each flap of the flap type valve gives optimal clearance as a rectangular elastic tang or tongue overlapping its neighbors along its unbonded edges, these positioned rectilinearly within and bonded along the outer edge to die surround, which is rectangular with rounded-corners, the long axis parallel to that of the substrate ductus. Both type valves must resist microfractures, fatigue, retain resilience, and remain pliant to allow deflection in either direction when and only when encountering the minimum design force. For vascular applications, achieving the minimal thrombophilic (or thrombofilic, the term in this connection denoting a surface conducive to clotting) propensity of the material in a flap type valve is no less important as achieving the required mechanical properties and endurance.

Anticlotting medication best minimized if not eliminated, various materials and surface treatments are available for reducing if not eliminating this tendency (see, for example, Nilsson, P. H., Engberg, A. E., Back, J., Faxälv, L., Lindahl, T. L., Nilsson, B., and Ekdahl, K. N. 2010. “The Creation of an Antithrombotic Surface by Apyrase Immobilization,” Biomaterials 31(16):4484-4491; Gorbet, M. B. and Sefton, M. V. 2004. “Biomaterial-associated Thrombosis: Roles of Coagulation Factors, Complement, Platelets and Leukocytes,” Biomaterials 25(26):5681-5703; Spijker, H. T., Graaff, R., Boonstra, P. W., Busscher, H. J., and van Oeveren, W. 2003. “On the Influence of Flow Conditions and Wettability on Blood Material Interactions,” Biomaterials 24(26):4717-4727; Hong, J., Nilsson Ekdahl, K., Reynolds, H., Larsson, R., and Nilsson, B. 1999. “A New in Vitro Model to Study Interaction between Whole Blood and Biomaterials. Studies of Platelet and Coagulation Activation and the Effect of Aspirin,” Biomaterials 20(7):603-611).

Such jackets usually permanent, temporary measures as incorporated into drug eluting stents may apply during placement and until endothelialized, but are not adequate over the life of the device (see, for example, Palmerini, T., Biondi-Zoccai, G., Della Riva, D., Stettler, C., Sangiorgi, D., D'Ascenzo, F., Kimura, T., and 12 others 2012. “Stent Thrombosis with Drug-eluting and Bare-Metal Stents: Evidence from a Comprehensive Network Meta-analysis,” Lancet 379(9824):1393-1402; Aggarwal, R. K., Ireland, D. C., Azrin, M. A., Ezekowitz, M. D., de Bono, D. P., and Gershlick, A. H. 1996. “Antithrombotic Potential of Polymer-coated Stents Eluting Platelet Glycoprotein IIb/IIIa Receptor Antibody,” Circulation 94(12):3311-3317).

Degradation or failure necessitating invasive reentry, valves of either type must provide a long service life without significant change in mechanical properties (see, for example, Shaw, M. T. and MacKnight, W. J. 2005. Introduction to Polymer Viscoelasticity, Hoboken, N.J.: Wiley Interscience; Boresi, A. P. and Chong, K. P. 2000. Elasticity in Engineering Mechanics, New York, N.Y.: John Wiley and Sons; Nielsen, S. E. and Landel, R. F. 1994. Mechanical Properties of Polymers and Composites, New York, N.Y.: Marcel Dekker). For valves in vascular applications, and flap valves in particular, long term repeated abrupt or transient flow at onset and cutoff will eventually induce unfavorable adaptation if not injury.

This is averted by shaping the valve sectors, flaps, or tongues to gradually or incrementally decrease in girth from the outer margin to the center. This method and/or the insertion of narrowed neck or waist sections allow gradual or incremental opening and closing of the flaps, and therewith, control over the rate of delivery or extraction through the valve as a fluid resistor. This not only moderates the volumetric flow rate at onset and cutoff but allows control over this rate by the microcontroller. For this reason as well, the use of materials that are not susceptible to degradation with a loss in resilience over time is important.

When not provided by a single material, the required combination of properties and long life are obtained through lamination, imbrication, overlapping, impregnation, embedment, and combination of various bioinert materials (see, for example, Karbhari, V. M. (ed.) 2013. Non-destructive Evaluation (NDE) of Polymer Matrix Composites: Techniques and Applications, Sawston, Cambridge England: Woodhead Publishing Limited; Kaw, A. K. 2006. Mechanics of Composite Materials, Boca Raton, Fla.: Taylor and Francis Group, Chemical Rubber Company Press; Abington, Cambridge England: Woodhead Publishing Limited; Owen, M. J., Middleton, V., and Jones, I. A., 2000. Integrated Design and Manufacture Using Fibre-reinforced Polymeric Composites, Matthews, F. L. and Rawlings, R. D. 1999. Composite Materials: Engineering and Science, Woodhead Publishing Series in Composites Science and Engineering, Boca Raton, Fla.: Chemical Rubber Company Press).

By embedding or laminating magnetically susceptible ferrous matter in the flaps to respond to the field force of an extracorporeal tractive electromagnet under the control of the master microcontroller, for example, such an elastomeric barrier can, however, be made adjustable. Opening the barrier magnetically, however, is usually disqualifying, because lumen contents then extravasate indiscriminately. Thus, in an ambulatory analyte extraction application such as leukapheresis to be described, for example, the incorporation of ferrous matter in the flaps disallows the use of an external electromagnet to differentially extract the susceptible particle bound target analyte. The vacuum force is used to excise a plug of tissue by drawing the ductus wall outward over the cutting die or trepan leading edge-surround of the side-connector.

Once severed, the vacuum pulls the excised tissue plug out through the opened flaps of the membrane, thicker elastomeric flat stock, or sheeting, and the vacuum removed, closes, sealing the opening created in the side of the ductus, substantially truncating further exsanguination. When a cabled device such as a guidewire or fiberoptic scope is passed through such a flexible barrier, the flaps restrict the passable opening to that occupied by the cabled device. Placement thus will be described in reference to the use of a double arm side-connector as shown in FIG. 7 with longitudinally extended distal terminus and elastic slit membrane for continuous high volume analyte extraction such as essential for ambulatory apheresis. By comparison, an impasse-jacket with extraction grating is suited to lower volume extraction.

Situated forward and engaged instead by a rubbery surround, elastic slit membrane and mechanical valve-plugs, which are not permanently bonded in place at the distal terminus, offer the advantage that these can be advanced or retracted along the line to any level. Retracted, it allows flow past it into or out of the lumen. A mechanical type valve-plug that is remotely controllable, as addressed below, can be opened and closed to allow antegrade or forward or retrograde or reverse flow as is essential for aspiration through it and the opening in the ductus, for example. Both types of valve-plug will seal off the open end of a line to prevent spillage. The elastic slit membrane, which to be slid along the lumen of the catheter requires to be mounted at the end of a cylindrical annulus or surround much as the head of a drum, easily passes through a cabled device such as an angioscope or laser and is opened by raising the pressure at the pump.

Specified herein for drug vial inlets and outlets and the endings of drug reservoir inlet hoses to be described as well as slidable valve-plugs, the material, thickness, and form of each slit membrane is chosen for the applicable range of threshold opening pressure. Thus, a membrane that due to the intrinsic elasticity of its material and thickness is highly elastic might have a simple diagonal cut, whereas one made of a less elastic material and/or thicker might have a star-shaped cut. The mechanical valve is adjustable, and if remote controlled as addressed below, can be adjusted, fully opened, or fully closed, in flow-through cross-sectional area by a clinician at a remote location in response to an emergency, for example, who can also adjust the pump setting.

A mechanical valve-plug can be repositioned by sliding it along the catheter. This is accomplished by remotely closing and then driving the valve-plug forward or backward at the head of a column of water or gel, which action can be included in the program. When the accuracy of repositioning is significant, the catheter used is ribbed at intervals along the internal surface and the pump pulsed to exceed the threshold pressure for forcing the valve-plug from one such detent to the next. The susceptibility to be driven thus is increased by forming the ends of the rubbery surround to include a circular recess or indentation (depression, trench, trough).

When the valve-plug is positioned as shown in FIGS. 23 and 24, where to seal off the opening in the ductus it has been intentionally restrained by trapping its forward or adductal portion in the space ahead of the forward edge of the water jacket, whether the valve-plus can be dislodged thus depends upon the force of retention associated with its diameter, restorative force and surface friction of the elastomeric surround, and so on. Depending upon the application, valve-plugs are made in different gradations of resistance to dislodgement or retention; however, an alteration in physical properties of the surround over time can necessitate the use of a manual guidewire such as shown in FIG. 26. Valve-plugs reduce the caliber of the cabled devices that can pass and the flow-through cross-section of the lumen; however, when the program controls the valve-plug, it can, if necessary, compute and apply the offsetting increase in pressure to achieve the same flow rate.

Other types of valve that use vane or iris shutters are more intricate and subject to malfunction, expensive, and offer no advantage over the types just delineated. The vacuum continued as the gel is delivered, the plug is simultaneously drawn outward, that is, pulled, by the vacuum to its fore and forced outward, that is, expelled, pushed by the gel to its rear, forcing it out through line 13. Any difficulty in extracting the tissue plug is corrected by introducing an aspiration catheter, corkscrew-tipped guidewire, or a hybrid corkscrew-tipped aspiration catheter, through a clean-out type inline port fitting shown in FIG. 30. The fitting is entered at the outer surface of the pump-pack through an upper entry hole affording downward tracking toward the pump, and a lower entry hole affording upward tracking toward the jacket.

Extracorporeal parts, that is, the external parts preceding the port at the body surface, are made of clear, tough, transparent plastic, the pump-pack housing of polycarbonate plastic, for example. When tracking will be frequent, both pump inline port in the pump-pack and side-connector are of the double arm type. Referring to FIGS. 29 and 32, the additional insertion of an elastic slit membrane in line 13 (not shown) prevents the tissue plug from reaching and clogging or jamming pump 46 in FIG. 29 or pump 56 in FIG. 32. The trapped plug is readily retrieved by means of an aspiration catheter, corkscrew-tipped guidewire, or a hybrid corkscrew-tipped aspiration catheter, introduced through the upper entry arm of the inline port or clean-out, entered through the one of four holes at the back of the pump-pack leading to the upper arm of pump 56, each covered by a spring cap when not in use.

In FIG. 29, pump 46 is stopped and pump 47 continues to force the gel past the opening cut in the ductus, restraining bleeding or leakage and forcing the plug out through the mainline. If the plug hangs at the opening and the side-entry connector is not loosenable for use as a circle-cutter, then an corkscrew-tipped guidewire, or a hybrid corkscrew-tipped aspiration catheter, introduced through the lower entry hole of clean-out type inline port 69, entered through the hole at the back of the pump-pack and any elastic slit membrane in the line to extract the plug. The tacky hydrogel generally includes antimicrobial and anti-inflammatory medication, and by switching the pump intake from the gel reservoir to the vial containing the drug or combination of drugs to be used, can position the initial dose.

Pumping from the fill-gel reservoir is usually to eliminate any voids, that is, to fill segments of the line between drugs. Both pumps are switchable to fill-gel reservoir 54 and when necessary, can be driven at relatively high speeds to deliver the drug at the head of the gel column quickly. Steps in the installation procedure prior to the foregoing, to include implanting and routing the lines and jacket, and steps following the foregoing, to include fixing the port in position are addressed in the detailed description. When the viscosity of the substance to be propelled through the water-jacket line necessitates a force sufficiently larger than that of the vacuum, the flow rates through the lines are regulated by separate pumps under the coordinated control of a microcontroller.

To expedite extraction or washout of the tissue plug and to suppress extravasation, the water or hydrogel pressure through the water-jacket can be increased. When elastic slit membrane valves with a fluid resistance greater than that of the back pressure, here the expulsive force of the blood passing the opening, are placed along the lines, water irrigation can stop with the lines remaining filled with water to deny entry to the native luminal contents, hence, extravasation. Otherwise, irrigation through the sideline and water-jacket is continuous with the delivery of the initial dose of medication in the form of a tacky and thick or viscid syrup or crushed tacky gel, for example. This suppresses bleeding and retrograde flow out through the lines at the same time that the medication is positioned for delivery through the opening once adductal or antegrade mainline pumping is initiated.

Depending upon the specification of the gel or alternative viscid substance, its use may also eliminate the need for an elastic slit or woven membrane to span across the adductal end opening of the side-entry connector. When the medication is less viscid, irrigation can conclude when elastic slit membrane or flap barrier resistors are inserted into each line. Especially in vascular applications, the use of therapeutic substances in the form of variably tacky hydrogels reduces if not eliminates extravasation whether leakage or exsanguination (bleeding) out of the opening in the ductus created, as well as spillage from lines not provided with an elastic slit membrane barrier fluid resistor when the line is disconnected.

To avoid gas embolism and spillage, disconnection is preferably avoided. The formulation of drugs as syrups, jellies, cellulose gums, or hydrogels, for example, to meet mechanical properties of viscosity and tackiness as specified is well understood. Viscous fluids and gels containing or omitting a drug or drugs, and/or other therapeutic or diagnostic substances can be formulated for use with the apparatus described herein to treat any point along an artery with a tissue plug excised, for example, regardless of the expulsive force of the blood. The side-entry connection line or mainline or mainlines 13, but not the sideline or water-jacket supply line or lines 11, can be transited by a cabled device, such as a fine fiberoptic endoscope, laser, or intravascular ultrasound probe.

Access to the line is through a double arm inline port or clean-out as shown in FIG. 30 and indicated in FIG. 32. The line or clean-out port incorporates an elastic slit membrane that covers the opening into the lumen of the pump line at the junction of the inlet tubes. The membrane allows an inserted device to pass to either arm directing the device in either direction while minimizing leakage as a check valve. For such use, the special guidewire described below and shown in FIG. 26 for retrieving a valve-plug positioned downstream along the catheteric line must have its centering ring 32 slid back and off at the proximal end. During installation of the jacket with lines, port, and pump attached, a valve-plug previously inserted into any line filled with a substance that would leak out the open end is used when the lines are cut off or trimmed flush to the front of the port.

A mechanical valve-plug such as that shown in FIGS. 23 thru 25 and described below, is continuously variable between fully open and fully closed, and can be controlled from outside the line. By contrast, a slit membrane or flap type valve passively opens and closes gradually or incrementally in response to the force of the pressure head. When the an elastic slit membrane valve or a valve-plug with its elastic surround engaged by the abductally inclined prongs at the distal end of the side-entry connector has been positioned over the opening created in the side of the ductus, the valve can be retracted so that its distal face is level with the forward edge of the water-jacket to allow flow through the water-jacket. If the valve is a valve-plug, the flow rate can be regulated or metered by adjusting the cross-sectional area of the opening through the plug.

It can therefore be adjusted by the pump microcontroller in coordination with the variable speed pump to throttle the volumetric flow rate through the line. Entry is always with the cabled device or guidewire wetted with an antimicrobial, an anticoagulant or platelet blocker for entry into a blood vessel, for example. One or more valve-plugs can be positioned anywhere along the lines to either side of the port, that is, those extracorporeal connected to the pump and, those intracorporeal connected to the jacket, to throttle or shutoff flow through the line. Closing a valve-plug along the mainline accelerates delivery through the opening in the ductus of medication sent through a sideline, while reversing the bidirectional pump accelerates aspiration therethrough.

As shown in FIG. 31, a valve-plug is extracted by passing the valve guidewire shown in FIGS. 24 and 26 through the lower hole at the back of the pump-pack, which leads through the lower arm of double arm inline port shown in FIG. 30, and up through pump line 13. Before the local entry wound used to place the jacket has been closed, a proximal single or double arm clean-out flap-valve type inline port of the kind shown in FIG. 30 can be used to pass through a guidewire or cabled device such a fiberscope through lines 11 or 13 and into the native lumen. Thereafter, invasive reentry is avoided by routing the guidewire or cabled device as shown in FIG. 31.

When use thus is contemplated, the valve is not of the slit membrane but rather the mechanical, spring-loaded duplex butterfly hemispherical vane or double doors type shown in FIGS. 23 thru 25. The mainline need not be emptied to allow the device to pass through and into the lumen. The clean-out type inline port as a port also allows the intermittent or emergency administration of other drugs. The delivery of medication in the form of viscid gels, jellies, and syrups, for example, eliminates the need to use the water-jacket lines to restrain luminal contents from leaking. Also avoided is the need to use a service channel line to restrain the leakage of luminal contents through pressurized irrigation when the line is already filled with medicinal contents that would result in a sudden overdose.

The formulation of gels that incorporate drugs is well established (see, for example, Peppas, N. A. 2004. “Hydrogels,” in Ratner, B. D., Hoffman, A. S., Schoen, F. J., and Lemons, J. E. (eds.), Biomaterials Science: An Introduction to Materials in Medicine, New York, N.Y.: Academic Press, pages 35-42, updated in 2012). Passive elastic slit membrane valves allow a cabled device to pass but otherwise act as nonadjustable shutoff valves. In comparison, mechanical shutoff and throttling valve-plugs as shown in FIGS. 15 and 16 limit the diameter and impede passage of cabled devices but can be adjusted from outside the body individually or in groups, different groups adjusted using different radio control frequencies. As many valves of either type can be positioned along a mainline or larger diameter sideline as necessary.

While injectable hydrogels are more often formulated to be liquid at ambient temperature and gel at body temperature to reduce dispersion or diffusion from the injection site or to remain as a tissue-engineered ing scaffold whether to serve as a stem cell culturing matrix (see, for example, Klouda, L. and Mikos, A. G. 2008. “Thermoresponsive Hydrogels in Biomedical Applications,” European Journal of Pharmaceutics and Biopharmaceutics 68(1):34-45; Yu, L. and Ding, J. 2008. “Injectable Hydrogels as Unique Biomedical Materials,” Chemical Society Reviews 37(8):1473-1481; Ekenseair, A. K., Boere, K. W., Tzouanas, S. N., Vo, T. N., Kasper, F. K., and Mikos, A. G. 2012. “Synthesis and Characterization of Thermally and Chemically Gelling Injectable Hydrogels for Tissue-engineered ing,” Biomacromolecules 13(6):1908-1915; Lian, S., Xiao, Y., Bian, Q., Xia, Y., Guo, C., Wang, S., and Lang, M. 2012. “Injectable Hydrogel as Stem Cell Scaffolds from the Thermosensitive Terpolymer of NIPAAm/AAc/HEMAPCL,” International Journal of Nanomedicine 7:4893-4905), for the present purpose, where the drug is contained and directed after it has been introduced, the control problem is extracorporeal, so that the reverse transition is preferred.

Medication delivered in the form of a thermoreversible injectable hydrogel which sol transitions and flows rather than gels at body temperature allows better control over pump or hypodermic delivery compared to medication in a liquid state (see, for example, Morita, C., Kawai, C., Kikuch, A., Imura, Y., and Kawai, T. 2012. “Effect of Amide Moieties for Hydrogelators on Gelation Property and Heating-free pH Responsive Gel-Sol Phase Transition,” Journal of Oleo Science 61(12):707-713; Nguyen, M. K. and Lee, D. S. 2010. “Injectable Biodegradable Hydrogels,” Macromolecular Bioscience 10(6):563-579; He, C., Kim, S. W., and Lee, D. S. 2008. “In situ Gelling Stimuli-sensitive Block Copolymer Hydrogels for Drug Delivery,” Journal of Controlled Release 127(3):189-207). This minimizes running, aids in maintaining dose accuracy, and makes keeping air out of the line less difficult.

Gel-sol transition upon entry into the ductus causing the gel to sol transition and flow at the opening created in the side of the ductus (ostium, vasculostomy opening), can be facilitated through the application of heat or use of a service-channel to deliver a heated pH gel solvent (Nguyen and Lee 2010, Op cit.). The gel can be warmed upon entering the port and along the side-entry connection line by a resistance wire running the length of the line or by a heating coil inside a valve-plug described in the specification to follow at the ostium, while the chemical solvent can be delivered through a service channel, for example. Along nonvascular ductus, the nominally designated water-jacket can deliver a pressurized gas, ordinarily air, which can be heated or chilled depending upon which end of a vortex tube or ‘cold air gun,’ for example, is used as the input.

These properties minimize the outflow of luminal contents through the opening in the side of the ductus to be created and at the access port placed at the body surface. In some situations, it will be desirable to disconnect a side-entry line filled with medication in order to pass through a cabled device, which as explained below, may also contain a valve-plug that must be retrieved to allow the cabled device to pass through. In this situation, consecutive fluid therapeutic columns to pass through the line are best controlled to assure proper dosing when flow requires the application of propulsive force. A jacket with more than one side-entry connector affords better isolation among more freely flowing drugs. Simple drug targeting by direct delivery can be used to avoid adverse drug interactions.

For example, the mechanism of calcium channel blocker function is not dependent upon metabolism in the liver, but can interfere with the metabolism of other drugs when allowed to pass into the liver. In the case of an end arterial epicardial coronary artery, direct delivery as depicted in FIG. 16 avoids the circulation. Direct delivery into the inferior and/or superior vena cava of a calcium channel blocker, for example, avoids interaction in the liver with drugs administered orally, a trace amount if any recirculated. Placing a side-entry jacket about the inferior and/or superior vena cava allows post-hepatic introduction of any drug in liquid form into the circulation, so that if fully taken up, the drug is not returned to the liver. The use of an impasse-jacket allows a superparamagnetic nanoparticle bound drug to be actively removed from the circulation.

In the treatment of hereditary amyloid cardiomyopathy (see, for example, Quarta, C. C., Kruger, J. L., and Falk, R. H. 2012. “Cardiac Amyloidosis,” Circulation 126(12):e178-82), which is probably far more prevalent in congestive heart failure than is currently diagnosed (Falk, R. H. 2011. “Cardiac Amyloidosis: A Treatable Disease, Often Overlooked,” Circulation 124(9):1079-1085), direct targeting of the liver can avert the extrahepatic adverse side effects, drug-drug interactions, and certain other problems posed by antisense oligonucleotides and ribonucleic acid interference drugs, for example (see, for example, Rayburn, E. R. and Zhang, R. 2008. “Antisense, RNAi, and Gene Silencing Strategies for Therapy: Mission Possible or Impossible?,” Drug DiscoveryToday 13(11-12):513-521; Weyermann, J., Lochmann, D., and Zimmer, A. 2004. “Comparison of Antisense Oligonucleotide Drug Delivery Systems,” Journal of Controlled Release 100(3):411-423).

Non Val30Met TTR familial amyloid cardiomyophathy, meaning the most prevalent form, in which mutant transthyretin protein binds the amino acid methionine rather than valine at position 30 (see, for example, Ohmori, H., Ando, Y., Makita, Y., Onouchi, Y., Nakajima, T., Saraiva, M. J., Terazaki, H., and 8 others 2004. “Common Origin of the Val30Met Mutation Responsible for the Amyloidogenic Transthyretin Type of Familial Amyloidotic Polyneuropathy,” Journal of Medical Genetics 41(4):e51, available at http://jmg.bmj.com/content/41/41e51.long or http://www.ncbi.nlm.nih.gov/pmc/articles/PMC1735751/pdf/v041p00e51.pdf) progresses despite a liver transplant (Benson, M. D. 2013. “Liver Transplantation and Transthyretin Amyloidosis,” Muscle and Nerve 47(2):157-162), but can be treated by direct delivery to the liver of gene therapy (see, for example, Suhr, O. B., Holmgren, G., and Lundgren, E. 2004. “Gene Therapy: Lessons Learned from Liver Transplantation for Transthyretin-amyloidosis,” Liver Transplantation 10(12):1551-1553) newer ribonucleic acid interference drugs (see, for example, Guan, J., Mishra, S., Falk, R. H., and Liao, R. 2012. “Current Perspectives on Cardiac Amyloidosis,” American Journal of Physiology. Heart and Circulatory Physiology 302(3):H544-H552), or antisense oligonucleotides (see, for example, Benson, M. D., Kluve-Beckerman, B., Zeldenrust, S. R., Siesky, A. M., Bodenmiller, D. M., Showalter, A. D., and Sloop, K. W. 2006. “Targeted Suppression of an Amyloidogenic Transthyretin with Antisense Oligonucleotides.” Muscle and Nerve 33(5):609-618) for example, delivered directly from a surface port to the liver.

In fact, the amyloidoses are systemic multi-organ diseases, so that aiming medication at the liver targets the source of the unstable monomers that improperly fold and accumulate in organs and tissues, while systemic circulation of a drug that disintegrates the amyloid should allow deposition to be prevented. At the same time, severely affected organs might benefit from the more concentrated upstream delivery of a successful drug to the arteries supplying the organ if not directly to the organ. The drug regimen for ameliorating the symptoms of an amyloid impaired heart dependent upon the type of amyloid (see, for example, Falk, R. H. 2011 cited above, page 1082), these drugs can be delivered directly to the heart.

Where a particular organ such as the heart is severely affected, the direct and concentrated delivery to and takeup within that organ or the arteries leading to it of prospective systemic medication to accomplish the prevention or even the eradication of existing amyloid deposits (Bodin, K., Elimerich, S., Kahan, M. C., Tennent, G. A., Loesch, A., Gilbertson, J. A., Hutchinson, W. L., and 13 others 2010. “Antibodies to Human Serum Amyloid P Component Eliminate Visceral Amyloid Deposits,” Nature 468(7320):93-97; Gillmore, J. D., Tennent, G. A., Hutchinson, W. L., Gallimore, J. R., Lachmann, H. J., Goodman, H. J., Offer, M., and 4 others 2010. “Sustained Pharmacological Depletion of Serum Amyloid P Component in Patients with Systemic Amyloidosis,” British Journal of Haematology 148(5):760-767; Kolstoe, S. E. and Wood, S. P. 2010. “Drug Targets for Amyloidosis,” Biochemical Society Transactions 38(2):466-470; Pepys, M. B., Herbert, J., Hutchinson, W. L., Tennent, G. A., Lachmann, H. J., Gallimore, J. R., Lovat, L. B., and 16 others 2002. “Targeted Pharmacological Depletion of Serum Amyloid P Component for Treatment of Human Amyloidosis,” Nature 417(6886):254-259) should allow much if not all function to be restored.

A successful drug for local interference in the production or removal of existing amyloid deposits would be facilitated were side-entry jackets placed along the internal carotid arteries for concentrated delivery of the drug to the blood shortly before entry into the brain (Moreno, J. A., Halliday, M., Molloy, C., Radford, H., Verity, N., and 6 others 2013. “Oral Treatment Targeting the Unfolded Protein Response Prevents Neurodegeneration and Clinical Disease in Prion-Infected Mice,” Science Translational Medicine 5(206):206ra138; Halliday, M. and Mallucci, G. R. 2013. “Targeting the Unfolded Protein Response in Neurodegeneration: A New Approach to Therapy,” Neuropharmacology Sep pii: S0028-3908(13)00401-00402; Urn, J. W., Kaufman, A. C., Kostylev, M., Heiss, J. K., Stagi, M., and 8 others 2013. “Metabotropic Glutamate Receptor 5 is a Coreceptor for Alzheimer αβ oligomer Bound to Cellular Prion Protein,” Neuron 79(5):887-902; Urn, J. W. and Strittmatter, S. M. 2013. “Amyloid-β Induced Signaling by Cellular Prion Protein and Fyn Kinase in Alzheimer Disease,” Prion 7(1):37-41; Kolstoe, S. E., Ridha, B. H., Bellotti, V., Wang, N., Robinson, C. V., Crutch, S. J., Keir, G., and 7 others 2009. “Molecular Dissection of Alzheimer's Disease Neuropathology by Depletion of Serum Amyloid P Component,” Proceedings of the National Academy of Sciences of the United States of America 106(18):7619-7623). Preventing the diversion of protein essential for normal synaptic function (Moreno et al. 2013, Op cit.) and the binding of soluble amyloid-β oligomers from binding to cellular prion protein (Um et al. 2013, Op cit.) now appear more important for preventing symptoms than does eliminating the accumulated misfolded deposits of prior protein.

Side-entry jackets, patch-magnets, and if necessary, an impasse-jacket to eliminate any residue, would critically improve upon systemic distribution of any drug that should be drawn outward toward the adventitia, fibrosa, or outer layer of any vessel or organ treated. Provided the problems of in vivo stability and cellular takeup are overcome, the liver can be targeted as described below with respect to directing immunosuppressive drugs to an organ transplant—by formulating the drugs for superparamagnetic nanoparticle carry with parenchymal penetration by patch-magnets engaged in the hepatic visceral peritoneum. A drug eluting stent has no comparable ability to draw a drug radially outward to the adventitia, nor to sustain the delivery of any drug or drugs prepared in the form of a ferrofluid.

Immunosuppressive drugs are used to prevent the rejection of transplants and to treat autoimmune diseases of localized expression. However, systemic immunosuppression increases the risks of infection, the development of malignancies, adverse drug-drug interactions that interfere with or prevent the treatment of regionally distinct comorbidities, and adverse side effects that would be avoided were the drug or drugs limited to the tissue that required these. The degree to which a given immunosuppressive drug can be targeted or focused to protect an allograft from rejection depends upon its mechanism. Unless reversible at or somewhat in advance of the transplant, immunizing factors that originate systemically, such as in the bone marrow, or remotely, such as in the thymus gland, require that any drugs to reverse these factors if essential be provided as background therapy in the circulation.

However, drugs that in time and distance are progressively more immediate in effect can be delivered less in advance of and closer in proximity to the transplant in the corresponding degree. Existing immunosuppressive drugs for preventing transplant organ rejection include lymphocyte gene expression, cytokine signal transduction, and nucleotide synthesis inhibitors (see, for example, Coico, R., Sunshine, G., and Benjamini, E. 2003. “Transplantation,” in Immunology: A Short Course, Hoboken, N.J.: John Wiley and Sons, page 265).

For optimal targeting, drugs requiring the least lead time and space to reverse immunizing factors at the inlets to the transplant are selected for delivery in situ. To support organ transplants and their anastomoses, side-entry connection jackets are placed on the inlet and possibly outlet stumps of donor organs before harvesting. Means to secondarily allow the targeting of immunosuppressive drugs to organs that had been transplanted in the past will alleviate the consequences of systemically immunocompromising transplant patients. Any drug with local action not dependent upon metabolism in the liver can be targeted.

Placement of a side-entry jacket can be used, for example, to alleviate the pain of refractory Prinzmetal's vasospastic, or variant, angina pectoris (see, for example, Cannon, C. P. and Brauwald, E. 2005. “Unstable Angina,” in Braunwald's Heart Disease, Philadelphia, Pa.: Saunders; Cannon, C. P. and Brauwald, E. 2005. “Unstable Angina and Non-ST-Elevation Myocardial Infarction,” in Harrison's Principles of Internal Medicine, New York, N.Y.: McGraw-Hill, page 1448; Merck Manual of Diagnosis and Therapy, 2006. “Coronary Artery Disease,” Chapter 73, page 635) in a patient who a. Shows a tendency to develop an arrhythmia that could lead to a sudden arrest, and/or b. Shows no evidence of atheromatous obstruction, and/or c. Does not respond to or should not be treated by performing an angioplasty, or d. Is unable to tolerate the long term use of the drugs commonly prescribed, to include nitrates, such as nitroglycerin; calcium channel blockers, such as the nondihydropyridine drug diltiazem and the phenylalkylamine drug verapamil; rho kinase inhibitors, such as fasudil; and other type drugs, such as amiodarone or papaverine hydrochloride if introduced into the systemic circulation. All are associated with adverse side effects and drug-drug interactions when introduced into the systemic circulation. Nonvasospastic angina can be treated by this means through the direct delivery of beta blockers, nitrates, calcium channel blockers, ranolazine, and amlodipine, for example. Should delivery of a primary drug other than antispasmodic elicit a spasmodic reaction, antispasmodic medication is delivered along with the primary drug.

Targeted drug delivery to the problem coronary artery or arteries not only implements the suppression of myocardial infarction within five years in the twenty percent of patients susceptible thereto, but allows the use of elevated levels of calcium channel blockers and therewith, the effective prevention of spasm while avoiding the risks in long term use of short acting calcium channel blockers, to include tachycardia, bradycardia, hypotension, dizziness, gingival edema, headache, constipation, leg edema, drowsiness, and associated more with immediate release, or short acting calcium channel blockers, and breast cancer in postmenopausal women (Coogan, P. F. 2013. “Calcium-Channel Blockers and Breast Cancer: A Hypothesis Revived,” available at “http://archinte.jamanetwork.com/article.aspx?articleid=1723870; Li, C. I., Malone, K. E., Weiss, N. S., Boudreau, D. M., Cushing-Haugen, K. L., and Daling, J. R. 2003. “Relation between Use of Antihypertensive Medications and Risk of Breast Carcinoma among Women Ages 65-79 Years,” Cancer 98(7):1504-1513; Sica, D. A. 2006. “Pharmacotherapy Review: Calcium Channel Blockers,” Journal of Clinical Hypertenions (Greenwich). 8(1):53-56; Opie, L. H., Yusuf, S., and Kübler, W. 2000. “Current Status of Safety and Efficacy of Calcium Channel Blockers in Cardiovascular Diseases: A Critical Analysis Based on 100 Studies,” Progress in Cardiovascular Diseases 43(2):171-196; Gillman, M. W., Ross-Degnan, D., McLaughlin, T. J., Gao, X., Spiegelman, D., Hertzmark, E., Goldman, L., and Soumerai, S. B. 1999. “Effects of Long-acting versus Short-acting Calcium Channel Blockers among Older Survivors of Acute Myocardial Infarction,” Journal of the American Geriatrics Society 47(5):512-517; Noll, G. and Lüscher, T. F. 1998 “Comparative Pharmacological Properties among Calcium Channel Blockers: T-channel versus L-channel Blockade,” Cardiology. 89 Suppement 1:10-15; Massie, B. M. 1998. “The Safety of Calcium-channel Blockers,” Clinical Cardioloogy 21(12 Suppement1 2):II12-II17; Opie, L. H. 1997. “Calcium Channel Blockers for Hypertension: Dissecting the Evidence for Adverse Effects,” American Journal of Hypertension 10(5 Part 1):565-577; Thulin, T. 1990. “Calcium Antagonists—Assessment of Side Effects,” Scandinavian Journal of Primary Health Care Supplement; 1:81-84; Russell, R. P. 1988. “Side effects of Calcium Channel Blockers,” Hypertension 11(3 Part 2):II42-II44; Hedner, T. 1986. “Calcium Channel Blockers: Spectrum of Side Effects and Drug Interactions,” Acta Pharmacologica et Toxicologica (Copenhagen) 58 Supplement 2:119-130).

The spasmic contraction of Prinzmetal's angina usually occurs about 1 centimeter distal (antegrade, downstream) to an epicardial coronary artery atheroma but can appear with endothelial dysfunction and abnormalities in vascular smooth muscle cell function in the absence of any treatable lesion, eliminating transluminal intervention as a remedy, such a procedure incapable of eradicating the endothelial dysfunction to which the problem is attributed. The delivery of nitroglycerin and a calcium channel blocker directly to the spastic artery or arteries eliminates the side effects and drug interactions associated with these drugs when introduced into the systemic circulation.

Although portions of side-entry connection jackets not required to prevent leakage following removal can be made absorbable or to spontaneously disintegrate over an interval, side-entry connection jackets are usually intended for long term or lifelong treatment of chronic conditions and not made for short term use or to be absorbed or removed. For example, placing a side-entry connection jacket on the recipient inlet stump with supply line or lines and surface port before resection of a diseased organ to be replaced by a transplant organ allows delivery to the transplant of immunosuppressive drugs such as cyclosporine, or ciclosporine; nonantibiotic macrolides, such as sirolimus (rapamycin), tacrolimus; azathioprine; mycophenolate mofetil; and glucocorticoids from the moment the transplant is sutured in place.

Since the jacket is upsteam to the anastomosis, the transplant organ is medicated in its entirety. Temporary closure where the condition may reemerge is by closing a shutoff and throttle valve-plug as described below. Primarily intended for chronic and incurable conditions and where an alternative approach cannot be used or would pose greater risk, the removal of a side-entry connection jacket and surface port as described below apply only when an adverse tissue reaction or persistent irritation eventuate. Placement of a pump line to periodically deliver adverse reaction palliative or remedial medication depends upon the site of the sensor. Substances indicated are addressed below in the section Clasp-electromagnets under Description of the Preferred Embodiments of the Invention.

Except on the carotids, jugulars, and coronaries, jacket removal if necessary can be accomplished most quickly by endoscopic upstream cross-clamping, excision of the jacketed segment, and end to end anastomosis; however, removal should seldom prove necessary. Removal from a coronary artery such as shown in FIG. 16 necessitates bypass through off-pump or beating heart distal anastomosis with a pedicled internal thoracic artery or the use a cardiopulmonary bypass machine. Removal from a carotid artery follows the same precautions as for a carotid endarterectomy, to include the use of temporary shunts and bilateral encephalography, for example, to monitor cerebral perfusion (see, for example, Messina, L. M. and Zelenock, G. B. 1997. “Cerebrovascular Occlusive Disease,” Chapter 80 in Greenfield, L. J., Mulholland, M. W., Oldham, K. T., Zelenock, G. B., and Lillemoe, K. D. (eds.), Surgery: Scientific Principles and Practice, pages 1753-1756).

Unilateral and bilateral carotid jacketing may be undertaken to treat atherosclerosis local to the bifurcation, to set the entry point for targeting the brain with drugs, or both, heparin, aspirin, and apixaban, for example, all deliverable through the jacket. The risk of embolism is greater where plaque is excised; however, any cause of postprocedural stenosis can induce a stroke and must be monitored. When the drug is bound to a magnetically susceptible carrier, takeup within the brain is with the aid of an external electromagnet over the short term and if necessary over the long term, by patch-magnet implants, as addressed in copending continuation-in-part application Ser. No. 13/694,835, entitled Integrated System for the Infixion and Retrieval of Implants with or without Drug Targeting. Any nonradioactive residue that would continue into the systemic circulation will rarely if ever approach a concentration that could cause harm.

Unwanted residues can be eliminated by jackets placed about the jugulars to release a reversal agent. Where a reversal agent remains to be developed, the carrier bound drug is trapped and drawn radially outward by a magnetized jacket with extraction grid to allow the use of a powerful external electromagnet. The presence of a radiation shield such as depicted in FIGS. 5 and 6 obstructs the openings in an open grid essential for extracting a trapped radionuclide, for example. Exceptionally, when accumulation over time and extraction through an extraction grid underlying an outer radiation shield layer that disintegrates to expose the grid is impermissible, to allow immediate extraction, jackets used for radioactive residues omit a radiation shield. Placement of a jacket is expedited in an open field and even through the small albeit preexisting incision to perform an endarterectomy carotid or otherwise.

Exceptionally, the opening made in the side of the ductus can be covered over with adherent thin tissue-engineered ing scaffold seeded with autologous stem cells or treated to encourage overgrowth to replace the tissue that was removed and surgical cement. A side-entry connection jacket offers a means for the direct delivery of food to the gut and gastric aspiration of a neonate with esophageal atresia, a feeding function defect, or a proximal deformity that interferes with feeding, where repair must be deferred, long term intubation is not possible or contraindicated, and/or total parenteral nutrition should be avoided, for example. Direct connection to the gut bypasses food and airway structures all susceptible to injury and adverse reactions when intubated, the severity proportional to the duration.

The trauma that can result during intubation may be greater than that of placing a side-entry connection jacket with line and port through a small or ‘keyhole’ incision. These factors are confirmed by the established practice of surgically inserting a feeding tube through an abdominal incision when esophageal varices, severe head trauma, or a proximal obstruction, for example, is present. The use of a side-entry connection jacket and surface port are less susceptible to irritation, infection, and injury. Insertion of a nasogastric, orogastric, or a dubhoff tube in a neonate, even one with normal anatomy, is especially prone to iatrogenic complications, to include mucosal erosions and fistulization (Agarwala, S., Dave, S., Gupta, A. K., and Mitra, D. K. 1998. “Duodeno-renal Fistula Due to a Nasogastric Tube in a Neonate,” Pediatric Surgery International 14(1-2): 102-103).

It has, for example, resulted in irritation anywhere along the route and perforations of the visceral pleura (Thomas, B., Cummin, D., and Falcone, R. E. 1996: “Accidental Pneumothorax from a Nasogastric Tube,” New England Journal of Medicine 335 (17): 1325), esophagus, posterior wall of the stomach, left lobe of the liver and the spleen hilus (Gasparella, M., Schiavon, G., Bordignon, L., Buffo, M., Benetton, C., and 4 others 2011. “Iatrogenic Traumas by Nasogastric Tube in Very Premature Infants: Our Cases and Literature Review,” [in English] Pediatria medica e chirurgica 33(2):85-88; Sudhakaran, N. and Kirby, C. P. 2001. “Pitfalls of Gastric Intubation in Premature Infants,” Journal of Paediatrics and Child Health 37(2):195-197), urinary bladder (Mattar, M. S., al-Alfy, A. A., Dahniya, M. H., and al-Marzouk, N. F. 1997. “Urinary Bladder Perforation: An Unusual Complication of Neonatal Nasogastric Tube Feeding,” Pediatric Radiology 27(11):858-859), cribriform plate (van den Anker, J. N., Baerts, W., Quak, J. M., Robben, S. G., and Meradji, M. 1992. “Iatrogenic Perforation of the Lamina Cribrosa by Nasogastric Tube in an Infant,” Pediatric Radiology; 22(7):545-546), and rarely, the pericardium (Hanafy, Eel-D., Ashebu, S. D., Naqeeb, N. A., and Nanda, H. B. 2006. “Pericardial Sac Perforation: A Rare Complication of Neonatal Nasogastric Tube Feeding,” Pediatric Radiology 36(10):1096-1098).

Accidental perforations usually necessitate the immediate devising of an appropriate strategy to avert death (see, for example, Jackson, R. H., Payne, D. K., and Bacon, B. R. 1990. “Esophageal Perforation Due to Nasogastric Intubation,” American Journal of Gastroenterology 85(4):439-442; Grünebaum, M., Horodniceanu, C., Wilunsky, E., and Reisner, S. 1980. “Iatrogenic Transmural Perforation of the Oesophagus in the Preterm Infant,” Clinical Radiology 31(3):257-261). The foam lining of a side-entry connection jacket made deep (thick, broad) for placement along the highly motile gut in any event, considerable room for growth is provided. The use of oral and nasal intubation to meet both respiratory and nutritional requirements can result in human errors leading to significant trauma (Ebenezer, K., Bose, A., and Carl, S. 2007. “Neonatal Gastric Perforation following Inadvertent Connection of Oxygen to the Nasogastric Feeding Tube,” Archives of Disease in Childhood. Fetal and Neonatal Edition 92(5):F407), something the distinct placement and markings on the ports used with side-entry-connection jackets would eradicate.

Insertion is prone to complications with patients of all ages (see, for example, Pillai, J. B., Vegas, A., and Brister, S. 2005. “Thoracic Complications of Nasogastric Tube: Review of Safe Practice,” Interactive Cardiovascular and Thoracic Surgery 4(5):429-433). Also problematic are the complications and trauma the tube can impart when left in place on a prolonged basis, as classified and enumerated by Pillai et al., for example (Pillai, J. B., Vegas, A., and Bristera, S. 2005. “Thoracic Complications of Nasogastric Tube: Review of Safe Practice” Interactive Cardiovascular and Thoracic Surgery 4 (5): 429-433; Vielva del Campo, B., Moráis Pérez, D., and Saldańa Garrido, D. 2010. “Nasogastric Tube Syndrome: A Case Report,” [in English and Spanish], Acta Otolaringologica Españiola 61(1):85-86; Brousseau, V. J. and Kost, K. M. 2006. “A Rare but Serious Entity: Nasogastric Tube Syndrome,” Otolaryngology—Head and Neck Surgery 135(5):677-679; Apostolakis, L. W., Funk, G. F., Urdaneta, L. F., McCulloch, T. M., and Jeyapalan, M. M. 2001 “The Nasogastric Tube Syndrome: Two Case Reports and Review of the Literature,” Head and Neck 23(1):59-63).

Target organ takeup of drugs prepared for superparamagnetic nanoparticle drug-carrier delivery is optimized for by positioning patch-magnets, described in copending application Ser. No. 13/694,835, about the periphery of the transplant or a diseased native organ or the placement along a native ductus or transplant of an impasse-jacket or jackets, likewise described therein. Whether transplantation is orthotopic or heterotopic, placing the side-entry connection jacket or jackets upstream from the inlet anastomosis or anastomoses assures that no portion of the transplant is left outside the medicated zone, as would be the case were the jacket applied to the donor organ when harvested. Targeting the transplanted organ seeks to minimize if not eliminate immunocompromise of the patient as a whole.

When no trace of the drug or drugs should be allowed to circulate, an impasse-jacket likewise described in copending application Ser. No. 13/694,835 is positioned upstream of the transplant. If the drug is radioactive, a shielded impasse-jacket is used. If depleted over a shorter interval, a disintegrable shield is used. If not, or the unshielded impasse-jacket residue is removed endoscopically. This approach is the more valuable when an elderly patient presents comorbidities, for example. In general, immediate delivery of a drug to only the tissue intended optimizes its potency as well as eliminates adverse drug interactions and the complications that result from misapplication of the drug elsewhere.

Substitution for conventional central catheters or in-hospital central lines placed for relatively brief periods in a nonambulatory patient is unintended. Such jackets can replace central catheters, but should afford significantly improved long-term performance, due both to the stability of the junction and the fact that unlike a central venous such as a Hickman, Groshong®, or Quinton® catheter, for example, the lumen is left clear. Another advantage of a side-entry connection jacket is that the diameter of the line and opening or ostium into the conduit can be made larger to support a higher flow rate, which is advantageous for applications such as leukapheresis. For bypassing vessels, luminal uniformity of caliber moving into, through, and past the junction supports laminar flow to lessen thrombogenesis.

A side-entry connection jacket is no less suitable for placement along the gut, renal artery, a ureter, or any other bodily conduit large enough to allow the jacket to be placed, microsurgical methods accepted. Transluminal placement and the use of a guidewire (see, for example, Rupp, S. M., Apfelbaum, J. L., Blitt, C., Caplan, R. A., Connis, R. T., Domino, K. B., and 6 Others 2012. “Practice Guidelines for Central Venous Access: A Report by the American Society of Anesthesiologists Task Force on Central Venous Access,” Anesthesiology 116(3):539-573) are sources of injury that are avoided. That peripherally inserted catheters and transluminal access is necessarily safer than endoscopic access is misconceived.

Both injuries incurred during placement (see, for example, Amerasekera, S. S., Jones, C. M., Patel, R., and Cleasby, M. J. 2009. “Imaging of the Complications of Peripherally Inserted Central Venous Catheters,” Clinical Radiology 64(8):832-840; Kusminsky, R. E. 2007. “Complications of Central Venous Catheterization,” Journal of the American College of Surgeons 204(4):681-696; Eulmesekian, P. G., Pérez, A., Minces, P. G., Lobos, P., Moldes, J., and Garcia Monaco, R. 2007. “Internal Mammary Artery Injury after Central Venous Catheterization,” Pediatric Critical Care Medicine 8(5):489-491; Hamilton, H. 2006. “Complications Associated with Venous Access Devices,” Nursing Standard Part One 20(26):43-50; Part Two 20(27):59-65), while indwelling (see, for example, Gonsalves, C. F., Eschelman, D. J., Sullivan, K. L., DuBois, N., and Bonn, J. 2003. “Incidence of Central Vein Stenosis and Occlusion Following Upper Extremity PICC and Port Placement,” Cardiovascular and Interventional Radiology 26(2):123-127), and consequences that can follow placement (Ge, X., Cavallazzi, R., Li, C., Pan, S. M., Wang, Y. W., and Wang, F. L. 2012. “Central Venous Access Sites for the Prevention of Venous Thrombosis, Stenosis and Infection,” Cochrane Database of Systematic Reviews 3:CD004084), to include life-changing restrictions upon movement essential to avoid accidents in which a life-long wearer would be highly vulnerable, should be less than is risked with conventional catheters (see, for example, Children's Mercy Hospital, Kansas City, Mo. 2010. “Central Line at School,” care card, available at http://www.childrensmercy.org/content/uploadedFiles/Care_Cards/CMH-11-384p.pdf; Zeigler, S. A. 2007. “Prevent Dangerous Hemodialysis Catheter Disconnections,” Nursing 37(3):70, available at http://www.fda.gov/MedicalDevices/Safety/AlertsandNotices/TipsandArticlesonDevice Safety/ucm064634.htm).

Catheter insertion into the subclavian vein medial to the border of the first rib is associated with kinking, or pinch-off, which can lead to pinch-off syndrome or pinch-off sign (Mirza, B., Vanek, V. W., and Kupensky, D. T. 2004. “Pinch-off Syndrome: Case Report and Collective Review of the Literature,” American Surgeon 70(7):635-644; Andris, D. A. and Krzywda, E. A. 1997. “Catheter Pinch-off Syndrome: Recognition and Management,” Journal of Intravenous Nursing 20(5):233-237), rarely resulting in spontaneous fatigue fracture (Hou, W. Y., Sun, W. Z., Chen, Y. A., Wu, S. M., Lin, S. Y. 1994. “Pinch-off Sign” and Spontaneous Fracture of an Implanted Central Venous Catheter: Report of a Case,” (in Chinese with English abstract in Pubmed), Journal of the Formosan Medical Association 93 Supplement 1:S65-S69), to which a junction accomplished by placement of a side-entry jacket with direct line to a pectoral port is less quickly established but not susceptible.

Use of a more pliant catheter would prevent fracture but promote kinking, and use of the internal jugular vein alleviates kinking and fracture, but the greater variability in anatomy necessitates the use of ultrasound imaging, undoing some of the advantage in speed of access (Jensen, M. O. 2008. “Anatomical Basis of Central Venous Catheter Fracture,” Clinical Anatomy 21(2):106-110). Access through the subclavian, internal jugular, or femoral veins pose overall about equal risk (Ge et al. 2012, cited in the preceding paragraph; Ruesch, S., Walder, B., and Tramèr, M. R. 2002. “Complications of Central Venous Catheters: Internal Jugular versus Subclavian Access—A Systematic Review,” Critical Care Medicine; 30(2):454-460) albeit nonidentical as to type, femoral access found by some to be more susceptible to infection (Hamilton, H. C. and Foxcroft, D. R. 2007. “Central Venous Access Sites for the Prevention of Venous Thrombosis, Stenosis and Infection in Patients Requiring Long-term Intravenous Therapy,” Cochrane Database of Systematic Reviews (3):CD004084) and subclavian to kinking.

Distinction in the rate of infection based upon access route has been brought into question (Marik, P. E., Flemmer, M., and Harrison, W. 2012. “The Risk of Catheter-related Bloodstream Infection with Femoral Venous Catheters as Compared to Subclavian and Internal Jugular Venous Catheters: A Systematic Review of the Literature and Meta-analysis,” Critical Care Medicine 40(8):2479-2485; Parienti J J, Thirion M, Mégarbane B, Souweine B, Ouchikhe and 12 Others 2008. “Femoral vs Jugular Venous Catheterization and Risk of Nosocomial Events in Adults Requiring Acute Renal Replacement Therapy: A Randomized Controlled Trial,” Journal of the American Medical Association 299(20):2413-2422; Deshpande, K. S., Hatem, C., Ulrich, H. L., Currie, B. P., Aldrich, T. K., Bryan-Brown, C. W., and Kvetan, V. 2005. “The Incidence of Infectious Complications of Central Venous Catheters at the Subclavian, Internal Jugular, and Femoral Sites in an Intensive Care Unit Population,” Critical Care Medicine 33(1):13-20; discussion 234-235).

The tight junction afforded by a side-entry connection jacket is, moreover, no less applicable to systolic pressures and thus usable intra-arterially no less than intravenously. With the venous junction established by means of a side-entry connection jacket, the fixed junction and position of the catheter outside the native conduit prevents erosion when access must be maintained over a long period, much less for to the of life (see, for example, Duntley, P., Siever, J., Korwes, M. L., Harpel, K., and Heffner, J. E. 1992. “Vascular Erosion by Central Venous Catheters. Clinical Features and Outcome,” Chest 101(6):1633-1638) or migration (see, for example, Oguzkurt, L., Tercan, F., Torun, D., Yildirim, T., Zümrütdal, A., and Kizilkilic, O. 2004. “Impact of Short-term Hemodialysis Catheters on the Central Veins: A Catheter Venographic Study,” European Journal of Radiology 52(3):293-299; Foust, J. 2004. Blood Flow Simulation Past a Catheter Positioned in the SVC-IVC-RA Junction: Steady and Unsteady Flow Considerations, Master's Thesis, Lehigh University, Bethlehem, Pa.).

The need to remove a central catheter is usually due to mechanical problems (see, for example, Darbyshire, P. J., Weightman, N. C., and Speller, D. C. 1985. “Problems Associated with Indwelling Central Venous Catheters,” Archives of Disease in Childhood 60(2):129-134). That complications are common with entry needle puncture is attributable to the needle, guidewire, and dilator, none of which are used to place a side-entry connection jacket. As a permanent junction, a side-entry connection jacket is safer than an indwelling catheter. A double lumen, fully intracorporeal dialysis or apheresis line leading to a surface port with a switch to open and close the circulation is suitable for use with a home machine.

Not situated in the bloodstream, a side-entry connection jacket should rarely if ever induce an arrhythmia (Shah, K. B., Rao, T. L., Laughlin, S., and El-Etr, A. A 1984. “A Review of Pulmonary Artery Catheterization in 6,245 Patients,” Anesthesiology 61(3):271-275). The addition to a side-entry connection jacket of a concentric magnetized layer allows the direct piping of drugs to the jacket as a magnetized collar for drawing of superparamagnetic nanoparticle carrier bound drugs into and through the lumen wall. The caliber of the opening or ostium into the native conduit and the line led to it from the surface of the body can be made larger than that of a central venous catheter.

This not only reduces the risk of vascular complications upon insertion of a larger catheter (see, for example, Wicky, S., Meuwly J. Y., Doenz, F, Uské, A., Schnyder, P., and Denys A. 2002. “Life-threatening Vascular Complications after Central Venous Catheter Placement,” European Radiology 12(4):901-907), but when the port is not implanted beneath as to be covered over by the skin, allows the passage of narrower catheters and cabled devices and various diagnostic sensors such as a fine fiberoptic endoscope or angioscope in addition to the infusion or injection by syringe of drugs. Unless analog to digital conversion is accomplished within each, the microcontroller must include an analog to digital converter. Similarly, where the motor of each pump in each pump-pair is analog and without an inmate digital to analog converter, the microcontroller must include a digital to analog converter.

Side-entry jackets that incorporate a concentric magnetized layer, or piped impasse-jackets, can be used to target superparamagnetic drug-carrier nanoparticles (see, for example, Akbarzadeh, A., Samiei, M., and Davaran, S. 2012. “Magnetic Nanoparticles: Preparation, Physical Properties, and Applications in Biomedicine,” Nanoscale Research Letters 7(1):144; Frey, N. A., Peng, S., Cheng, K., and Sun, S. 2009. “Magnetic Nanoparticles: Synthesis, Functionalization, and Applications in Bioimaging and Magnetic Energy Storage,” Chemical Society Reviews 38(9):2532-2542; Wahajuddin, M. and Arora, S. 2012. “Superparamagnetic Iron Oxide Nanoparticles: Magnetic Nanoplatforms as Drug-carriers,” International Journal of Nanomedicine 7:3445-3471; Silva, A., Silva-Freitas, É., Carvalho, J., Pontes, T., Araújo-Neto, R., Silva, K., Carriço, A., and Egito, E. 2012. “Magnetic Particles in Biotechnology: From Drug Targeting to Tissue-engineered ing, Chapter 13 in Petrie, M. (ed.), Biochemistry, Genetics and Molecular Biology: Advances in Applied Biotechnology, New York, N.Y.: In Tech Publishing Company; McBain, S. C., Yiu, H. H., and Dobson, J. 2008. “Magnetic Nanoparticles for Gene and Drug Delivery,” International Journal of Nanomedicine 3(2):169-180; Pankhurst, Q. A. Connolly, J., Jones, S. K., and Dobson, J. 2003. “Applications of Magnetic Nanoparticles in Biomedicine,” Journal of Physics Part D. Applied Physics 36:R167-R181; Tartaj, P., Morales, M. P. Veintemillas-Verdaguer, S., Gonzalez-Carreno, T., and Serna, C J. 2003. “The Preparation of Magnetic Nanoparticles for Applications in Biomedicine,” Journal of Physics D: Applied Physics 36, R182-R197; Soppimath, K. S., Aminabhavi, T. M., Kulkarni, A. R., and Rudzinski, W. E. 2001. “Biodegradable Polymeric Nanoparticles as Drug Delivery Devices,” Journal of Controlled Release 70(1-2):1-20) through the jacketed segment of the lumen wall.

Occlusion of the vasa vasorum is believed to induce a compensatory angiogenic response of neovascularization where the immature microvasculature is especially susceptible to hemorrhage (Galili, O., Herrmann, J., Woodrum, J., Sattler, K. J., and Lerman, L. O. 2004. “Adventitial Vasa Vasorum Heterogeneity among Different Vascular Beds,” Journal of Vascular Surgery 40(3):529-535; Moreno, P. R., Purushothaman, K. R., Fuster V., Echeverri, D., Truszczynska, H., Sharma, S. K., Badimon, J. J., and O'Connor, W. N 2004. “Plaque Neovascularization is Increased in Ruptured Atherosclerotic Lesions of Human Aorta: Implications for Plaque Vulnerability,” Circulation 110(14):2032-2038). While antiangiogenic drugs can be delivered through the jacket to suppress neovascularization, occlusion through compression of the intact primary or mature vasa vasorum may well have been the factor that initiated the disease cascade and is best kept to a minimum.

Atherosclerotic degradation of the encircled native conduit or ductus is averted by lining the jackets with biostable viscoelastic polyurethane foam. Rather than to place the vasa and nervi vasora, or fine microvasculature and nervelets of the larger ductus, under compression or tamponade, the foam enfolds or invests to accommodate these. A high density memory foam, variously referred to as a viscoelastic flexible polyurethane foam, slow recovery foam, or temper foam, of lower indentation force or load deflection and higher phase relaxation or phase change at body temperature in a thickness sufficient to minimize if not eliminate perivascular compression. A properly selected foam shape adapts to accommodate the vasa vasora quickly enough that compression is too brief to initiate the process of degeneration and compensatory neovascularization that can result in hemorrhaging and thromboembolism.

While some of the fine vasa vasorum externae must be broken to encircle the substrate ductus, for others and most of the vasa vasorum internae, even the temporary compression during placement and adaptation of the foam to the body temperature is avoided, because the jackets are provided with perforations. These are shown in the drawing figures as part number 19, which pass entirely through the layers of the jacket to the adventitial or fibrosal outer surface of the ductus or included periadventitial fat except where the tissue plug is to be removed, so that compression of the vasa vasorum is minimized and gas exchange between the adventitia and the foam and the surrounding body cavity is little if at all affected (see, for example, Sun, Z. 2014. “Atherosclerosis and Atheroma Plaque Rupture: Normal Anatomy of Vasa Vasorum and Their Role Associated with Atherosclerosis,” Scientific World Journal 2014:285058; Ritman, E. L. and Lerman, A. 2007. “The Dynamic Vasa Vasorum,” Cardiovascular Research 75(4):649-658; De Meyer et al. 1997 cited below; Williams, J. K. and Heistad, D. D. 1996. “The Vasa Vasorum of the Arteries,” (article in French; English abstract at http://www.ncbi.nlm.nih.gov/pubmed/8984146) Journal des Maladies Vasculaires 21 Supplement C:266-269).

These properties in an open cell foam facilitate permeation through the foam of a liquid therapeutic substance when wetted at the time the jacket is applied or continued to be delivered inside the jacket shell through a separate line. An open cell structure also less obstructs gas exchange in the interior environment. The condition of compression or tamponade imposed upon these fine structures is therefore brief if it arises at all. Depending upon the application, a side-entry jacket of the kind shown in FIGS. 3 thru 6 with magnet layer can be used to draw an anti-inflammatory, angiogenic, antiangiogenic, anticoagulative, thrombolytic, or antispasmic, or any other magnetically susceptible particle bound drug against or into the wall surrounding the lumen.

While earlier polyurethanes gradually deteriorated in the internal environment (see, for example, Sinclair, T. M., Kerrigan, C. L, and Buntic, R. 1993. “Biodegradation of the Polyurethane Foam Covering of Breast Implants,” Plastic and Reconstructive Surgery 92(6):1003-1014; Sinclair, T. M., Kerrigan. C L., and Sampalis, J. 1995. “Biodegradation of Polyurethane Foam, Revisited, in the Rat Model,” Plastic and Reconstructive Surgery 96(6):1326-1335), more recent formulations show good biostability (see, for example, Pinchuk, L. 1994. “A Review of the Biostability and Carcinogenicity of Polyurethanes in Medicine and the New Generation of ‘Biostable’ Polyurethanes,” Journal of Biomaterials Science. Polymer Edition 6(3):225-267; Szycher, M. and Reed, A. M. 1992. “Biostable Polyurethane Elastomers,” Medical Device Technology 3(10):42-51; Santerre, J. P., Woodhouse, K., Laroche, G., and Labow, R. S. 2005. “Understanding the Biodegradation of Polyurethanes: From Classical Implants to Tissue-engineered ing Materials,” Biomaterials 26(35):7457-7470). Biostable foams are available from Salviac Limited, Dublin, Ireland. Newer formulations and coatings will further extend the biostability of foams.

The secure connection afforded by the cinching jacket and compliance imparted by the combination of the spring hinges and foam lining accommodate intrinsic motility of the pulse or peristalsis, so that connection to an artery bodes no more risk than does connection to a vein. Depending upon whether a large or small ductus is treated, the release of 2,4-toluenediamine (TDA) from the foam lining is small to minute compared to that from a breast implant and too little to act as a carcinogen (see, for example, Vázquez, G. and Pellón, A. 2007. “Polyurethane-coated Silicone Gel Breast Implants Used for 18 Years,” Aesthetic Plastic Surgery 31(4):330-336; Shanmugam, K., Subrahmanyam, S., Tarakad, S. V., Kodandapani, N., and Stanly, D. F. 2001. “2,4-Toluene Diamines—Their Carcinogenicity, Biodegradation, Analytical Techniques and an Approach towards Development of Biosensors,” Analytical Sciences 17(12):1369-1374; Kulig, K. 1998. “Lifetime Risk from Polyurethane Covered Breast Implants,” Environmental Health Perspectives 106(11):A526-A527; Luu, H. M., Hutter, J. C., and Bushar, H. F. 1998. “A Physiologically Based Pharmacokinetic Model for 2,4-toluenediamine Leached from Polyurethane Foam-covered Breast Implants,” Environmental Health Perspectives 106(7):393-400; Hester, T. R. Jr., Ford, N. F., Gale, P. J., Hammett, J. L., Raymond, R., Turnbull, D., Frankos, V. H., and Cohen, M. B. 1997. “Measurement of 2,4-toluenediamine in Urine and Serum Samples from Women with Même or Replicon Breast Implants,” Plastic and Reconstructive Surgery 100(5):1291-1298).

Significantly, means for encouraging or forestalling hydrolysis, enzymatic breakdown, and attack by the immune system allow the rate of breakdown and persistence of implanted polyurethane to be widely adjusted. If any, 2,4-toluenediamine leached from current state of the art polyurethane foams should be too little to affect the adventitia and would be blocked from access to the native lumen by the sides of the side-entry connector. In that TDA is liberated as the material breaks down, biodegradation in vivo and the release of TDA are related, so that a biostable foam can overcome both problems. For this reason, enclosing the foam within an impermeable, biocompatible, and durable metal containing film that nonbrittle, prevents microfractures from forming when the foam is compressed and expands both prevents its disintegration and the release of TDA.

The firm of PFM Medical Aktiengeselshaft, Nürmberg can apply a no-coating free zone composite plastic and metal thin film of niobium, hafnium, zirconium, tantalum, titanium or titaniferous material, or a combination thereof over the surface of the foam by plasma-assisted chemical vapor deposition (Breme, F., Güther, V., and Osten, K-U van 2003. Composite Material, European Patent 0897997, also published as U.S. Pat. No. 6,057,031 and Deutsche Patent 19,736,449). This chemical barrier film must cover all portions of the enclosed foam, to include the sides of the foam bounding the internal surface of the outer shell where it dips down to line any perforations or fenestrations. A sputter coating process that similarly encloses the foam within an outer film of high ductility, hence, flexibility, with little if any alteration in the mechanical properties of the underlying foam could also be used.

The potential toxicity of some susceptible drug-carriers means that an accumulation thereof small enough and noninjurious if extracted no farther than into the foam lining of the side-entry jacket can be left to remain in the foam. Unless due to disease or a genetic defect the adventitia or fibrosa is sufficiently malacotic for the magnetic layer within the jacket to extract the residue into the foam, the intrinsic magnet, which must conform to the prior constraint that it be lower in mass and thickness than would cause discomfort if not tissue injury, requires an assist from an external electromagnet. Extraction into the foam and no farther radially outward, does not necessitate radial through and through perforations through the jacket wall in the form of an extraction grid or grating. Permanent and electromagnet jackets without the field strength to completely extract a toxic residue should be avoided and an electromagnetic extraction jacket such as shown in FIGS. 13 and 15 used in lieu thereof. While the response to an adverse tissue reaction can usually be obtained with the aid pharmaceuticals, toxicity not abolished or reduced to a tolerable level by such means demands extraction.

If toxic, or inducing of an adverse tissue reaction (see, for example, Singh, R. K., Kim, T. H., Patel, K. D., Knowles, J. C., and Kim, H. W. 2012. “Biocompatible Magnetite Nanoparticles with Varying Silica-coating Layer for Use in Biomedicine: Physicochemical and Magnetic Properties, and Cellular Compatibility,” Journal of Biomedical Materials Research. Part A. 100(7):1734-1742; Hong, S. C., Lee, J. H., Lee, J., Kim, H. Y., Park, J. Y., Cho, J., Lee, J., and Han, D. W. 2011. “Subtle Cytotoxicity and Genotoxicity Differences in Superparamagnetic Iron Oxide Nanoparticles Coated with Various Functional Groups,” International Journal of Nanomedicine 6:3219-3231; Narayanan, T. N., Mary, A. P., Swalih, P. K., Kumar, D. S., and 5 Others 2011. “Enhanced Biocompatibility of Ferrofluids of Self-assembled Superparamagnetic Iron Oxide-Silica Core-Shell Nanoparticles,” Journal of Nanoscience and Nanotechnology 2011 11(3):1958-1967; Kunzmann, A., Andersson, B., Vogt, C., Feliu, N., Ye, F., Gabrielsson, S., and 8 Others 2011. “Efficient Internalization of Silica-coated Iron Oxide Nanoparticles of Different Sizes by Primary Human Macrophages and Dendritic Cells,” Toxicology and Applied Pharmacology 253(2):81-93; Ahamed, M., Akhtar, M. J., Siddiqui, M. A., Ahmad, J., Musarrat, J., Al-Khedhairy, A. A., AlSalhi, M. S., and Alrokayan, S. A. 2011. “Oxidative Stress Mediated Apoptosis Induced by Nickel Ferrite Nanoparticles in Cultured A549 Cells,” Toxicology 283(2-3):101-108; Naqvi, S., Samim, M., Abdin, M., Ahmed, F. J., Maitra, A., Prashant. C, and Dinda, A. K. 2010. “Concentration-dependent Toxicity of Iron Oxide Nanoparticles Mediated by Increased Oxidative Stress,” International Journal of Nanomedicine 5:983-989; Witasp, E., Kupferschmidt, N., Bengtsson, L., Hultenby, K., Smedman, C., Paulie, S., Garcia-Bennett, A. E., and Fadeel, B. 2009. “Efficient Internalization of Mesoporous Silica Particles of Different Sizes by Primary Human Macrophages without Impairment of Macrophage Clearance of Apoptotic or Antibody-opsonized Target Cells,” Toxicology and Applied Pharmacology 239(3):306-319), or excessive in volume when trapped inside the open cell foam, an extraction-electromagnet such as shown in FIGS. 13 and 15 is used to extract the residue.

To allow a path for an adverse buildup to be removed from the subadvential or subfibrosal harbor with the aid of an external electromagnet, perforations are introduced from the internal surface of the foam lining to the external surface of the side-entry jacket. Extraction when necessary is to the closest location for innocuous deposition. If regardless of the intracorporeal tissue depth to which withdrawn, an unavoidable residue remains toxic or induces an adverse tissue response that does not subside and is irreversible in situ, then extraction is through the extraction grating and entirely outside the body. This allows access to the chambers of the heart for monitoring, for example, without the need to leave the device in place between readings or the need to reenter every time a reading is needed, a guideway having been prepositioned and the risks of entry not requiring to be confronted each time.

When access to the side-entry connection jacket is for no more than conventional injection or infusion, the port implanted at the body surface is ordinarily subcutaneous, or placed beneath the skin. Unless given open access to the surrounding environment, a vessel will undergo atherogenesis, or atherosclerotic degeneration (see, for example, De Meyer et al. 1997 cited below), and it is probable that if fully enclosed, other type native conduits or ductus might deteriorate. The literature does address the remedial effect of placing perforations through the jacket but not the amelioration thereof with a foam lining. This makes it advantageous that the side-entry connection jacket for use along the vascular tree not completely enclose the vessel but incorporate perforations entirely through the jacket to include the outer shell, magnet if present, and foam lining.

The perforations can be circular or linear, and if small will not disrupt magnetic performance, certainly not to an extent that cannot be compensated for, even though the outer shell must line the perforations through the magnet to isolate the magnetic material, which is toxic. By the same token, only the enclosed segment is affected thus, and serious lesions may relegate the atherogenic consideration to a secondary status. If radiation shielding requires that the vessel be completely enclosed for an indeterminate time, then the foam lining notwithstanding, the same line used to convey the radionuclide to the lesioned segment is used to deliver magnetic drug carrier bound antiatherosclerotic drugs. If the radiation shield and antiatherosclerotic drugs can later be dispensed with, then a shield is used that will disintegrate.

In that case, the jacket configuration shown in FIG. 4 without a radiation shield is encircled within a shield of unperforated shielding material such as tungsten in the form of an overlapping particulate shown in FIG. 6 wherein each particle is encapsulated to safely fall away and remain in the body once the absorbable bonding agent breaks down. Where the site of implantation would not afford the hydrolytic and/or enzymatic action required to break down the particle binder over the interval desired, the need for an additional invasive procedure to wet these with a suitable breakdown accelerant should be avoided. Instead, additional ingredients to accelerate breakdown are included in the binder, usually water and/or an enzyme.

The tungsten particles can also include ferrous matter before encapsulation and be bonded together with an organic solder having a low flow or denature temperature so that it disintegrates when placed in a magnetic field alternated at radio frequency. While a radiation shield must not be perforated; to shield low dose rate radionuclides it can, however, be made to safely disintegrate spontaneously due to hydrolysis and enzymatic attack or through the application of a solvent or heat to the particle bonding agent. Shortening the period for shielding is beneficial from the standpoints both of preventing atherosclerotic degradation in the encircled vessel, and targeted delivery notwithstanding, minimizing the period for administering anti-inflammatory drugs such as steroidal, antihyperlasic drugs such as everolimus, and pleiotropically acting antiatherosclerotic drugs such as a statin.

For this reason, a radiation shield that can be disintegrated once the administration of radioactive material is no longer needed is provided. Such a shield, addressed under Description of the Preferred Embodiments of the Invention, consists of tungsten heavy alloy particles encapsulated within an insoluble polymer and compacted so that particles in many layers overlap radially in relation to the ductus long axis. The encapsulated particles can be separated by application of a solvent. For example, if the particles were bonded together by means of a cyanoacrylate cement, wetting the shield with acetone, or dimethyl ketone, would dissolve the bonds. Also to compensate for the enclosure by the shield, antiatherosclerotic and anti-inflammatory drugs can be included in the medication delivered to the junction and/or vessel through a side-entry connector if available or a service channel.

The synthetic line or lines used to deliver a radionuclide, for example, to the jacket must also be shielded, even though the line itself will usually continued to be needed following radiation treatment to deliver nonradioactive substances. The inducement to eliminate the shielding once treatment has ended is not governed by the considerations pertinent to a native conduit, but rather the alleviation or prevention of discomfort without the need for a second invasive procedure. The outer shielding of jacket delivery lines can be made to disintegrate in the same way as the jacket shield. Preferably, however, a second invasive procedure is avoided by selecting materials that will allow the discretionary disintegration of the shield when examination reveals it can be removed preferably through lithotripsy or alternatively, through the application of heat.

For susceptibility to shock wave lithotripsy, the shield is internally organized into layers bonded by a matrix of low ultimate shear stress (see, for example, Rassweiler, J. J., Tailly, G. G., and Chaussy, C. 2005. “Progress in Lithotriptor Technology,” European Association of Urology Update Series 3:17-36; Xi, X. and Zhong P. 2001 “Dynamic Photoelastic Study of the Transient Stress Field in Solids during Shock Wave Lithotripsy,” Journal of the Acoustical Society of America 109(3):1226-1239). The bonding of encapsulated tungsten beads in a disintegrable line shield must afford pliancy as well as disintegrability.

Placing the patient in a radio frequency alternated magnetic field allows heating the magnet to a sub-Curie and sub-injurious temperature that will denature a specially formulated proteinaceous cement, eutectic protein solder, or mucilaginous substance with ferrous content as a binder matrix. The use of a drug-carrier itself magnetized negates such use. Alternatively, when the period over which treatment must continue can be predicted, a biodegradable substance such as one of blended polycaprolactone or polylactide, which can be accelerated to melt through the addition of heat, or of a glycolic acid based polymer can be used.

When a fluid conduction or water-jacket is used as a followup service channel to deliver nutrients, for example, the passage of transluminal diagnostic instruments, hemodialysis, or leukapheresis, for example, access is through a surface port with synthetic cover with antimicrobial precautions applied. The risk of bloodstream infection and the precautions to prevent such an eventuality should be about the same as for a conventional central line. Use of a side-entry jacket fed from an automatic ambulatory, or portable, throughput volume metered pump or syringe driver through a port implanted at the body surface can deliver a vasospasm-suppressing nitrate and/or calcium channel blocker such as nimodopine to the artery immediately at a higher dose than would be allowed to enter the circulation.

Microchannel pumps for separately targeting different sites are currently not made in small enough sizes for the patient to wear. A multichannel plug and implanted port receptacle or socket will expedite the treatment of chronic comorbidities of which the drugs. Such a pump is ideally miniaturized, integrated with the port implanted at the body surface as a permanent part thereof that can be worn beneath the clothing, and is not removed, but rather replenished by inserting a new refill cartridge, larger volumes of the therapeutic substance or substances delivered from a separately worn pump. In some patients, targeted drug delivery is recommended due to adverse interaction with other drugs used to treat a comorbidity or adverse side effects.

Treatment of Kounis syndrome, for example, is with the addition of an antihistamine and corticosteroid. The same connection made with an atherosclerosed coronary artery can be used to directly deliver a statin, proprotein convertase subtilisin/kexin type 9 inhibitor, or any other drug at a concentration in excess of the background or basal dose introduced into the circulation, atherosclerosis, for example, systemic. Diagnosis and automatic targeted treatment for the patient who is asleep, a child, senile, or otherwise incompetent using the means described has many applications exigent and routine, and should not be understood in a limiting sense as pertaining only to the application specified.

As explained in copending application Ser. No. 13/694,835, treatment of a non end-arterial artery, for example, with a magnetized collar, or impasse-jacket, placed at the antegrade or downstream end of the segment to be treated allows reducing the level or levels to that allowed to circulate at the distal limit of the segment defined, or exit. Where metabolic dysfunction arises in the liver, such a connection can be used to deliver drugs such as apolipoprotein 3C, for example, directly into the portal vein. The portable, or ambulatory, pump used to deliver the medication can be manually controlled by the patient.

In Prinzmetal's or variant angina however, because the vasospasm tends to occur at rest, often at night, and sometimes when the patient is asleep (Yasue, H., Nagao, M., Omote, S., Takizawa, A., Miwa, K., and Tanaka, S. 1978. “Coronary Arterial Spasm and Prinzmetal's Variant Form of Angina Induced by Hyperventilation and Tris-buffer Infusion,” Circulation 58(1):56-62, page 61; Voronin, I. M. and Belov, A. M. 2001. “Why Do Prinzmetal's Angina Attacks Occur in Sleep?,” (in Russian), Klinicheskaia meditsina (Moscow).; 79(8):64-65; Shappell, S. D. and Orr, W. C. 1975. “Variant Angina and Sleep: A Case Report with Therapeutic Considerations,” Diseases of the Nervous System 36(6):295-298), drug delivery is best automated.

To prevent the pain and the risk the spasm may pose, a is incorporated into the side-entry jacket to detect the vasospasm noninvasively from outside the vessel the instant contraction begins. One or more of the symptoms or indicia associated with vasospasm is used, that addressed below mechanical, a piezoelectric wafer about the foam lining of the jacket used to trigger delivery from the pump of medication the instant the magnitude of the signal from the wafer drops below a threshold level. More specifically, signaled is a reduction in the outer diameter of the artery on the systoles, measured as a threshold reduction in the root mean square force of outward compression when the pressure waves of the pulse pass through the jacket.

The thin and sensitive piezoelectric wafer is interposed between the foam and outer shell, or if present, the magnet layer. The cuff conformation of the jacket allows continuous monitoring of the pulse and blood pressure; however, as indicated above where limitation of sensor positioning to the jacket itself is discounted, this function can be performed anywhere along the same or another vessel by a separate cuff made specifically for this purpose (see, for example, Sideris, D. A., Vardas, P. E., Chrysos, D. N., Toumanidis, S. T., Michalis, L., and Moulopoulos, S. D. 1987. “An Extravascular Hydraulic System to Control Blood Pressure by a Feedback Regulation of the Venous Return,” Cardiovascular Research 21(5):337-341).

Within the cuff-configured jacket, a networked matrix of minute pressure sensors positioned as a layer between the internal surface of the jacket shell and foam lining can monitor peristaltic forces, velocity, and frequency, or pulsatile action and blood pressure. To allow the measurable transfer of the outward force exerted by the pulse through the foam, compliant projections in the form of small rubbery pillars fastened to the internal face of the wafer pass through the foam to its internal (adluminal, adaxial) surface. The foam lining and spring hinges that join the half-cylinders of the jacket urge the jacket closed about the native conduit or ductus without injury to the small vessels and nerves that enter and depart the adventitial or fibrosal surface while complying with the pulsatile or peristaltic expansion and contraction of the ductus. Once body temperature is reached, placement of the jacket shown in FIG. 21 should not cut off the flow of blood through the pericardiacophrenic artery, for example.

The pillars are contrast marked, such as with tantalum, to allow these to be positioned off to a side of small vessels and nerves supporting the native conduit to be jacketed. Alternatively, a microminiature laser range finder or distance sensor can be used. The pain of ischemia immediate, detecting chemical indicators such as thromboxane or acetylcholine in anticipation of spasm would allow preventing spasm and pain, so that provided testing does not necessitate entry into the lumen, detection thus would be no less effective. The continuous monitoring of oxygen delivery passing through the artery intrinsically provided by the aortic and carotid bodies along with adjustment of the luminal diameter through endothelial function remain the subject of much research. For conditions to which the body is unable to respond on its own, spontaneous response under autonomic intelligent control is sought to be supplemented with a prosthetic response system.

A suitable sensor allows signaling the pump whether the cause is thromboembolic or vasospasmic but shortly after pain has already set in (see, for example, Russell, D. M., Garry, E. M., Taberner, A. J., Barrett, C. J., Paton, J. F., Budget, D. M., and Malpas, S. C. 2012. “A Fully Implantable Telemetry System for the Chronic Monitoring of Brain Tissue Oxygen in Freely Moving Rats,” Journal of Neuroscience Methods 204(2):242-248; Bazzu, G., Puggioni, G. G., Dedola, S., Calia, G., Rocchitta, G., and 5 Others 2009. “Real-time Monitoring of Brain Tissue Oxygen Using a Miniaturized Biotelemetric Device Implanted in Freely Moving Rats,” Analytical Chemistry 81(6):2235-2241). Concurrent telemetric warning of hospital staff is likewise enabled.

Where the aortic body is injured or destroyed, the incorporation into large-scale reconstructions such as total arch replacement or various hybrid schemes that add a stent graft (see, for example, Vallabhajosyula, P., Szeto, W. Y., Desai, N., Komlo, C., and Bavaria, J. E. 2013. “Type II Arch Hybrid Debranching Procedure,” Annals of Cardiothoracic Surgery 2(3):378-386; Appoo, J. J and Pozeg, Z. 2013. “Strategies in the Surgical Treatment of Type A Aortic Arch Dissection,” Annals of Cardiothoracic Surgery 2(2):205-211, available at http://www.annalscts.com/article/view/1696/2373; Kent, W. D., Herget, E. J., Wong, J. K., and Appoo, J. J. 2012 “Ascending, Total Arch, and Descending Thoracic Aortic Repair for Acute DeBakey Type I Aortic Dissection without Circulatory Arrest,” Annals of Thoracic Surgery 94(3):e59-e61) of side-entry jackets incorporating oxygen and blood pressure sensors to trigger the direct delivery of drugs such as anticoagulants and nitrates from an ambulatory pump connected to a port or ports implanted at the body surface to prevent clotting and a lead to stimulate the respiratory centers in the medulla and pons allows approximation of normal dynamic functional response, or aortic reflex, to increase oxygenation.

Needle type oxygen microsensors, for example, made by Precision Sensing GmbH are adaptable for incorporation into the side-connector. By extension, any condition that generates anticipatory or prodromal chemical, such as enzymatic, hormonal, or cytokine, and/or mechanical symptoms at the jacket for which a sensor is available to register the change can be applied to control an ambulatory pump to preclude, suppress, or truncate upon onset, what would otherwise have been the ensuing crisis. This approach assumes a serious if not life-threatening chronic episodic condition that justifies implantation of the jacket and port at the surface.

Any such condition, to include severe migraine, variant angina, arrhythmia, or regional enteritis, for example, where a detectable marker is present for which a microminiature sensor to detect an anticipatory blood chemical marker (Ngoepe, M., Choonara, Y. E., Tyagi, C., Tomar, L. K., du Toit, L. C., Kumar, P., Ndesendo, V. M., and Pillay, V. 2013. “Integration of Sensors and Drug Delivery Technologies for Early Detection and Chronic Management of Illness,” Sensors (Basel) 13(6):7680-7713; Lalauze, R. (ed.) 2012. Chemical Sensors and Biosensors, Hoboken, N.J.: John Wiley and Sons; Sadana, A. and Sadana, N. 2011. Handbook of Sensors and Kinetics, Oxford, England: Elsevier; Rosen, Y. and Gurman, P. 2010. “MEMS [Micro-Electro-Mechanical-Systems] and Microfluidics for Diagnostics Devices,” Current Pharmaceutical Biotechnology 11(4):366-375; Elman, N. M. and Upadhyay, U. M. 2010. “Medical Applications of Implantable Drug Delivery Microdevices Based on MEMS [Micro-Electro-Mechanical-Systems],” Current Pharmaceutical Biotechnology 11(4):398-403; Staples, M. 2010. “Microchips and Controlled-release Drug Reservoirs,” Wiley Interdisciplinary Reviews. Nanomedicine and Nanobiotechnology 2(4):400-417; Comeaux, R. and Novotny, P. 2009. Biosensors: Properties, Materials and Applications, Commack, N.Y.: Nova Science Publishers; Joshi, R. 2006. Biosensors, Delhi, India: Isha Books; Eggins, B. R. 2002. Chemical Sensors and Biosensors, West Sussex, England: John Wiley and Sons; Turner, A. P. F., Karube, I., and Wilson, G. S. 1987. Biosensors: Fundamentals and Applications, Oxford, England: Oxford University Press), for example, for incorporation into the side-entry jacket can be provided may be used thus to avert an impending onset or exacerbation before the absolute threshold of sensation is reached.

Such a marker or markers are likely to be detectable in only a subset of each population, need only appear before the experiential correlates do, and may or may not go to the ultimate etiological basis for the condition. By comparison, an unpiped impasse-jacket while preloaded and made to discharge automatically, is limited to a lower rate volume of drug delivery. The signal whether used to initiate remedial drug delivery can also be used to alert local emergency medical services or if used for the latter purpose alone, does not require a line leading from the jacket to a port at the body surface with an ambulatory pump connected. When the cause is unknown, the pump can deliver a thrombolytic drug as well as a vasodilator. The volume of these drugs in relation to that of the circulatory system is minute and incapable of inducing problem bleeding should a interventional procedure or surgery be necessary.

The microminiature oxygen, or lambda, sensor must be washed over by the passing blood and therefore positioned to a side of the opening or ostium of the side-entry connector. Such a sensor can be used to initiate drug delivery regardless of the cause of the ischemia. Eventually, chemical and/or electrical sensors for detecting aortic, para-aortic, and carotid body outputs indicating hypoxia, used by the body to increase the respiration rate, or out of normal range acidity, can be applied to the immediate delivery of drugs to reverse thrombus or vasospasm as causes of the hypoxia in patients with chronic conditions. Less preferred is the detection of creatine kinase or troponin when the patient is already in pain; ideally, the patient if asleep is not awakened. Even when shutoff and throttle valve-plugs to be described is inserted in the line, the same catheter as is used to deliver the medication can run wires between the sensor or sensors and the pump.

However, feedback devices, sensors, and sensors mounted to shutoff and throttle valve-plugs are generally monitored telemetrically, and control adjustments to the openings through valves preferably accomplished by radio remote servo control, thus eliminating the need to slide the valve-plug along the side-entry connection line catheter containing a wire. Wires are generally reserved for higher current demand requirements, such as to heat a coil within a valve-plug to warm the passing medication and stringent space constraints disallow providing a local or inmate source of power. Since a previously positioned valve-plug would block the extraction of the tissue excised from the side of the ductus, a side-entry connector continuous with the line connected to it cannot also have a valve inserted in the line prior to placement. Therefore, when the need or benefit of a valve-plug is evident before placement, the plug is still positioned immediately after the side-entry jacket has been applied.

Certain procedures, to include the passage of a cabled device, such as a narrow endoscope, laser, or a linear or rotator thrombectomizer or atherectomizer, for example, are difficult to accomplish with fluid, or sol state medication already in the line, especially when a valve-plug must first be retrieved to clear the way. If the cabled device is narrow enough and the valve openings relatively large in cross-sectional area, then the cabled device can pass through the valve-plug. This maneuver is best reserved for devices such as an endoscope that allow passage through the valve to be observed. Medication which for better control, is pumped as a thermoreversible gel which liquefies at higher than body temperature and must be warmed upon approaching the vasculostomy is liquified by a heating element within a valve-plug.

Where a nichrome or other resistance wire coated for internal use is used to warm the medication flowing through the catheteric line not within a valve-plug but rather along the entire length of the line, so that the elastomeric seal surround of the valve-plug addressed below slides over it, the wire is stiff and taut. For this purpose, the elastomer surround of the valve-plug should have a lower coefficient of friction. A surround less resistant to being slid along the line is also used when the valve-plug must allow expeditious extraction from the line in order to pass a cabled device through the side-entry connector and into the native lumen. When the side-entry connection jacket is applied to a blood vessel, extraction thus without the need to insert a guidewire is by using the water-jacket to force the valve-plug out of the line.

In that case, the valve-plug has a less frictional surround which to clear the way from the water-jacket has been trimmed back at the vessel end. A hydrogel prepared drug not welling down the internal surface of the line tubing, then depending upon the frictional values required, the line can be made of a low friction fluoropolymer, usually polytetrafluoroethylene. To avert leakage or extravasation through the side-entry opening, the force to extract the valve-plug must be greater than the blood pressure to a magnitude that allows forcing the valve-plug out of the line but not so great as to cut off or significantly reduce flow through the vessel.

By reserving the use of wire for a valve-plug-inmate heating coil and instead using a stiff wire that enters the valve-plug at the rear, the problem of jamming by seizing and snagging the wire when run through the catheter and the valve-plug is moved is eliminated. A stiff wire connected to the back of the plug to deliver power to a component within the valve-plug to adjust the vanes or to warm a resistance wire inside the valve-plug eliminates the need for a wire affixed to the internal surface of the catheter wall as a continuous sliding contact or ‘hotrail.’ The hotrail is not also usable to warm the fluid line contents, which necessitates high resistance wire. For warming line contents, such as a thermoreversible drug hydrogel to flow all along the length of a side-connector or service channel line, a nichrome or other resistance wire is used, and can be run adjacent to the hotrail conductor.

Since the medication delivered through side-entry connector and service channel lines is usually in the form of a thermoreversible gel, the ability to warm the valve-plug or the line from end to end, peripherally liquid filming the gel, often expedites repositioning the valve-plug or passing a cabled device down the line and into the ductus. If delivery of electrical power to the valve-plug is through a wire that enters the valve-plug at its rear and trails behind it rather than through contact between a hotrail and sliding boot or shoe on the side of the valve-plug, then a keyhole at the center bottom of the valve-plug seen as part number 28 in FIG. 24 for insertion of a guidewire must be kept clear. Such a trailing wire must therefore be inflexible enough as not to jam the valve-plug when withdrawn and enter the rear of the valve-plug off to a side of the keyhole.

Except to warm the medication along the entire length of the catheter line when warming within the pump is not possible, wires used other than to power components within valve-plugs are seldom needed. In the clinic, placement of the patient in a radiofrequency alternated magnetic field can be used to warm magnetically susceptible matter in the drug and/or the side-entry jacket. With the aid of a sensor, response to acute cardiac events are supplemented by the immediate and direct delivery of drugs when signaled by an implanted cardioverter defibrillator. Reference to vascular applications should not be understood in a limiting sense, the potential uses of side-entry connection jackets pertaining to bodily conduits of any kind. The number of drugs separately delivered from an ambulatory pump depends upon the diagnostic comprehension afforded by the sensors used, delivery of several drugs independently and automatically controllable.

For patients lacking suitable graft vessels, the direct continuous delivery of anticoagulant and, if necessary, antihyperplastic drugs makes possible the use of catheters and existing synthetic blood vessels as coronary or other vascular bypass conduits, or ductus (see, for example, Lin, P. H., Chen, C., Bush, R. L., Yao, Q., Lumsden, A. B., and Hanson, S. R. 2004. “Small-caliber Heparin-coated ePTFE Grafts Reduce Platelet Deposition and Neointimal Hyperplasia in a Baboon Model,” Journal of Vascular Surgery 39(6):1322-1328; Chen, C., Lumsden, A. B., and Hanson, S. R. 2000. “Local Infusion of Heparin Reduces Anastomotic Neointimal Hyperplasia in Aortoiliac Expanded Polytetrafluoroethylene Bypass Grafts in Baboons,” Journal of Vascular Surgery 31(2):354-363).

Another area where elimination from the lumen, safe and stable mechanical connection with the conduit, and the ability for drug delivery targeted at the junction improve upon the long term use of a central venous catheter pertains to venous shunts such as peritoneovenous shunts used to drain peritoneal and pleural fluid into the circulation, to include those considered improved (see, for example, Kawaratani, H., Tsujimoto, T., Kubo, T., Aihara, Y., Takaya, T., and 5 Others 2013. “Liver Abscesses after Peritoneal Venous Shunt,” Case Reports in Gastroenterology 7(2):245-250; Martin, L. G. 2012. “Percutaneous Placement and Management of Peritoneovenous Shunts,” Seminars in Interventional Radiology 29(2):129-134; Perera, E., Bhatt, S., and Dogra, V. S. 2011. “Complications of Denver Shunt,” Journal of Clinical Imaging Science 1:6; White, M. A., Agle, S. C., Padia, R. K., and Zervos, E. E. 2011. “Denver Peritoneovenous Shunts for the Management of Malignant Ascites: A Review of the Literature in the Post LeVeen Era,” American Surgeon 77(8):1070-1075; Tomiyama, K., Takahashi, M., Fujii, T., Kunisue, H., Kanaya, Y., and 5 Others 2006. “Improved Quality of Life for Malignant Ascites Patients by Denver Peritoneovenous Shunts,” Anticancer Research 26(3B):2393-2395; Hu, R. H. and Lee, P. H. 2001. “Salvaging Procedures for Dysfunctional Peritoneovenous Shunt,” Hepatogastroenterology. 48(39):794-797).

Ductus side-entry connection jackets allow body surface-to-ductus, ductus-to-surface, ductus-to-ductus shunting, and ductus segment bypass connections. The delivery of drugs these afford is direct, immediate, and targeted, minimizing entry into the systemic circulation, and thus avoiding exposure to other tissue and adverse interactions with drugs used elsewhere in the body. Junctions with relatively thick-walled conduits or ductus such as the gut or with blood vessels for the delivery of drugs from outside the body (but not those used to channel the circulation) through a narrow vasculostomy, can be joined through a T-joint configured junction perpendicular or normal to the long axis of the receiving or discharging vessel. Although of value along thick-walled conduits rather than vessels, rectilinear junction of the side-entry connector and native conduit means that the adluminal end of the connector is usable as a manual tissue plug circle-cutter.

However, to least disrupt the laminar flow of blood and thus minimize thrombogenesis and shear stresses through the junctions of vascular bypasses and shunts that would induce endothelial dysfunction and atherosclerotic degradation in the tissue—and if in the arterial tree promote restenosis and the formation of atheromatous plaque—the points at which vascular branches converge and diverge are suitably angled (see, for example, Reneman, R. S. and Hoeks, A. P. 2008, “Wall Shear Stress as Measured in Vivo: Consequences for the Design of the Arterial System,” Medical and Biological Engineering and Computing 46(5):499-507; Stroev, P. V., Hoskins, P. R., and Easson, W. J. 2007. “Distribution of Wall Shear Rate throughout the Arterial Rree: A Case Study,” Atherosclerosis 191(2):276-280; Reneman, R. S., Arts, T., and Hoeks, A. P. 2006. “Wall Shear Stress—An Important Determinant of Endothelial Cell Function and Structure—in the Arterial System in Vivo. Discrepancies with Theory,” Journal of Vascular Research 43(3):251-269; Painter, P. R., Edén, P., and Bengtsson, H. U. 2006. “Pulsatile Blood Flow, Shear Force, Energy Dissipation and Murray's Law,” Theoretical Biology and Medical Modeling; 3:31).

Combining a reduction in turbulence with the administration of heparin or other such drug allow a plastic catheter to serve as a vascular bypass. This means for averting the formation of thrombus is probably enhanced through the application of new surface treatments to the synthetic tube used (see, for example, Breme, F., Güther, V., and Osten, K-U van 2003. Composite Material, European Patent 0897997, also cited above). Junction at an acute angle is also less radially protrusive and therefore more readily accommodated without encroachment upon and irritation to neighboring tissues. Moreover, when the caliber of the catheter is large enough, the avoidance of right angular junctions makes it possible to pass catheteric diagnostic and therapeutic instruments, such as a Swan-Ganz catheter and cabled devices such as a fiberoptic angioscope, through the junction.

The need to exit and reenter a native vessel such as an obstructed coronary artery at suitable divergent and convergent angles is no less significant when the segment bypassed is totally occluded. Intended to connect synthetic to native conduits for drug delivery or to withdraw diagnostic test samples, and native to synthetic to native to bypass or shunt rather than the direct connection by anastomosis of native conduits where no synthetic or catheteric line is involved, side-entry connection jackets are not limited to blood vessels and differ from vascular connectors devised to allow direct and sutureless end to end anastomosis between blood vessels, for example (see, for example, Tozzi, P., Como, A. F., and von Segesser, L. K. 2002. “Sutureless Coronary Anastomoses: Revival of Old Concepts,” European Journal of Cardiothoracic Surgery 22(4):565-570).

Because it allows flow in either direction, the bidirectionality of junctions provided by side-entry connection jackets can be used to withdraw blood upsteam and return it downstream or the reverse between any takeoff or origin and outlet or insertion large enough in caliber to be jacketed and situated to allow access without significantly traumatizing dissection. For example, surface ports, whether adjacent or separated, implanted for the purpose of introducing medication can be connected to an extracorporeal pump to shunt blood between these in either direction over a range of volume transfer rates limited only by the calibers of the lines and speed of the pump. If the flow rate is high enough, the majority of the blood passing through the circulation will be shunted from the intake to the outlet. Shunting can be passive by direct connection of the shunt takeoff and outlet jackets, but shunting from a small to a large vessel can be assisted by interposing a pump between inlet and outlet jackets.

This is usually with the pump extracorporeal by connection between the surface ports, but with the implantation of a miniature inline pump, can be intracorporeal. Shunting may serve only to redirect flow to bypass the intervening sections of the circulatory system or to apply an apheretic or hemodialytic function extracorporeally at the shunt, the rate of this process then a determinant of a suitable shunt flow through rate. The shunting of blood flow between distant points has the potential to further expand the use of drugs otherwise contraindicated due to adverse drug-drug interactions. For example, the concentration of a drug can be reduced over a segment of the circulatory system whether occupied by an organ or gland, by shunting a significant fraction of the blood around the segment, organ, or gland.

When a nondiseased vessel for grafting is unavailable, the ability of the patient to withstand a longer procedure requiring that a suitable graft first be harvested, or the longer time under anesthesia would best be minimized, side-entry connection jackets allow the connection of lines from the body surface and interpositioning of segments to be joined with optimized luminal continuity, minimal leakage, and if a vessel, angularly configured for minimal endothelial damage and exposure to the blood of nonintimal surface area (Gummed, J. F., Opfermann, U., Jacobs, S., Walther, T., Kempfert, J., Mohr, F. W., and Falk, V. 2007. “Anastomotic Devices for Coronary Artery Bypass Grafting: Technological Options and Potential Pitfalls,” Computers in Biology and Medicine 37(10):1384-1393). Because atherosclerosis is systemic, autologous vessels may be suspect in any event. Currently, when a native graft would be unusable, the condition of the patient militates against harvesting it, or a catheter must be joined to a native conduit, synthetic materials must be used.

For use in the vascular tree, however, synthetic conduits tend to encourage coagulation as to require anticoagulant serum levels in proportion to their narrowness. Until synthetic materials become available that will function more like the equivalent native conduits, the use of these materials will be limited to larger prostheses not requiring the use of anticoagulants at dangerous levels. At the same time, a homograft or allograft not harvested from an identical twin, or an isograft, requires immunosuppressive medication for life. If to a downstream point along the same ductus to bypass the intervening segment, or to another ductus as a shunt, the synthetic conduit or pipeline used will usually be made of polytetrafluoroethylene (Teflon,® E.I. duPont de Nemours and Company) or polyethylene terephthalate (Dacron,® Invista, Incorporated). While incipient and not filled with clot, a side-entry connection jacket can prevent continued enlargement and deliver medication.

Conduits of these materials in expanded or woven form (Stollwerck, P. L., Kozlowski, B., Sandmann, W., Grabitz, K., and Pfeiffer, T. 2011. “Long-term Dilatation of Polyester and Expanded Polytetrafluoroethylene Tube Grafts after Open Repair of Infrarenal Abdominal Aortic Aneurysms,” Journal of Vascular Surgery 53(6):1506-1513; Walker, T. G., Kalva, S. P., Yeddula, K., Wicky, S., Kundu, S., Drescher, P., d'Othee, B. J., Rose, S. C., and Cardella, J. F.; 2010. “Clinical Practice Guidelines for Endovascular Abdominal Aortic Aneurysm Repair,” Journal of Vascular and Interventional Radiology 21(11):1632-1655), chemically treated, and/or synthetic/native composite forms (see, for example, Naoum, J. J. and Arbid, E. J. 2012. “Bypass Surgery in Limb Salvage: Polytetrafluoroethylene Prosthetic Bypass,” Methodist Debakey Cardiovascular Journal 8(4):43-46; Khalil, A. A., Boyd, A., and Griffiths, G. 2012. “Interposition Vein Cuff for Infragenicular Prosthetic Bypass Graft,” Cochrane Database of Systematic Reviews 9:CD007921; Bastounis, E., Georgopoulos, S., Maltezos, C., Alexiou, D., Chiotopoulos, D., and Bramis, J. 1999. “PTFE-vein Composite Grafts for Critical Limb Ischaemia: A Valuable Alternative to All-Autogenous Infrageniculate Reconstructions,” European Journal of Vascular and Endovascular Surgery 18(2):127-132) all fall within the scope of the type catheters and prostheses to which the use of side-entry connection jackets applies.

In vascular applications where discontinuities of wall compliance results in adverse shear forces with consequent intimal hyperplasia, this material can be used in less stiff, expanded forms (Li, L., Terry, C. M., Shiu, Y. T., and Cheung, A. K. 2008. ““Neointimal Hyperplasia Associated with Synthetic Hemodialysis Grafts,” Kidney International 74(10):1247-1261; Loth, F., Jones, S. A., Zarins, C. K., Giddens, D. P., Nassar, R. F., Glagov, S., and Bassiouny, H. S. 2002. “Relative Contribution of Wall Shear Stress and Injury in Experimental Intimal Thickening at PTFE End-to-side Arterial Anastomoses,” Journal of Biomechanical Engineering 124(1):44-51; Ojha, M. 1994. “Wall Shear Stress Temporal Gradient and Anastomotic Intimal Hyperplasia,” Circulation Research 74(6):1227-1231; Bassiouny, H. S., White, S., Glagov, S., Choi, E., Giddens, D. P., and Zarins, C. K. 1992. “Anastomotic Intimal Hyperplasia: Mechanical Injury or Flow Induced,” Journal of Vascular Surgery 15(4): 708-717).

The “ . . . flow stagnation point along the arterial floor resulting in a region of low and oscillating shear where the second type of intimal thickening developed . . . ” described by the last of the references cited may prove unavoidable with mechanical means as to necessitate the continued administration of hyperplasia-suppressive medication, such as sirolimus and everolimus. The delivery of such medication through a second side-entry connector, second jacket, or a water-jacket inlet used as a service channel should alleviate this problem as a potential cause of bypass failure. The targeted delivery of these drugs in what corresponds to a minute dose in systemic terms averts the many side effects these can produce.

A silicone coating has been reported to lessen hyperplasia (Lumsden, A. B., Chen, C., Coyle, K. A., Ofenloch, J. C., Wang, J. H., Yasuda, H. K., and Hanson, S. R. 1996. “Nonporous Silicone Polymer Coating of Expanded Polytetrafluoroethylene Grafts Reduces Graft Neointimal Hyperplasia in Dog and Baboon Models,” Journal of Vascular Surgery 24(5):825-833). The targeting by direct delivery of an anticoagulant to tubing made of existing materials at concentrations that would not be circulated makes practicable the use of these materials as vascular conduits in narrower calibers. Metal tubing is thrombofilic (thrombophilic) and lacks pliancy, and expanded polymeric fabrics remain less compliant than the walls of native vessels.

While suture, clips, and staples (see, for example, Garitey, V., Rieu, R., and Alimi, Y. S. 2003. “Prostheto-prosthetic and Aorto-prosthetic Anastomosis Using Stents, Threads, Clips and Staples. In Vitro Comparative Study,” (English abstract in Pubmed), Journal des Maladies Vasculaires 28(4):173-177), can pose problems of leaking and hyperplasia, direct native to native conduit surgical anastomoses with suture allow healing. However, means for achieving more natural tissue integration of prostheses made of expanded polymerics without hyperplastic hindrance, currently limited to larger caliber reconstructions (Appoo, J J et al. 2013 and Kent, W. D. et al. 2012 cited above) where direct synthetic to native conduit anastomoses with suture can perpetuate the problems posed by suture (as well as destruction of the aortic body and the dynamic oxygenation response it provides), remains under investigation.

While the junction itself must be inconsistent in compliance, the use of side-entry connection junctions in lieu of surgical anastomoses avoids certain pitfalls associated with suture in general and as regards post-junction or post-anastomosis continuity of wall compliance (see, for example, Hollier, L. H. and Towne, J. B. (eds.) 2004. “Anastomic Aneurysms,” in Complications in Vascular Surgery, Chapter 6, pages 155, 156 New York, N.Y.: Marcel Dekker; Tiwari, A., Cheng, K. S., Salacinski, H., Hamilton, G., and Seifalian, A. M. 2003. “Improving the Patency of Vascular Bypass Grafts: The Role of Suture Materials and Surgical Techniques on Reducing Anastomotic Compliance Mismatch,” European Journal of Vascular and Endovascular Surgery 25(4):287-295; Ballyk, P. D., Walsh, C., Butany, J., and Ojha, M. 1998. “Compliance Mismatch May Promote Graft-artery Intimal Hyperplasia by Altering Suture-line Stresses,” Journal of Biomechanics 31(3):229-237; Dobrin, P. B., Mirande, R., Kang, S., Dong, Q. S., and Mrkvicka, R. 1998. “Mechanics of End-to-end Artery-to-PTFE Graft Anastomoses,” Annals of Vascular Surgery 12(4):317-323).

Blood vessels representing but one type of conduit contemplated, numerous types of artificial blood vessels are under development. Producing artificial blood vessels poses formidable complications (see, for example, Fink, H. 2009. Artificial Blood Vessels: Studies on Endothelial Cell and Blood Interactions with Bacterial Cellulose, Doctoral Thesis, University of Gothenburg, Göteborg, Sweden). Existing smaller caliber synthetic lines such a those for replacement of diseased coronary arteries tend to induce coagulation and clog. To administer an anticoagulant systemically in order to prevent this renders the patient susceptible to problem bleeding; plainly, to restrict the anticoagulant to the conduit intended, especially with minimal if any entry into the wider circulation of little more by volume than a trace dose, would confer an element of safety.

For narrower conduits to convey blood, the side-entry connection jackets described will be available for a prospective synthetic vascular material. Tending to become clogged due to coagulation, these and related materials have proven disappointing as replacements for smaller caliber vessels. That a synthetic coronary bypass catheter, for example, can have anticlotting drugs piped directly to it in a targeted way should overcome this limitation until better artificial vessels become available. Coronary artery bypass surgery can thus be made practicable for patients without vessels suitable for use as grafts or unable to withstand conventional autologous transplantation without the risk of deferring bypass surgery until permanent and functionally sufficient artificial vessels become available.

Placement of a side-entry jacket just upstream or close to the retrograde margin of a synthetic coronary bypass to release an anticoagulant and/or platelet blockade can enable the use of existing catheters as bypasses. The advantages and disadvantages of the physiological conduit are replaced with those of a synthetic conduit; however, a native graft can remain less predictable and so pose as great if not greater a risk of adverse complications than would an artificial bypass or replacement. Magnetized jackets for encircling a conduit to trap or release drugs bound to magnetically susceptible particles, or impasse-jackets, piped to the surface allow drug replenishment when an unpiped impasse-jacket cannot be preloaded to deliver the volume of the drug or drugs required and continued delivery would otherwise necessitate the use of reinvasive means in a clinic each time the drug had to be replenished.

Side-entry connection jackets used to join conduits afford capabilities for communicating with the junction so formed not obtained with native to native anastomoses. Conventional suture when properly applied with or without a surgical adhesive or tissue cement is superior to nonabsorbable vascular connectors for joining natural vessels. However, conduits joined with a side-entry jacket are not both native; rather, that jacketed is native, while that retained within the connector is synthetic or tissue-engineered but whether due to disease or trauma, lacking in a secure base to connect with suture. In contrast, known vascular connectors seek to join one natural vessel to another. Side-entry connection jackets are secure when placed, are not contingent for long-term dependability upon successful healing after the patient has been closed, and can be used to make end-to-side junctions between native and synthetic or tissue-engineered bypass conduits where an autologous graft is unobtainable or its secure healing not assured.

The establishment of a secure, nonleaking junction between synthetic and natural conduits is valuable when the use of an homologous graft in place thereof would impose a lifelong burden of having to take immunosuppressive drugs, and thus, to confront the constant reality of being immunocompromised. Drug targeting by the means described should, however, substantially restrict the immunosuppression to the graft and tissue in contact with it, averting the need to immunocompromise the patient as a whole. A side-entry connection jacket allows direct delivery to an autologous graft of the immunosuppressive medication, and a magnetized collar, or impasse-jacket, as described in copending application Ser. No. 13/694,835 allows the medication to be drawn into and taken up within the lumen wall of the graft. The delivery side-entry connection jacket can itself incorporate a magnet, so that the medication is drawn into the lumen wall upon arrival, or a separate impasse-jacket can be positioned downstream.

Moreover, provided a reversal agent is available, any residue that passes beyond the level for cutoff can be neutralized. Whether the agent is delivered through another side-entry jacket or is released by an impasse-jacket depends upon the amount of the agent to be made available. In most instances, the drug will have been taken up within the graft, and if not brought down to zero residue, than to close enough to zero as has no medical significance, so that no reversal agent will be needed. Attachment of the conduit to the connector is by pushing the conduit over the free end of the connector. To prevent disconnection of an elastic or rubbery catheter, the outer surface of the connector is provided with recurved prongs, and the joint additionally secured by pushing the end of the catheter over the connector, if necessary, banding or lashing it about.

Since the side-entry connector is synthetic and connected to a synthetic conduit and the angles and calibers of the side-entry connectors are selected to minimize shear stresses, shear stress at the entry to and exit from the bypass where the native and synthetic components meet is much reduced. When the angle at the insertion is favorable and the caliber of the lumen consistent, the synthetic line can be bonded to the internal surface of the side-entry connector by secure means to include plastic welding and adhesives. Sudden step-ups or step-downs in luminal diameter due to a ledge presented by the free edge of the inner tube is eliminated by using thin stock to make the side-entry connector and the synthetic line to be bonded to it, any ledge that could induce turbulent flow or damage blood cells progressively thinned or feathered out into the internal surface of the outer tube.

Deviations in luminal diameter along a service channel and the jacket used to join it to a native vessel have no medical significance; however, junctions that join synthetic to native or native to synthetic lines through which the bloodstream is redirected must, to the extent possible, be undisruptive to flow. That is, these should be uniform in internal or luminal diameter, properly angled, and without internal in- or outstepping ledges or protrusions that would cause turbulent flow (see, for example, Melih Guleren, K. 2013. “Numerical Flow Analysis of Coronary Arteries through Concentric and Eccentric Stenosed Geometries,” Journal of Biomechanics 46(6):1043-1052; Tan, F. P., Wood, N. B., Tabor, G., and Xu, X. Y. 2011. “Comparison of LES of Steady Transitional Flow in an Idealized Stenosed Axisymmetric Artery Model with a RANS Transitional Model,” Journal of Biomechanical Engineering 133(5):051001; Avila, K., Moxey, D., de Lozar, A., Avila, M., Barkley, D., and Hof, B. 2011. “The Onset of Turbulence in Pipe Flow,” Science 333(6039):192-196; Varghese, S. S. and Frankel, S. H. 2003. “Numerical Modeling of Pulsatile Turbulent Flow in Stenotic Vessels,” Journal of Biomechanical Engineering 125(4):445-460).

By comparison, native vessels have adaptive responses that allow flow into the coronary arteries from the far larger aorta, for example, so long as the person is otherwise healthy; should hyperlipidemia enter the picture, however, and an unmistakable underlying, covert intolerance for the shear stresses produced will quickly reveal itself, making the coronary arteries among the most common sites for the inducement of atherosclerotic lesions. A synthetic tube is not susceptible to lesioning, but is to the formation of thrombus, clogging, and causing damage to blood cells, any ‘adaptation’ necessitating the use of drugs, here delivered through a service channel or channels.

This is true at both the origin, or takeoff, and the flow rejoining or insertion jackets. At the same time, the delivery of an anticoagulant through a fluid conduction or water-jacket inlet used as a followup service channel suppresses coagulation and the formation of thrombus within the bypass and the delivery of sirolimus (rapamycin), or everolimus (Cagiannos, C., Abul-Khoudoud, O. R., DeRijk, W., Shell, D. H. 4th, Jennings, L. K., Tolley, E. A., Handorf, C. R., and Fabian, T. C. 2005. “Rapamycin-coated Expanded Polytetrafluoroethylene Bypass Grafts Exhibit Decreased Anastomotic Neointimal Hyperplasia in a Porcine Model,” Journal of Vascular Surgery 42(5):980-988), through the same or another jacket inlet reduces any propensity toward the formation of intimal hyperplasia.

Where disconnection would bode hemorrhaging or the leaking of septic contents, the synthetic line is made continuous with the side-entry connector, or no one means for establishing such connections between lines and side-entry connector are depended upon; rather, a cement is used with synthetic conduits, and prongs and/or banding, tying about, or lashing used to assure that the joint will be permanent. When a radiation shield of woven tungsten heavy alloy can be slid with little resistance over the underlying outer shell enclosing the magnet, a unitized or continuous line and jacket allow removal of the shield when no longer needed by disconnecting the surface port and pulling the shield out.

A woven sheath of fine tungsten wire in the number of layers required affords pliancy for routing the line for least discomfort due to any significant weight. Since a valve-plug would block the way to allow extraction of the tissue removed from the side of the ductus, a side-entry connection line continuous with the line connected to it cannot also have a valve inserted in the line prior to placement. The weave can tie in loops or eyelets at points along the periphery for passing suture to distribute any significant weight by apposition and suspension at several points.

The distal end of the woven sheath abuts upon the lock bushing, which the proximal end abuts against the rear of the port membrane, front plate, or equivalent internal surface. Use of a compacted encapsulated tungsten bead shield eliminates the need for even this superficially invasive procedure, and to avoid deeper reentry, the jacket itself is bead shielded. The encapsulated tungsten beads in a disintegrable line shield must be bound together to yield pliancy as well as disintegrability. Since the shield ensheaths a synthetic conduit, the means for effecting disintegration are less stringent as to the temperature or chemicals that can be used.

Generally, connection to a catheter made of a nonelastic and slippery polymeric, such as polytetrafluoroethylene, polyethylene terephthalate, or these in expanded or woven form is by inserting the ends of the side-connector and the catheter in an elastic or rubbery sleeve that clings to the slippery material so that the edges of each meet flush. End to end connection of the side-entry connector or the distal end of a catheter connected to the side-connector to a native conduit is similar to that used to join the side-connector to a nonelastic catheter, in that an external elastic connecting sleeve is used. The side of the sleeve to go over the native conduit is lined with viscoelastic foam and if disconnection is a concern, has recurved prongs protruding through the foam from a surrounding shell to undercut the adventitia, fibrosa, or serosa.

Delineation of the procedure for placement of a side-entry connection jacket, lines, and port is deferred until the need for various components has been made clear. Ordinarily, line connections to a jacket are made before the jacket is positioned along the ductus or a jacket with integral connector and line used; since once positioned the lines must be connected to the side-entry connector to allow excision of the tissue plug, reversing these steps gains nothing. Connection to the lines of the jacket allows the correct length to port of each line to be determined with confidence and eliminates the difficulty of connecting the lines to the jacket when placed at an awkward angle in relation to the approach.

FIG. 5 shows a side-entry jacket extended in the antegrade direction with a magnet layer that incorporates a tungsten radiation shield for use as a piped impasse-jacket, while FIG. 6 shows such a jacket with shielding formulated to disintegrate once the radiation has been depleted. As with all side-entry jackets, those smaller for placement along thinner walled ductus, do not require a rotatable side-connector with lock bushing for use as a circle-cutter. Radiation shielding must completely envelop the jacket and the line leading to it. In larger jackets such as those for use along the digestive tract, the screw lock bushing used to tighten the side-connector in position after use as a circle-cutter is bonded to the surrounding layers so that this segment at the jacket end of the line rotates together as a unit knob.

As depicted in FIG. 16, the most basic installation includes two lines, the side-entry connector line, or mainline 13, and a fluid conduction or water-jacket line, sideline 11 as a subsidiary or accessory line. Were the sideline only used after placement to deliver supplementary doses of an additional drug or drugs, an adjuvant, or a reversal agent, for example, these could usually be added to the medication delivered through the side-entry connector line and thus eliminated. However, the water-jacket line is essential during placement of the jacket along a blood vessel, for hemostasis and to assist in washing out the tissue plug, and during placement along another type ductus, to deliver immediate pressure irrigation, preventing septic contents from leaking into the surrounding cavity, which is important during installation in an emergency with no opportunity to purge the ductus.

After placement, the service channel remains available for the suppression of bleeding or leakage by pressure irrigation if the side-entry connection line is wanted emptied; the immediate delivery of a second medication that would otherwise not reach the ductus until it had passed entirely through the line with the medication already filling it; and to allow the aspiration of diagnostic test samples, for example. Immediately present in encircling and facing relation to the opening in the ductus, the delivery of any other medication is immediate rather than by travel through the side-entry connection line. The water-jacket line also aids in the quick evacuation of the side-entry connection line at the same time that it prevents a vacuum at the opening in the ductus. The water-jacket inlet line, available after placement as a service channel, is relatively flexible as to orientation, and is led to the entry port at the body surface as best routed to avoid encroachment on neighboring tissue.

Vessels pose a greater risk of complications than do other type ductus which pose less of a leakage problem, are usually larger, and more accessible. Nonmanipulative or passive circle cutting (trephination) of the entry plug for a side-entry connection jacket along the vascular tree not only reduces the risk of trocar gouging, but makes it possible for junctions at convergences and divergences to be set at the proper angle, the elliptical cutting edge not rotatable as a, and allows the jacket to be placed with lines already attached, further reducing the need for manipulation. Connection is never by means internal to the lumen, or channel. A central desideratum pertaining to the connectors and related ductus jackets is elimination from the lumen: left clear, the normal physiology of the ductus is far less if at all affected, and should a transluminal intervention become necessary, the lumen will be unobstructed.

By avoiding the placement of a foreign object in the lumen, extraluminal jackets, to include those incorporating a side-entry connector, avoid a central drawback of conventional means of intervention. While the side-entry jacket as a means for joining a synthetic to a native ductus pertains to all ductus, there are some distinctions in the application of this concept to vessels as opposed to other type ductus such as the gastrointestinal tract. Placement of a side-entry jacket along a nonvascular ductus is by suction while manually circle-cutting with the front razor edge of the side-entry connector, and usually, irrigation or flushing the opening created with water fed through the water-jacket rather than a tacky crushed hydrogel to prevent leaking. If clearance of the surrounding anatomy demands that the side-entry connector join at an angle, then water is still used although placement follows the vascular procedure, a more powerful vacuum needed to incise through the thicker ductus wall.

In contrast, placement of a side-entry jacket along the vascular tree is by ductus wall plug removal using suction without manual circle-cutting and a tacky viscid crushed hydrogel rather than water fed through the water-jacket. Whereas nonvascular side-entry connectors may be angled to avoid encroaching upon neighboring tissue, vascular side-entry connectors must be set at an angle to converge with the native lumen with minimal shear stress. When nonvascular side-entry jackets must be angled, placement is the same as it is for vascular jackets. The angular junction elliptical, the side-entry connector cannot be rotated as a circle-cutter (plug cutter, trephine), so that extraction of the tissue plug must be left to the sharp cutting edge of the side-entry connector and the sudden application of vacuum pressure, followed by the outward force of the blood and continued wash water, which restrains extravasation and reverses direction to flow out through the mainline or side-entry connection line driving the excised plug of tissue before it.

Lines with an elastic membrane slit remain filled, denying entry by luminal contents. The initial dose delivered in the form of a crushed tacky hydrogel then stops any bleeding as well as positions the drug for delivery when antegrade pumping through the mainline is begun. When the access port implanted at the body surface that leads through the connecting line and side-entry jacket into the native lumen is of the open type described below, valve-plugs and cabled devices such as a fiberoptic angioscope, intravascular ultrasound probe, or laser, for example, can be passed through to examine or treat the lumen. Slightly tacky hydrogel that adheres to the far end of a cabled viewing device is easily brushed away, even without first wetting the outer surface of the device with a lubricant such as ACS Microslide®, Medtronic Enhance®, Bard Pro/Pel® or Hydro/Pel®, Cordis SLX®, or Rotaglide®.

Once the tissue plug has been extracted, the line can be closed leaving it filled to deny entry into the line of luminal contents or extravasation prevented by inserting a valve-plug. A valve-plug if used is of the active type in open position. To minimize impeding the continued outflow of the crushed tacky hydrogel or liquid used to prevent extravasation and prevent a buildup of pressure that would force the gel or liquid into the opening made in the side of the ductus, the valve-plug is slowly advanced through the line outflow until positioned. To allow the operator to observe the position of the valve-plug through the endoscopic incision with an endoscope, the line must be transparent. Regardless of the type ductus to be jacketed, the endoscope also allows the use of a long handled pliars to loosen and tighten the lock bushing.

Unless radiation shielded, the jacket can incorporate perforations for adventitial or fibrosal gas exchange with its internal milieu. It long known that complete envelopment of an arterial segment within a jacket that lacks perforations induces atherosclerosis (see, for example, De Meyer, G. R. Y., Van Put, D. J., Kockx, M. M., Van Schil, P., Bosmans, R., Bult, H., Buyssens, N., Vanmaele, R., and Herman, A. G. 1997. “Possible Mechanisms of Collar-induced Intimal Thickening,” Arteriosclerosis, Thrombosis, and Vascular Biology 17(10):1924-1930) and that a radiation shield for use with radionuclides rules out perforations, the use of a radiation shield imposes requirements as to duration of use and the addition of drugs to suppress atherosclerosis. Shielded side-entry jackets and lines are intended for use with localized tumors in the lumen wall, for example.

However, if detected early, a femoral Ewing sarcoma of a long bone, for example, may be treated by jacketing the diaphyseal nutrient arteries and/or cutting through the compact bone and into the medullary cavity to directly deliver chemotherapeutic drugs to the intramural primary tumor at higher levels than can be circulated, and if the jacket is shielded, a radionuclide; however, due to the pronounced propensity toward metastasis with this diagnosis, a background systemic dose is essential. Advantage can also be taken of the graft versus tumor effect by using a simple junction type side-entry jacket without magnet layer such as shown in FIGS. 1 and 2 to deliver harvested hematopoietic stem cells (see, for example, Kolb, H. J. 2008. “Graft-versus-leukemia Effects of Transplantation and Donor Lymphocytes,” Blood 112(12):4371-4383).

Provided abnormal cells can be differentially bound to a susceptible carrier, an electromagnetic extraction jacket with flush line or an electromagnetic clasp-magnet with flush line directed at the central venous sinus can be used to intercept and extract such cells from the circulation, thus assisting the circulated drug to suppress metastasis. In such therapy, it is significant that the systemic dose can be reduced. The object is to target the bulk of the antineoplastic agents at the tumor as to allow a reduction in the systemic doses and thus avert the side effects. The foregoing is substantially limited to premetastatic disease, the need for resection and the ability to avoid raising systemic drug levels thereafter lost. If discontinuous or unextended in length or width, slit or slot shaped perforations to allow gas exchange between adventitia and the interior milieu need not significantly interfere with uniformity of drug-carrier takeup by the magnet.

To fully enclose the toxic magnetic material, outer shell 4 must line any perforations to the same depth as at the axial ends of the jacket, meaning from the external surface of the side-entry connection jacket up to the internal surface of foam lining 3. A jacket with radiation shield 12 is not perforated. To allow the use of a radiation shield that need not be recovered in a second invasive procedure, a shield is used that will safely disintegrate to expose the adventitia, fibrosa, or serosa through perforations through the more central magnet and foam layers. A shield that will spontaneously disintegrate can be made, for example, of biocompatible or chemically inert polymer encapsulated tungsten heavy alloy beads compacted to overlap in multiple layers bonded together with a thin coating of an absorbable adhesive, such as one of polyglycolic acid.

Such a jacket effectively isolates the tungsten heavy alloy by encapsulation without the need for a continuous outer shell but must incorporate a shell subjacent or long axially central to the magnet to fully enclose it once the radiation shield has disintegrated. The shell must not only enclose the magnet concentrically but line the perforations incorporated to allow the adventitia to ‘breathe.’ Since enclosure-induced atherosclerosis probably involves obstruction of the minute vasa supplying the artery treated, should perforations along prove inadequate to suppress atherosclerotic deterioration, a service channel is used to deliver anti-inflammatory and antihyperplasia medication.

The application of a side-entry connection jacket interrupts the internal surface of a native conduit—in an artery, the endothelium—by no greater an area than the opening or ostium leading into the side-entry connector, the impairment to endothelial function thus small and focal. No distal end of a catheter is suspended within the lumen to disrupt flow, to irritate the entry rim, or erode the intima over time, and there is no transition in the internal surface moving through the jacket and past the connector. Unless the opening into the lumen is too large, flow is not significantly disturbed or endothelial function disrupted briefly and not so that drugs cannot alleviate the temporary local trauma.

Connection of neither the side-entry connector to the conduit nor a water-jacket inlet to the line connected to it is fastened end-to-end with suture. The ductus is completely enclosed so that it cannot ‘breathe’ only when radiation shielding necessitates and then during the shortest effective duration of treatment and with the administration of antiatherogenic medication. The use of a synthetic conduit becomes necessary when no autologous vessel is suitable and when an autologous stem cell generated tissue-engineered replacement is unavailable or cannot be secured in position with suture. Surface-to-ductus pipeline catheters routinely incorporate more than a single lumen and can be used bidirectionally to deliver medication, draw diagnostic samples, pass testing sensors to the junction, or contain wires as necessary.

Enabling the use of synthetic materials to form bypasses and shunts and thus eliminate the need to harvest native tissue or stem cells to tissue-engineered a replacement, side-entry jackets placed beside native-native end to end anastomoses can also be used to target medication to the anastomosis and angled, allow a fiberoptic angioscope, for example, to be passed through the jacket to monitor the anastomosis, for example. The advantage compared to a conventional transluminal approach is that the prepositioned conduit with entry through a port implanted at the body surface eliminates the need for entry by incision and the risks of the transluminal method. It is not suggested that such a line is placed where the prospective uses therefor do not justify placement.

Side-entry and impasse-jackets seek to advance medical surgery, or medical management assisted by minor surgery, whereby a relatively low risk invasive procedure is used to position implants that make it possible to access and target, or substantially limit, medication to certain tissue while avoiding exposure to other tissue or drugs. A primary area for the application of such treatment is lifelong episodic severe disease, whereby detection of onset markers is applied to trigger delivery of counteracting medication, thus suppressing symptoms before these start. Junction by means of side-entry jackets is amenable to robotic placement and allows followup access not allowed by sutured anastomoses.

Whereas surface-to-ductus lines can be multiluminal, side-entry jackets used in ductus-to-ductus connections in lieu of bypass and shunt grafts and anastomoses are usually monoluminal. To allow different drugs or other therapeutic substances to be administered independently or simultaneously to a ductus, a line from the body surface to the side-entry connection jacket encircling the target ductus can be multiluminal, entry into the jacket through a side-entry connector and/or a fluid conducting or water-jacket inlet line, a jacket able to incorporate a number of either or both type inlets. where the pathology along a ductus differs in different segments, different jackets positioned along the ductus to treat each condition can be accessed through the same or different entry points at the body surface.

A side-entry connection jacket retains a synthetic bypass or shunt in its side-entry connector. The bypass or shunt is always monoluminal. Because the conduit or line is synthetic, the need for supportive medication through a supply line from a port at the body surface is substantially reduced to an anticoagulant and an antimicrobial ordinarily delivered though a water-jacket inlet used as a followup service channel rather than through another side-entry jacket placed on or upsteam to the bypass or shunt. Venous insufficiency that uses a synthetic conduit or ductus to divert blood from a diseased to a competent vein or to bypass an occluded segment along the vein is normally from a side-entry connector on the source vein, usually one of larger caliber, to a side entry connector on the destination vein or distal segment.

A side-entry jacket is not limited to a single connector. Rather, multiple connectors can be radially and/or longitudinally separated along the jacket. Thus, by connecting catheteric conduits to two or more side-entry connectors on a single jacket, the flow through the jacketed ductus can be diverted to two or more receiving ductus, and a ductus with two or more side-entry connectors can receive flow from two or more source ductus. Moreover, any one side-entry connector can be provided with more than one water-jacket inlet that can be used as a service channel to deliver drugs or aspirate biopsy samples, for example, once the jacket has been placed. Jacket supply lines from a port implanted at the body surface are catheteric, preferably synthetic.

In the drawing figures, a line connected to a side-entry connector, or mainline, appears in the drawing figures as part number 13, while a line connected to a fluid conduction or water-jacket, which can be continued in use once the jacket has been positioned as a service channel, or sideline, appears as part number 11. The water-jacket is used to minimize if not prevent spillage through the opening created just after the plug of tissue has been cut from the side of the ductus. Along the gastrointestinal tract, this is usually by flushing or irrigating the opening with pressurized water. Along a blood vessel, leakage is usually accomplished not with water but rather a tacky crushed hydrogel, which like the water, can incorporate drugs such as a broad spectrum antibiotic or antiseptic.

In subsequent use, mainlines and sidelines can be used to deliver the same or different therapeutic substances, which can be separated by segments of broad spectrum antibiotic-containing or inert nondrug containing crushed tacky hydrogel. With a blood vessel, a drug hydrogel is advanced by applying pressure to it, allowing control at the pump without the need for local valving. If the delivery medium is a slightly tacky crushed gel that does not flow unless driven, then control over leaking and dosing is much improved. Gels are more suitable for delivery into blood vessels as these expedite hemostasis. With such a delivery medium, if necessary, an actively controlled butterfly shutoff and throttle valve-plug is used.

If the delivery medium is a free flowing fluid, then control is attained with the aid of a passive elastic slit membrane or fine fiber spandex stretch valve-plug at the adductal end of the side-entry connector, thus covering over the opening into the ductus. Passage through the valve-plug requires a threshold minimum pressure. Along the gastrointestinal tract or a ureter, for example, leakage of septic or potentially septic contents must be prevented, and this can be accomplished by flushing the opening with antimicrobial containing water or a gel. With a backup valve-plug positioned with its adductal face level with the edge of the water-jacket and its vanes open, a drug or drug hydrogel under antegrade pressure and delivered through the mainline advances into the ductus.

Depending upon the viscosity of the crushed hydrogel or syrup, for example, its tackiness, the size of the opening into the water-jacket, and the resistance posed by a slit or elastic weave membrane or sieve type valve-plug if present, some of the drug will enter into the water-jacket or sidelines if these are not already filled. Provided the drug is in the form of a tacky crushed gel, the water-jacket or sideline will quickly clog, so that dosing will substantially equate to that delivered through the mainline. Opening the valve-plug allows delivery of the same or different drugs through the mainline and sidelines. Under these same conditions with the valve-plug open, a drug delivered through a sideline or the water-jacket as a service channel only will back up, that is, flow into the mainline at the head of the column of the primary drug hydrogel to follow, where it can serve as neoadjuvant or preparatory adjuvant.

Closing the backup valve-plug allows drug delivery through the sidelines alone. In aspirative or retrograde flow, closing the valve-plug limits aspiration to the water-jacket, whereas opening it allows aspiration through both the mainline and sidelines. When present, a valve used to throttle flow through the line is fully opened when the rate of delivery is controlled by a pump; otherwise, when hard wire powered or remote controlled as described below, such a valve can be used as a rate of delivery trimmer throttle. Multiple lumen lines serve to separate components that become active when combined or are best kept apart during delivery up to the valve-plug, which may be situated at the distal terminus of line 13 or 11 just where the native lumen or water-jacket inlet respectively are entered.

Another option is to flush through the line between components or fractions to be kept separate until delivered by placing water or solvent cartridges or connecting a hose feed into the pump turret between the components. For example, the side-entry connector line is flushed outward by pumping water through the water-jacket. A single valve is used with a double or larger number multiple lumen line, but the valve itself must be lodged within a single lumen segment along the line; however, to prevent directional reversal at the valve where the hydrogel drug moves through one vane and returns through the other, the delivery lumen or lumina must be aligned to one of the two vanes or leafs of the valve and return lumen or lumina the other vane.

A valve-plug for use within a side-connection line having two lumina where either lumen must be aligned to a respective vane incorporates an internal medial septum to the outer or ductus abaxial side of the vanes that clears the leadscrew or servo connector and keying the distal end of the dual lumen line to the septum. However, since spanning a valve-plug across more than one lumen prevents its removal or repositioning, the use of a dual-lumen line can be applied only where it is known with confidence that the valve-plug will not have to be removed or resituated. Since as described below, adjustment of the vane angles can be by wired or remote electrical means volumentric flow rate through such an immobilized valve-plug is not prevented.

Where the port is not of the subcutaneous membrane type but rather configured to allow the passage of devices wider than an injection needle, the water-jacket is used to irrigate the opening in the ductus under pressure. A valve cannot be used because the plug of tissue excised or a cabled device must be passable, and a valve would block the way from the line into the ductus. To restrain luminal contents from spilling out into the surrounding cavity, either the water-jacket is used to irrigate the opening in the ductus under pressure, or the pressure of the fluid medication itself accomplishes this; both side-entry and water-jacket lines are not used at the same time. To prevent medication from entering the water-jacket thus cycling around and flowing out through the service channel line or lines, water-jacket lines are kept filled with water or if already in use as service channels, then the therapeutic fluid these conveyed.

Conversely, when the water-jacket is used to pressure irrigate the opening in the ductus, the side-entry connection line must be clear to allow the wash water to flow out. Antegrade or adductal flow can consist of fluid medication with or without adjuvant medication delivered through a service channel or channels, in which case all lines remain connected to the pump. Some pumping arrangements will require that for the opening in the ductus to be irrigated, the main or side-entry connection line must be vented to the exterior or opened at the pump end to allow the wash water to flow out. In addition to the need to restrain lumen content from leaking when the opening is incised in the side of the ductus, the need for a clear passageway arises whenever a throttle or shutoff valve-plug or a cabled device must be inserted or withdrawn.

A segment of an artery that requires mechanical or laser angioplasty, stenting, or examination with the aid of a fiberoptic angioscope or intravascular ultrasound probe is thus made accessible. Access to the lumen of the ductus is through the lower of two entry holes at the back of the pump-pack, through the lower arm of the inline port or clean-out shown in FIG. 30, up through the pump line seen as part number 13 in FIGS. 29, 31, and 32, and through the side entry connector. The irrigation continues as a cabled device is passed through or removed from the opening in the side of the ductus. Ordinarily, incremental delivery is apportioned by means of a valve of the type used in a secondary intravenous line intravenous piggyback port, or a one arm inline port or clean-out of the type shown in FIG. 30. Further to minimize leakage, the fluid column in the line leading to the side-entry opening or the water-jacket inlet can include a gelling or thickening agent.

In conventional use, the diameter of the opening through the valve-plug can be equal to the internal diameter of the line proximally and distally to the valve-plug, because the valve itself can be larger in diameter overall; here, however, the valve must be placed inside and toward or at the distal end of the supply line catheter, so that no such latitude is available. When incorporation of a spring-loaded ball type valve-plug untenably reduces the luminal or internal diameter of a line that must be small in caliber to begin with, the terminal valve-plug consists of either a duplex disc or wafer style butterfly valve or an elastic polymer woven fabric or spandex like weave that will yield to column pressure over a threshold level which is fit over the adductal or ductus-adaxial end of the delivering line and side-entry connector to cover over the distal end, proximal portions of the slit membrane valve bonded to the outer surface of the line by means of a suitable adhesive. Of these types, that mechanical is shown in FIGS. 15 and 17 with a passive slit elastic membrane type shown in FIG. 25.

When the drug is administered by pump, the volumetric flow rate can be set at the pump; however, the valve can function as a throttle whether delivery is by infusion or injection and not limited to the bistable, that is, either fully open or fully closed. For use along the gastrointestinal tract, the entry of air into the ductus lumen is inconsequential, so that the valve alone can be used to check inflow through a side-entry or if sufficiently large in diameter, a service channel line. However, along the vascular tree whether the drug is fluid or in the form of a gel, any unoccupied segment of the line is filled with a nondrug or neutral gel or water or a liquid therapeutic to prevent the introduction of air into the vessel. In some instances, a nonwoven or continuous sheeting material or fabric of suitable elasticity with a slit or slits over the distal end can be used if a sock is continued back over and bonded to the outer surface of the delivery line.

Along the gastrointestinal tract where the shear forces of peristalsis might part the weave or slits of the elastic retaining valve to allow seepage into the delivery line, the antibiotic containing water can be preceded at the distal end by a column of stable but readily gel-converted or dissolved blocking material, such as a biocompatibly disintegrated aerogel or hydrogel (see, for example, Lutolf, M. P. 2009. “Biomaterials: Spotlight on Hydrogels,” Nature Materials 8(6):451-453; Jeon, O., Bouhadir, K. H., Mansour, J. M, and Alsberg, E. 2009. “Photocrosslinked Alginate Hydrogels with Tunable Biodegradation Rates and Mechanical Properties,” Biomaterials 30(14):2724-2734; Peppas, N. A. 2004. “Hydrogels,” in Ratner, B. D., Hoffman, A. S., Schoen, F. J., and Lemons, J. E. (eds.), Biomaterials Science: An Introduction to Materials in Medicine, New York, N.Y.: Academic Press, pages 35-42) until needed.

Medication for delivery into a vessel should not be formulated as an aerogel when the accumulation of liberated gas can become sufficient to constitute a gas embolism, If offsetting factors override this generalization, then the dose is adjusted to compensate for the change in volume. Any jacket can be directly fed different drugs timed together or separately through a multiluminal catheter entered through a port at the body surface and connected to a single side-entry connector. delivery from an automatic ambulatory pump with timing controls, for example. Jackets must be suited to the proposed site for placement, so that a jacket for placement about the ascending aorta to connect synthetic coronary artery bypass conduits, for example, must be positioned and configured to minimize encroachment upon the pulmonary trunk and superior vena cava.

It is therefore minimized in outer diameter, achieved primarily by reducing the thickness of the foam layer but not to a thickness less than that of the ductus wall as would not allow the tissue plug to be cut through entirely, compliance with intrinsic movement in the encircled ductus then more dependent upon the restorative force of the spring hinges. By contrast, a side-entry connection jacket for use with a long bone such as mentioned above in connection with Ewing sarcoma must adapt for a cross-sectional radial asymmetry of the bone, accomplished by using a jacket with a thicker foam lining. The variability in jacket proportions and configuration, to include the number of side-entry connectors, and the ability to variably apportion jackets to the aorta and its large derivatives is such that the highly variable anatomy is readily accommodated. The placement of the jacket and the positions, angles, and length of the connectors are taken into account.

Given that the input or body surface-to-ductus lines or catheters can be plural, can be each multiluminal, and that plural side-entry jackets can be positioned along a given ductus to receive plural catheters, it is apparent that the possibilities for ductus interconnection thus outstrip, much less satisfy, medical necessity. For example, dividing the outflow of a ductus between two other like type ductus through separate side-entry connectors of a single jacket has uses, but except for a line from the surface to a ductus, for either line to include more than a single lumen does not. The practicality of multiple or multiluminal lines from the body surface may be justified when each drug is best targeted to certain tissue and substantially kept out of the systemic circulation, as will be addressed, and/or the different drugs can be used only because the targeting sufficiently isolates the drugs from one another that adverse drug-drug interactions are avoided. The capability should stimulate implementation.

When the ductus is a blood vessel, an additional line from the surface is connected from a pump to a water-jacket in the side-entry connector which is used to irrigate the plug excision entry wound under pressure, restraining bleeding and assisting to remove the plug. Once the side-entry connector is placed, the water-jacket, not limited to the one entry line used to assist in vasculostomy, can be used to deliver drugs independently or to withdraw test samples from the lumen. Not only can multiple lines deliver fluids to and from the water-jacket, but any line connected to the connector or water-jacket can connect subsidiary lines through side-entry connection jackets, for example.

Whether between native vessels or catheters connected by side-entry jackets, secondary or supporting surface-to-ductus connections can be used to maintain or monitor primary ductus-to-ductus junctions. For example, anticlotting drugs can be delivered directly to junctions between vessels prone to thrombose. Used to secure a synthetic bypass or one tissue-engineered where additional support is essential to form a secure junction, the jackets can be sent anticlotting medication, and if necessary, a bactericide, viricide, and/or anti-inflammatory drug, for example. To support organ transplant end to end anastomoses, the inlet and outlet stumps, or stubs, of donor organs are provided with side-entry jackets before harvesting, or excision.

This allows the direct delivery to the transplanted organ and anastomoses of anticlotting, antiatherosclerotic, and if homologous, immunosuppressive drugs to the substantial exclusion of the rest of the body. The use of synthetic or otherwise tissue-engineered catheters that require secure connection not obtainable with suture alone as bypass grafts joined by side-entry connection jackets may eliminate tissue reactions, reducing the need for medication to that anticlotting. Drug targeting seeks to limit medication to the tissue that requires it. Where achieved, drug targeting allows the circumscribed and focused application of a drug to a diseased part or lesion at a concentration that if circulated could prove injurious if not toxic to other tissue, result in unwanted side effects, and/or adverse interactions with drugs intended for other tissue.

The ability to target tissue not only eliminates systemic limitations on concentration and thus the efficacy of a drug where needed, but eliminates the waste of greatly diluting costly drugs throughout the circulatory system to achieve the dose intended for the target tissue when this concentration can even be permitted. There is no disease in which the circulatory system is uninvolved and local dysfunction not signaled to higher control centers, often initiating a cascade of dysfunction. Direct-to-lumen side-entry connection as described herein allows the targeting of drugs to a definable segment of a tubular anatomical structure, or ductus, and therewith, the tissue supplied from or drained through the segment.

Medication piped directly to a specific level along the tubular structure bypasses the lumen upstream and any tissue branches thereof supply, and is limited in extent by positioning complementary or counterpart means downstream for taking up and/or neutralizing the drug. A reversal agent can be delivered to the ending level through a second side-entry jacket, so that only the intervening segment is exposed to the drug. When bound to magnetically susceptible drug-carrier particles, a magnetized jacket, or impasse-jacket, encircling the ductus downstream at the level for takeup can be used to draw the medication into and through the lumen wall.

An especially potent or radioactive drug that could injure nontargeted tissue can be eliminated by placing a backup side-entry or impasse-jacket to eliminate any of the drug not neutralized by the jacket positioned at the first ending level. The structures can be entering or departing vessels, ducts, or any other type of tubular channel along the circulatory, digestive, genitourinary system, or upper airway. Likewise, the omission of a segment specifically eliminates that segment and the tissue supplied by its branches from exposure to the drug. Drug targeting by such means can fulfill a central role in the treatment of disease. A side-entry connector is a small tube open to an anatomical lumen. The connector extends out from the lumen through the side of the side-entry connection jacket which attaches and positionally stabilizes the ductus side-entry connector without leakage while complying with the motility intrinsic in any tubular anatomical structure.

Accessed through a port implanted at the body surface, the connector serves as an entry point for connection of a drug delivery catheter, passageway for a diagnostic sensor (see, for example, Hu, W., Lu, Z., Liu, Y., Chen, T., Zhou, X., and Li, C. M. 2013. “A Portable Flow-through Fluorescent Immunoassay Lab-on-a-chip Device Using ZnO Nanorod-decorated Glass Capillaries,” Lab on a Chip 13(9):1797-1802; Patel, S., Park, H., Bonato, P., Chan, L., and Rodgers, M. 2012. “A Review of Wearable Sensors and Systems with Application in Rehabilitation,” Journal of Neuroengineering and Rehabilitation 9:21; Pantelopoulos, A. and Bourbakis, N. G. 2010. “A survey on Wearable Sensor-based Systems for Health Monitoring and Prognosis,” IEEE Transactions on Systems, Man, and Cybernetics, Part C: Applications and Reviews 40(1):1-12; Yilmaz, T., Foster, R., and Hao Y. 2010. “Detecting Vital Signs with Wearable Wireless Sensors,” Sensors (Basel) 10(12):10837-10862), or as a pathway for withdrawing diagnostic testing samples.

For ease of manipulation and because connection of the line will necessitating clearing neighboring tissue in any event, ductus side-entry connector is made as long but no longer than necessary and when the line is to be connected after the jacket has been placed, coated with contrast for quick location. To allow use of the side-entry connector as a manual circle-cutter to expedite plug removal or vasculotomy, side-entry connectors to or from relatively thick-walled conduits such as the gut are perpendicular (normal, at right angles) to the long axis of conduit and jacket. Right angular entry and exit side-connection are used when bloodstream confluence or splitting are uninvolved.

With blood vessels, the side-entry connector is attached to the jacket at an acute angle as most attenuates shear stress, so that when the delivery line is round in cross-section as affords optimal omnidirectional flexibility to expedite subcutaneous tunneling during placement, the sharp adluminal edge will be elliptical. In a vascular bypass or shunt application such as shown in FIGS. 21 and 22, the elliptical forward die-cutting edge of side-connector 6 prevents its rotation, and thus the creation of breaches at the apices about the cut that would allow exsanguination, or seepage, more problematic with anticoagulants.

However, the combination of the razor sharp front edge of the side-entry connector, application of suction, outward force exerted by the blood pressure, and that of the water ejected from the fluid-conducting or water-jacket will usually excise and extract the tissue plug without the need for the operator to loosen a rotatable side-connector. If the plug ‘hangs,’ then it is pulled away by a hook ended guidewire passed through a proximal clean-out or inline port such as that shown in FIG. 30. A proximal clean-out or inline port is usually one armed and unidirectional. Highly calcified or hydroxyapatite-capped lesions are often self contained, stable, not vulnerable or prone to rupture, and best left alone.

The roughened surfaces on the outside surface of side-entry connector 6 and inside surface of locking bushing or collar 5 are therefore arranged vertically to detent connector 6 at the cutting forward (adaxial) and installed positions. In a side-entry connected line where a future need to extract a valve-plug to be described, such as to pass through a narrow endoscope or laser, can be ruled out with confidence, the valve-plug can be of the nominally permanent type and the internal surface of the side-entry connector configured to retain the valve-plug in place indefinitely. Thus, when the valve-plug is to remain in place, the internal surface of the side-entry connector also has small prongs or dentations on its internal surface distal or adaxial to the native lumen-adaxial front cutting edge of the side-connector.

These dentations assist in retaining a valve-plug as described below in position when the junction must be sealed, the means for doing so addressed toward the end of this specification. When the valve-plug is to recoverable noninvasively through the application of water pressure, the portion of its peripheral surround of rubbery material at its forward (adductal, ductus-adaxial) end is snipped away so that it will not lodge thus between the free (forward, ductus adaxial) edge of the water-jacket and the prongs. By contrast, the round conformation of side-connector 6 in a jacket intended for use along a thick-walled conduit such as the gut allows rotation as a to assist in excising the enterostomy plug. A narrow connector for joining a catheter of fine caliber to a vessel to admit medication rather than to divert flow through a vessel can enter at right angles and be rotated as a trepan or circle-cutter so that the entry plug can be actively removed.

Side-entry connectors for use along the vascular tree are fixed in rotatory angular position by the elliptical shape at the adluminal end. The degrees and freedom of movement of the side-entry connector set by the locking collar or bushing at the base of the connector, reciprocating movability of the side-entry connector is limited to that little greater than the thickness of lumen wall essential for complete excision of the plug. When passage through the jacket is at an angle so that the cutting edge of the side-connector is elliptical and its use as a circle-cutter is denied, the greater thickness of the foam lining over that of the lumen wall allows the vacuum pressure to be increased effecting tissue plug excision. Turning now to FIG. 1 thru 7, 13 thru 15, 17 thru 19, 21 thru 23, 29, 31, and 32, viscoelastic polyurethane foam lining 3 is incorporated to protect small vessels and nerves that support the wall surrounding the ductus, is beneficial if not essential in almost every application.

To protect the small vessels and nerves, the foam envelops or wraps around these tiny structures investing them in a fairly static relationship relative to the compression and expansion in the jacket as a whole. The two sources of jacket compliance to expansion and contraction of the encircled conduit are spring-hinges 14 used to join the half-cylinders along meeting inner edges or joint 15 comprising the jacket and foam lining 3. In an artery, for example, a significant difference in excursion of the foam and the adluminal end of side-connector 6 as the pulse entered the retrograde or upstream end of the jacket and traveled to connector 6 and then moved out the antegrade end of the jacket would tend to wrench away and separate the adluminal end of side-connector 6 from the adventitia.

Except in a young patient treated for a chronic condition, the traveling swell of the pulse is probably never, and the bulge of peristalsis as seen in the esophagus, rarely, defined so sharply by an abrupt rise and fall as to make sudden jerking at the side-entry connector/foam interfaces of more than passing concern. The means described should not pose a problem of intimal hyperplasia that would lead to failure; however, where this contingency is a concern, a water-jacket inlet or service channel is available to deliver antihyperplastic drugs. Side-entry connection jackets used to deliver these drugs at surgical or conventionally sutured anastomoses use the side-entry connector for drug delivery. To allow for growth without the need for a second invasive procedure, a side-entry jacket or an impasse-jacket for use in a child is increased in the width of foam lining to the extent the anatomy allows.

Protrusion of the adluminal end of the side-entry connector 6 into the lumen would disrupt laminar flow and its separation would result in bleeding into the foam about the connector. Separation of side-entry connector 6 from the surrounding foam is prevented by bonding the outer surface of connector 6 to surrounding foam lining 3. To avoid this contingency, the jacket is limited in length so that differences in excursion along its length as the traveling wave passes through are minimal and lining 3, usually viscoelastic polyurethane foam, is selected in a density, usually higher, as responds to pulsatile or peristaltic compression with a delay or lag time equal or greater than the intervals of the passing expansions and contractions.

Also to achieve a compressibility that best resists forces that would separate the adluminal end of connector 6 and the adventitia, foams of different densities and chemistry can be used or combined as concentric layers. Locking collar or bushing 5, outer shell 4, foam lining 3, and lumen wall 2 are substantially locked together and move as a unit, riding up and down with the jacket as it expands and contracts with the encircled conduit. As shown in FIG. 3, to the extent that the end caps of shell 4 cannot encroach upon the adventitia, shell 4 is made to wrap around the sides of foam lining 3. Magnet 8 must be isolated thus, foam lining 3 made thicker as necessary.

Side-entry connector 6 is fixed to shell 4 by locking collar or bushing 5, so that when shell 4 rests against the adventitia or fibrosa, the adluminal edge of side-entry connector 6 expands or contracts with the margins of the jacket. The shell substantially inflexible, the shell then expands and contracts as a whole, so that connector 6 reciprocates together with the antegrade or upstream and retrograde or downstream ends of the jacket. The jacket ends are then made thicker and rounded as to be nonincisive. Extension lengthwise of the jacket to ensheath more than one passing pulsatile or muscular wave and variation in the foam density in coordination with this change in dimensions will also alter the forces seen by side-entry connector 6.

Side-entry connectors used to form bypass or shunt divergences and convergences where the angles for optimal flow characteristics through the junction are acute and which must function as plug circle-cutters or plug-cutters are provided with a locking collar or bushing having an internal bore complementary to the angle of the connector but no different in structure and function than the locking bushing of a side-connector at right angles. Thinner walls are drawn against and pass over the cutting edge, vacuum pressure, the blood pressure, and pressure washing through the water-jacket, removing the plug even should it briefly hang or adhere as a flap.

Vascular junctions are therefore angled to minimize shear stress and least interfere with streamline or laminar flow through the junction (see, for example, Loth, F., Fischer, P. F., and Bassiouny, H. S., 2008, “Blood Flow in End-to-Side Anastomosis,” Annual Review of Fluid Mechanics 40:367-393; Freshwater, U., Morsi, Y. S., and Lai, T. 2006. “The Effect of Angle on Wall Shear Stresses in a LIMA to LAD Anastomosis: Numerical Modelling of Pulsatile Flow,” Proceedings of the Institution of Mechanical Engineers Part H. Journal Engineering in Medicine 220(7):743-757; Leva, C. and Engström, K. G. 2003. “Flow Resistance over Technical Anastomoses in Relation to the Angle of Distal End-to-side Connections,” Scandinavian Cardiovascular Journal 37(3):165-171).

To avoid the superfluous aspiration of blood as well as to preclude trocar type gouging or incision into the lumen wall opposite the point of entry, vessels are entered using the least functional vacuum pressure. The instant the wall is breached, the vacuum pressure is transferred from the wall to the blood, which is also expelled due to the blood pressure, so that the vacuum is then eliminated as a cutting force. These factors and the use of contrast dye as necessary substantially eliminate the risk of gouging injury. A hanging flap due to a lesion of unanticipated physical properties in the lumen wall at that point is forced out by the blood pressure and pulled off by the pressurized irrigation of the water-jacket, which can be increased in pressure briefly for the purpose.

Like other lumina introduced, the water-jacket can be used to aspirate diagnostic samples. Side-entry connectors at junctions with vessels for connection to a port at the body surface to infuse drugs through a tiny ostium can join the vessel normal to or at right angles (perpendicularly). The fluid-conducting or water-jacket can be used to deliver or withdraw a liquid or gas. Since the fluid-conducting or water-jacket and the side-entry connector can be used as separate channels in either direction or to create a circuit, the water-jacket is not divided into independent channels. Use along the gut does not require a side-entry connector with a fluid-conducting or water-jacket at the time of placement.

However, the potential uses for the fluid-conducting or water-jacket for postoperative maintenance and to deal with sequelae makes including the fluid-conducting or water-jacket in every side-entry connection jacket prudent. Uniformity thus also serves to reduce the cost of production. The front edge of the ductus side-entry connector, then not needed as a manual circle or plug cutter or trephine (trepan) and punch, can be angled to avoid encroachment upon neighboring tissue, the opening or ostium into the lumen then likewise angled, hence, ellipsoidal with the edge honed in the long axial direction of the connector.

Ductus side-entry connection jackets may be magnetized and used in combination with impasse-jackets, which placed in encircling relation about a tubular anatomical structure, apply magnetic force to magnetically susceptible carrier particle-bound drugs in a passing ferrofluid (see, for example, Ruuge, E. K. and Rusetski, A. N. 1993. “Magnetic Fluids as Drug-carriers: Targeted Transport of Drugs by a Magnetic Field,” Journal of Magnetism and Magnetic Materials 122(1-3):335-339), for example. Drugs indissolubly bound to the carrier are drawn against or through the lumen wall, whereas those dissolubly bound are released to pass downstream. A side-entry jacket can define the starting, and if necessary, another side-entry jacket or an impasse-jacket, the ending level, along a tubular anatomical structure, thus describing the intervening segment for receiving medication or that level wherein drugs dissolubly bound to the carrier particles will be released to pass downstream.

Side-entry connection jackets can also be used to deliver, and impasse-jackets to set the point for releasing a reversal agent along the lumen. The distinction between side-entry and impasse-jackets actually represents the extremes along a spectrum wherein many combine the features of either type in pure form as both piped to an entry port implanted at the body surface and magnetized. Any side-entry or impasse-jacket that incorporates ferrous matter can be heated by placing the patient in a radiofrequency alternated magnetic field to affect the physiology of the ductus or the chemistry of the medication and/or other therapeutic substances employed. Side-entry connection can also be used to connect like or different type conduits internally without an entry port at the body surface.

For example, side-entry jackets allow off-pump, or beating-heart, coronary artery bypass using synthetic or tissue-engineered conduits, without the need to harvest and injure uninvolved tissue even when useable autologous conduit is available, as well as to reduce the durations of the overall procedure, anesthesia, and when employed, cardiopulmonary bypass, hypothermia, and cardioplegia, or circulatory arrest. Jacket placement can be on the ascending aorta with bypasses taken off from the same or different jackets, and an additional bypass taken. Once outside the polyether ether ketone or similar tough polymer jacket shell or casing, the angled connectors curve to avoid encroachment upon surrounding tissue. A common longitudinally narrow side-entry jacket with radially arranged side-entry connectors, or more than one jacket, can be placed about the ascending aorta, each synthetic coronary artery bypass catheter connected to side-entry jackets at both the origin and insertion, or destination.

Depending upon the anatomy, a second jacket is positioned on the aorta or the near brachiocephalic (innominate) trunk, for example. Optionally, to allow direct access to the side-entry connected junctions with drugs such as clopidogrel and/or aspirin in an artery or warfarin, heparin, or apixaban in a vein, a fluid-conduction or water-jacket inlet or a second side-entry connector to any such side-entry connection jacket can be led to a port implanted at the body surface. Such drugs not only suppress clotting but clogging due to ingrowth and hyperplasia (see, for example, Lin, P. H., Chen, C., Bush, R. L., Yao, Q., Lumsden, A. B., and Hanson, S. R. 2004. “Small-caliber Heparin-coated ePTFE Grafts Reduce Platelet Deposition and Neointimal Hyperplasia in a Baboon Model,” Journal of Vascular Surgery 39(6):1322-1328).

An anatomically or extra-anatomically positioned aortofemoral or ileofemoral bypass to relieve the ischemia of advanced (Type 4) atherosclerotic aortoiliac obstructive, or aortoiliofemoral occlusive disease (see, for example, Koksal, C., Kocamaz, O., Aksoy, E., Cakalagaoglu, C., Kara, I., Yanartas, M., and Ay, Y. 2012. “Thoracic Aortobifemoral Bypass in Treatment of Juxtarenal Leriche Syndrome (Midterm Results),” Annals of Vascular Surgery 26(8):1085-1092; Capoccia, L., Riambau, V., and da Rocha, M. 2010. “Is Femorofemoral Crossover Bypass an Option in Claudication?,” Annals of Vascular Surgery 24(6):828-832),” Annals of Vascular Surgery 26(8):1085-1092) in a patient without a suitable autologous graft is an example of an arterial application, while a crossover saphenous vein bypass graft (see, for example, Haas, G. E. 1989. “Saphenofemoral Vein Crossover Bypass Grafting in Iliofemoral Vein Obstruction,” Journal of the American Osteopathic Association 89(4):511-518; Jørgensen, P. E., Lundsgaard, C., Jelnes, R., and Frimodt-Møller, C. 1986. “Iliofemoral Bypass Surgery for Lower Limb Ischaemia. A Follow-up of 62 Patients,” Annales chirurgiae et gynaecologiae 75(3):155-159; Ehrenfeld, W. K., Levin, S. M., and Wylie, E. J. 1968. “Venous Crossover Bypass Grafts for Arterial Insufficiency,” Annals of Surgery 167(2):287-291) is an example of a venous application.

These patients are normally elderly with distributed atherosclerotic disease that discourages the additional surgery required to harvest autografts likely to prove poor prospects for continued patency in any event (see, for example, Davidović, L. B., Lotina, S. I., Kostić, D. M., Cinara, I. I, Cvetković, S. D., and 5 others 1997. “Factors Determining Late Patency of Aortobifemoral Bypass Graft.” [in Serbian; English abstract in Pubmed] Srpski Arhiv za Celokupno Lekarstvo 125(1-2):24-35; Davidović, L. B., Lotina, S. I., Kostić, D. M., Cinara, I. I, Cvetković, S. D., and 5 others 1997. “Dacron and Polytetrafluoroethylene Aorto-bifemoral Grafts,” [in Serbian; English abstract in Pubmed] Srpski Arhiv za Celokupno Lekarstvo 125(3-4):75-83). This combination of circumstances makes a viable alternative which allows the use of prosthetic tubing with means for averting occlusion appropriate.

Side-entry connection jackets can serve as a useful adjunct whether native grafts or synthetic prostheses are to be targeted with drugs. When a polymeric prosthetic is used as a bypass, targeting the bypass for an anticoagulant and/or other drugs using a bypass entry jacket to deliver the drug and if necessary, a bypass exit jacket to deliver a reversal agent or counteractant restrains a systemically small dose from circulating. Several fluid-conduction passages or water-jacket passages can be incorporated into a side-connector and used together to aspirate, or to deliver the same, or separately to deliver different drugs. The water-jacket in a side-entry jacket used to divert blood from a vessel is not made of metal as would promote the accumulation thrombus necessitating the use of an anticoagulant but rather polymeric.

Solid objects requiring a clear path such as diagnostic catheters or sensors are threaded or ‘snaked’ through the conduit to the junction through the side-connector from a port implanted at the body surface. This allows the use of a fiberoptic angioscope to examine the junction and view the delivery of the drug through the fluid-conduction or water-jacket, for example. Provided the angle of the side-entry connector to the jacket is acute or steep enough, the same path can be used to pass through a rotational thrombectomizer or a rotational or linear atherectomizer, for example, significantly expediting the possibilities for preventing, diagnosing, and treating any later acute event.

Using fluid-conduction or water-jackets, accessory fluid inlets and outlets can be led from a port at the body surface to the native-to-synthetic and synthetic-to-native junctions. The targeted administration of medication to each of several systemically unrelated side-entry junctions will usually be different as to drugs used and scheduling. Since the catheter used as a bypass or shunt is synthetic, delivery at the junction of origin will treat the junction itself and the blood flowing past the junction, but the lumen wall only once the destination junction is crossed. That anticlotting or antibiotic medication, for example, will be of benefit throughout the course to include that synthetic is clear.

However, because not only thrombus but atherosclerotic plaque tends to develop at sites of increased shear stress such as branches, the anti-inflammatory or pleiotropic effects of statins should be exploited by delivering the statin to the junction of origin, not that of insertion however distant, if not upstream, atherosclerosis systemic as to recommend a lesser background dose in the systemic circulation at all times. A downstream side-entry jacket or side-entry impasse-jacket having its fluid-conduction or water-jacket inlets connected to a port at the body surface, or if needed only briefly, an unpiped impasse-jacket, can be used to neutralize any residue of a drug and thus prevent it from further circulation.

This allows the use over a circumscribed segment of a statin at a dose that if circulated would induce myopathy. Incorporating one or more fluid-conduction or water-jacket inlets on each side-entry connector affords flexibility in allowing different joints and segments or stretches to be medicated differently as well as alike as the need arises. Bypass grafts using harvested vessels anastomosed with suture are preferable to the use of synthetic materials. Where such a graft would benefit from the direct delivery of medication, means set forth herein can prove of value in a subsidiary, supportive role. A side-entry connection jacket receiving a catheter from a port at the body surface placed to precede a bypass graft can deliver drugs for takeup within the bypass.

Any residue to be prevented from passing downstream can be neutralized by a reversal agent delivered through a downstream line or by release from an impasse-jacket just past the bypass outlet or insertion anastomosis. Targeted delivery of drugs in higher than circulated concentration is intended to suppress restenosis, shrinkage, sclerosis, and the formation of thrombus. Autologous vascular grafts used in the arterial tree are targeted primarily with a platelet blockade and a statin, whereas those used in the venous tree are primarily targeted with an anticoagulant such as warfarin, preference given to those for which an effective reversal agent is available. The ability to target anticoagulants is of value, because the use of these drugs must often precede the need for surgery, and the risk of problem bleeding is also alleviated should the patient suffer accidental trauma.

The ability to substantially restrict medication, such as immunosuppressants and antibiotics to an homologous graft considerably reduces the risk of systemic complications, to include adverse drug-drug interactions and side effects. When the drug acts upon contact, the side-entry connection jacket can be placed on the vascular and functional inlet stumps of transplant organs when harvested and before placement, or insertion of the graft; when the drug requires some lead before acting, the jacket is placed upstream in the recipient, delivery through the same jacket as that used to make the transplant junction then downstream thereof. In a parallel manner, an outlet jacket needed to delivery a reversal agent can enter the transplant exit jacket, or if lead time is required, downstream thereto.

Because the overall dose is small compared to dosing for systemic circulation, targeting substantially eliminates the advantage of warfarin over direct Factor Xa inhibitors such as apixaban (Eliquis®, Bristol-Myers Squibb/Pfizer), dabigatran etexilate (Pradaxa®, Boehringer Ingelheim), and rivaroxaban (Xarelto®, Bayer HealthCare AG) as reversible with vitamin K. Reversal agents for Factor Xa inhibitors such as PRT4445 (Portola Pharmaceuticals) (see, for example, Lu, G., DeGuzman, F. R., Hollenbach, S. J., Karbarz, M. J., Abe, K., and 7 others 2013. “A Specific Antidote for Reversal of Anticoagulation by Direct and Indirect Inhibitors of Coagulation Factor Xa,” Nature Medicine 19(4):446-451; Rupprecht, H. J. and Blank, R. 2010. “Clinical Pharmacology of Direct and Indirect Factor Xa Inhibitors,” Drugs 70(16):2153-70) for systemic dosing are currently undergoing trials.

In the unlikely circumstance that a drug would have to be limited to a defined segment of a native conduit whether intrinsic or used as a bypass or shunt, a second jacket at the cutoff level can be used to remove any residue from the circulation. Once a reversal agent is available, conversion from the oral form for the Factor Xa inhibitor and its reversal agent should pose little difficulty. Targeting in different situations can spare numerous complications such as muscle impairment, problem bleeding, and the risks associated with immunocompromising the patient as a whole. An unwanted residue for which a reversal agent is unavailable can be prevented from further passage by bonding it to magnetically susceptible micro or nanoparticles, if not directly, then to an affinitive substance that will seek it out.

Provided the drug works locally and need not be processed by the liver, a bypass graft to divert or shunt around a site of chronic venous insufficiency, such as a crossover saphenous vein bypass (see, for example, Greenfield, L. J. 1997. “Chronic Venous Insufficiency,” in Greenfield, L. J., Mulholland, M. W., Oldham, K. T., Zelenock, G. B., and Lillemoe, K. D. (eds.), Surgery: Scientific Principles and Practice, page 1965) to avert and compensate for affected side deep vein thrombosis, for example, may similarly be targeted for medication. A side-entry jacket upstream to, or one with two side-entry connectors at the inlet connection to a synthetic venous bypass can target an anticoagulant such as apixaban to the bypass, thus overcoming the thrombotic propensity of a catheteric (synthetic, prosthetic) venous bypass that is the primary deterrent to such application. The prevention of subarachnoid hemorrhage alone makes this approach advantageous.

### 2. SUMMARY OF THE INVENTION

A biocompatible, viscoelastic polyurethane foam-lined polymeric jacket or collar comprising complementary spring-hinged half cylinders and incorporating a radially extending ductus lumen side entry connector for attaching a catheter or hose is placed through a small endoscopic incision to surround a segment of a tubular anatomical structure or the territory (region, area) supplied or drained by a branch from or to the segment to be treated. The thickness of the ductus wall widely variable, the razor sharp forward or adductal die-cutting edge of the side-entry connector is generally one third the outer diameter of the ductus or the diameter of its lumen. With the jacket encircling the ductus and cutting off any path of leakage or exsanguinations into the surrounding cavity or tissue, a vacuum is used to draw a plug of tissue from the side of the ductus wall.

When the wall is too thick for suction alone to excise the plug, the jacket side-connector can be used as a trepan or circle-cutter and locked in position. When the disease warrants, the pump or pumps connected to the ductus side-entry jacket or switchable among, multiple jackets is tied in a closed loop control system. Comorbidities are treated concurrently with different types and sizes of jackets according to the sizes of the different system ductus to be jacketed, the drugs used for each targeted and isolated from the others and other tissue as the situation requires. When necessary, the control system is programmed to simulate ‘learning’ ability, delivering drugs in response to sensor-detected symptoms in anticipation or predictively with the objective of suppressing critical events before these appear as pain or discomfort.

### 3. OBJECTS OF THE INVENTION

A primary object of the invention is to provide a means for joining synthetic or tissue-engineered conduits to native ductus which leaves the lumen free and clear without medically significant leakage or reverse flow, direct and continuous connection of a catheter entered through a port implanted at the body surface to the lumen of a tubular gastrointestinal, circulatory, respiratory, or genitourinary conduit allowing the delivery of drugs, radionuclides, or other therapeutic substances at that level while avoiding the upstream lumen and tissue supplied by branches thereof.

Another primary object of the invention is to provide the patient with a means of ambulatory therapy that functions automatically, least interferes with freedom of movement, and in particular, spares the patient from being tethered to a therapeutic apparatus, bedridden, or an in-patient.

Yet another object of the invention is to allow the direct and immediate translation of chemical, electrical, and immunoassay feedback diagnostics into automatic drug delivery around the clock, constituting no more than a slight impediment to free movement, whether to the locus of detection, the site of the symptom, and/or the etiological origin, under the control of a hierarchical or complex control system capable of predictive or anticipatory control and further adaptable through ‘learning’ ability, and in so doing, apply such control to the practice of internal medicine.

Another object of the invention is to make possible the coordination, and usually the collocation, of drug need detection and delivery means so that drugs can be targeted directly to the anatomical point of detection or a point functionally related thereto, thereby enabling the implementation of prosthetic disorder response systems, to include those employing hierarchical control.

Yet another object is to provide a kind of joint or junction that also allows forming synthetic or tissue-engineered bypasses and shunts between native conduits which can, but need not, communicate with a port implanted at the body surface, averting the need for, or the need to harvest, a suitable autologous graft.

Another object is to allow a safe and secure connection of a synthetic to a native conduit that unlike an indwelling catheter, provides a ready-made direct path into the native conduit for treatment and testing to allow the patient complete freedom of movement while connected to an automatic portable pump or diagnostic testing instrument.

Another object is to provide a connection to an anatomical ductus that fully compliant with the intrinsic motility of the ductus, secure, and less prone to infection, affords better opportunity for healing without complications.

Whether placed as a precaution to deliver drugs in the event of a medical emergency or to allow the use of testing equipment, the connection is usually meant to be permanent. For this reason, periodic diagnostic testing or treatment does not require the reintroduction of an indwelling needled catheter for each treatment or test, the risk associated with placing such a catheter and the need to immobilize the patient therefore averted.

A related object is to allow the use of a magnetized side-entry jacket to draw a magnetically susceptible carrier bound drug into the lumen wall at the side-entry junction, jacket magnetization is generally circumferential with the strength increased in the downstream or antegrade direction for more uniform takeup along the entire length.

Another related object is to provide a junction that will serve as the level at which the lumen is entered in relation to a magnetized jacket encircling the structure downstream used to truncate further transport of magnetically susceptible carrier particles. In this way, the segment for exposure to medication introduced in the form of a ferrofluid can be limited.

A related object is to allow the use of the downstream jacket to suspend a reversal agent in the lumen, so that depending upon whether the drug-carrier bond is soluble, the drug can be restricted to the segment designated and the territory supplied by any side branches to or from the segment. Jackets that interfere with flexion if too long are divided, that downstream continuing in strength of magnetization where that upstream had left off. For treating eccentric lesions, magnetization is limited to the arc requiring treatment. Such a lesion might be, for example, a benign tumor, or if malignant, then one itself palliated or the site of its resection medicated.

Other objects are to accomplish connection to the structure through a junction that places nothing, such as an indwelling catheter, in the lumen, so that—consistent with stent-jacket and impasse-jackets of like object disclosed in copending application Ser. No. 13/694,835—the lumen is completely unobstructed, little if at all affected in normal flow past the junction while not in use, and any transluminal interventional procedure that may be needed, especially one exigent, will have a clear pathway.

Another object is to situate all parts of the jacket and its supply pipeline extraluminally so that parts of the jacket no longer needed to prevent leakage through the ostium created can be made disintegrable or absorbable.

Yet another object is to provide a junction that allows equality of luminal diameter between the synthetic or tissue-engineered and natural lines, allowing bidirectional application with consist volumetric flow rate between synthetic and native conduits or the reverse, and, if necessary, the entire flow to be cross-clamped and shunted. Yet another object is to provide a direct channel for withdrawing diagnostic testing samples or for passing an analytical sensor from outside the body, such as a port implanted at the body surface.

Yet other objects are to accomplish the foregoing by means and methods that allow endoscopic application with minimal invasiveness under local anesthesia with anticlotting medication substantially limited to the entry site, and little if any in the systemic circulation.

### 4. DESCRIPTION OF THE DRAWINGS

FIG. 1 shows a longitudinal section through a segment along a tubular anatomical structure with an magnetized and unshielded, or simple junction type, side-entry connection jacket having an internal fluid-conducting or water-jacket, shown placed about a ductus before the side of the lumen wall is drawn under vacuum pressure past the sharp leading edge of the side-entry connector to cut and extract therefrom a plug of tissue.

FIG. 2 shows the side-entry connection jacket of FIG. 1 after the sharp leading edge of the side-entry connector has been used to cut a plug from the side of the lumen wall and the plug extracted so that the leading edge has been advanced to be planar with the internal surface of the lumen.

FIG. 3 shows a longitudinal sectional view of a segment along a tubular anatomical structure with a side-entry connection jacket as that shown in FIG. 1 but with the addition of a magnet layer outside of and concentric to the long axis of the jacket.

FIG. 4 shows a longitudinal sectional view of a segment along a tubular anatomical structure encircled by a longitudinally extended, field strength-graduated, or magnetic gradient-type side-entry connection jacket, or piped impasse-jacket.

FIG. 5 shows a side-entry connection jacket of the same kind as that shown in FIG. 4 with the addition of a wraparound tungsten ‘heavy’ alloy radiation shield for use with a relatively low dose-rate short half-life radionuclide along a nonvascular ductus.

FIG. 6 shows a disintegrating radiation shield as the outermost layer of the jacket, applied outside the perforated shell surrounding the magnet layer seen in FIG. 4.

FIG. 7 shows a ductus side-entry jacket with a double arm or branched side-entry connector before the sharp leading edge of the side-entry connector has been used to cut a plug from the side of the lumen wall and the plug extracted, which configuration expedites the steering of a cabled device into the native lumen in either direction and allows the infusion of drugs or a ferrofluid or transfusion at a higher flow rate.

FIG. 8 shows a side view of a clasp-electromagnet with its pole directed away from, rather than facing, the tissue attaching base as would apply to a clasp impasse electromagnet or a clasp extraction electromagnet.

FIG. 9 shows an overhead view of the clasp-electromagnet shown in FIG. 8.

FIG. 10 is an outer perspectival view of a tissue-engineered ductus with magnetically susceptible plates mounted on a thick spandex or similar stretchable rubber backing 82 lined with viscoelastic polyurethane foam 3, the opposing magnet poles directed at the center of each draw-plate seen in the cross-section shown in FIG. 11, such a formation used to compress successive segments of the ductus interposed between the magnets and plates in a coordinated sequence to simulate peristalsis.

FIG. 11 is a cross-sectional view through an electromagnetic impasse-jacket that by adding a magnet draw-plate on the opposite outer surface of the ductus, functions as a contraction or peristalsis jackets in the compound jacket shown in FIG. 10, which can compress the ductus interposed between the electromagnet-plate pairs in an advancing sequence.

FIG. 12 shows a longitudinal section through an electromagnetic impasse-jacket to which a draw-plate has been added on the opposite side of the ductus to function as one of the electromagnet-plate pairs along a tissue-engineered ductus as shown in FIGS. 10 and 11.

FIG. 13 is a longitudinal section through an extraction double arm electromagnetic ductus side-entry jacket for analyte exchange with the lumen rather than the wall surrounding the ductus, with integral trap or collection chamber and flush-through or purge line, the magnet with bent around core and coil beneath the plane of the drawing.

FIG. 14 shows a series of extraction electromagnet-jackets spaced along a ductus with common flush-through line connected in series.

FIG. 15 is a longitudinal section through a ductus with an extraction jacket having two or four extraction electromagnets positioned circumferentially about the jacket, the bent around cores and coils unseen outside the plane of the drawing but configured as that of the individual electromagnet-plate pairs of the multi-paired jacket shown in FIGS. 10 and 11.

FIG. 16 shows a catheteric or tissue-engineered line from a port such as shown in FIGS. 27 and 28 at the body surface and schematically shown in FIGS. 21, 22, 29, 31, 32, and 38 to a native conduit, such as a vasospasm-susceptible (angiohypertonic, angiospasmic) artery, depicted here as the left anterior descending epicardial coronary.

FIG. 17 is a perspectival cross-section through a simple junction, or unmagnetized and nonelongated, thin-walled, side-entry connection jacket such as shown in FIGS. 1 and 2, with two side-connectors everted and inclined caudally in relation to the transverse plane of the jacket, for placement about a native conduit to accept synthetic catheters or artificial arteries as shown in FIGS. 21 and 22.

FIG. 18 is a perspectival cross-section through a jacket of the kind shown in FIG. 3 configured as the jacket without magnet layer shown in FIG. 17 with two arms and the addition of a concentric magnet layer.

FIG. 19 shows the side-entry connection jacket of FIG. 17 placed about the ascending aorta diagrammatically, FIG. 21 providing a more anatomical view.

FIG. 20 is a left side view of the side-entry connection jacket shown in FIGS. 17 and 19 represented as level to its horizontal or transverse plane.

FIG. 21 is an anterior or facing view of the heart with the jacket shown in FIGS. 17, 19, 20, and 22 placed proximal to the root of the ascending aorta to join synthetic or tissue-engineered epicardial coronary artery bypass conduits for insertion distal to the blockages in the left anterior descending or anterior interventricular branch artery to the right and the right coronary artery to the left, through smaller single arm ductus side-entry connection jackets shown without accessory or side lines.

FIG. 22 is a pictorial schematic or circuit diagram of the side-entry connection jacket shown in FIG. 21 in greater detail, showing the connection of each side-connector to a synthetic or tissue-engineered native artery bypass line from the ascending aorta to smaller single arm ductus side-entry connection jackets shown without accessory or side lines but with each bypass connected by accessory or water-jacket inlets to a port at the body surface, thereby to deliver an anticoagulant, essential to prevent current state of the art catheters and synthetic blood vessels from thrombosing.

FIG. 23 is a longitudinal sectional view through an adjustable obturator type or stopper shutoff and throttle valve-plug in use to close off or to adjust the volumetric flow rate through the opening or ostium in the side of a native ductus created when placing a side-entry connection jacket, shown in place within the side-entry connection jacket.

FIG. 24 is a longitudinal sectional view through an adjustable obturator or stopper shutoff and throttle valve-plug in use to close off or to adjust the volumetric flow rate through the opening or stoma in the side of a native ductus created when placing a side-entry connection jacket, with the distal segment of the guidewire shown in FIG. 26 used to place, advance, remove, and in this mechanical embodiment, adjust the valve-plug.

FIG. 25 is a full face cross-sectional partially ghost view of the rear, that is, the underside or proximal end, of the shutoff obturator or stopper and throttle valve-plug shown in FIG. 13.

FIG. 26 is a detailed view of the distal end of the guidewire shown in FIG. 24 for advancing, retracting, and adjusting the cross-sectional area for flow-through of a shutoff obturator or stopper and throttle valve-plug.

FIG. 27 is a full face view of the upper surface of the base plate of the port shown in FIG. 28 for positioning at the body surface for connection of electrical, fluid mainlines, and side-entry connection mainlines and water-jacket sidelines also used as accessory or service channel lines.

FIG. 28 is a side view partially in section of the port with base plate fastened to the body surface for connection of one or more side-entry connection mainlines and water-jacket or sidelines, shown to a side of a plane passing through the suture holes, with the port cap screwed on.

FIG. 29 is a diagrammatic representation of a pump-pair plug-in module without inlet or outlet line switching turrets inserted into a single pump-pair power and control module, wherein one pump is connected to the side-connector and the other pump to the accessory inlet or water-jacket of the same single side-entry connection jacket.

FIG. 30 is a detailed view of a double arm, branching or forked type inline port or clean-out used to facilitate the bidirectional insertion of a cabled device such as a fiber endoscope or laser, or a debris extraction aspiration catheter or hook-tipped guidewire into the lumen of a catheteric fluid line, as shown in FIG. 31.

FIG. 31 shows a double arm or forked type inline port connector or clean-out as shown in FIG. 30 placed along a pump-line inside the pump-pack for extracorporeal access that allows the insertion into the pump line of a cabled or catheteric device such as an aspiration catheter, guidewire, laser, intravascular ultrasound or ablation probe, or a fiber endoscope, for example, through the pump line in either direction, with a double arm side-connector as shown in FIG. 7 at the jacket to allow insertion and passage through the lumen of the cabled device into the ductus in either direction, as well as for drug delivery purposes.

FIG. 32 shows right-hand pumps in a standardized pump-pair wherein line switching using turrets allows any drug or line rotated into alignment with the pump intake by the pump intake line switching means shown as a turret to be delivered through any one line rotated into alignment with the pump outlet by the pump outlet switching means also shown as a turret but without drug vials for simplicity.

FIG. 33 shows the elastic slit membrane at the junction of a therapeutic substance supply reservoir hose where the hose connects by means of a lip undercutting or click-on collar to the top of the system standardized vial used to insert the hose into one of the vial receiving receptacles in the pump intake turret.

FIG. 34 is a side view of a system standardized therapeutic substance turret vial, here used as a reservoir hose connector, for insertion into one of the vial receiving receptacles in the pump intake turret.

FIG. 35 shows a longitudinal sectional view of a system standardized therapeutic substance vial connected to the end of a drug reservoir supply hose for engaging the hose in one of the vial receiving receptacles in the pump intake turret as both the connector and starting dose.

FIG. 36 is a diagrammatic representation of a pump with pump intake drug turret.

FIG. 37 is a diagrammatic schematic or circuit diagram of the control train when a single pump-pair and jacket set is inserted in the pump-pack, shown in the abstract as to the positioning of the parts as inside or outside the body, the train constituting a hierarchical control system.

FIG. 38 is a simplified diagrammatic schematic or circuit diagram of the interconnections within a hierarchical control system and positioning as inside or outside the body when a second pump-pair and jacket set is added to the first in the pump-pack.

### 5. DESCRIPTION OF THE PREFERRED EMBODIMENTS OF THE INVENTION

### Simple Junction Side-Entry Jackets

Simple junction side-entry jackets are intended to replace indwelling catheters for long term use. Simple junction jackets omit magnetization and except when used to convey a radioactive substance, radiation shielding. FIGS. 1 and 2 depict ductus side-entry jackets, with side-connector 6 and accessory or water-jacket inlet 10, which, as shown in FIGS. 16, 21, 29, 31, and 32, respectively serve to connect mainline 13 and side-connector sideline or accessory 11. Whereas—as shown in the application depicted in FIG. 16—sideline 11 goes to a port 16 at the body surface 18, mainline 13 may go to the port, or as shown in the application depicted in FIG. 21, it may be used instead to connect a prosthetic line used to replace a bodily conduit. Connection of mainline 13 to port 16 denotes its use to transmit medication to or withdraw diagnostic test samples from the native lumen through the side-entry jacket to which line 13 is connected.

By contrast, connection of mainline 13 other than to port 16 denotes its use as a prosthetic conduit to convey the materials of the ductus it replaces. In the simple junction jacket shown in FIGS. 1 and 2, part number 1 is a native lumen, 2 the wall surrounding the lumen, 3 a viscoelastic polyurethane foam lining, and 4 a strong outer shell or casing 4 made of polyether ether ketone (PEEK) or another biocompatible nonallergenic material. All types of jackets—with or without side-connector, permanent magnet or electromagnet based, contraction, extraction, and the variants of these—have a viscoelastic polyurethane foam lining, and unless radiation shielding prohibits it, the jacket is fenestrated throughout its thickness.

This factor promotes the disintegrable shielding shown in FIG. 6 which once dispersed, exposes perforations 19 previously encircled over that nondisintegrable shown in FIG. 5 when possible. In FIG. 1, the ductus side-entry jacket has been placed to encircle the structure before the razor sharp trepan or die-cutting leading edge of the side-entry connector 6 has cut through the wall so that it is flush planar with the internal surface of the lumen with the plug having been extracted from the side of the ductus. Viscoelastic foam lining 3 serves to protect fine vessels and nerves at the interface with the substrate conduit, provide motional compliance or depth of excursion according to the intrinsic motility of the conduit encircled, and accommodates an irregularity in the diameter of the ductus such as caused by the lesion treated.

FIG. 16 shows the application of a side-entry jacket for gaining access to a native lumen, where both lines connect to the native lumen, here to allow one-way epicardial coronary infusion through mainline 13 with collateral or intermittent adjuvant medication delivered through accessory or sideline 11, the administration of drugs contemplated as proceeding automatically under programmed sensor input-prompted control. To prevent a passive slit-membrane or flap-valve or a mechanical valve-plug described below from protruding into the native lumen despite the intrinsic pulsation or peristalsis, side-entry connector 6 is provided on its internal surface in front of or ductus-adaxial to the forward edge of water-jacket 7 with small recurved or backward directed or ductus-abaxially bent prongs 20.

Thus, accessory or sideline 11 almost always connects a native ductus through port 16 at the body surface 18, to an extracorporeal pump contained in a wearable pump-pack. Side entry connector 6 slidably and rotationally friction fits through the journal formed by outer shell of casing 4 and the raised interdigitating landings between it and locking collar or bushing 5 and a round hole through foam ling 3 in the side of and entirely through the jacket. When jacket shell 4 is extruded, collar 5 or bushing 5 is fused with or bonded to it as to allow a right angular side-entry connector 5 to be rotated for use as a trepan. Locking collar or bushing 5 has circumferentially spaced apart, raised, and roughened areas oriented in the long axis of connector 6 on its inner surface as the complement to corresponding or mating areas on the outer surface of side-entry connector 6.

These areas are positioned to mesh and lock the connector in the radial position required when front circle-cutting edge 9 of side-entry connector 6 is planar (flush, even, level) with the internal surface of the lumen so that these areas overlap. To radially redirect inlet 10, side-entry connector 6 is pulled out of the jacket and reinserted at the radial angle needed to allow as direct as possible a line 11 to the port 16 at the surface 18. Any additional inlets 10 will then be rotated likewise, different orientation among lines 11 only beneficial when each must be connected to a surface port at a different location, which improbable, can be accommodated by producing side-connector 6 with inlets 10 positioned thus. Because a later need for direct access to the jacket in order to provide medication, for example, is not predictable, it is preferable to install the jacket with lines led to the surface.

The line connecting a given side-entry jacket to a port at the body surface can be a service channel as 11 or a synthetic conduit 13 connected to a second side-entry connector 6 of the same side-entry connection jacket. The incorporation of fluid conduction or water-jacket 7 with inlet 10 for connection of irrigation line 11 during placement and as service channel thereafter as standard means that in use to connect one native lumen or segment thereof to another via a catheter, both of the side-entry jacket connectors 6 will incorporate means for connection of a service channel or bypass junction supply catheter leading to the port 16 implanted at the body surface 18.

Internal fluid-conducting or water-jacket 7 may be visualized as comprised of a top-hat configured insert within side-entry connector 6, with brim ductus abluminally disposed to create a closed off fluid-tight cylindrical collar shaped space within and in concentric relation to the adluminal or circle-cutter ended segment of side-entry connector 6. The internal diameter of the passageway or channel through side-entry connector 6 is thus reduced to the internal diameter of water-jacket 7 over the ductus adluminal segment occupied by fluid-conducting or water-jacket 7, so that a catheter passed through side-entry connector 6 to position its distal tip in the lumen must be narrower than a hose fit over side-entry connector 6 by the difference in the two internal diameters.

Fluid-conducting or water-jacket 7 can be used to pass any fluid in either direction and is ordinarily connected to a pump capable of propelling a hydrogel, air, or water through it in either direction. Any fluid introduced through fluid-conducting or water-jacket 7 hose or catheter 11 attached to fluid-conducting or water-jacket 7 connector or inlet 10 flows around and then up through the concentric cylindrical space of fluid-conducting or water-jacket 7 to discharge through the circular gap formed by the outer surface of ductus side-entry connector 6 and fluid-conducting or water-jacket 7. The pressure and temperature of the fluid, usually water, is set externally at the pump. The parts of the side-entry connection jacket are generally molded of polyether ether ketone (PEEK), graphene, or if fabricated from nonmagnetic stainless steel tubing, then bonded together by continuous-bead, or non-spot, resistance welding.

Usually used in a subsidiary role to minimize lumen spillage when the conduit wall plug is cut during placement and to deliver adjuvant medication whether under automatic control thereafter, water-jacket and service channel 7 with inlet 10 and line 11 serve in a subsidiary or secondary support role with a flow volume smaller than that of the primary channel flowing through side-entry connector 6. However, depending upon the application, the service channel line comprised of water-jacket proper 7, inlet thereto 10, and line 11 might be equal if not larger in diameter than the primary flow though side-entry connector 6. This is also the case with the alternative double arm side-connector jacket shown in FIG. 7, configured to allow expeditious passage of a guidewire or cabled device into the native lumen in either direction.

When the side-entry connection jacket is to be placed along the gut of a larger vertebrate, such as a human adult, a vacuum pump hose attached to the proximal or free end of the side-entry connector is used to draw the outer surface of the lumen wall against the razor sharp front or adluminal edge of the connector, thereby maintaining the tissue in contact with the cutting edge to assist the operator in using the side-entry connector as a circle-cutter to cut a plug through the side of the ductus wall. Since unlike the situation with tubular anatomical structures other than the gut, the plug can be disposed of by pushing it into the lumen, the pump is reversed to blow or forcibly wash the plug into the lumen under air or water pressure.

In FIGS. 16, 21, 22, 29, 31, 32, and 38, accessory or sideline 11 is connected to a native ductus, which occupies the jacket lumen. In FIGS. 21 and 22; however, side-connector 6, rather than used to hold mainline 13 for connection to port 16 at the body surface 18, is used instead to connect a prosthetic shunt or bypass for connection in turn to a native ductus, whether by anastomosis or as shown here, by distal side-entry jackets. FIGS. 21 and 22 show such an application, with side-connector 6 used to secure both the left and the right anterior descending prosthetic coronary artery tissue-engineered or synthetic bypasses.

Unlike the applications depicted in FIGS. 17 thru 22, in this application, so long as any adjuvant medication is not to be delivered separately, the water-jacket and its supply line (sideline, accessory line) 11 will be needed only during placement. Since accessory lines 11 are provided to the aortic jacket, drugs can be delivered through the replacement arteries at a distance close enough that the distal or end-arterial jackets need not retain lines 11 after these have been used to place the arterial jackets. While not all applications require an internal fluid-conducting or water-jacket, the significant post-implantation expansion in versatility of emergency responses it affords and the offsetting economy of avoiding different embodiments is considered to outweigh the negligible reduction in cost of omitting it.

For this reason, the port implanted at the body surface is routinely provided with two openings, one communicating with the side-entry connector, the other with the fluid-conducting or water-jacket. In internal ductus-to-ductus, or native lumen to native lumen use as a bypass or shunt—depicted in FIG. 16 as used to bypass the stenosed proximal segments of the left anterior descending, or anterior interventricular descending branch of the left, and the right coronary arteries—by connection of the ascending aorta to each artery distal to the obstruction, side-entry connectors 6 are attached to tissue-engineered prosthetic arteries or catheters leading to the side-entry connectors 6 at the arteries. Interluminal use is not limited to interconnection between ductus of like type such as arteries or veins but can include arteriovenous connection, for example.

Even with the expectation of uncomplicated healing in the placement of tissue-engineered graft arteries using suture, accessory lines 11 to automatically detect the need for and target medication directly to the grafts under closed loop adaptive control with the patient ambulatory will continue to increase the odds for successful healing with less physical discomfort and distress. As shown in FIGS. 1 thru 6, side-connector 6 incorporates a hydrogel, fluid conduction or water-jacket 7 entered through line 11 and inlet 10 to assist in its placement. Thereafter it remains available to serve as an accessory or service channel for aspiration or the delivery of medication directly to the jacket from the pump through port 16 at the body surface 18.

When the side-entry jacket shown in FIG. 2 is used before (upsteam, or proximal to) a cross-clamp to initiate, or after (downstream or distal to), a cross clamp to terminate, a bypass to the same or a shunt to another ductus, connector 6 will have been used to attach the catheteric line to serve as the bypass or shunt and will therefore no longer be available for surface-to-ductus connection for the purpose of piping medicatisim or communicating directly with lumen 1. In such use, water-jacket side branch 10 is used as a secondary or accessory side-entry connector or connector 6 is provided with additional secondary or accessory side-entry connectors similar to water-jacket side branch 10. These secondary side-connectors can pass through water-jacket 7 and directly into the lumen of primary connector 6. Delivery to this jacket, which lacks a surrounding magnet as would make it an impasse-jacket rather than a simple junction jacket, excludes drug-carrier particle bound drugs.

Side-entry jackets not made for magnetic use can incorporate ferrous materials. Catheters or artificial vessels used to shunt the flow of blood such as shown in FIGS. 21 and 22 should be the same in caliber as the native vessel, fed heparin and/or another anticoagulant or anticoagulants through a service channel shown as part number 11, any additional medication through the same or another service channel, and angled for streamline or laminar flow with minimal thrombogenic turbulence or churning of the blood. Lines not used to conduct native luminal contents are used to deliver therapeutic substances. FIG. 16 shows catheteric line 13 from port or port 16 at the body surface 18 to a native ductus, here a vasospasm-susceptible left anterior descending coronary artery. Such a line can be used to immediately target the artery with a vasodilator, such as nitroglycerin, when, as is usual, the spasm is of atherosclerotic inducement, a statin, as well as preventive medication such as a calcium channel blocker.

Where such a line had previously been placed, the central infusion of saline ice or Ringer's lactate slurry, for example, at a higher rate of delivery can be promptly initiated should the need arise (see, for example, Arrich, J., Holzer, M., Havel, C., Müllner, M., and Herkner, H. 2012. “Hypothermia for Neuroprotection in Adults after Cardiopulmonary Resuscitation,” Cochrane Database of Systematic Reviews 9:CD004128; Knapik, P., Rychlik, W., Siedy, J., Nadziakiewicz, P., and Cieśla, D. 2011. “Comparison of Intravascular and Conventional Hypothermia after Cardiac Arrest,” Kardiologia Polska 69(11):1157-1163; Taccone, F. S., Donadello, K., Beumier, M., and Scolletta, S. 2011. “When, Where and How to Initiate Hypothermia after Adult Cardiac Arrest,” Minerva Anestesiologica 77(9):927-933; Polderman, K. H. and Herold, I. 2009. “Therapeutic Hypothermia and Controlled Normothermia in the Intensive Care Unit: Practical Considerations, Side Effects, and Cooling Methods,” Critical Care Medicine 37(3):1101-1120; Polderman, K. H., Rijnsburger, E. R., Peerdeman, S. M., and Girbes, A. R. 2005. “Induction of Hypothermia in Patients with Various Types of Neurologic Injury with Use of Large Volumes of Ice-Cold Intravenous Fluid,” Critical Care Medicine 33(12):2744-2751; Vanden Hoek, T. L., Kasza, K. E., Beiser, D. G., Abella, B. S., Franklin, J. E., Oras, J. J., and 7 others 2004. “Induced Hypothermia by Central Venous Infusion: Saline Ice Slurry Versus Chilled Saline,” Critical Care Medicine 32(9 Supplement):S425-S431; Kasza, K., Fisher, B., Shareef, F., Oras, J., Chang, J., Tentner, A., Fischer, P., and 7 others 2008. “Medical Ice Slurry Coolants for Inducing Targeted-Organ/Tissue Protective Cooling,” at http://www.ne.anl.gov/capabilities/sinde/biomed/IceSlurry Cooling.pdf; Shikanov, S., Wille, M., Large, M., Razmaria, A., Lifshitz, D. A., Chang, A., Wu, Y., Kasza, K., and Shalhav, A. L. 2010. “Microparticulate Ice Slurry for Renal Hypothermia: Laparoscopic Partial Nephrectomy in a Porcine Model,” Urology 76(4):1012-1016, at http://www.ne.anl.gov/capabilities/sinde/biomed/LaparKidneySurgerySlurryCoolingAUA.pdf.

The medication can be administered manually from a syringe when the patient feels pain or automatically from the portable (ambulatory, wearable) pump-pack when a chemical or mechanical sensor associated with the jacket signals the pump through conductors passed through the same catheteric line 13 to the pump at port 16 before the threshold of pain sensation is reached. The coronary artery end-arterial, the targeting is complete, thereby minimizing adverse side effects and drug-drug interactions associated with drug delivery through the systemic circulation. With the scheme shown in FIG. 16, drug delivery can be initiated manually by the patient at the onset of pain or automatically by a sensor connected by a wire passed through delivery line 13, for example, to a miniature portable (ambulatory, wearable) pump.

Preferably, however, a blood gas or mechanical sensor in the jacket signals the pump preemptively or prodromally, that is, before the patient senses pain. Targeting minimizes adverse side effects, addressed above under Background and drug-drug interactions associated with drug delivery through the systemic circulation. Here, avoiding the liver minimizes if not eliminates interactions of calcium channel blockers such as diltiazem and verapamil, with other drugs meant to treat a comorbid condition elsewhere in the body. Any vessel or other bodily conduit of adequate caliber to allow application of a side-entry connection jacket can be made to deliver any fluid medicinal substance. A larger jacket placed about the pulmonary artery can be used to deliver decongesting drugs to the pulmonary capillaries.

Smaller jackets placed about the internal carotid arteries of a patient showing signs of vascular dementia can be used to deliver platelet blockers, antiatherosclerotic medication, other cholesterol reducing drugs, and antihypertensives at a concentration higher than would be allowed to circulate to treat systemic atherosclerosis. Where the disease is systemic, a background dose is circulated as well. Delivery directly to the brain of an antihypertensive might aid in suppressing an advancing subcortical hypertensive leukoencephalopathy or Binswanger disease. Disorders involving the carotid and coronary arteries are cited as exemplary; by this means, the vascular and/or luminal inlets and if necessary outlets of any discrete organ can be jacketed for treatment to the substantial exclusion of the rest of the body. If necessary, a downstream jacket is used to deliver a reversal or neutralizing agent if available to eliminate any residuum from further circulation.

FIG. 21 is an anterior view of the heart with the double side-connector with water-jacket inlets shown in FIGS. 17, 19, 20, and 22 having been placed about the ascending aorta to join synthetic coronary artery bypass conduits for distal insertion at the respective coronary arteries. The distal connection is made with smaller side-entry connection jackets distal to the occluded segments represented in FIG. 22 as 17. Connection to a port 16 at the body surface 18 through lines 11 connected to the side-entry connector 6 accessory or water-jacket inlets 10 allows delivery of an anticoagulant essential to prevent state of the art synthetic blood vessels from clogging with thrombus. Whereas in FIGS. 21 and 22 the proximal jacket secures synthetic catheters 13 used as coronary artery bypasses, in the application depicted in FIG. 16, the jacket encircles the coronary artery, and the vasodilator is fed through side-entry connector 6 line 13.

More specifically, in FIG. 16, the flow is through a catheter or synthetic line and the junction created with the side-entry connection jacket to a coronary artery, where the angle and caliber of the lumen expedite laminar flow of the blood stream. In FIG. 16, line 11 is essential to minimize if not prevent exsanguination during placement of the jacket, but the native artery is not so prone to become obstructed by clot as is the synthetic artery depicted in FIG. 21, where an anticoagulant and possibly antihyperplastic drugs must continue to be fed to the synthetic bypasses by lines 11 from the surface through the water-jacket inlets 10 of each side-entry connector 6 continued in use as a post implantation service channel. Nevertheless, because a need for drug delivery separately from that delivered through mainline 13 might always arise, line 11 is never made absorbable.

In FIG. 21, flow is through a tissue-engineered artery or catheter, and the junction made by the side-entry connection jacket into a coronary artery, where the angle of entry for the small volume delivered allows perpendicular junction without the need for approach through a side-connector of the proper angle or a caliber equal to that of the artery. Essentially then, the configuration of FIG. 21 replaces the aorta for the surface port implanted at the body surface as the source of flow. The coronaries end-arterial, the segment targeted starts at the jacket, here of the kind without magnet shown in FIGS. 1 and 2, and ends within the myocardial supply area or territory of the artery.

Where bidirectional flow is contemplated, a side-entry connector 6 line 13 is generally preferred to a water-jacket inlet line 11 for the larger caliber and greater flow rate. When an eventual need for additional side-entry connectors 6, side-entry connector lines 13, water-jackets 7, water-jackets inlets 10, and water-jacket inlets lines 11 cannot be predicted, the number potentially required are prepositioned in one procedure. Primary lines such as those shown in FIGS. 16 and 21 are often dedicated and committed to a specific constant use. By contrast, additional lines can usually be used to deliver different drugs or to withdraw samples, for example, in a consecutive manner, so that the number added is small. FIGS. 16 and 21 are exemplary and not to be interpreted in a limiting sense as to conclude that analogous treatment might be less applicable along the digestive tract, urinogenital system, or the airway, for example.

### Types of Electromagnet-Jackets

Electromagnet-jackets are of four basic types:

1. Impasse electromagnet-jackets without pipe or a side-entry, analogous to permanent magnet impasse-jackets described in copending application US 20140163664 A1.
2. Impasse electromagnet-jackets with a side-entry or access into the native lumen, analogous to the permanent magnet impasse-jackets shown in FIGS. 3 and 4; which except for a hard outer shell and the lack of a draw-plate are the same in essence as the contraction electromagnet-jackets shown in FIGS. 11 and 12.
3. Extraction jackets, shown in FIGS. 13 thru 15, with an entry into the native lumen used primarily to extract magnetically susceptible, commonly, superparamagnetic nanoparticle carrier-bound drugs into an integral collection chamber or trap; and
4. Contraction-electromagnets proper, such as those shown in FIGS. 10 thru 12, used to compress interposed tissue, which controlled in an iterative sequential pattern function as the peristalsis simulation jacket shown in FIG. 10.

To these basic types can be added permanent radiation shielding, individual contraction-jackets, used to simulate sphinteric function, and extraction jackets with an integral collection chamber or trap and modified double arm side connector, as shown in FIG. 5, applied to the extraction of high volume debris as in ambulatory leukapheresis, for example, can be connected in series with a common flush line emptying into a wearable collection tank or reservoir contained in the pump-pack. Once flushed through, a flush line serving an individual extraction jacket can also be used to deliver drugs.

Various combination jackets serve exceptional purposes. For example, a contraction and extraction electromagnet-jacket combining the features of the contraction jacket shown in FIGS. 11 and 12 having a pliant shell and draw-plate and the extraction jacket shown in FIG. 13 would constrict the ductus to extract magnetically susceptible particle-bound drugs from the lumen. This is done with adjuvant medication administered systemically to slow down the volumetric flow rate through the lumen during the process.

### Magnetized Side-Entry Jackets, or Piped Impasse-Jackets

### Piped Impasse-Jackets Using Permanent Magnets

Ductus side-entry or piped impasse-jackets with magnetization may incorporate permanent or electromagnets, to incorporate both types exceptional. When the side-entry connection jacket is not just piped but provided with a permanent or dc electromagnet to draw drug-carrier particles delivered through the pipe into the lumen wall, outer shell 4 is wrapped completely around magnet 8, magnetized in separate segments and bonded together, to isolate the toxic and brittle magnetic material. Since the side-entry connection jacket shown in FIG. 1 has neither magnetic nor radiation shielding layers interposed, foam lining 3 and outer shell or casing 4 are in direct contact. For clarity, the ductus is shown stripped of adherent tissue; in fact, some perivascular fat, serosa, or mesentery, for example, can be encircled within the fenestrated jacket if the microcirculation is not cut off.

Longitudinal extension or elongation essentially integrates a simple junction side-entry jacket with an impasse-jacket for the purpose of acting upon the drug or other therapeutic substance delivered through the native lumen and carried forward by the lumen contents immediately upon delivery. As shown in FIG. 4, longitudinal extension is usually to add a permanent magnet which for uniformity of takeup is usually magnetized in separate segments which are then bonded together to present a long central axis axifugally directed field intensified in a graduated manner in the antegrade or downstream direction. The actuability and adjustability of electromagnets affording superior functional versatility, most practical applications for extension using electromagnets also call for the inducement by each consecutive magnet of a field stronger than that of the magnet preceding or upstream to it.

Longitudinal extension is embodied in jackets such as shown in FIGS. 4 thru 6 and/or jackets connected together into a train, with strong polymeric or stainless steel wire, and radiation shielding added, shown as part number 12 in FIGS. 5 and 6 if treatment involves the use of a radionuclide. Fixed in the direction of its graduated field strength, a permanent side-entry magnet jacket cannot simply be reversed in direction to take up a carrier bonded drug delivered upstream whereas an electromagnet jacket can. While a single jacket can incorporate both a side-entry antegrade antegrade-directed gradient to take up a carrier-bound drug arriving from upstream and a retrograde-directed gradient to take up a carrier-bound drug arriving through the side-entry, the use of separate jackets is preferred as affording flexibility and a break in ensheathment.

The jackets shown in FIGS. 3 and 4 differs from the simple junction jacket shown in FIGS. 1 and 2 in having a magnetized layer 8 outside concentric to foam lining 3. Rather than uniformly magnetized through the thick dimension to direct the field radially toward the long axis of the lumen without change in field strength from one end of the jacket to the other, layer 8 is assembled from adjacent segments from separately magnetized high energy product neodymium iron boron paired half-cylinders. Each half-cylinder pair is magnetized through its thick dimension to direct the field radially toward what will be the long axis of the lumen with progressively greater intensity and sectioned into half-rings. One half-ring from each pair is then bonded to the next in order of increased field strength to reconstitute half-cylinders, but now with increasing field strength directed toward the long axis of the lumen from one to the next segment or section along the length of the sectional half-cylinder. The half-cylinders are then joined into an openable cylinder by bonding to spring-hinges 14.

The openable cylinder produced thus presents a magnetic field which is graduated by sectors from one end of the cylinder to the other. This gradient is intended to facilitate uniform takeup against, into, or through the subjacent lumen wall of a drug or other therapeutic substance delivered through ductus lumen 2 or jacket connector 6 or 10 as a ferrofluid wherein the medicinal substance is bound, such as molecule to molecule, to a magnetically susceptible carrier particle, such as a superparamagnetic nanoparticle. Unlike an impasse-jacket without a side-entry connector which must be marked to indicate the direction of increasing field strength, the side-entry of the completed jacket serves to indicate the end of lesser field strength. The jacket is placed with the increasing force directed downstream, that is, in the forward or antegrade direction of flow through the native lumen.

Such a magnetized jacket with side-entry connector, or piped impasse-jacket, is placed to encircle a lesion to be treated in order to draw the medication against or into the lesion. The jacket side-entry connector can receive magnetically nonsusceptible or ordinary drugs and superparamagnetic particle-bound drugs together or as mixed, or delivery of the different drugs can be offset, only the magnetically drug bound fraction detained at the jacket. Drugs or other therapeutic substances that should not mix before entry into the native lumen under treatment can be delivered to the side-entry connector through a multiluminal catheter simultaneously or at intervals. Separate catheters can be led to different side-entry connectors on any one jacket, each catheter can be multiluminal, and different water-jacket inlet or service channels used for further segregation, the primary object in this being to allow for future pharmaceutical developments.

Alternatively, a single line to conduct drugs to be kept separate during delivery is flushed through with water or a solvent, an intervening pump turret refill cartridge, several of these, or insertion of a hose feeding into the turret socket used to separate these substances. Were the segment to be treated instead limited to a length along an artery that can be encircled within one continuous jacket, then the jacket shown in FIG. 4 is used, and if radioactive then a jacket with radiation shield as shown in FIG. 5 as permanent and FIG. 6 as disintegable. FIG. 4 shows a longitudinal section through a ductus with side-entry connection jacket before the side-connector has been advanced to cut a plug out of the structure wall with its leading sharp edge brought into level alignment with the internal surface of the lumen wall. The jacket has a concentric layer of high energy product neodymium iron boron with the magnetic strength gradually increased in the downstream or antegrade direction. Such a jacket constitutes a piped impasse-jacket.

Perforations 19 may be circular, slits, or slots not so extended as would significantly interrupt the magnetic gradient. Outer shell 4 lines the perforations down to the foam, but not more adaxially or closer to the adventitia as would allow the inner edge of shell 4 to encroach upon the adventitia. In FIG. 4, the radially inner or adaxial edges at the ends of shell 4 have been brought down into contact with the outer surface of foam lining 3, whereas in FIG. 5, the foam is kept clear of shell 4, the plastic and metal composite film used to chemically enclose and isolate foam 3 assumed in FIG. 5 as lacking sufficient puncture or shear resistance to avoid its perforation, even were the edges rounded. The bond of the drug to the carrier particles can be dissoluble or indissoluble. When the bond is broken, the drug is released to flow downstream and the carrier taken up in the lumen wall. When the bond persists, the drug is drawn with the carrier into the wall.

Different formulations can thus be used to cause the drug and/or other therapeutic substance or substances to flow past, penetrate for a distance into, or completely perfuse through the segment wall. For example, where takeup into the lumen wall is not sought, nonmagneted jackets such as those shown in FIGS. 1 and 2, each connected to its respective socket in the port implanted at the body surface define the starting and ending levels with delivery by the upstream jacket of the drug and the downstream jacket of the counteractant (reversal agent, neutralizing agent, antidote). When not accomplished automatically during the prodromal phase as addressed above under the section entitled Background, introduction of the drug through port 16 can be through syringe injection by the patient when experiencing anginal pain attendant upon vasospasm induced ischemia. Provided the patient is awake and mentally competent, manual actuation thus may be satisfactory.

Otherwise, such function is best automated to accomplish drug delivery as soon as a measurable parameter indicates spasm. Such means were briefly addressed in the section above entitled Background. FIG. 17 shows an unelongated, unmagnetized, and unshielded, relatively thin-walled side-entry connection jacket of the simple junction type shown in FIGS. 1 and 2, but with two side-entry connectors 6. The jacket shown in FIG. 17 is suitable for placement toward the aortic root to allow the connection of coronary artery synthetic bypass lines as shown in FIGS. 21 and 22, where the great vessels are juxtaposed limiting jacket thickness, and dissection to gain access best minimized. Existing synthetic tubes, especially those of a caliber suitable for use as artery bypasses, require the delivery of an anticoagulant to prevent clotting. Line 11 allows the anticoagulant and any other drug or drugs needed, to be targeted to the end arterial coronary artery so that entry of the drug or drugs into the general circulation is avoided.

Viscoelastic polyurethane foam lining 3 conforms to and reduces trauma to the fine nervelets and vessels that support the ductus, and when thick enough, is compliant as to alleviate shaping the site, reducing the need for dissection and secondarily, the procedural duration. The foam lining allows the jacket to conform to irregularities in external diameter of the ductus and according to the thickness allowed by the clearance available, allows some periadventitial or other adherent tissue to be included for encirclement when physiologically desirable or fine dissection would significantly extend the procedural duration. Such jackets are longitudinally extended to incorporate the magnetic layer, which according to the field strength applied, are used to detain or draw the drug-carrier bound affinate against or into the wall surrounding the lumen.

Detention may pend delivery of a second substance that acts upon the first to break the carrier bond or to modify the drug, for example. A drug-carrier remaining bonded to the drug draws the drug against, and dependent upon the magnetic field strength, into the wall. When the bond is broken, the susceptible carrier is drawn alone, freeing the drug to continue through the circulation. FIG. 3 shows a side-entry connection jacket such as that shown in FIG. 2 with concentric magnet layer 8 interposed between viscoelastic polyurethane foam lining 3 and outer protective and magnet isolating shell or casing 4 of polyether ether ketone, for example, after sharp adluminal edge 9 of connector 6 has been brought to level alignment with the internal surface of lumen wall 2. At this time, the magnet would be made of high energy product neodymium iron boron.

Casing or shell 4 is wrapped about the sides of the jacket not only to protect, but to isolate the brittle magnetic material, and if incorporated, a tungsten heavy alloy radiation shield which are toxic, from the neighboring tissue. Depending upon the volume of delivery required, when, as shown in FIGS. 17 thru 22, jacket side-entry connector 6 is taken up to attach a synthetic bypass or shunt, a second side-entry connector or water-jacket side-branch connector 10 is used as the inlet for drugs in fluid form to include drug-carrier ferrofluids. Water-jacket inlet 10 and its line 11 are normally smaller in diameter than side-entry connector 6 but can be made as large as the application requires.

Magnet 8 is magnetized over the lesioned area in the segment jacketed whether radially symmetrical or encircling and can be omitted over an arc in which it is not essential and to reduce jacket thickness assists in the avoidance of neighboring tissue. The magnetized area of magnet 8 is that over which the magnetically susceptible particle bound drug is to be drawn from lumen 1 radially outward against, and when the field strength is sufficient, through native conduit wall 2, making the jackets shown in FIGS. 3 and 4 not just infusion junctions but piped impasse-jackets, and those in FIGS. 5, and 6 with radiation shielding, usually for the susceptible particle bound infusate; however, the infusate can be magnetized, another susceptible particle bound substance detained.

Side-connector 6 is generally positioned opposite the lesion, such as a tumor along the gut, with the magnet or portion of the magnet layer in fact magnetized on the opposite side to draw superparamagnetic drug-carrier nanoparticles delivered through connector 6 into the lesion. Foam layer 3 accommodates the lesion according to its thickness. Intended for takeup immediate to side-entry connector or native lumen inlet tube 6, magnetization is not of a segment along the lumen as recommends progressively increasing the strength of magnetization in the antegrade direction to obtain more uniform distribution of takeup along the length of a longitudinally extended jacket. Alternatively, the susceptibility of the drug-carrier particles can be varied spectrally.

Magnetization can, however, be graduated about the circumference to increasingly concentrate drug delivery to the center of the lesion, a sector of the magnet can be omitted, or the magnet otherwise configured or magnetized to match the lesion to be treated. When the magnetizer or the size of the jacket prohibit finer distinctions in field strength, the magnet is assembled from separately magnetized rings, arcs, or segments as necessary, these pieces bonded to constitute the half-cylinders joined by spring loaded hinges 14. A need to compose the magnet of different magnetic materials is not contemplated. When connector 6 can be positioned diametrically or opposite to the lesion, a side-entry jacket is selected in which magnet layer 8 is increased in strength of magnetization moving radially or about the circumference away from connector 6 to the opposite side where it is at the maximum in superjacent relation to the lesioned tissue.

Omission of an arc in the otherwise concentric magnet layer or graduation in the magnetic field strength is attained by proportional graduation in magnet thickness only when necessary to avoid encroachment upon neighboring tissue with patient discomfort. Using suture to impart lifting force will often serve to avert discomfort. Otherwise, standardization in general and uniformity of thickness in the magnet and other layers is less costly. Magnetic side-entry connectors must not themselves be magnetically susceptible; however, this does not exclude the use of nonmagnetic stainless steels. FIGS. 3 and 4 show side-entry connection jacket with surrounding concentric magnet layer 8 but not radiation shielding as shown in FIGS. 5 and 6.

When applied to a jacket for use on a blood vessel, the strength of magnetization is gradually increased in the antegrade or downstream direction in proportion to the blood pressure and susceptibility of the particles. The uptake of superparamagnetic drug-carrier nanoparticles delivered into the circulation through the side-entry connector is then as uniform as the volume of ferrofluid, blood pressure, susceptibility of the particles, and strength of magnetization allow. For treating radially asymmetrical or eccentric lesions, side-entry jacket magnets such as shown in FIGS. 3 thru 6 can be made to complement the eccentricity, or radial asymmetry, of the leasion by situating nonmagnetic blank sectors in the magnet layer.

In FIGS. 3-6, toxic neodymium iron boron magnet 8 is completely enclosed within shell 4, which also protects the brittle material in the event of a direct blow in an accident, for example. The native adluminal or adaxial surface of magnet 8 is covered by foam lining 3. In FIG. 5, showing the side-entry or jacket before side-entry connector 6 has been advanced to cut a plug out of the ductus wall 2 and its leading cutting edge 9 brought to level alignment with the internal surface of the lumen wall, radiation shield 12 allows the delivery of a drug-carrier bound low dose rate radionuclide for drawing into the lumen wall 2, likewise toxic, has shell 4 extended about the jacket ends to enclose it with the inner surface of radiation shielding 12 surrounding magnet 8.

Side-entry connection jacket connector subsidiary fluid conduction or water-jacket inlet pipelines 11 can be accessed through a conventional membrane port or port 16 implanted subcutaneously or onto the fascia at the body surface 18 to administer the anticoagulant and/or other drugs directly to the bypasses by injection or release by an ordinary syringe, automatic ambulatory syringe driver, or an infusion pump, thus avoiding the systemic circulation. Such a membranous port for leakless perforation by an injection needle eliminates problems of spillage out of the line should it require to be opened at the proximal end, and for that reason, is to be preferred whenever the application permits. However, a port that must allow insertion and removal of a cabled device or a valve cannot be of the conventional subcutaneous membrane type but must be configured to minimize spillage when opened.

To minimize its size in general and to avoid puncturing through aortic bodies in particular, the jacket is kept short in axial length. When applied as shown in FIGS. 21 and 22 to rechannel the flow of blood in double vessel disease, adjuvant medication is delivered through subsidiary (fluid conduction, water-jacket) service channel lines 11. Side-entry connector lines 13 are usually larger in caliber than service channel lines 11, which convey blood only when used to draw a blood sample. Use of a double side-entry connector jacket to allow synthetic bypass in single vessel disease would allow use of the second side-entry connector 6 for the delivery of medication from a port implanted at the body surface.

Along a straight ductus, extension in the long axis proportionally suppresses any tendency for angling or levering relative to the long central axis of the native conduit encircled. In the application depicted in FIGS. 21 and 22, however, the simple junction double side-connector jacket shown in FIG. 17 is positioned along the arch just above the root of the aorta and therefore nestled amid surrounding structures that spare it from levering of any significance. The foam lining must have sufficient thickness and compressibility, and the jacket a spring hinge closing force that complies with the pulse instantly. Because foam lining 3 affords additional protection from levering stresses, the jacket is used only to provide a secure junction with the aorta, and avoiding extension up and around the aorta would injure important baroreceptors and chemoreceptors, the jacket shown in FIG. 17 is made shorter.

The double side-connector jacket shown in FIG. 18 is not used only to provide a secure junction with a native conduit as shown in FIGS. 19, 21, and 22, but incorporates magnet 8 for the purpose of drawing a magnetically susceptible particle-bound drug or other therapeutic substance into the wall surrounding the lumen. Any jacket with a magnet layer must span across the lesion and then some as ‘extension for prevention;’ unless the diseased segment of the conduit is much smaller in length than the jacket must be to provide the junction, it will be greater in length than a jacket in the same location only used to form a junction. The simple junction type double side-connector jacket shown in FIG. 19 does not require magnet or radiation shield layers. However, a jacket with both magnet layer, concentric or eccentric, and a radiation shield, like that shown in FIG. 5 or 6 but with two or more side-connectors is considered one of numerous variants according to the medical requirements.

### Unpiped Electromagnet Impasse-Jackets

Unpiped electromagnet impasse-jackets lack a side-entry connector. Such jackets are able to generate greater magnetic field force focused at a circumscribed area. Multiple electromagnets, small as possible to minimize the size and dimensions of the jacket, are positioned at intervals along the jacket to achieve a graduated increase in field strength from the retrograde to the antegrade end. The magnets can be arranged in a helical pattern to distribute the weight. Helically arranged jackets also allows the core and coil of each magnet to be oriented longitudinally rather than circumferentially in relation to the long central axis of the native lumen and jacket. Unpiped electromagnet impasse-jackets are used where an intervening permanent magnet impasse-jacket would interfere with movement past it of magnetically susceptible particle-bound drugs meant to target tissue downstream therefrom, for example, and where very strong attractive force is needed to attract a susceptible particle or affinate carrying a drug or analyte to the tightly circumscribed area of the magnet pole.

Where a permanent magnet based impasse-jacket is usually preferable for uniform takeup over the length of the jacket, an electromagnet-based impasse-jacket is preferable for uniform takeup confined to a small segment. With the exceptions that a hard outer shell and no draw-plate is used, unpiped electromagnet impasse-jackets are configured as is the peristalsis jacket shown in FIG. 10, with individual magnet cross sectional views provided in FIGS. 11 and 12. When better to distribute the weight, the magnets are positioned in a helical pattern, the cores and coils will be offset, allowing these to be aligned to the long axis of the ductus and jacket. Situating the magnet pole in an opening or each of several magnets in openings respective of each in the jacket wall to reach down to the outer surface of the adventitia as shown in FIG. 12 reduces the magnetic gap, allowing some reduction in the size and weight of the magnet or magnets.

### Piped Electromagnet Impasse-Jackets

Piped electromagnet impasse-jackets are configured as is the train of contraction-electromagnets depicted in FIGS. 10 thru 12, except that the mounting substrate is a hard shell as shown for permanent magnet jackets in FIGS. 1 thru 6, and no draw-plate is used. The various medical desiderata specified in the preceding section for piped impasse-jackets using permanent magnets—such as the need to avoid completely enclosing the ductus by incorporating spaced openings (apertures, fenestra) that pass entirely through the jacket from its outer surface to the outer surface of the native ductus, and in the section that follows for clasp-magnets—such as the preventive and palliative use of adverse tissue reaction substances—apply no less to piped electromagnet-jackets. Absorbable and permanent radiation shielding if necessary is also the same as shown for permanent magnet side-entry jackets in FIGS. 5 and 6.

For simplicity of control as a unit, gradual intensification in the field strength of each successive series-wired identical magnet in the antegrade (anterograde) direction from one magnet to the next along the linear array can be accomplished by inserting resistors between each. In an apparatus to be worn, however, this poses factors of needless mass and wasted power consumption, in that every magnet in the array must match that of the one magnet which must present the strongest field. With larger jackets where these considerations become significant in terms of the size and weight of the battery and each implant, patient comfort, and wearing time (meantime to discharge), it is preferable to use nonidentical magnets which differ in the number of coil turns and/or core permeability from one series-wired magnet to the next. In either case, the average field strength of the array as a whole is then adjusted as a unit. While different affinates may require different field strengths, separate wiring to allow adjusting the amperage to each magnet should not be necessary.

### Radiation Shielded Jackets

Shielded jackets generally call for shielded supply lines; however, if the radioisotope is weak and immediately flushed through, this is sometimes avoided. Depending upon the placement and type medication to be administered, shielded jackets can be flushed for this purpose alone. Jacket shielding 12 is no more thick or extended about the jacket than the dose-rate of the radionuclide makes necessary. Delivery of a radionuclide is usually direct to the side-entry jacket through a line leading to port 16 implanted at the body surface 18, thus avoiding the circulation.

Tungsten heavy alloy radiation shielding 12 like neodymium iron boron magnet layer 8, is toxic, so that both are completely enclosed within chemically isolating and protective outer shell 4, typically made of polyether ether ketone, or PEEK, polymer and prospectively, of graphene. Complete enclosure of vessels promotive of atherosclerotic change, shell 4 wraps around perforations through the jacket down to the outermost layer of the vessel, or adventitia. To prevent noncompliant contact of the edges of shell 4 with the outer surface of native conduit wall 2 as would reduce the compliant excursion allowed by foam lining 3 and risk incisional or gouging injury, shell 4 must not extend adluminally adaxially past magnetic 8 and radiation shield 12 layers.

The jacket shown in FIG. 5 is suitable for the long term delivery of lower dose-rate radioisotope or radionuclide along a ductus less susceptible to deterioration when enclosed thus, where the line is flushed with water to clear any significant radioactive residue. The jacket is also used to deliver drugs such as a steroid, statin, calcium channel blocker, or nitrates to counteract degradation the result of enclosure. By contrast, the jacket shown in FIG. 6 has a shield layer formulated to disintegrate once the radiation has been depleted, clearing ‘breathing slits’ 19 to allow normal gas exchange in the internal environment. A side-entry jacket for a blood vessel must not completely enclose the vessel for more than a brief time or atherosclerotic degredation will ensue. If clinical judgment favors irradiating the lesion over permitting this consequence, then sideline 7 is used to deliver antiatherosclerotic medication.

Alternatively, where radiation therapy will be short term and the jacket by its weight or diameter causes discomfort, shielding layer 12 consists of overlapping tungsten particles encapsulated for chemical isolation and bound with an adhesive having a spontaneous hydrolytic and enzymatic breakdown time matched to the radiation exposure time. In such a jacket, the particulate shield is without an outer shell as shown in FIG. 5. Shielding 12 is formulated to disintegrate once the radiation is depleted or removed, exposing perforations 19 through underlying jacket shell 4. That is, shielding 12 encloses a jacket of the kind shown in FIG. 4, so that once the shielding has disintegrated, the exposed jacket shell 4 relieves the obstruction to the vasa and nervi vasora and allows the adventitia or fibrosa to ‘breathe,’ permitting the level of degradation counteracting drugs to be reduced if not eliminated.

Higher dose rate materials necessitate corresponding increase in the thickness of shielding 12 and shielding of the line leading up to the side-entry connection jacket. Provided sharp edges about the periphery of the jacket have been eliminated, the jacket will usually be couched amid tissue that will support and stabilize it without abrasive contact, even when the mass of the shielding 12 is significant. To minimize sliding movement against surrounding tissue, outer shell 4 is given a nonabrasive uneven surface. Heavier shielded jackets may require additional support with a polymeric halter or harness that may be provided with eyelets to pass through suture.

To preclude the need for a second invasive procedure to recover the jacket, the radiation shield, which must not include perforations (apertures, fenestrations) essential to prevent atherosclerotic degeneration in the substrate vessel, is normally formulated to disintegrate, the shielding in the supply line or lines, whether subsidiary or service channel or side-entry connector lines allowed to remain. Due to the mass of tungsten heavy alloy, jackets fed from shielded lines are minimized in weight to prevent patient discomfort. If higher dose rate radioisotopes necessitate the use of a thicker shield, suture is used to suspend and distribute the load. More heavily shielded jackets provide eyelets that extend from the outer surface of the shield to pass suture.

The radiation shield for long-term use shown in FIG. 5 is nondisintegrating. To prevent atherosclerotic degeneration in the substrate vessel which remains enclosed necessitates the delivery of anti-atherosclerotic, antihyperplasic, and/or anti-inflammatory drugs. In FIG. 16, line 11 remains following use to place the jacket and thereafter remains available as an accessory line for the separate transmission of substances to the target ductus. The side-connector conducts drugs toward the target vessel, not blood away from it as in FIG. 21, both lines 13 and 11 flowing into the target ductus. By contrast, the jacket shown in FIG. 21 is not placed about the target vessel but rather uses lines 13 to shunt blood away from the aorta as the source to which it is mounted for delivery of the shunted blood to the jackets placed distad about the target vessels.

In FIG. 21, drugs for delivery to the distal jackets is passed through lines 11. While placed, the distal jackets also required lines 11, which then sealed off, do not appear in the figure, drawn for pictorial clarity rather than optimal functionality. Radiation shield 12 is therefore enclosed within outer shell 4. In contrast, the shield in a shield disintegrable jacket encloses the magnet, that is, is interposed between the magnet and the shield of compacted encapsulated beads, chemical isolation already afforded by the polymeric coating applied to each bead. Whether in a jacket with nondisintegrating shield as shown in FIG. 5 or one with a disintegrating shield as shown in FIG. 6, locking bushing 5 is coplanar with surrounding portions of the shield and consists of solid or continuous, not particulate, heavy tungsten alloy.

In a shielded jacket, to allow side-connector 6 to be used as a trepan or circle cutter, the shielding bonded to side-connector lock bushing 5 is discontinuous with the shielding surrounding it as a rotatable knob. FIGS. 19 and 20 show the double side-connector jacket of FIG. 17 placed about a vessel where the side-entry connectors 6 have been set at a downward angle for optimized or least turbulent flow-through within the space available. The jacket is of the simple junction type shown in FIG. 2 without a magnet to constitute an impasse-jacket as shown in FIGS. 3 and 18 for the purpose of drawing medication outward into and/or through the luminal wall. In FIG. 21, the same jacket, used to form a secure junction with the aorta, is used to give origin to synthetic coronary arteries in a patient unable to provide suitable autografts and/or for whom the bypass procedure must be accomplished with minimal anesthesia or trauma, so that the additional procedure of harvesting grafts is avoided.

### Double Arm Side-Entry Connector Jackets

Rather than a simple junction type side-entry jacket such as shown in FIGS. 1 and 2 with a side-connector and an accessory inlet, a dual or double arm side-connector as shown in FIG. 7 is one with two connecting arms 70 and 71 that diverge at an angle from a common adductal shaft where either connecting arm can connect the mainline or sideline. While the arms are shown as equal in diameter, no such limitation applies. The differences between ordinary and double arm jackets are limited to the side-connector, other embodiments to include the addition of magnetization permanent or electromagnetic and shielding no less applicable. The ductus-encircling or ensheating portions of a double arm side-entry jacket and the part numbers shown in FIG. 7 are the same as for the simple junction jacket described above and are no different than those described for permanent and electromagnet impasse-jackets shielded or unshielded.

Much as the inline port shown in FIG. 30, any cabled device, whether fiberoptic endoscope, laser, intravascular ultrasound probe, linear or rotatory atherectomizer, thrombectomizer, aspiration catheter, or guidewire, is effortlessly directed into either the antegrade or retrograde direction by insertion through the line, whether treated as the mainline or sideline, which leads that way. The double arm conformation side-connector thus serves to expedite frequent fiberoptic or angioscopic inspection of the junction or transluminal or catheteric examination or treatment of the native lumen which the jacket encircles.

Moreover, more than one double side-connector can be incorporated into a single jacket, so that except for the side connectors, a jacket with a double side connector is constructed no differently than the jackets shown in FIGS. 17 thru 19. If justified, such a side-connector can be made triple or quadruple. For example, where the sum of therapeutic and diagnostic requirements justify, but space constraints disallow, the use of a jacket with more than one side-connector, the one side connector can be made triple or quadruple. Where space allows, double and plural arm side connectors can be added to jackets already having one or more side-connectors with sideline.

The larger caliber of the arm used to connect the line used as the accessory or sideline, still designated 11, allows not only easier passage of cabled devices but a higher volumetric flow rates. In FIG. 7, the double arm side-connector is shown in mid-longitudinal section, rotary joint 72 indicating that shaft or trunk 6 is round, allowing it to be rotated to whatever angle is best accommodated by the anatomy. Double arm side-connectors with elliptical or rectangular ductus side-entry openings or ostia, with or without flap-valve, such as shown in FIGS. 13 thru 15, cannot rotate unless the shaft encircling the ostium is made round; however, this severely limits the ostium in length.

### Clasp-Electromagnets

Clasp-electromagnets and extraction-electromagnets are configured to attach to the surface of nonductal tissue and cannot where a collar or jacket configuration cannot be used. Rather, these are attached by means of prongs treated to avert adverse tissue reactions and formed to encourage tissue infiltration. FIG. 8 shows a side view and FIG. 9 an overhead view of a clasp-electromagnet suitable for use as indicated just above or for positioning about the outer surface of a gland or organ to draw a bloodborne magnetically susceptible particle-bound drug radially outward through the parenchyma, for example. The mounting of a clasp-electromagnet is preferably molded in a single piece in polyether ether ketone (PEEK), graphene, or another polymer likely not to evoke an adverse tissue reaction. Various orientations of the electromagnet in relation to its mounting are addressed in the section above entitled Background of the Invention. The use of a clasp-electromagnet or an array thereof with a magnetically susceptible plate or plates applied to the far side of tissue across a gap from the array can be used to place that tissue in tension as a reverse type contraction magnet. Such an array can take any shape. Contraction magnets are addressed below in the section of like title.

Mountings other than that shown, which provides an opening for the magnet pole, consist of a simple plate with rounded and blunted edges configured to avoid abrasive contact with neighboring tissue. If necessary, the mounting can be placed in a hot sand box and bent to the exact shape needed in the catheter laboratory or clinic. To minimize procedural time, the clasp-electromagnet is configured to be pushed down against tissue to which it is to be attached and self-engage without further effort. In FIGS. 8 and 9, mounting magnet pole-surround face-plate 70 is continuous with and branches radially outward into a number of flexible prong-arms 71 terminating in prongs 72. To encourage tissue infiltration and integration, prongs 72 are textured and include a central perforation.

Clasp-electromagnets can be used where the ductus to be treated cannot be dissected free for encirclement with a jacket or where an eccentric lesion is on the facing side of the ductus. Clasp-electromagnets can also be placed to temporarily, periodically, or permanently supplement and boost the field force of a primary extraction-electromagnet with or without a flush-through line when the anatomy does not afford the clearance needed for a primary electromagnet to not encroach upon neighboring tissue. Abrasive and gouging encroachment must be avoided as the potential cause of incisions, fistulae, and ulcers. For this reason, the electromagnets are made as squat and unobtrusive as possible, housed in a smooth enclosure, and mounted for minimal obtrusion. The prongs are textured and perforated to encourage tissue infiltration and integration and are passivated by wetting with substances that suppress adverse tissue responses.

Substances typically used for this purpose include dexamethasone (see, for example, Vacanti, N. M., Cheng, H., Hill, P. S., Guerreiro, J. D., Dang, T. T., and 5 others 2012. “Localized Delivery of Dexamethasone from Electrospun Fibers Reduces the Foreign Body Response,” Biomacromolecules 13(10):3031-3038; Bhardwaj, U., Sura, R., Papadimitrakopoulos, F., and Burgess, D. J. 2010. “PLGA/PVA Hydrogel Composites for Long-term Inflammation Control Following S. C. [Subcutaneous] implantation,” International Journal of Pharmaceutics 384(1-2):78-86; Patil, S. D., Papadmitrakopoulos, F., and Burgess, D. J. 2007. “Concurrent Delivery of Dexamethasone and VEGF for Localized Inflammation Control and Angiogenesis,” Journal of Controlled Release 117(1):68-79; Patil, S. D., Papadimitrakopoulos, F., and Burgess, D. J. 2004. “Dexamethasone-loaded Poly(lactic-co-glycolic) Acid Microspheres/Poly(vinyl alcohol) Hydrogel Composite Coatings for Inflammation Control,” Diabetes Technology and Therapeutics 6(6):887-897).

The adverse tissue reaction retardant can be prepared in the form of implant-coated or embedded particles, microspheres, or nanorods (see, for example, Mercanzini, A., Reddy, S. T., Velluto, D., Colin, P., Maillard, A., Bensadoun, J. C., Hubbell, J. A., and Renaud, P. 2010. “Controlled Release Nanoparticle-embedded Coatings Reduce the Tissue Reaction to Neuroprostheses,” Journal of Controlled Release 145(3):196-202; Bhardwaj, U., Papadimitrakopoulos, F., and Burgess, D. J. 2008. “A Review of the Development of a Vehicle for Localized and Controlled Drug Delivery for Implantable Biosensors,” Journal of Diabetes Science and Technology 2(6):1016-1029; Bhardwaj, U., Sura, R., Papadimitrakopoulos, F., and Burgess D. J. 2007. “Controlling Acute Inflammation with Fast Releasing Dexamethasone-PLGA Microsphere/PVA Hydrogel Composites for Implantable Devices,” Journal of Diabetes Science and Technology 1(1):8-17; Patil, S. D., Papadimitrakopoulos, F., and Burgess, D. J. 2004. “Dexamethasone-loaded Poly(lactic-co-glycolic) Acid Microspheres/Poly(vinyl alcohol) Hydrogel Composite Coatings for Inflammation Control,” Diabetes Technology and Therapeutics 6(6):887-897; Hickey, T., Kreutzer, D., Burgess, D. J., and Moussy, F. 2002. “In Vivo Evaluation of a Dexamethasone/PLGA Microsphere System Designed to Suppress the Inflammatory Tissue Response to Implantable Medical Devices,” Journal of Biomedical Materials Research 61(2):180-187).

Another adverse tissue reaction retardant is phosphorylcholine (see, for example, Goreish, H. H., Lewis, A. L., Rose, S., and Lloyd, A. W. 2004. “The Effect of Phosphorylcholine-coated Materials on the Inflammatory Response and Fibrous Capsule Formation: in Vitro and in Vivo Observations,” Journal of Biomedical Materials Research. Part A 68(1):1-9; Chen, C., Lumsden, A. B., Ofenloch, J. C., Noe, B., Campbell, E. J., Stratford, P. W., Yianni, Y. P., Taylor, A. S., and Hanson, S. R. 1997. “Phosphorylcholine Coating of ePTFE Grafts Reduces Neointimal Hyperplasia in Canine Model,” Annals of Vascular Surgery 11(1):74-79; Whelan, D. M., van der Giessen, W. J., Krabbendam, S. C., van Vliet, E. A. Verdouw, P. D., Serruys, P. W., and van Beusekom, H. M. M. 2000. “Biocompatibility of Phosphorylcholine Coated Stents in Normal Porcine Coronary Arteries,” Heart 83(3):338-345).

A coating of zinc oxide, especially in the form of nanorods, can moderate an inflammatory immune response (see, for example, Zaveri, T. D., Dolgova, N. V., Chu, B. H., Lee, J., Wong, J., Lele, T. P., Ren, F., and Keselowsky, B. G. 2010. “Contributions of Surface Topography and Cytotoxicity to the Macrophage Response to Zinc Oxide Nanorods,” Biomaterials 31(11):2999-3007). Hydrogel polymers incorporating phosphorylcholine can be used as a bioinert medium for this medication (Lewis, A. L. 2006. “PC [Phosphorylcholine] Technology as a Platform for Drug Delivery: From Combination to Conjugation,” Expert Opinion on Drug Delivery 3(2):289-298).

### Clasp Extraction-Electromagnets

Where most clasp-electromagnets are mounted with the pole fastened to the mounting and directed away from the subjacent tissue as is clear from FIGS. 8 and 9, a clasp-extraction-electromagnet is secured to its mounting base at the rear, opposite its working pole. That is, a clasp-electromagnet usually looks away from its mounting in assisting a piped impasse-jacket with additional attractive force. Also, whereas a clasp electromagnet as shown in FIGS. 8 and 9 corresponds in function to an unpiped electromagnetic impasse jacket, a clasp extraction-electromagnet corresponds to an extraction jacket such as shown with a single magnet in FIG. 13. Unless the organ is enclosed within a capsule or rind that significantly increases the field force necessary to extract the unwanted residue, no opening or window is made through the capsule into the subjacent tissue.

Since there is no flap-valve to close the flush-line at the adductal end of the side-connector and tissue interface or contact area, the flushing fluid comes into direct contact with and washes over this tissue, allowing its treatment by including therapeutic substances in the flushing fluid. When the outer surface of the organ is fenestrated, the adductal edge of the side-connector is made to protrude into the fenestration to aid in maintaining the margin free of tissue ingrowth. If also necessary to prevent regeneration of the excised capsule by second intention, which would have the effect of increasing the magnetic field strength needed to effect extraction, then the flushing fluid has added to it a substance to counteract this process. Provided it is safe to do so, a small proportion of the flushing fluid can be replaced with sodium hypochlorite which is then itself promptly flushed away with plain water, for example.

Since flushing fluid must not be allowed to leak about the edges of the adductal end of the side-connector despite its pressure, the side-connector is not allowed to lift away. The prongs are therefore of a size and penetrate to a depth as will prevent leakage. When such treatment is exclusive of other extraction jackets and clasp-electromagnets, a separate supply reservoir, flush-line, and catch reservoir are used. When exclusive thus, the contents of the waste flushing fluid is available for diagnostic testing. Otherwise, a single flush-line can course through extraction jackets and clasp extraction-electromagnets at different sites. Ordinarily, the same flush-line courses through a circle of clasp extraction-electromagnets positioned about the surface of an organ, for example.

When the subjacent outer surface of the organ is not fenestrated, to provide the extractive force required, the magnet must be powerful enough to extract the residue before it can exert a toxic effect. Formulation of the drug-carrier particulate to retard this consequence thus bears directly upon the size and weight of the electromagnet or electromagnets needed. An impasse-jacket or clasp-electromagnet is ordinarily placed to draw a carrier particle-bonded analyte, or extractate, against and through the ductus wall, and extraordinarily—when the debris to be accumulated will be small and innocuous or can be neutralized with the addition of a followup substance—out through the adventitia to adhere to the pole until the magnet is turned off.

Since at the current state of iron oxide-based drug carrier particle formulation, the debris may be toxic or become toxic after an interval, it is more common for the debris to be completely extracted from the tissue and purged or expunged from the body. With a permanent magnet impasse-jacket, a powerful extracorporeal electromagnet is used to pull out the susceptible particles through an extraction grating. However, an automatic ambulatory system must be able to purge the debris without a need to visit the clinic. With an extraction electromagnet-jacket or clasp extraction electromagnet, a pole flush-line is provided to carry away the accumulated debris to a remote tank or waste reservoir in the pump-pack. Accordingly, the pole of the clasp-electromagnet is withdrawn from the surface of the tissue to no greater a distance than is essential to interpose the flush-line path, which passes through the magnetic gap.

### Sphincteric Jackets

Sphincteric dysfunction involves a condition of laxity or constriction along the digestive tract. Until its electromagnet is energized, a prosthetic sphincter assist device keeps the lumen fully closed. When energized, the magnet fully opens the lumen. It is therefore able to remedy a condition of either laxity or constriction. Unless so hypertrophied and stenosed that an assist device would have to be excessively large and heavy, a stenosed sphincter in a neonate, for example, should not require preparatory surgery. This notwithstanding, the device is not preferred over surgical correction that would impart a permanent cure.

Ductus chokes are not sphincteric, prosthetic, or physiological, but rather contraction-jackets used to facilitate system placement and maintenance by clamping the ductus from outside the body. When sphincteric and/or peristaltic jackets with control electronics are applied to the treatment of isolated motile dysfunction rather than as one module in a system used to treat multiple disorders, the omission of fluid lines allows implantation without the need for a belt borne pump-pack. A local control module and rechargeable battery can be implanted to assist only peristalsis or a sphincter, or the native or a tissue engineered graft esophagus together with the lower esophageal sphincter, or the pyloric sphincter and portion of the gut, for example, as a unit.

### Native or Tissue Engineered Graft Sphincter Assist Device

When the condition of weakness or the absence of contractive function is irremediable and the sphincter muscle thick, if and only if necessary to allow reduction in the magnetic gap and therewith, the need for a more powerful and heavier magnet, a lower esophageal or pyloric sphincteroplasty, for example, is performed to prepare the native segment for placement of the jacket. While a larger electromagnet will increase the weight and rate of battery drainage, within the functional magnetic gap, greater tolerance for deviations from cylindrical can be accommodated by increasing the thickness of the foam lining. If for any reason placement of an assist device or graft is preferred over surgical treatment for a stenotic sphincter, the permanent magnet is omitted. A sphincter that is lax requires the permanent magnet.

A native sphincter such as the lower esophageal, pyloric, or ileocecal maintains the lumen closed at rest and opened when stimulated by the autonomic nervous system; that is, contraction of the smooth muscle of the sphincter opens the lumen. A sphincter assist device must therefore open (distend, dilate, expand) the lumen when energized. A lapband sized for placement about a sphincter with port at the body surface not only works in reverse to contract the substrate structure, but cannot respond within the response time required. This stands in contrast to the action of peristalsis, a traveling wave of contraction produced by contraction of the circular and longitudinal smooth muscles in the wall of the conduit; however, a series of lapbands is likewise unadaptable to serve as an assist device for an impaired native or a graft segment.

A prosthetic sphincter best mimics a native sphincter in limiting the expenditure of energy to the exceptional condition, that of opening the lumen only following ingestion, conserving energy. That is, a contraction jacket that kept the lumen closed at all times except when deenergized following ingestion, allowing the lumen to open, would soon drain even a large and heavy battery. Peristalsis is likewise exceptional, in that it is activated only during ingestion. In a prosthesis, emulation of this energy conserving means optimizes the consumption of battery power, allowing the apparatus to function while portable over a much longer period, with a pump-pack that is smaller and lighter in weight, and less an impediment to free movement.

An impaired native and graft sphincter assist device is generally produced as a component in and for immediate incorporation into a prosthetic disorder response system of more encompassing scope as necessary. A prosthetic sphincteric jacket has a hard polymeric, such as polyether ether ketone (PEEK), outer shell with viscoelastic foam lining and perforations such as shown in FIG. 17 which pass through both shell and foam lining to expose the outer surface of the native or tissue engineered sphincter to the surrounding environment. A neodymium magnet is bonded to, inlaid into the outside, or embedded within the hard jacket shell at one side. Diametrically opposite thereto, an electromagnet capable of inducing greater field strength than the permanent magnet is mounted at the outside of the shell as shown for the contraction jacket in FIG. 12. As other jackets and clasp-electromagnets described herein, the jacket is lined with high density open cell viscoelastic polyurethane foam.

A perforated magnetically susceptible stainless steel draw-plate is sutured to the side of the ductus opposite the permanent magnet to face the electromagnet, its center along the diametrical line passing through the center of the permanent magnet and electromagnet so that the three components are coaxial. Provided the perforations in the draw-plate are monolithically or continuously lined with the encapsulating layer that encloses the rest of the draw-plate, a draw-plate of iron can be used. The permanent magnet has sufficient attractive force that so long as the electromagnet is not energized, the draw-plate, sutured at the opposite or far side of the ductus in relation to it, is pulled toward the permanent magnet. The tissue-engineered or native ductus is therefore collapsed between the two against the foam lining of the jacket.

To allow the subjacent ductus to ‘breathe,’ perforations are made through the jacket and draw-plate. So long as the electromagnet is not energized, the ductus is held closed passively with no expenditure of battery power. When energized, the electromagnet overpowers the retentive ability of the permanent magnet, causing the lumen of the ductus to open (expand, dilate). The perforated plate, made of magnetically susceptible stainless steel, is positioned in diametrical opposition to the pole of the electromagnet. Further to reduce the overall magnetic gap separating the magnet pole from the plate, the magnet pole is positioned within an opening in the side of the jacket, that is, through the hard shell and foam lining. To minimize encroachment upon neighboring tissue, the electromagnet coil or winding is bent around the jacket.

### Ductus Chokes

System chokes are individual contraction-jackets used to facilitate prosthetic disorder response system placement and maintenance by briefly suppressing intrinsic motility and preventing the continued movement of luminal contents into or out of the segment addressed. A ductus choke is a contraction-jacket equivalent to one of the segmental draw-plate and multiple electromagnet pair peristaltic magnets shown in FIG. 10, wherein the draw-plates are shown above the compound peristaltic jacket and in view while the electromagnets are below in diametrical opposition thereto and out of sight. These are, however, shown in the cross sectional views of FIGS. 11 and 12.

However, whereas the individual draw-plate magnet pairs of the peristalsis jacket shown in FIG. 10 function as a unit, the independent contraction-jacket or system choke is individually actuated. FIG. 11 shows such a jacket in encircling relation to a native ductus in diametrical and FIG. 12 in longitudinal cross section, with lumen 1 and surrounding tissue wall 2. The control system applies the input from proximally positioned strain gauge bolus sensors to the control of the magnet. A contraction-jacket has pliant tube 77 made of a rubbery implantable polymeric material rather than a hard shell 4, perforated through to the outer surface of the naïve ductus or graft.

Pliant tube 77 is lined with viscoelastic polyurethane foam 3 and elastic, with restorative force to reopen the lumen when the lumen-contracting attractive force of electromagnet 74 on draw-plate 76, coaxial with electromagnet 74 mounted at the opposite outer surface of the pliant tube is released. Perforated plate 76, made of magnetically susceptible stainless steel is positioned in diametrical opposition to electromagnet pole 75. As shown in FIG. 12, further to reduce the overall magnetic gap separating magnet pole 75 from plate 76, magnet pole 75 is positioned in an opening in the jacket side. To minimize encroachment upon neighboring tissue, electromagnet coil or winding 74 is bent around the jacket.

### Compound, or Peristalsis, Contraction Jackets

Peristalsis jackets position individual contraction electromagnet-jackets at intervals along a common substrate tube of the same kind as an individual contraction jacket. When mounted to a common substrate as a linear array and controlled to contract in advancing paired consecution leap-frog style with strictly coordinated timing, the set of contraction-jackets mounted, such as a tissue-engineered esophagus incapable of peristalsis, can be made to simulate peristalsis. As shown in FIG. 10, the common jacket mounting the set is termed the electromagnet-jacket, the component magnets then controlled as a unit. In a prosthetic disorder response system directed to more than one disease condition, timing control of the jacket as a unit can be accomplished either locally by a timing module mounted to the jacket or remotely by the respective control node.

Except that a hard shell without draw-plate is used, and the magnets are energized to present equal rather than progressively greater field strength from the retrograde to the antegrade end of the jacket, peristalsis jackets are the same in general structure or homologous with electromagnet impasse-jackets. Ductus obtained through tissue-engineered ing with autologous cells currently incapable of intrinsic peristalsis to provide a prosthetic esophagus, for example, peristaltic function can be imparted by applying impasse-jackets at intervals along the ductus with a magnetically susceptible plate positioned in diametrical opposition to each magnet. Unless means for withdrawing the plates are provided, the impasse-jacket can no longer function as other than a contraction or peristalsis jacket.

Addition of the plates 76 thus changes the function of each magnet from an impasse-jacket, used to detain a passing superparamagnetic nanoparticle-bound drug, to a contraction or peristalsis jacket. Such a compound jacket differs from a more conventional impasse-jacket magnet also in that its energization by the microcontroller is sequential within the set rather than independent A peristalsis jacket, with paired electromagnet and ferromagnetic plates arranged diametrically at intervals along its length, is shown in FIGS. 10 thru 12. Peristaltic action is simulated by energizing each consecutive magnet and plate pair while that preceding continues energized under the sequential timing control of a microcontroller. Such a jacket can be applied anywhere along the digestive tract, for example, where peristalsis of the native ductus is impaired or missing.

Autologous grafts and prostheses along the digestive tract having a history of failure, the application of such a jacket is substantially limited to instances of weakened peristalsis. Prosthetic esophagi of autologous gut, alloplastic, or nonbiological, materials having a record of rejection at the anastomoses, and those tissue-engineered not developing peristaltic function, a jacket mounting electromagnets in a sequential formation under coordinated control can serve to impart motility until a better tissue-engineered replacement ductus is developed. Whether a tissue-engineered replacement would be less susceptible to failure than an autologous graft remains to be seen. The magnetically susceptible draw-plates 76 shown in FIG. 10 are fastened to a thick spandex or similar stretchable rubber backing 82, a reinforcing band 83 with hook and loop ends binding about each draw-plate 76. Thick spandex or similar stretchable rubber backing 82 is lined with an inner layer of viscoelastic polyurethane foam 3.

Only the foam comes into contact with the outer surface of the dysfunction native or tissue-engineered ductus with lumen 1 and surrounding wall 2. The encircled ductus consisting of living tissue whether native dysfunctional or tissue-engineered, plates 76 and jacket or wrap 82 include perforations 19, which extend entirely through draw-plates 76, thick spandex or similar rubber backing 82, and high density viscoelastic polyurethane foam lining 3 to expose the surface of the ductus. Fastening of the magnets with coils 74, cores, 84, and poles 75 and draw-plates 76 to spandex or similar rubbery backing 82 is by means of bands 83. Draw-plates 76 can be fastened with small rivets (not shown) and a strong cement made for internal (intracorporeal) use, whereas the magnets are bonded along the side of the coil proximate to the band by means of a strong nonbiodegradable cement. Electromagnet-jackets are thus distinct from clasp-electromagnets in fastening to the substrate tissue on a backing that wraps the tissue about nonincisively, rather than by means of inserting prongs.

While shown in a linear arrangement, plates 76 can distribute the weight of the jacket by positioning these in a spiraling or helical arrangement about the substrate jacket, or the weight offset by esophagopexy or suspension of the prosthesis with suture. The jacket contains perforations 19 to allow exposure of the outer tunic to the internal environment. When sequentially energized in the distal direction, each electromagnet compresses the tissue interposed between plates 2 and pole 75 of the magnet. To prevent regurgitation, the magnet proximal (cephalad, superior) to each magnet remains energized until the magnet distal to it is energized. Alternative applications can employ one or more separately jacketed electromagnets to compress intervening tissue or simulate peristalsis in nonductal organs, such as along the renal capsule. As shown in the cross-section of FIG. 11, the core and coil have been bent around to least encroach upon neighboring tissue and more evenly distribute the weight while standing.

### Unpiped Electromagnet Impasse-Jackets

Unpiped electromagnet impasse-jackets function as do permanent magnet impasse-jackets to draw a magnetically susceptible particle-bound drug, drugs, or other therapeutic substances passing through the ductus lumen against the lumen wall until a second substance arrives, for example, or into the lumen wall. Where the permanent magnet jacket will attract any magnetically susceptible particle bound substance, the electromagnet does so only when energized. For this reason, any number of susceptible particle-bound substances can pass the jacket until that to be attracted arrives. An unpiped electromagnet impasse-jacket shown in FIGS. 11 and 12 but has a hard outer shell and no draw-plate.

The multiple electromagnets are small as possible, usually arranged in a helical pattern to better distribute the weight, and identical, but graduated in amperage, hence, field strength from the upstream to the downstream end. Another type of electromagnet-jacket, an extraction jacket, used for noncentrifugal ambulatory cytapheresis where a ferrofluid infused upstream through a simple junction jacket binds with a type cell to be extracted is described below. To the extent that intrinsic motility assist jackets serve only to replace dysfunctional or missing sphincteric, and multimagnet contraction jackets weak or missing peristaltic function, only electrical, not fluid, connections are necessary.

This allows the entire prosthesis to be implanted. When a simple junction jacket is needed upstream to supply synthetic mucus or other digestive substances, an extracorporeal pump-pack must be added. When fluid delivery is needed from the outset, the control and power components are relegated to a pump-pack, allowing access without an invasive procedure. Fluid delivery may be direct to a sphinteric or peristaltic jacket that is piped, or indirect, to simple junction jacket placed upstream. However, when synthetic mucus and enzymes, for example, are to be delivered, the jacket must be provided with one or more side-connectors and fluid lines led from a supply reservoir and pump in the pump-pack under the control of the microcontroller.

### Power Source

A central object to confer freedom of movement, the tethering and detention of a power cord is reserved for recharging. Added weight notwithstanding, the battery compartment in the pump-pack should include a fully charged spare battery. For direct power, to recharge batteries, and avert fouling of the battery compartment due to battery leakage should it occur, the pump-pack includes an internal power supply with ac power cord. In general, because it introduces contingency of availability, the use of an external power supply or ac adapter is not preferred despite the reduction in weight.

### Hybrid Impasse and Extraction-Jackets

If allowed to remain within the lumen wall or other target tissue, current superparamagnetic iron oxide nanoparticles for use as drug-carriers may pose a problem of toxicity (see, for example, Wahajuddin and Arora, S. 2012. “Superparamagnetic Iron Oxide Nanoparticles: Magnetic Nanoplatforms as Drug-carriers,” International Journal of Nanomedicine 7:3445-3471). With continued research into the formulation of drug carriers, this should prove less a problem, but means must be available for dealing with such a contingency. An unpiped electromagnet impasse-jacket as described in the preceding section can be used to draw the carried drug into the wall using an initial range of field strengths, any toxic residue then drawn entirely out of the wall with the average field strength of the magnets from one end of the jacket to the other increased.

Since the residue will accumulate at the magnet poles 75, this is satisfactory only when a small amount of the affinate is drawn out, as in some noncytapheretic extraction. One type of jacket that allows the drug to be drawn into the lumen wall and then extracted is a permanent magnet impasse-jacket with extraction grating as described in copending application US 20140163664. Such a jacket requires the use of a powerful external electromagnet to draw the unwanted residue out of the wall. This requires a visit to the clinic, and must be repeated where treatment is on a continued basis and/or the amount of the residue necessitates its frequent removal. With a chronic myeloproliferative disease, missing a visit to the clinic can bode grave consequences, recommending the use of an automatic ambulatory apparatus.

The type of hybrid extraction jacket used depends upon the rate volume of extractate removal and accumulation. For small volumes, an electromagnetic impasse-jacket with magnets having poles outside the adventitia such as shown in FIG. 12, and with sufficient strength to extract the bound analyte can be used if the buildup and remaining of residue at the poles poses no risk. The jacket fixes the magnetic gap separating the magnet pole from the motile adventitia; however, a residue toxic for the ductus wall will usually be equally toxic for surrounding tissue, and unless adherent to the pole or poles, the residue will drop away and drift off when the magnet is turned off. Moreover, when the accumulation is more than negligible, magnets able to generate the field strength required will tend to be larger and heavier.

These will necessitate additional dissection to place, suture or harness to stabilize, limiting the use of such jackets to sites that afford the necessary clearance, and adding to intraprocedural duration and the added risk of encroachment upon neighboring tissue. For these reasons, a more practical form of hybrid impasse and extraction jacket for most applications alternates electromagnets mounted as shown in FIG. 12, where the magnet poles remain outside the wall of the ductus, with those mounted as shown in FIG. 13 or 15 with trap and flush-line that passes through the consecutive extraction-magnet traps interposed between the magnets mounted as shown in FIG. 12, where a plug of tissue has been removed from the ductus wall.

Thus, whereas the flush-line in such a train of extraction jackets suitable for automatic ambulatory cytapheresis as shown in FIG. 14 and described below in the section that follows passes from the trap of one separate consecutive extraction jacket to the next, the flush-line passing through a hybrid jacket passes from one trap to the next within the length of a single jacket, passing over the intervening impasse electromagnets. Another type of hybrid jacket facilitates the extraction of a potentially harmful magnetically susceptible residue. Since native lumen wall 2 is fenestrated to position flap-valve 81 in face to face relation with magnet pole 75, wall 2 facing magnet pole 75 is missing. Extraction of a potentially harmful residue from within lumen wall 2 is by a permanent magnet impasse-jacket with extraction grating and the aid of an external (extracorporeal) electromagnet or by an extraction jacket with magnets able to generate the tractive force necessary to pull the residue through wall 2.

Such a jacket mounts magnets as shown in FIG. 12 where wall 2 is intact but like FIGS. 13 thru 15 in that each jacket has flush-line connection arms 80 such that flush-line 79 can pass through each jacket. The space separating the face of magnet pole 75 from flap-valve 81 in an extraction jacket or the outer surface of the ductus in a hybrid impasse and extraction jacket is positioned directly in the path of flush-line 79. The volume of debris that may be accumulated before flushing becomes necessary depends upon the magnetic gap and the rate at which the residue is accumulated. Periodic cytapheresis performed continuously over an interval allows the use of multiple extraction jackets such as shown in FIG. 14 wherein the magnets are smaller in size and weight. Further to implement noncentrifugation residue cytapheresis, or automatic ambulatory cytapheresis, another type of hybrid electromagnet jacket has multiple smaller electromagnets such as that shown in FIGS. 13 and 15.

### Extraction Jackets

Extraction jackets, shown in FIGS. 13 thru 15, allow magnetically susceptible particles such as superparamagnetic iron oxide nanoparticles, or SPIONs, to be withdrawn from a native lumen 1 and accumulated in a collection chamber, or trap 78, for removal. In profile, the jacket of FIG. 13 resembles that of FIG. 11 with the magnet core 84 and coil 74 bent around to minimize its protrusion. However, unlike the contraction-jacket shown in FIG. 11, the jacket is not pliant and no draw-plate is used. In FIGS. 13 thru 15, magnets 74 do not protrude to the extent the drawings imply: for descriptive purposes, the components of the extraction jackets, which wrap around and out of sight, have been laid relatively flat to the plane of the drawings and enlarged. In FIG. 14, trap 78, situated along flush-line 79, is connected to flush-line 79 by means of inlet and outlet connecting arms with convoluted fluid line or hose retentive ends 80.

Flush-line 79 conducts a flushing solution, wash water, or a flushing hydrogel from a clean supply reservoir in the pump-pack, through trap 78, to a waste reservoir in the pump-pack when periodically directed to do so by the control system program, which is based upon the rate of accumulation. FIG. 14 shows that when more than a single extraction jacket is used, flush-line 79 consecutively passes through each successive trap in the series, which is functionally bidirectional and geometrically symmetrical. An advantage in the use of electromagnets is that other type jackets addressed herein, such as electromagnetic impasse-jackets, can be interposed between the extraction jackets shown in FIG. 14 for use independently of or coordinated with the use of the extraction jackets.

In a compound jacket such as the peristaltic jacket shown in FIG. 10 or a series (chain, train) of jackets meant to function as a unit, such as the extraction jackets shown in FIG. 14, the magnets are wired in series and not separately adjustable. Interposed magnets when not components in another series are separately controllable. Individual magnet control makes possible the discretionary targeting of the carrier bonded drug or drugs delivered upstream to a specific jacket or subset of jackets and the type lesion these encircle at a given time. For any drug or drugs delivered by infusion through a simple junction jacket located upstream, administration is fully automatic, the patient unaware of the continuous process whereby condition-specific sensors signal the respective node to initiate drug delivery.

Optimally, this takes place before symptoms emerge. The single magnet extraction jacket shown in FIG. 13 has hard outer shell 4, made of a polymer well tolerated in the internal environment, such as polyether ether ketone, is made with rounded edges, minimal protrusion of magnet 74, compact as the thickness of viscoelastic polyurethane foam shell lining 3 will allow to avoid encroachment upon neighboring tissue, and includes perforations 19 which expose the outer surface of ductus wall 2 to the environment. Extraction jacket flap-valve 81 as shown in FIG. 13 is addressed above in the section entitled Background of the Invention. Flap-valve 81 is biased in pliancy to be more resistive to the entry into native ductus lumen 1 of fluid from line 79 than the extraction past its flaps of magnetically susceptible particles.

This bias is the product of flap geometry and the mechanical properties or deformation moduli of the material or materials, such as laminated, of which the flaps are made. The internal surface of flap-valve 81 is in direct contact with the contents of lumen 1 and must be constituted of a material or materials such as synthetic least likely to induce an adverse reaction. Flap-valve 81 at the adductal end of the passageway (throat, corridor) leading up to the adventitial or fibrosal outer tunic is flush planar to it along the outer surfaces of the ductus wall and foam lining flap-valve, or if falling short thereof, the sides of the passageway isolate the foam from the opening in the ductus wall.

### Jacket Placement

As shown in FIGS. 16 and 21, for example, the jackets with fluid and electrical lines connected are introduced through surface port 16 via an incision at the body surface shown in FIG. 27. To minimize trauma and the risk of infection, this incision is made no larger than is necessary to pass through the jackets and lines without excessive force. At the leading end of its lines, each jacket is then subcutaneously tunneled over the subjacent fascia to the site overlying that for its placement. Deeper or plunging dissection to gain access to the site for placement is limited to that essential. Whether periadventitial or fibrosal fat is left substantially intact depends upon its functional relation to the ductus, the avoidance of needless trauma, and the availability of the greater field force necessary to support the specific application over the wider magnetic gap that results. To allow for unanticipated contingencies, the magnets and battery are always somewhat overrated.

Once the plug is cut from the vessel wall, the connector is locked in position with its front edge level with the internal surface of the lumen. Used along the gut, the vacuum pump is then reversed to force the plug into the lumen. In a vessel, the plug must be extracted. This is accomplished with the vacuum. Should the tissue plug hang or resist extraction due to circumferential enclosure by surrounding tissue, a guide wire with hooked front tip is passed through an inline port as described below. When the combination of the foam lining and vacuum pump are not sufficient to hold the outer wall of the structure against the internal surface of the jacket, preliminary administration of polyethylene glycol-electrolyte solution for evacuation and opiates to truncate peristalsis is routine, as is the administration of antihypertensives to reduce the blood pressure.

As soon as the plug is excised, the vacuum pump is shutoff as promoting bleeding, and the pump switched to pump inlet hose 11 to ductus side-entry connector-internal fluid-conducting or water-jacket inlet 10, ductus side-entry connector-internal fluid-conducting or water-jacket 7 used to direct pressurized water against the breach, thereby suppressing bleeding (exsanguinations, extravasation) and forcing the plug out through side-entry connector 6. If necessary, a suction catheter is passed through side-entry connector 6 or the hose connected to side-entry connector 6 to pull out the plug.

The hemostatic or bleeding suppressive irrigation is stopped, the catheteric line leading to the automatic portable pump delivering medication through port 16 implanted at the body surface 18 quickly connected to side-entry connector 6, and fluid medication started with the object of establishing continuous flow, eliminating the entry of air into the line. Because it will have been prepositioned for any number of routine foreseeable and unforeseeable contingencies, a second catheteric line is always connected between fluid-conducting or water-jacket 7 connector or inlet 10 and its respective socket in port 16 implanted at the body surface 18.

When the vacuum is applied to the outer surface of the ductus, the sharp trepan front edge of the side-connector is drawn through the lumen wall, compressing the surrounding foam. This action effectively seals the jacket from native lumen contents, minimizing leakage. Once the forward sharp edge of the side-connector is aligned to the internal surface of the ductus wall, the foam decompresses, removing the brief compression on the fine nervelets and vasa vasora which the foam serves to protect. To prevent leakage of septic contents into the surrounding body, or peritoneal, cavity, journaling of the connector in the side of the jacket is tight.

Blood instantly spurting out of a breach in an artery on the systoles and continuing to drain on the diastoles to initiating clotting, the connector may be wetted with heparin lock flush, or ‘hep-lock,’ solution and provided with an internal circumferential fluid-conducting or water-jacket open at the front which irrigates the opened plug hole with pressurized and medicated tacky hydrogel, water, or heparinized water, for example, to restrain bleeding and reduce the risks of infection or inflammation. Once the front or adluminal trepan edge of the connector has been advanced to be level with the internal surface of the lumen, the fluid-conducting or water-jacket remains as a second lumen for other uses addressed below in this section.

The medicated tacky hydrogel or water, pressurized to minimize bleeding without significant entry into the bloodstream, is turned on an instant after the plug has been cut, and assists the vacuum to force the plug out through the side-entry connector. The combination of expulsive forces assures that the plug is safely extracted and cannot enter the lumen as an embolism. To assure that the plug does not catch on the distal or adluminal free edge of the fluid-conducting or water-jacket, the edge is rounded or rolled to form a rim and sufficiently receded in relation to the edge surrounding it as not to clog or interfere with cutting the plug.

A simple catheter, narrow hose connected to a vacuum pump or larger diameter thrombus aspiration catheter, the aspiration line serves first to retain the adventitia against the razor front edge of the side-entry connector, and is therefore introduced through the connector and turned on before the plug is cut. The vacuum line then cuts or assists the operator in cutting and withdrawing the plug, so that it is left on throughout plug cutting and extraction. Should the vacuum overpower the fluid or water pressure so that some blood issues from the vessel, the duration of the operation, hence, the absolute amount of blood loss is medically insignificant. When application under vacuum pressure of the sharp front or adductal edge of the connector to a thin walled vessel is sufficient to cut the plug, the need for the operator to manipulate the connector as a circle-cutter is eliminated, so that a locking collar or bushing is unnecessary.

The aspiration line and water ejected from the fluid-conducting, flushing solution, tacky hydrogel, or water-jacket are then used to withdraw the plug from the side-entry connector. Shutting off of the plug removal aspiration pump and turning on of the fluid-conducting or water-jacket pump are controlled with the same switch. The side-entry connector water-jacket ringing around to line the distal interior of the connector and its feed line attached to the connector at right angles, the connector lumen is clear. Irrigation is continued as and after the plug removal aspiration line has been withdrawn. Water spilling into the body cavity is removed with an ordinary aspiration line. Connection of the multilumen water or medication-filled catheter leading to the port implanted at the body surface to the side-entry connector at the same time that the fluid-conducting or water-jacket pump is turned off prevents air from entering the line and completes the procedure.

To prevent gas from entering, lines are kept filled, usually with a medicated tacky hydrogel. Each line has a small one way bleed valve to eliminate gas from the line. Any seepage of sterile wash water through such a valve is reabsorbed into the body and without medical significance. The pieces of crushed tacky hydrogel, fill or medicated, are generally too large to exit thus and could rarely if ever result in complications. Separating doses of water-insoluble or immiscible medication with water, for example, additionally assures dose accuracy, as well as prevents extravasation. A hypodermic syringe, infusion catheter, automatic infusion or similar ambulatory (wearable, portable) automatic pump is connected at the port to deliver medication directly to the jacketed segment. To assure that each dose is accurate, doses can separated by water or a hydrogel, for example.

The connection is suitable for pheresis, such as leukapheresis or dialysis, for example, circulation between the lumen and pump at one level, which can be accomplished with a dual lumen catheter, or from side-entry jackets at different levels. Circulation after the cutting edge has been brought level to the lumen wall and the connector locked in position can continue from one level with the fluid-conducting or water-jacket and the channel or passageway through the connector used in either direction. For this purpose and when the fluid-conducting or water-jacket way is to be used to withdraw diagnostic testing samples and the passageway through the connector to deliver drugs or the reverse, the relative diameters of fluid-conducting or water-jacket and passageway are chosen with this purpose in mind when the side-entry connection jacket is selected.

To avert backup inflow, when water rather than a tacky crushed hydrogel—which can also position the initial dose—is used to prevent leaks or extravasation during placement, lines can be filled with a higher viscosity substance such as a hydrogel without being capped off as would necessitate invasive reentry to recover their use. When the formation of a lesion is anticipated, unused jackets and lines ending at the port, without insertion of a corresponding pump-pair plug-in module in the pump-pack, can be prepositioned at points such as just downstream to the bifurcation in the common carotid artery. To attach or remove a hose or catheteric supply line, side-entry connector 6 can either be temporarily locked in position in the side-entry connection jacket or removed. FIG. 2 depicts the same side-entry jacket as that shown in FIG. 1 after the adluminal end of side-entry connector 6 has been advanced into level planar alignment with the internal surface of lumen wall 2.

### Valve Plugs

Should for any reason the side-entry connection jacket require to be disabled, prongs 20 shown in FIGS. 1 thru 3 are configured to engage and securely retain the forward or ductus adaxial portion of the silicone or other suitable elastomeric full length outer cylindrical surround or full length annulus of a shutoff and throttle valve-plug against the forward edge of water-jacket 7, thus covering over the opening made in the side of the ductus. This plug, especially when engaged thus, is never a solid mass of rubbery material as would hinder quick response to an exigent circumstance. From the moment the tissue plug is removed from the side of a blood vessel in particular, the plug must be extracted at the same time that leakage, if not eliminated, must be kept to a minimum.

Since the plug must be extracted through the side-entry connection line, keeping the line filled is not an option. The opening is therefore irrigated with water delivered at greater than the blood pressure through the water-jacket while the tissue plug is extracted. Because the side-entry connection line is needed to allow the irrigation water or other fluid to escape, the line cannot be filled while irrigation continues, although closing off the outlet at the instant irrigation is stopped will leave the line filled. However, introduction into the line of a valve-plug affords not only control over hemostasis but the rate of drug delivery. Valve-plugs are either passive, using an elastic membrane with slits, slats, or pinholes, or spring-loaded ball or vanes that open in response to the applied pressure to determine the volumetric flow rate therethrough, or are active, that is, mechanical and adjustable, so that the rate can be varied independently of the applied pressure by opening and closing a gate, such as in a butterfly valve.

Either type of valve-plug can be used with a side-entry connection jacket whether the jacket is of the simple junction, magnetized junction, or shielded and magnetized junction type. An active or vane adjustment type shutoff and throttling valve-plug is continuously variable between fully closed and fully open positions, so that in combination with the setting at the pump, for example, such a valve-plug can be used to throttle the volumetric flow rate through the line. Elastic membrane valves are suitable for use with fluids but not crushed hydrogels. Elastic membrane slit valves such as shown in FIG. 33 are used on small caliber catheteric lines that would otherwise empty of costly medication when the line was disconnected, for example. Provided the material of the valve is surface treated to minimize clotting, preferably without necessitating the infusion of anticlotting medication, elastic membrane valves can be made suitable for use with blood; however, these are generally without an incisive forward edge and limited to use in apparatus not in direct contact with native tissue.

The use of washing fluid and drugs in the form of slightly tacky hydrogels rather freely flowing liquids also reduces spillage, preventing lines from emptying when disconnected. When slid past the forward edge of water-jacket 7, the forward portion of the elastomeric surround 33 expands to the wider internal diameter of side-connector 6 to become pierced and engaged by recurved prongs 20. A wire passed through the line to supply current to a heating coil inside should be sufficiently inflexible as not to be overridden by the retreating valve-plug causing it to jam along the line within the gamut moved. The operator views the tantalum coated plug and drives it flush against the opening in the side of the ductus. Now the elastomeric surround is caught between the front edge or ledge of water-jacket 7 but prevented from moving forward by prongs 20, thus firmly fixing it in position. This prevents the plug from migrating into the lumen of the ductus.

Valve-plugs are either active mechanisms that regulate flow-through regardless of the applied fluid pressure or passive fluid resisters that respond to the applied pressure. It is also possible to combine these principles of operation in a single plug where the diameter of the slit elastic membrane that passively responds to the applied pressure is adjusted by the incorporation thereof within a mechanical operation 1 type plug. Closing openings in the plug essential to move it through the fluid column partially rather than fully allows such a plug, shown in FIGS. 23 thru 25 and described below, to be used as a throttle as well as a shutoff valve-plug. Used as a throttle, the valve-plug can be positioned anywhere along a side-entry connector line 13 or a service channel line 11. Elastomeric valve-plug surround 33 is, however, sufficiently compressible that a plug held securely as indicated can be retracted by an operator applying the required pulling force at the end of the guidewire to remove it.

The stopper and valve-plug is long and snugly fitting as to prevent veering from the long axis of the line in which it is inserted, cocking and jamming, or migrating. The plug pushed into the lumen, the pump is disconnected from and the permanent line leading to the port 16 implanted at the surface 18 connected to side-entry connector 6. The medicinal fluid is then passed into the lumen. Larger muscular arteries excepted, when placed along a blood vessel, the ductus wall will usually be thin enough that suction and the razor sharp front edge of the connector alone will be sufficient to cut the plug with no effort on the part of the operator. The blood pressure prevents the plug from reentering the vessel where it would embolize but is not adequate to force the plug entirely through the vacuum line with the pump off.

Since side-entry connection jackets are used to articulate a native conduit, lines 13 connected thereto convey native luminal contents unidirectionally. By contrast, flow through subsidiary or service channel lines 11 is often bidirectional. FIGS. 21 and 22 show a side-entry connection jacket applied to the ascending aorta to allow blood to be diverted into catheters or artificial arteries that bypass occluded segments of the intrinsic coronary arteries in a patient who lacks adequate native grafts and/or could not withstand the lengthier surgery and anesthetization to complete a conventional bypass harvesting and grafting procedure. FIG. 21 provides an anatomical overview of the jacket shown in FIGS. 17, 19, and 20 in use to attach catheters as coronary artery bypasses, while FIG. 22 provides a nonanatomical schematized view of the repair shown in FIG. 21.

To bypass endoluminal obstructions 17, catheters 13 are joined to the ascending aorta above and to respective distal segments of the native coronary arteries below by insertion of either end into side-entry connectors 6. Since the segment of the arteries shown sd bypassed in FIGS. 21 and 22 change little in caliber, upper and lower side-entry connectors are shown as equal in diameter, although the use of tapering or progressively narrowed catheters would allow mimicking the anatomy. While the upper and lower side-entry connectors are depicted as alike, the jackets differ in overall diameter and in that the delivery from a port 16 implanted at the body surface 18 of adjuvant medication, here an anticoagulant, through service channel lines 11 has been applied only at the upper jackets.

Placement thus allows sizing and joining of side-entry connectors 6 at either end of catheters 13 before or after endoscopic entry, the choice based upon patient anatomy. Whereas the lines that connect to side-entry connectors 6 and water-jacket inlets 10 from surface port 16 are synthetic, the jackets encircles native conduits 2, here the large aorta and the narrow coronaries. That is, whether the line from the port 16 at the body surface 18 is directly to a native conduit or indirectly to a synthetic conduit, bypass, or shunt by connection to and/or for junction with a native conduit, it is normally a native conduit 2 that is encircled by the jacket and the synthetic line that is connected to side-connector 6, any accessory or subsidiary lines 11 connected to water-jacket inlet 10 synthetic as well.

Whether used to unidirectionally conduct luminal contents through a bypass or shunt or to communicate with the surface bidirectionally, lines 13 connected to side-entry connectors 6 are synthetic with the jacket applied to a native conduit. Fluid conduction or water-jacket inlet service channel or subsidiary lines 11 are also synthetic but used to channel native luminal contents only exceptionally when used to draw test samples. The use thereof is normally unidirectional to deliver medication to a bypass or shunt jacket junction from a port 16 implanted at the body surface 18. When water-jacket lines 11 lack sufficient caliber to move contents directly to and from the native conduit, either a jacket with larger inlets 10 or a jacket with an additional side-entry connector is used to connect a line of larger caliber. In that instance, the line from the first side-entry connector joins the synthetic bypass, while the line connected to the second side-entry connector is connected at the surface.

Coagulation a deterrent to the use of current synthetic materials as bypasses or shunts shown in the accompanying drawing as part number 13, the side-entry connector water-jacket inlets 10 are used to connect the synthetic bypasses 13 to a port 16 implanted at the body surface 18 for the delivery of an anticoagulant and/or other liquid medication over water-jacket inlet lines 11 shown in FIGS. 21 thru 22. The bypasses and distal or insertion jackets are usually connected to the connectors before the jackets are placed, the lines from the body surface to the proximal or epicardial jackets for the delivery of the anticoagulant and any other liquid medication through a water-jacket inlet lines 11 connected thereafter. Placement about the ascending aorta is usually more expedient when bypass catheters 13 are attached to side-entry connectors 6 after the jackets have been placed. This may not be so for other locations.

Mechanical Valve-Plugs Manually Translatable and Adjustable and/or Radio Remotely Adjustable

Should for any reason the opening into the opening or ostium made in the side of the native conduit or ductus require to be closed off, a mechanical occlusion device in the form of a shutoff obturator or stopper and throttle valve-plug is used. FIG. 23 is a longitudinal section through a shutoff and throttle valve-plug in use to completely seal off the passageway through the passage created between the jacket and the native conduit, wherein lumen 1 is bounded by surrounding wall 2 and the side entry connector 6. The plug, shown in fully closed position, is seated at the ductus end of side-entry connector 6 to close off the opening into the ductus. One or more valve-plugs introduced into a line during placement of a jacket with lines and pump attached, as addressed below, may be positioned anywhere along the line, whether intra- or extracorporeally.

Once the operator has determined the best anatomical path or routing—and therewith the best lengths for the intracorporeal mainline and sideline between jacket and port—which need not be adjoined, or tunneled and routed together, this shutoff ability allows the port to be slid up to the skin and the lines cut flush at the port faceplate without medically significant spillage, for example. If remotely controlled as described below, flow through the valve-plug or plugs can be throttled or stopped at any distance from the patient, allowing the clinician to affect the rate of flowthrough in response to a call from a distant wearer. Midline as opposed to endline valving that uses an elastic slit membrane requires spanning the membrane across the longitudinal is not remotely controllable thus and less common than electromechanical valve-plugs.

Where the side-entry connection jacket must be removed or is no longer needed, complete removal of the jacket, lines, and port requires a second invasive procedure in which the ductus if a vessel is cross-clamped upstream long enough to place a graft or several turns with a hydrogel adhesive tape coated to encourage regrowth over the opening. The special treatment required for the carotid and coronary arteries is addressed above under Background. If the opening is small enough, tape is used with an absorption rate slower than that of endothelial regrowth. Referring now to FIGS. 23 and 24, valve-plugs can be configured to permanently close off the opening made in the side of the ductus, to do so while allowing in- and outflow through the side-entry connector but not the water-jacket, or to allow continued flow through the water-jacket as well as the side-entry connector, and can be made only so retentive in level that the program can reposition a valve-plug by sliding it at the head of a column of water or gel as programmed or as the clinician chooses.

Since line 13 must be kept filled with fluid to deny entry into line 13 by any contents of lumen 1 that would leak through the opening, the obturator must be advanceable, and if necessary retractable or withdrawable, through a fluid column. Special handling and viewing equipment needed at very small calibers, a slit membrane valve or mechanical shutoff obturator or stopper and throttle valve-plug is no less usable in a service channel as shown in FIGS. 1, 2, and 17 thru 22 as it is in a line 13 connected to a side-entry connector. As a throttle, even when used to temporarily stop flow, the plug can be situated anywhere along the line. Use in a side-entry connector attached line is with the front of the plug flush against the opening in the ductus to seal the ductus with no fluid between the plug and opening. When used as a long-term or permanent shutoff valve-plug to seal off the opening, the front of the plug must fit flush over the opening in the ductus with no fluid between these.

Effecting shutoff involves no invasive procedure as would placing a graft over the opening, which would begin to leak luminal contents were the jacket removed. If the jacket is to be removed, then the ductus is cross-clamped upstream, the special requirements pertaining to the carotid and coronary arteries addressed above under Background. Once the plug has been seated in position to cover the opening at the ductus-adluminal end of side-entry connector 6, completely closing continuously adjustable vanes (shutters, leaves, wings) 21 of the miniature duplex butterfly valve closes off flow-through. Whereas a valve-plug leaves open the possibility for inflow or outflow, covering over the opening or ostium created in the side of the ductus can also be with an autograft or absorbable hydrogel breach tape, the internal surface of which is treated to encourage the regeneration of tissue that closes the breach.

When opened, semicircular discs or vanes 21 allow fluid to pass through the plug in either direction, allowing the antegrade delivery of medication or the withdrawal of laboratory test samples. Continuously variable adjustment in elevation of the vanes, hence, the cross-sectional area of the apertures through the plug, allows continuous adjustment in the volumetric flow through the plug in either direction as a throttle. The throttle feature is pertinent to drug self-administration with a manually operated syringe, especially by an elderly patient with impaired motor control. When a pump is used to deliver medication through a side-entry connector line 13 or service channel 11, the dosing and rate of delivery can be set at the pump, averting the risks of drug delivery that is too fast or too slow.

In FIGS. 23 and 24, semicircular vanes or duplex butterfly valve discs 21 are tantalum contrast coated for improved fluoroscopic visibility and to prevent leakage when fully closed, edged with a durable watertightening rubbery or compressible elastomeric material, such as silicone. When fully elevated and thus closed, the outer edges of valve discs or vanes 21 are in contact with the internal surface of side-entry connector 6. Valve discs or vanes 21 meet flush at common midline watertight folding hinge joint 22 by rotation about axle 23, which is a pin fastened with no external presentation to the sides of side-entry connector 6. Valve discs or vanes 21 are pushed upwards into closed position and pulled downwards into open position by links 24.

Like vanes 21, links 24 are not strips but rather flats extending entirely across the semicircular space each extends over or subtends, edged with an elastomer where these are in contact with adjacent surfaces for watertightness, and joined for rotation at axle 25, which passes through follower block 26 and is fixed by resistance welding at either end to without extension outside side-entry connector 6. Links 24 therefore deflect and cause to diverge ductus-adaxially advancing, or in the orientation depicted in FIGS. 23 and 24, upwards flowing fluid, to either semicircular outer side edges of links 24, directing the fluid toward the side openings cleared by vanes 21. At their opposite or ductus-adaxial ends, links 24 connect to the underside of either respective vane 21 by rotatory joints toward the central outer edge which are able to slide from side to side within an enclosed way.

In a mechanical embodiment, links 24 are driven forward, or ductus adaxially, and pulled backward, or ductus-abaxially, when threaded shaft or leadscrew follower block 26, to which axle 25 is mounted, is advanced, or raised in FIGS. 23 and 24, by rotating leadscrew 27 clockwise and retracted or lowered by rotating leadscrew 27 counterclockwise. To maintain watertight contact between the outer surface of follower block 26 and the inward or medial edges of links 24, follower block 26 is oblate, so that its sides extend into the increasing space that would be opened between links 24 and the sides of follower block 26 as the central level in elevation of links 24 is approached were the sides of follower block 26 straight vertical.

At its ductus-abaxial or outer end, leadscrew 27 is fixedly inserted into journaled rotary bearing 28, having keyed entry hole 29 with entry opening into the keyhole at its ductus-abaxial or lower end as seen in FIGS. 23 and 24. FIG. 26 shows the distal or keyed end of guidewire 29, configured to fit into the opening or keyhole and the vertical and circumferential ways cut or molded into the internal wall of valve-plug journaled rotary bearing 28. In a valve-plug that is mechanically adjusted in vane elevation, hence, the volumetric flow rate, by rotating leadscrew 27, the sidewise or circumferentially extending keyways or tracks within rotatory bearing 28 allow rotation as well as advancement and withdrawal of the valve-plug.

In a remotely controlled servo embodiment, a leadscrew is not used. Instead, the direct drive armature of a linear servomotor or a rod connected to it and the bottom of follower block 26 replaces leadscrew 27 and rotatory bearing 28 to adjust vanes 21, a keyed guidewire and insertion keyhole still needed to advance or withdraw the valve-plug but not to adjust vanes 21. Thus, even though the rod extending from the armature or the ductus-adaxial end of the direct drive linear armature is itself fixedly connected to the keyed receptacle in lieu of a journaled bearing 28, the side or circumferential tracks or keyways remain essential to engage the sidewise extensions or side pieces of guidewire key crosspiece 30 for valve-plug advancement and withdrawal through side-entry connector 6 and line 13 connected to it, or along a service channel line 11.

Whenever a line is entered at the port so that luminal matter would be ejected and flow back through the opening or stoma and out through the line to cause the hydrogel to spill out at the port, the water-jacket lines should be used to forcibly irrigate the stoma, restraining luminal contents from exiting. If following placement, the water-jacket line or lines had been used as service channels to deliver medication, and to feed water through the line or lines would cause the medication in the service channel or channels to enter the lumen resulting in an overdose, then the side-entry line or lines is plugged with a stopper and the medication aspirated from the service channel or channels so that they can be used again as a water-jacket.

As seen in the proximal (rear, underside, ductus abaxial) view of a valve-plug in FIG. 25, rotary bearing in journal 28 is suspended by frame 31 at the center toward the valve-plug bottom, the valve-plug otherwise open below. The distal end of guidewire 29, shown in FIG. 26 is rounded or domed and has centering ring 32 with a diameter equal to the internal diameter of the catheteric line, so that the key at the distal tip of guidewire 29 slides into the underside keyhole of journal bearing 28 without hunting. Once the tip of guidewire 29 enters journal 28, it is stopped and must be rotated to admit crossbar key 30 before it can fully seat within the cavity at the bottom of journal 28. Leadscrew 27 and rotary bearing in journal 28 are self-locking, so that the rotary angle to which leadscrew 27 is set is held until intentionally changed.

A plug inserted along a double lumen or multiluminal line preserves segregation of the contents of the lumina to pass through either vane and to prevent delivery contents from reversing direction with concurrent outflow through the return vane and lumen by separating entry into either vane by means of a septum. In a mechanical embodiment, rotary bearing in journal 28, hence, leadscrew 27, is rotated by inserting a guidewire with keyed tip into a cavity open at the bottom of rotary bearing in journal 28. The key at the distal end of the guidewire consists of a small crossbar that fits into the complementary female notching within the cavity. The two longitudinal notches or ways are diametrically opposed and allow the tips of the crossbar 30 to slide up and down. Links 24 are connected together by common axle rotatory joint 25 at the center of leadscrew follower block 26.

Threaded shaft or leadscrew follower block 26 is raised by rotation of leadscrew 27 clockwise and lowered by rotation of leadscrew 27 counterclockwise. At the distance from the closed end of the cavity equal to the distance between the tip and crossbar on the guidewire, the longitudinal notch to either side is extended to either side by a notch at right angles. Rotating the guidewire when fully inserted within the cavity thus causes the tips of the crossbar to slide within these side notches until the ends of the notches prevent further rotation that does not also rotate rotary bearing within journal 26, thus rotating leadscrew 27, raising or lowering follower block 26 and therewith links 24 and vanes 21. The plug can thus be moved with two degrees of freedom, consisting of longitudinal advancement or withdrawal (retraction) and rotation to either side.

Access to a side-entry line 13 or service channel 11 by hypodermic needle is through a conventional subcutaneous or fascia set membrane port. For insertion or removal of a plug to serve as a shutoff or throttle valve or for passing through a fine caliber cabled device such as a fiberoptic angioscope, such a conventional port is not usable. An alternative port that affords the patency essential must provide an open passage into the body with minimal risk of infection. Since a plug if not a fine cabled device the same diameter as is the lumen of the line, means must be provided so that a wire or wires passed through the line from a sensor or heating coil inside a shutoff valve or throttle plug do not interfere with or become damaged by passage through the lumen.

While it still requires entry into the line with a guidewire to insert or retrieve a shutoff and throttle valve, a remotely controlled valve eliminates the need to insert a guidewire into the line in order to adjust the valve, making it possible to effect make the adjustment while the patient remains upright and the competent patient to do so on the basis of guidance over the phone. Another advantage in remote closed loop servo control is that the feedback signal is clearly displayed, so that the vanes need not be visually confirmed to have been set to the angle wanted by viewing the tantalum contrast coated vanes with the aid of imaging equipment. In a remotely controlled embodiment, a pulse width modulated microminiature linear remote controlled servo with a linear potentiometer as the feedback device is used. Alternatively, a valve-plug inmate vane mover can be powered by a conductor run down a side of the fluid line, usually a side-entry connected line.

, In a remotely controlled embodiment, the leadscrew and follower are replaced by a shaft connected directly or to a rod connected to the servo armature. Completely avoiding the need to reenter when the valve is in use, such as to pass through a cabled device, is accomplished by placing a second jacket and line along the same ductus. To use the same line and jacket, the valve must be removed to clear the way, leakage out of the opening in the side of the ductus prevented by using the water-jacket to irrigate the opening under pressure, just as is done when placing a side-entry connection jacket. Removal and insertion of a remotely adjustable shutoff and throttle valve-plug is by means of a guidewire, as described for a manually adjusted valve.

When not required for passing through cabled devices or to support high volumetric flow use, the surface port for a vascular side-entry jacket line can be of the portacath or conventional subcutaneously coursed or ‘tunneled’ central venous catheter type with implanted septum or membrane accessed through the skin with a hypodermic needle. However, for greater volumetric flow rate, to pass through cabled devices such as a fiberoptic angioscope or laser, and to expand the scope of applications beyond the vascular tree, the line is usually made patent from end to end. A side-entry jacket placed for the purpose of providing a passageway for cabled devices is positioned to allow the farthest travel through the lumen.

When a need for periodic visual examination by fiberoptic angioscope or intravascular ultrasound probe, for example, is anticipated, or when the probability is high that a transluminal procedure such as an angioplasty will become necessary, a second line and jacket can be placed and valved or kept filled with a durable hydrogel, for example, to prevent backflow. When the need to use a cabled device or devices is unanticipated, so that placement of a separate jacket or side-entry line for this purpose was not accomplished, a second invasive procedure is averted by passing the cabled device through the existing side-entry mainline. Provided the line is suitably valved, the cabled device can be passed through a piggyback port and the substance in the line, and provided an ambulatory pump-pack is securely fastened to the patient when moved between upright and recumbent positions, lines uninvolved in the procedure can continue to function without interruption.

### Body Surface Port

FIGS. 27 and 28 show a port for placement at the body surface, usually positioned pectorally as is a portacath, with lines of uniform diameter from end to end, allowing the passage therethrough of cabled devices as well as fluid pharmaceuticals. When use for cabled devices is not the primary or an initial purpose, the inability of a conventional subcutaneously placed port, or ‘portacath,’ for example, is properly disregarded only when an eventual need for more versatile function can be discounted with confidence. The lightweight port is minimized in size and number of parts, simplifying placement, reducing the opportunities for malfunction and complications, to include infection and adverse tissue reactions, making simple home maintenance by patients, and preventing pulling or pushing of the lines inside the body.

To avoid interference with clothing, least draw notice, and minimize the risk of collision, the port has a low pancake or squat truncated cone profile with rounded edges. A wide base minimizes depression of the body wall when pushed from the front, and inclined sides deflect collisions from any side. Essential sutures are internal and blanketed beneath antimicrobial and anti-immune agent wetted gauze. These features may make possible self sufficient care by the very young and old. Turning now to FIG. 27, shown is a port baseplate 34, made, for example, of nylon-carbon or nylon-glass fiber composite titanium or a nonmagnetic stainless steel.

That the port is shown with four lines through lines conduit 37 is purely exemplary, the same arrangement used for any number of lines, the diameter of the port adjusted to accommodate a larger number. The most elementary or basic configuration, suitable for the application depicted in FIG. 16, for example, requires but one mainline and one sideline, each connected to a bidirectional pump Port baseplate 34 is surface coated with an antimicrobial compound, such as one silver-based, and rests against the skin on cushion 35, made of a suitable rubbery plasticizer-free nonallergenic material such as silicone so that no portion of baseplate 34 can come into contact with the skin.

Cushion 35 must encircle to the outer side of suture holes 36 to fully encircle baseplate 34 thereby closing off the space overlying the skin to deny access to environmental pathogens, and should include openings or fenestrations sufficiently large to allow gauze 44 to protrude down between brace arms 38, but not extend over portions of the underside of baseplate 34 through which suture must be passed, or present angled edges as would cause irritation. Rather than to completely close off the suture points from the surrounding air, at least one of the perforations should admit a fine hypodermic needle to inject an antimicrobial if necessary. Suture holes 36 can pass through suture that to allow full enclosure of the wound by port cap 43 wraps inward around baseplate 34.

Suture can be passed through each local suture hole independently or made to cross over a neighboring brace arm 38 and/or lines conduit 37 suspension brace arm 38. Some combination and overlap of these three suture patterns provides the most stable and secure fastening of the port down to the integument. To expedite eventual removal of the port if necessary, bonding of lines conduit 37 to lines 13 and 11 and any other lines to the same side-entry connection jacket together by means of cyanoacrylate cement is accomplished only after the operator correct has found the optimal length and is limited to the more outward portions of the interfaces where these are in contact with one another and the internal surface of lines conduit 37. This allows the port to be removed by crushing the lines and conduit closer to the skin with a pliers before cutting the suture used to attach the port to the body

Where it passes through single small incision 39, lines-conduit 37 is coated to promote healing of the cut edges, hydrogen peroxide, povidone iodine, chlorhexidine gluconate, and hexachlorophene suitable antimicrobial agents. The binding of port components surrounding lines-conduit 37 is by compression cinching when port cap 43 is screwed down to baseplate 34. Incision 39 is completely enclosed by port cap 43 and can be protected by a small antiseptic soaked temporary dressing. How best to encourage the prompt healing of incision 39 by second or third intention depends upon its absolute size and is achieved by conventional means, to include the use of surgical cement. The central ends of lines-conduit 37 suspension brace arms 38 are bonded to the sides of lines-conduit 37 with an adhesive such as a cyanoacrylate cement.

Lines-conduit 37 made of polyether ether ketone (PEEK) or another implantable plastic binds together side-entry connector lines 13 and/or service channel lines 11 and extend through incision 39 into the internal cavity. Final fitting is least complicated when port, lines 11 and 13 encircled by lines-conduit 37, and jacket or jackets are preassembled and lines 11 and 13 and jacket or jackets are passed through incision 39 from the outside. Ports, lines, and jacket or jackets for common applications over a range of common sizes sold as preassembled are passed through the incision jacket first and moved into position endoscopically. As shown in FIG. 18, the proximal or outer end of lines-conduit 37 is encircled by port faceplate flange 40 having a downwardly expanding (inclined, beveled) outer edge complementary to the reversely inclined internal side of the center hole in port cap 43 when port cap 43 has been removed.

Faceplate flange 40 is sufficiently elastic that screwing on port cap 43 progressively places the lines (catheters, tubes) it encircles under greater compression as a compression fitting. Screwing port cap 43 onto port base plate 34 thus seals off gauze compartment 45 from air and waterborne microbiota about the outer surface if the port, while cushion 35 provides antimicrobial sealing at the bottom or skin contact surface. To allow the length of the intrracorporeal lines to remain freely adjustable during placement, port 16, which includes lines conduit 37, must remain freely slidable along lines 13 and 11 until cinched by screwing down port cap 43. To this end, lines conduit 37 is permanently and strongly bonded only to baseplate 34.

Lines made of a material amenable to roughening surface deformation by mechanical abrasion or chemical etching, for example, are prepared for retention by compressive cinching thus. When the lines or catheters passed up through and bound to or fused with faceplate flange 40 are made of a low friction fluoropolymer, for example, the internal surfaces of these lines parallel to faceplate flange 40 have bonded along the internal surface thereof ferrules with a denticulated or serrated internal surface. Inelastic tubes or plugs for insertion into the open ends of the lines have bonded or fused about the outer surface a corresponding ferrule of complementary denticulations or serrations that mesh with the corresponding internal projections of the intracorporeal lines when these opposed surfaces are forced flush into apposition.

When the port is not in use, the entry of microbiota or debris into the port lines is prevented by inserting a rubbery plug to cover over the port top center opening, thus sealing the port off from the environment. This is done by partially unscrewing port cap 43 and inserting a rubbery plug with peglike projections that insert into the open end of each line. The back of the plug overextends and roofs over the top center opening of the port lines conduit 37. Screwing down port cap 43 then compresses port faceplate flange 40, cinching about each projection within its respective line. When fewer in number than the port lines, lines from a pump or syringe driver to be connected to the port pass through a hole in such a plug, which otherwise is the same as the rubbery plug just described.

When no line would be left open to the environment, multiple pump lines can each insert directly into their respective port line on a short term basis; otherwise, the lines pass through a rubbery plug to seal the top center opening of port lines conduit 37. It being crucial for the delivery of medication that pump and port lines be correctly aligned, keying is imparted by making the port lines slightly different in internal diameter so that the pump lines will fit into the port lines only when correctly aligned. If necessary, differences in line caliber can be compensated for by adjustment in the delivery rate of the respective line pump in the pump-pack.

Separate insertion of the pump lines without a plug is discouraged not only for increased susceptibility to contamination but because joint insertion of pump lines in a plug improves correct alignment of pump and port lines, since keying is of the group, not each pump line, which may appear to fit the wrong port line when the diameters of the port lines differ only slightly. Since a rubbery plug with all line positions in use has no projection that would insert into a port line to become cinched about when port cap 43 is screwed down, means must be provided to ensure that the plug will be held down tightly to cover over and seal the top center opening of port lines conduit 37. This is accomplished simply by causing the plug to cling strongly to the pump lines by making the holes in the plug through which each pump line passes slightly smaller in diameter than its respective line.

Insertion of lines leading from a pump or syringe driver, for example, is by loosening port cap 43, removing the plug seal, inserting the pump lines and securely screwing down port cap 43. Since patients will differ considerably in thickness of subcutaneous fat and muscle, when sold as already assembled, lines-conduit 37 is provided with additional length and a tube cutter. When passed through incision 39, the distal jacket or jackets are closed but the protrusion of side-entry connectors and lines connected thereto will necessitate cautious angling of the jacket or jackets through incision 39. The outside wall of baseplate 34 is raised in height to provide an encircling thread 41 complement to thread 42 circling about the inside base end of port cap 43.

To expedite screwing it on and off, port cap 43, made of polyether ether ketone (PEEK) or another strong and lightweight plastic, is ribbed about the periphery, and screws down to baseplate 34 by engagement of thread 41 about the perimeter of baseplate 34 and thread 42 complementary thereto, running about the inner surface of port cap 43 along the bottom thereof. Shaped gauze pads 44 are easily replaced by the wearer, who need only unscrew port cap 43, press gauze pad 44 in gauze compartment 45 over faceplate 40, which retains it. To fill port cap 43 so as to reach down between lines conduit 37 suspension brace arms 38 to disinfect incision 39, gauze pads 44 are sufficiently thicker while uncompressed and preferably dispensed in individually wrapped hermetically sealed sterile plastic packages.

The pads are preferably dispensed having been wetted or permeated with antimicrobial and anti-inflammatory solutions and a healing promoting substance, such as a 20% solution of zinc oxide, as appropriate. Alternatively, the wearer wets the gauze with solutions from separate drip-top bottles. Provided medication is conveyed from the pump to the side-entry connection jacket in the form of a gel with adequate colligative strength or hardness, leakage out the proximal end of the line when disconnected should pose no problem. The use of a cabled device may necessitate clearing the line of medication or a neutral line filler in the form of a gel. This is most readily accomplished by using the water-jacket alone to drive the gel out of the line with the pressure of the backflow through the side-entry connection line.

The water pressure directed at the opening in the side of the ductus then restrains ductus lumen contents from leaking as it forces out the gel or liquid medication from the side-entry connection line. Since only continued water pressure restrains the ductus lumen contents, the opening must be sealed as soon as irrigation is stopped, usually by queuing an inert or medical gel in immediate succession to the water or by advancing a valve-plug with vanes fully open up against the opening and then closing the vanes. Alternatively, if not already present and so positioned, a valve-plug is inserted and positioned flush against the opening in the ductus. Resituating the valve-plug along the line is normally with the vanes open. To clear the line of proximal, or abaxial, gel to the pump side of the valve-plug, the vanes are shut and the valve-plug withdrawn to act as a plunger that expels the gel.

Along the vascular tree, water pressure is not applied with the valve-plug vanes closed when the medicinal gel is tenacious, since depending upon the relative magnitude of the water and blood pressure, the back-pressure could force water into the native lumen. In combined use of a valve-plug and water-jacket with or without heat to clear the line, provided no injury would result, the pressure from the water-jacket is allowed to force out the valve-plug as well as wash down the inside of the line, no guidewire then needed to retract the valve-plug. Resistance by the gel is reduced when the valve-plug or a resistance wire running along the inside of the side-entry connection line is used to warm the gel. Combining water pressure, heat, and mechanical plunger action clear the line, leaving it free of a residue. The valve-plug, heated or unheated, can also be used first as a plunger, with the inside of the line thereafter washed down by the outflow from the water-jacket. A submersible pump-pack can continue to operate during bathing or swimming.

Installation consists of 1. Preparing the lines, usually before the jacket and lines are placed endoscopically or robotically; 2. Placement anatomically, that is, positioning the jacket about the ductus and finding the most favorable route for the lines from the jacket to the port to be positioned at the body surface; 3. Extraction of the tissue plug from the side of the ductus; and 4. Instituting postprocedural and ongoing main and sideline flow by the pumps under manual and/or microcontroller control. Port 16 must be situated for maximum comfort, convenience, and serviceability. This is usually in a higher pectoral location, requiring that lines 13 and 11 be tunneled from the body surface to the ductus. To assure quick identification, lines 13 and 11 are clearly marked and contrast coated.

The application depicted in FIG. 16, with one mainline 13 and one sideline, or water-jacket and service channel 11, without additional sideline represents the simplest case condition for pump configuration and control. Where the prospective use is limited thus, the apparatus can be unitized. A line with a single jacket includes the jacket, intracorporeal and extracorporeal main and sidelines, the port where the external lines are plugged into the internal lines, and a pump-pair with one pump for each line, within a dedicated pump-pack that contains the battery and microcontroller. Such a unitized pump-pack would seldom if ever include pumps that would be switchable to serve more than one jacket mainline or sideline.

Where the prospective need should be flexible to allow for the addition of one or more jackets, the pump-pack is preferably of the pump-pair plug-in module receiving type that is not tied to or unitized with any particular type or number of pump-pair plug-in modules and not limited to a program that is less adaptable if not fixed. Instead, the pump-pack is a separate plug-in module pump-pair receiver with battery, controller, and program that adapts to a range of coordinated functions that might be required to support the different number of plug-in pump-pairs inserted in the pump-pack at any one time.

The use of pump-pair plug-in modules that include pumps provided with a switching turret as will be described to allow delivery through any jacket mainline or sideline of any in a number of jackets is more appropriate in such a more capable system. Within the constraints of patient comfort and freedom of movement, a wearable pump-pack can be configured to support a single jacket or a number of jackets, less frequently used pumps relegated to stationary or tabletop equipment in the home or clinic. Wearable pump microcontroller selection is based upon low power consumption for extended battery life and wearing time, consistent with programmability that allows the range and precision of operation required. In jacketing a vessel to depicted in FIG. 16, for example, each line terminates proximally or at the extracorporeal end passes through a separate pump toward it's.

The vials are mounted on a turret with detents rotated by a rotary solenoid. Before the operator has determined the most favorable route, setting the tunnel length requires that port 16 freely slide along the lines that pass through it, bonding of the port to the lines therefore deferred until after this length has been determined. Due to the need to minimize bleeding and avoid gas embolism, placement along the vascular tree under local anesthesia with the circulation uninterrupted is more demanding than is placement along a ureter or the gastrointestinal tract, where either can be temporarily anesthetized to suppress peristalsis, cross-clamped, and flushed clear of debris by irrigation through a fine hollow needle or hypotube.

If the ductus would collapse during circle-cutting to remove the tissue plug, the temporary placement of one cross-clamp upstream and another downstream to the prospective opening allows the intervening segment to be injected with a crushed tacky hydrogel, for example, so that it poses sufficient resistance to the side-entry connector during use as a circle-cutter or trephine to allow quick and clean excision of the plug. By contrast, in treating a vessel, especially a coronary or a carotid artery, endoscopically as will normally be the case, rather than in an open field, confirming that the lines are correctly primed will be critical.

While the use of transparent lines and side-entry connector allow direct viewing to expedite examination with a lit boroscope or endoscope, correct priming and making any adjustments if necessary is more readily accomplished before rather than after entry and routing. Regardless of the type ductus jacketed, the lines appurtenant of the same jacket will usually be routed together; however, where this would encroach upon neighboring tissue, the lines can be routed separately between the port and the jacket. Lines to move together are jointly ensheathed or cinched at intervals. To allow immediate identification whether viewed directly or imaged, each line must be clearly marked. Placement in an open field to treat the same or a different condition avoids the manipulative impediments of placement endoscopically. However, placement does not require an open field.

Endoscopic placement is through two small incisions, the first close to the prospective entry level along the ductus to be jacketed, the other for the port. If superficial, the routing can be also be followed through palpation. When the overall distance from port to jacket necessitates, an intervening incision or two is made to route the jacket to the ductus. Provided the jacket has corners and edges rounded and passes through without abrading the port incision, the jacket with lines attached is passed through the port incision, led to, and placed to encircle the ductus. Once the operator is satisfied that the routing and tautness of lines 13 and 11 is optimal, port 16 is slid flush up against the skin, baseplate sutured in position, cement applied to bond the proximal segments of the lines 13 and 11 and other points to be fixed in position, and port-cap 43 screwed down, cinching together the components passed through faceplate 40.

The turret line switching means to be described can readily outstrip any legitimate need for complexity based upon evidence based medical benefit, and the use of the simplest and least costly effective embodiment is always to be preferred. While added complexity increases the chances for malfunction, the drug routing scheme using turrets to be described keeps all moving parts in the pump-pack outside the body, allowing expedited servicing. For patients with simple embodiments remote from a repair technician, redundancy is used to allow for safe failure. Because the pump and turrets are more susceptible to failure, two identical pump-pairs are worn in a double socket power and control housing able to support either at a time. If the risk warrants, then each pump-pair is provided with an independent power source and microcontroller.

Whether an automatic transfer switch is used depends upon the probability of consciousness during malfunction. Referring now to FIGS. 29, 31, and 32, the jackets in a set supplied from the same or either of two pumps, for example, might be placed at intervals along a single ductus or distributed to different ductus of the same or different systems, and may be interposed by jackets connected to another pump or pumps, this versatility facilitating the treatment of comorbid conditions. Unplugging one pump-pack at the port and immediately connecting another allows uninterrupted drug delivery during servicing. FIG. 29 shows a plug-in module pump-pair inserted in a single pump-pair power and control module that includes microcontroller 51, battery 52, and optional hydrogel auxiliary reservoir 54.

FIG. 29 assigns the right hand pump of the pair to drug intake and the left hand pump to pump output. The module also includes variable speed reversible peristaltic pump 46, shown as rotating clockwise and switched to side-connector 6 through mainline 13, reversible pump 47, shown as rotating counterclockwise through water-jacket line or sideline 11, a miniature armoured flexible ‘gooseneck’ or BX electrical type cable conduit protective sheath 48 enclosing mainline 13 and sideline 11 to protect these up to the port 16. This simplified view omits pump intake and outlet turrets to allow switching any drug to any jacket inlet.

### Bidirectional Inline Port

FIG. 30 provides a detailed view of a bidirectional inline port fitting for insertion in a pump line to allow bidirectional entry into the line as a clean-out type inline port or cabled transcatheteric or transluminal device access point. This fitting can also incorporate a one way valve to expel air from the line. As shown in FIG. 30, the integral connecting arms are usually male with a smooth bore and ribbed or convoluted outer surface to retain an elastic hose passed over it. Whether the connecting arms are male with ends presenting internal ledges facing outward into a connecting hose or female with the reverse conformation depends upon whether the fitting is used to pass a fluid not delivered through a catheter, so that the ledge would accumulate material. The latter circumstance is responded to by using external or female connecting arms as shown in FIG. 31.

To prevent gouging or seizure (catching) of a guidewire or cabled device introduced into the line, the bidirectional clean-out type inline port fitting is made of a hard polymeric material such as polypropylene or nylon and formed to extend from the opening in the pump line to line the line as an annulus. The fitting is configured to steer the distal tip of the introduced device either up or down and through a slightly elliptical rather than true circular elastic slit membrane covering the opening or ostium into the pump line, the slightly ellipsoidal shape of the opening and membrane resulting from and varying with the angle of the intersection of the two inlet tubes. In manufacture, insertion of the fitting shown in FIG. 30 in a pump line, ordinarily proximal to a pump intake or outlet, is by sliding the line over a mandrel and applying a surrounding die that transects the line and cuts the complementary cutouts usually parabolic semicircular, in either free end.

The halves of the line are then pulled off of the mandrel and the free ends thereof slid over the top and bottom tubular extensions of the separately cast bidirectional clean-out type inline port fitting. The fitting and lines can be bonded together by fusion bonding, laser welding, ultrasonic welding, or by means of a suitable adhesive (see, for example, Zhou, Y. N. and Breyen, M. D. (eds.) 2013. Joining and Assembly of Medical Materials and Devices, Cambridge, England: Woodhead Publishing Ltd.; Ratner, B. D., Hoffman, A. S., Schoen, F. J., and Lemons, J. E. 2012. Biomaterials Science: An Introduction to Materials in Medicine, Waltham, Mass.: Elsevier-Academic Press). In FIGS. 29, 31, and 32, 69 are clean-out type inline ports which to prevent gouging and seizure of the extraction guidewire corkscrew tip or jamming at the nose end of cabled devices such as a fine endoscope or angioscope, necessitate that the material of the pump intake and outlet pipes or the segments thereof containing the clean-out type inline ports respective of each pipe be made of a hard and strong polymer such as polypropylene or nylon.

Shown in FIG. 32 is a pump intake switching turret to the right and a pump outlet turret to the left. The intake turret and parts used to connect the source of the drug or other substance to be delivered, such as a medicated flushing water or hydrogel, are shown in greater detail in FIGS. 33 thru 36. When too small to provide the volume of medication required, the standardized drug vial shown in FIGS. 33 thru 36 for insertion into a turret drug vial receptacle serves as the connector attached to the end of a hose from the drug reservoir for engagement in the turret. The vial also provides the initial dose of the drug or another drug preparatory to delivery of the primary drug. The arrangement shown in FIG. 29 is practicable but simplified for illustrative clarity.

A more usual and versatile arrangement is shown in FIG. 32, wherein one of the pumps in a pump-pair and jacket set is furnished with turrets at both its intake and outlet to allow any drug delivered through the intake turret to be sent to any jacket in the set. The two jackets represented in FIG. 32 as equal in size and distance from the pump might be placed along the same ductus, or ductus differing not only in size and/or distance from the pump but belonging to different bodily systems. This might, for example, consist of a jacket placed along the digestive tract and another placed about the artery that supplies that segment of the tract, or each jacket might treat different diseases related or coincidental. Flexibility and speed in reconnection of the lines to and from each pump are often significant when line switching must be reconfigured quickly as during installation.

While the pump-pack provides controls to override the switching arrangement controlled by the microcontroller in the even of a malfunction, the detailed disconnections and connections required to treat complex diseases and combinations thereof make automatic control responsive to sensor implants distinctly preferable if not essential for averting human error. Closed circuit recirculation with the arrangement shown in FIG. 29 necessitates connecting the pump intake and outlet lines. The circuit is completed by connecting the pump reservoirs together. The arrangement shown in FIG. 29 allows each pump to recirculate through a jacket via a closed circuit. While the jackets may vary in size according to the ductus each is to encircle, a pump-pair and jacket set unit can be standardized as to size, with the intake to either pump served from an infusate-switching source vial or hose, here depicted as a turret.

More complex arrangements, whereby the pumps are reversed as intake and outlet, intakes are interchangeable between a separate drug reservoir and the turret used to consecutively rotate vials of different drugs into the inline pumping position, the pump intake and outlet turrets of the same pump are functionally reversed, the outlet line of one pump is inserted into the intake turret of its partner pump or a pump in a different pump-pair, and so on, are avoided as exceeding practical needs and promoting human error. In FIG. 29, the drug on the right is stored in a reservoir so that more frequent and/or larger doses can be delivered, whereas that on the left must deliver its drug in small doses. The pump arrangement shown in FIG. 29 might be used, for example, to support the application shown in FIG. 16, wherein the side-entry connection jacket is used to form a simple junction without extension as a magnetized layer of increasing field strength in the antegrade direction to attract a superparamagnetic particle-bound drug radially outward through the lumen wall as a piped impasse-jacket such as those shown in FIGS. 3 and 4.

Multiple drugs are then supplied to the pump by a switching means such as a turret, wherein the loading positions or sockets can accept a vial such as shown in FIG. 34, which can also be used as the connector to a hose leading to a reservoir. With respect to FIG. 31, when lines 13 and 11 have elastic slit membrane valves proximal to the pump intake and outlet, indexing the turrets as appropriate and reversing the direction of rotation of the pump allows the alternate delivery of medication through either line. Indicated as part number 48 in FIG. 29, the distance to port 16 once placed widely variable so that the pump and electrical lines would be exposed to the risk of damage from a snag or impact the longer these are, protective BX type conduit ensheathing guard 48, extending to port 16 protects the lines.

The length of the lines as sold having been entered into memory by the maker for the jacket placement process, the length of each line following placement must be manually entered to change the original values in memory. Here the pump-pack supports just one side-entry connection jacket with drug or wash water or flushing fluid reservoir 49 on the right hand side to supply the inlet to mainline pump 46, rotating clockwise as shown, and sideline pump outlet or vial or refill cartridge holding turret 50 feeding the inlet of sideline pump 47, rotating counterclockwise. The intake to pump 46 is not limited to a static drug or wash water supply reservoir as shown in FIG. 29, but can include a supply turret containing circumferentially arranged compartments for inserting drug refill vials or for coupling inlet lines from other pumps or reservoirs internal, that is, intracorporeal, or external.

Neither is the drug radioactive as would require radiation shielding such as shown in FIG. 5. Mainline drug or wash water reservoir 49 and sideline drug reservoir, or if different drugs are to be delivered, turret feed 50, allow use of a microcontroller 51. Mainline pump 46, sideline pump 47, and microcontroller 51 draw power from battery 52. When supplementary or adjuvant drugs are likewise best targeted rather than taken orally, the drug reservoirs are replaced with a turret having sockets for holding drug vials or refill cartridges. [0675] In such a standardized jacket with support pump-pack unit, replacement of either reservoir with a turret necessitates sockets that will receive either a reservoir or a turret.

Since the unit must accept turrets so that the time and rate of feed can change with each indexing of the turret, the reservoir and turret sockets must include sensors and conductors for relaying vial or refill cartridge control or prescription data to microcontroller 51, and the program must respond to the data. While a basic standardized embodiment might call for changing drugs and adjusting controls mounted to the pump-pack by hand, this would not only negate the advantage or dependability conferred by automatic control but severely limit the range of use. Few disease conditions requiring but a single drug, and limitation to a single standard basic embodiment allowing the cost of production to be minimized, the standard basic unit includes reservoir-or-turret sockets with supporting sensors, conductors, processor, and program.

Successive refills can contain the same or different drugs, each turret indexed by actuation of opposing rotary solenoids or a quick response stepper motor. Different drugs may require adjustment in the speed of pumps 46 and 47, increasing the complexity of control, possibly necessitating a more capable microcontroller. Due to the prevalence and number of disorders such unitized embodiments can be used treat, the unit is suitable for production in different standardized sizes, significantly reducing costs. Further to reduce the cost, over a midrange of sizes, a single type embodiment with loosenable side-entry connector can serve both vascular and nonvascular application. At the lower end of the size range, the side-entry connector is not adjustable further reducing the cost.

At the high end of the size range, those nonvascular are provided with a side-entry connector that can be loosened to allow its use as a manual or trephine. Standalone integral units made in different sizes may be adequate where little if any benefit would be gained from additional complexity and cost, typically, in the maintenance of conditions that are common but pose little threat of sudden decline or death. In a single pump-pair with the mainline pump connected to a single side-entry connector and the sideline pump connected only to the sideline or water-jacket connector, providing a turret at the intake to each pump allows the sequential delivery of any drug through the side-entry connector or its sideline inlet. The ability to direct the outflow from a given pump to different jackets, or line switching, is not required of a single pump-pair with single jacket unit.

Pump-packs for relatively simple applications to treat a stable condition generally support a pump-pair with two jackets and are self-contained or integral, whereas more complex applications use one or more pump-pairs where each plugs in as a module into a pump-pack receiver. Although most lines connecting pumps and jackets will be small in caliber, the length of the lines may be considerably greater than that of a conventional automatic insulin pump, for example. For this reason, because much tacky gel may be required for jacket placement, especially when these are multiple, when each successive dose is dispersed through or separated by it, a filler substance is usually required to take up any drug-intervening spaces in the line. As shown in FIG. 29, this need is satisfied by connecting a large filler gel reservoir 54 at the bottom of the pump-pack to a pump intake line, usually through a flip flop or turret switching mechanism 53.

In FIG. 29, the hose leading from filler reservoir 54 to one of the intake pipe positions provided by the pump intake switching mechanism 53 is not shown as able to follow any of numerous paths. For use during placement of the jacket, drug reservoir switch 53 can also connect the intake of pump 46 to the outlet of pump 47 feeding into water-jacket line 11 and fill-gel reservoir 54. Similarly, the intake to pump 47 can be switched fill-gel reservoir 54. Filler reservoir 54 can also be used for storing ductus tissue opening irrigation and line flushing water. Portability not a factor in the clinic, the hose leading from pump-pack filler reservoir 54 can be disconnected at switching mechanism 53 for connection to stationary sources of hydrogel or water. Nominally indicated in FIG. 19 is control feedback sensor 55, typically for reporting blood gas levels or physiological parameters such as smooth muscle tension, many types and positioning possible.

For conditions that require treatment less simple than that depicted in FIG. 16, the pump-pair shown in FIG. 29 is increased in capability by incorporating a turret at the pump intake and/or outlet. As shown in FIG. 29, lines 11 and 13 are independent as to disallow recirculation through a closed circuit that must include both. Recirculation not only allows the periodic flushing through of lines automatically by the microcontroller 51 to remove residues, but as a part of or to expedite installation. To form a continuous loop and thus allow recirculation, the in-line drug vial or refill cartridge socket in turret 50 is connected by a pipe to drug or wash water reservoir 49. Admittance into the pipe is by a solenoid driven drop gate or swing-over obturator open/close valve under the control of microcontroller 51. Completion of the circuit by direct communication thus eliminates the greater complexity and expense of interconnecting lines 13 and 11 by means of in line switching valves.

Forming a closed circuit for flushing lines, for example, can be accomplished using both pumps in a pair as shown in FIG. 29 or a single pump as shown in FIG. 32. In FIG. 29, pumps 46 and 47 are configured for function as a coordinated pump-pair, pump 46 drug intake supply reservoir 46 and pump 47 outlet drug supply and line coupling turret 50 assigned not to both but rather to either pump in the pump-pair. In the arrangement depicted in FIG. 32, one of the pumps in a given pump-pair is used independently. The outlet of the other pump in the pump-pair could be plugged into the intake or outlet turret of the other pump; however, the need for such cross-feeding between pumps in a pair is exceptional. Cross-feeding to pumps belonging to other pump-pair and jacket sets is avoided as needlessly complicated as to invite errors.

The utility of pump outlet line switching between jackets is beneficial when, for example, a very small dose of a costly drug must be delivered to two or more jackets, and especially, when the consecutive time of delivery to each jacket is significant. Equally important is that the capability supports the development of new drug regimens, to include the automatic targeting of drugs to different parts of the body in strategically timed succession throughout the day, not previously amenable to practical implementation. In dual or double pump-pair embodiments, increasing the number of side-entry connectors and sidelines of each jacket to match the number of pump inputs eliminates the need to place a second line switching means. Such include a turret at a single side-entry connector or branching the main and sideline entering a side-entry connection jacket toward the pump outlets so that each of two drugs accordingly move through separate lines.

Whereas lines supporting side-entry connection jackets placed along the vascular tree or the urogenital tract are small enough in caliber that placement should seldom encroach upon neighboring tissue as to cause pain by compression of a nerve or vessel, jackets placed along the gastrointestinal tract or airway might do so. Where anatomical or operative considerations discourage the placement of multiple lines to access a given jacket, the input line to each jacket is provided with a conventional miniature piggyback port with valve. FIG. 32 shows one of the two pumps in a pump-pair with switching mechanisms at both the pump intake and outlet to allow the sequential delivery of any drug to the mainline or sideline of any jacket.

In FIG. 32, crushed tacky hydrogel, drugs, drug hydrogels, and/or wash water for separate consecutive delivery to different jackets are delivered from one of the pumps in a pump-pair through the lines 13 and 11 and side-entry connector 6 of either jacket. Pump outlet flow lines (arms, runs) 11 are connected at intervals about outflow indexing turret plate 57, and pump intake lines 13 are connected at intervals about turret drug vials and/or vials used as drug reservoir hose connectors to pump intake sectional tray consisting of sectional tray 58 and hold-down plate 59. Each turret rotates one inlet vial or line into the in-line position at the same time that it rotates the preceding line out of the in-line position. Lines 13 and 11 are given enough slack that these do not interfere with rotation of the turrets.

FIG. 32 depicts the side-entry connection jacket at the top left as currently connected to pump 56, pump to turret outlet line 64 indexed, or switched by turret 57 motor 61 to the inline position, with accessory or sideline 11 connected to water-jacket or accessory inlet 10 of that jacket. The lines of the jacket to the top right are not currently indexed to the pump inline positions and are therefore disconnected from pump 56. Pump 56 is continuously adjustable in speed and reversible, allowing outflow to and inflow from either jacket over the range of drug volumetric flow rates without the need to switch to lines of different caliber.

Pump 56 is usually one of a pair, one usually connected to the sideline, the other to the side-connector as shown in FIG. 29. When more than one pump-pair is present, the connection of these to either jacket is through lines connected to the turret respective of each jacket. Reciprocally, jackets not shown in FIG. 32 may communicate with pump 56. The foregoing degrees of flexibility attest to a potential versatility able to respond to extraordinarily complex medical conditions. Pump and jacket relations are ordinarily simple. As shown in FIG. 29, means of switching such as turrets may not be necessary. The simplest functional arrangement is always to be preferred.

To prevent air from entering the lines in vascular applications, turrets 57 and 59 omit blank vial positions that leave a line open-ended; rather, pumping is stopped once the amount of the infusate has passed when the hose can be disconnected. As shown, the left-hand turret lacks a vial and reservoir hose plug in table seen at 58 on the right, indicating that in this application, only the right-hand turret loads drug vials or receives medicated hydrogel or other therapeutic substance reservoir hoses. Were, however, drugs to be supplied from the turret to the left or a tacky medicinal hydrogel, for example, to be recirculated through the closed pump circuit with pump 56 when rotated clockwise, then the turret on the left would be of the same kind as that on the right. If to fill the line then stop or recirculate the gel, a reservoir hose as shown in FIG. 36 would supply the gel necessary to fill the line.

Recirculation may be used during placement and during the flushing of a series of jackets such as shown in FIG. 10 flushing, however, ended by running the accumulated flush fluid out of the system. In FIG. 32, to allow pump inline positioning of either jacket mainline 13 as well as any drug in drug vial turret 58, right-hand turret top-plate 59 to which jacket mainlines 13 are connected and drug vial turret 58 must be independently rotatable. As depicted, a single turret motor 60 is engageable with line rotating top plate 59 and drug vial turret at 58, a conventional latching or intromitting pawl mechanism internal to turret stile or mounting shaft 62 used to effect switching engagement. Alternatively, a second turret motor can be placed directly beneath motor 60. In this application, the drug supply turret has lines connected to it because drugs are recirculated through the closed circuit.

When turret 58 is to receive a drug reservoir hose as shown in FIG. 36, line connection top-plate 59 and turret 58 must move together, and line 13 to the left will then be rotated out of the pump line, preventing recirculation through the closed circuit to the left. Because a hose from or to a reservoir supplying a drug or other therapeutic substance as shown in FIG. 36 necessitates insertion through top or hold-down plate 59 and into a vial receptacle, the hose, top-plate, and receptacle must move together. Positioning a reservoir hose inline with a pump intake or outlet denies that position for a sideline 13 or mainline 11. Such conflicts are minimized by standardizing a dual pump or pump-pair configuration to include turrets at the intake and outlet of either pump thereby providing an additional path. The configuration of FIG. 32 is simplified to allow treatment of an uncomplicated condition at less expense.

This limits any lines 13 and 11 also inserted to a single turret drug position. Placement of a jacket requiring both recirculation and reservoir feed is therefore expedited by providing each pump with an intake and an outlet turret and recirculating through the one pump while feeding from a reservoir through the other pump. The elimination of top-plate 59 eliminates the ability to reposition lines 13 and 11 in relation to the drug vials. Rotating pump 56 counterclockwise sends the inline drug in the right-hand turret through line 13 of whichever jacket has been rotated into the pump intake inline position. When pump 56 is rotated clockwise, the drug in the turret drug vial or hose receptacle to the right of FIG. 21 is drawn downward into pump 56 and pumped through sideline 11 of whichever jacket accessory or water jacket inlet 10 is connected thereto and rotated into the inline pump outlet position.

Pump reversibility can serve to avoid the need for a symmetrical turret and switching scheme, that even with only one pump would considerably increase the cost of the pump-pack and jacket set. Referring to FIGS. 32 and 36, the receptacles in turret 58 can each receive a vial containing a drug or other therapeutic substance. As depicted in FIGS. 33 thru 36, the larger of the two differently sized standardized vials 66 and 85 is also used as a connector or coupling to engage a hose led from a drug, drug gel, fill gel, or other therapeutic substance reservoir such as 49 or 54 in FIG. 29. Standardized vial 66 also provides an initial or preparatory dose, is therefore filled when removed from the sterile wrapping as dispensed, and to prevent the loss of contents, has slit membrane valves 67 and 68 to close off its upper and lower ends.

To prevent the loss of contents, membrane valves 67 and 68 remain closed except when forced open under pump pressure to allow the passage of contents therethrough. Connection to a reservoir is necessary, because an amount of the tacky gel used to quench bleeding immediately after the plug of tissue from the side of the ductus is extracted, or wash water, for example, is larger than fits into a vial. Line connection and drug refill insertion plate 59 has concentric openings that allow the insertion therethrough of drug vials into subjacent turret drug vial or reservoir loaded sectional tray 58 and are threaded or incorporate other conventional means for coupling or fastening the end of a fluid line from a fluid reservoir or another pump thereto.

The openings through connection and drug refill insertion plate 59 generally either connect a line from a remote reservoir or a different pump or contain a drug vial; for a pipe to flow through an interposed vial or reservoir of another drug in the sectional tray is exceptional. In FIG. 32, the turret to the right hand or pump intake side includes upper line coupling or connection and drug refill insertion and hold-down plate 59, which to position any line connected thereto into the pump intake position in any sequence, must be capable of rotating both clockwise and counterclockwise.

At the same time, to rotate any drug vial or reservoir inserted within turret drug vial or reservoir loaded sectional tray 58 into the pump intake position in any sequence, turret drug vial or reservoir loaded sectional tray 58 must also be capable of rotating in either direction, and must do so independently of line connection and drug refill insertion plate 59. Independent rotator indexing of plate 59 and turret drug vial or reservoir loaded sectional tray 58 is accomplished through the use of separate direct current stepper motor 60, used to rotate line connection and drug vial turret sectional tray 58, used to rotate turret drug vial or reservoir holding sectional tray 58. In FIG. 32, drivers 60 and 61 are depicted as through-bore or direct drive-configured for visual clarity, the use of a gear train not disallowed.

Line connection and drug refill insertion plate 59 may be fastened to turret mounting shaft or stile 62 by pressing and so expanding the upper end of stile 62 into an end cap that retains and allows plate 59 and turret drug vial or reservoir holding sectional tray 58 to rotate freely and independently between it and motor 60 beneath it; however, use of a removable fasterner such as a screw in cap or flange allows plate 59 and turret drug vial or reservoir holding sectional tray 58 to be replaced and therewith extending the sizes of the drug refills and lines that can be accommodated. However, most such apparatus is intended to treat chronic conditions that are generally stable over the long term, so that this additional degree of flexibility exceeds most practical needs.

Ordinarily, when the side-entry connectors belong to different jackets, it is the pump nominally assigned to each jacket that would deliver the drug to either jacket. However, when this is the same drug and a number of drugs must be administered, relegation to one pump spares duplication of the drug in the turret position of the other pump allowing an additional drug to be loaded therein. Consecutive delivery from one pump to two side-entry connectors on the same jacket, delivery to either of the side-entry connectors on the same jacket from pumps respective of each side-entry connector, delivery from one pump to one or both side-entry connectors on a different jacket are equally possible.

With line switching, the distance from the pump outlet to the side-entry connector of each jacket must be manually entered into the controller one time. To segregate drugs that should not be mixed before arrival, a pump outlet turret can switch to different lines that deliver the drug to different side-entry connectors of the same jacket. For delivery from a single pump to multiple jackets where the mixing of drugs is insignificant, the line switching mechanism is used to successively deliver different drugs through the same line to the same side-entry connector and jacket. Line switching may be used to change the drugs successively delivered to a given jacket or to isolate drugs for delivery to the same jacket by directing each through a different delivery line and jacket entry point.

Placing additional jackets, and when the drug is bound to a drug-carrier, magnetized jackets, or impasse-jackets, and capsule fastened patch-magnets at different locations in the body to coordinate the targeted receipt of drugs according to a timed sequence, for example, opens a new field for the development of coordinated drug strategies, those elucidated implementable in the form of standardized pump-pack and jacket package units. Even in a relatively simple embodiment such as that depicted in FIG. 6, drugs best not compounded to be targeted directly to the jacket can be kept apart for timed consecutive delivery under central control. This is done by replacing a singular fixed position pump inlet or entry line vial or refill cartridge receptacle with a turret feed mechanism to be described.

Also to isolate successive drugs, intervening boli of a drug compatible with those preceding and succeeding it in delivery or of an inert substance in the form of a crushed tacky hydrogel can be interposed to minimize if not eliminate premature mixing of the principle drugs as the result of delivery through the same line. A similar turret at the pump outlet would allow delivery to be switched to other jackets; however, for economy, the incorporation of a pump outlet turret in a single pump-pair and power and control module is generally limited to common conditions for which a benefit to be gained in synchronous delivery to different jackets is well established. Otherwise, the strategically timed delivery of multiple medications to multiple jackets is relegated to a pump-pair receiver pump-pack to be described.

Alternatively, the turret feed can direct the output of either pump in the pair through a second pair of main and sidelines to a second side-entry connector connected to the same jacket. The use of two or more side-entry connectors with a single jacket should seldom be needed and impedes placement. Rather. than multiple side-entry connectors, it is preferable to position another jacket or jackets at small intervals along the ductus. The relation among jackets placed thus can be made clear by passage to the same port, although numerous lines leading to numerous jackets can be connected to the same port. That with a turret the outlet of either pump in a single pump-pair could be directed to any number of side-entry connectors on any number of side-entry connection jackets is obvious.

FIGS. 33 thru 36 provide a detailed view of the two differently sized standardized drug or other therapeutic substance vials 66 and 85 for insertion in a turret. Of the two, that larger, 85, is received in a turret receptacle or well that can also be used to insert a hose from a drug or other therapeutic supply reservoir in the pump-pack during ambulatory use or from another source in the clinic. To admit and discharge fluid, the smaller of the two standardized drug or other therapeutic substance vials, 66, has elastic membrane 86 with slits 87 at both its top and bottom. For use of the larger standardized sized vial 85 as the insert end of a drug reservoir inlet hose in a turret well or recess, one end of vial 86 has lip 67 to engage the end of hose 88.

Connection of lip 67 to the end of hose 88 is by secure friction fit, cutting engagement by slip-on or click-over engagement. Except for connecting lip 67, vial 85 is the same whether used alone or to serve as the turret end segment and connector of a drug reservoir hose for insertion into and secure connection to one of the vial receptacles or wells in the turret. In FIG. 36, QR matrix or conventional barcode 89 on the side of drug vial 66 identifying the drug or other therapeutic substance contained seen through a window cut through turret wall 90 makes it possible for an optical reader to detect any error in turret loading or insertion.

### Ambulatory Prosthetic Disorder Response System Control

FIG. 37 provides a schematic of the control hierarchy for a single pump-pair in support of the four jackets in the pump-pair and jacket set, the control program not generalizable but rather determined by the specific condition or conditions to be treated. Unlike FIG. 38, only the control train is represented, anatomical distinction between intra and extracorporeal elements omitted. Also for pictorial clarity, where the jacket lines are actually separate and distinct channels, the electrical and fluid lines between nodes and jackets are shown as shared until finally led to each jacket.

Electrical connectors, remote sensors, and auxiliary drug supply reservoirs have been omitted. If provided with the requisite switching and valving, the lines shown as shared could support each jacket independently but not simultaneously, the utility thereof contingent upon the condition or conditions to be treated. In FIGS. 37 and 38, single lines are electrical, which serve to independently energize the electromagnet or electromagnets in each jacket and/or return sensor data arriving from the jacket to its respective node, and the double lines are fluidic, and the pumps bidirectional, so are the fluid lines. These electromagnets might be electromagnetic impasse-jackets, contraction-electromagnets, peristalsis contraction-electromagnets and/or extraction-electromagnets individual or dual as shown in FIG. 15 for high volume extraction as in leukapheresis, and coordinated as shown in FIG. 14.

FIG. 38 provides a schematic of the pump-pack and jacket set. For clarity, the electrical and fluid lines between nodes and jackets are shown as consolidated when these are actually separate and distinct; to apply to the large range of possible realizations, the electrical connectors are not shown pictorially as actual; and remote sensors and auxiliary drug supply reservoirs have been omitted. The lines could be shared, however, were the requisite switching and valving provided. The different jackets can be placed along the same ductus, different ductus belonging to the same organ system, or ductus belonging to different organ systems.

Each jacket may be of any type, whether a simple junction jacket with magnetization, such as shown in FIGS. 1 and 2, with magnetization, such as shown in FIGS. 3 and 4, or with magnetization and radiation shielding as shown in FIGS. 5 and 6. Each jacket can incorporate an electromagnet or electromagnets as shown in 10 thru 12, rather than permanent magnets, where some jackets are used to draw magnetically susceptible particle-bound drugs into the local lumen wall, while extraction electromagnet-jacket as shown in FIGS. 13 thru 15 draw magnetically susceptible particle-bound leukocytes out of the lumen, such as from the passing blood, for example.

At the same time and under the control of the same master node, some jackets can meter flow through the digestive tract, for example, to include time-coordinated separate sphincteric and compound multiple contraction-electromagnet jackets such as shown in FIG. 10. Peristaltic and/or sphincteric electromagnet-jackets can be controlled at the local level as an independent component by a timing module. As a standalone function, the controller is implanted as are the other components of such a system, which is equivalent to a node of the master microcontroller. The addition of another subsystem best coordinated with digestive function is then governed by the master controller through another node; otherwise, the initial implant can continue to function independently.

Generally, the addition of another subsystem responsive to another node will involve the addition of a pump-pack. Then, because it assures noninvasive access, a microcontroller chip in the pump-pack is assigned as the master, or central overall controller. A controller previously implanted is ordinarily left as its respective node and subsystem. When the esophagus to include the lower esophageal sphincter is assisted or replaced, both peristalsis and sphincteric function are controlled as a unit. If distal sphincters are likewise dysfunctional, the sphincteric jackets placed to assist these are likewise controlled with the proximal components. Accordingly, the individual jackets depicted in FIGS. 37 and 38 can represent any of the jackets types described herein and the set consist of these in any combination, and it is this versatility that allows the master node to execute programs to treat multicomponental disorders that involve different organ systems in a unified and coordinated manner as a prosthetic syndromal or comorbidity disorder response system.

1. A collar for encircling a tubular anatomical structure, said collar having a side tube to deliver drugs, pass cabled transcatheteric devices, and extract luminal contents.

2. A collar according to claim 1 which incorporates a magnet to assist in the detention and extraction of magnetically susceptible luminal contents.

3. A collar according to claim 1, said collar entered through a side tube with sharp front adductal edge such that placing said collar about said tubular anatomical structure allows said front sharp edge of said side tube to be used a trepan to excise a plug of tissue from the side said tubular anatomical structure thereby to join the lumina of said side tube and said tubular anatomical structure as a continuous passageway.

4. A collar according to claim 1, said collar extended in the longitudinal axis and augmented to include a magnetized layer in concentric relation to said collar.

5. A collar according to claim 1, said collar extended in the long axis and augmented to include a radiation shield in concentric relation to said long axis of said collar.

6. A collar according to claim 1 wherewith the pump supplying fluid medicinals to said collar is controlled according to a microcontroller program and in response to the output of at least one physiological parameter sensor, said program and said sensor related through a closed feedback loop.

7. A collar according to claim 1 mounting an electromagnet with pole oriented to exert a tractive force as to diametrically intersect with the longitudinal axis of said collar.

8. A collar according to claim 7 assisted by a separate electromagnet to draw a drug released from a collar according to claim 1 into a bodily organ.
